(19) United States
(12) Patent Application Publication
(10) Pub. No.: US 2013/0252890 A1
(43) Pub. Date: Sep. 26, 2013

(54) 6,7-DIHYDRO-5H-BENZO[7]ANNULENE DERIVATIVES, PROCESS FOR PREPARATION THEREOF, PHARMACEUTICAL PREPARATIONS COMPRISING THEM, AND THE USE THEREOF FOR PRODUCTION OF MEDICAMENTS

(71) Applicants: Tim Wintermantel, Koln (DE); Carsten Möller, Berlin (DE); Ulrich Bothe, Berlin (DE); Reinhard Nubbemeyer, Berlin (DE); Ludwig Zorn, Berlin (DE); Dirk Kosemund, Berlin (DE); Antonius Ter Laak, Berlin (DE); Rolf Bohlmann, Berlin (DE); Lars Wortmann, Berlin (DE); Donald Bierer, Haan (DE)

(72) Inventors: Tim Wintermantel, Koln (DE); Carsten Möller, Berlin (DE); Ulrich Bothe, Berlin (DE); Reinhard Nubbemeyer, Berlin (DE); Ludwig Zorn, Berlin (DE); Dirk Kosemund, Berlin (DE); Antonius Ter Laak, Berlin (DE); Rolf Bohlmann, Berlin (DE); Lars Wortmann, Berlin (DE); Donald Bierer, Haan (DE)

(73) Assignee: BAYER INTELLECTUAL PROPERTY GMBH, Monheim (DE)

(21) Appl. No.: 13/806,845

(22) PCT Filed: Jun. 21, 2011
(86) PCT No.: PCT/EP2011/060335

§ 371 Date: May 23, 2013

(30) Foreign Application Priority Data
Jun. 25, 2010 (DE) 102010030538.3

(51) Int. Cl. C07C 317/28; A61K 38/22; A61K 31/18; A61K 31/216; A61K 31/165; A61K 31/277; C07C 323/25; A61K 31/145

(52) U.S. Cl. CPC C07C 317/28; C07C 323/25; A61K 38/22; A61K 31/145; A61K 31/216; A61K 31/165; A61K 31/277; A61K 31/18
USPC 514/10.3; 564/337; 560/10; 564/202; 558/426; 564/99; 562/427; 514/650; 514/510; 514/630; 514/605; 514/562

(57) ABSTRACT

The invention relates to selective oestrogen receptor modulators (SERM) and methods of production thereof, use thereof for the treatment and/or prophylaxis of diseases and use thereof for the production of medicinal products for the treatment and/or prophylaxis of diseases, in particular of bleeding disorders, osteoporosis, endometriosis, myomata, hormone-dependent tumours, for hormone replacement therapy and for contraception.

The invention relates to selective oestrogen receptor modulators (SERMs) and methods of production thereof, use thereof for the treatment and/or prophylaxis of diseases and use thereof for the production of medicinal products for the treatment and/or prophylaxis of diseases, in particular of bleeding disorders, osteoporosis, endometriosis, myomata, hormone-dependent tumours, for hormone replacement therapy and for contraception.

SERMs are compounds that have, tissue-selectively, either an anti-oestrogenic/oestrogen-inhibiting or an oestrogenic or partially oestrogenic action, for example in the case of the uterus they inhibit the action of oestrogen, but in the case of bone they have a neutral or oestrogen-like action. Tamoxifen, raloxifene and bazedoxifene may be mentioned as examples of such compounds. SERMs are to be differentiated from pure anti-oestrogens, which have a purely antagonistic action, inhibiting the action of oestrogen, in all tissues and do not display any oestrogenic or partially oestrogenic action in a tissue. SERMs (selective oestrogen receptor downregulators) belong to the anti-oestrogens and lead at the protein level to complete degradation of the oestrogen receptor in the target cells. The compound fulvestrant may be mentioned as an example of a pure anti-oestrogen or SERD.

6,7-Dihydro-5H-benzo[7]annulene derivatives have been described already as SERMs and use thereof in the treatment of bleeding disorders, osteoporosis, endometriosis, myomata, hormone-dependent tumours, for hormone replacement therapy and for contraception (cf. WO 00/03979).

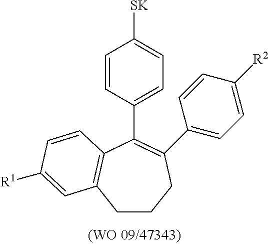

Further information on structurally more distantly related substances, SERMs or the use of particular SERMs in the treatment of specific diseases is given for example in EP 0584952, WO 96/21656; J. Endocrinol. 1994, 141, 335; EP 0124369; U.S. Pat. No. 6,645,951; Bioorg. Med. Chem. Lett. 2006, 14, 4803-4819; U.S. Pat. No. 6,153,768; Bioorganic & Medicinal Chemistry Letters 14 (2004) 4659-4663; DE 19521646 A1, Archiv der Pharmazie 333, (2000) 305-311; U.S. Pat. No. 6,147,105, DE 10117441, EP 138504, DE 19622457; DE 19636625, WO 98/07740, WO 99/33855, WO 00/14104, Mol. Pharmacol. 1991, 39: 421-428; J. Med. Chem. 1986, 29, 2053-2059; J. Med. Chem. 1988, 31, 1316-1326; WO 00/55137, US 20030105148, WO 2009047343, Indian Journal of Chemistry, Vol 25B, August 1986, 832-837; WO04/58682 or Bioorg. and Medicinal Chemistry 16 (2008) 9554-9573.

The problem to be solved by the present invention is to make available alternative substances acting as SERMs with improved physicochemical properties.

The present invention relates to compounds of formula (I):

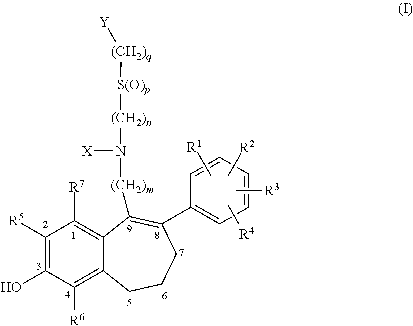

in which
R¹, R², R³ and R⁴ independently of one another stand for hydrogen or fluorine, wherein at least one substituent selected from R¹, R², R³ and R⁴ stands for fluorine,
R⁵, R⁶ and R⁷ independently of one another stand for hydrogen, fluorine, chlorine, bromine, methyl, ethyl, trifluoromethyl or nitrile
X is selected from the group comprising hydrogen, C₁-C₆-alkyl-, C₃-C₈-cycloalkyl-, C₂-C₆-alkenyl, C₂-C₆-alkynyl, C₁-C₆-alkyl-S(O)₂—, C₁-C₆-alkylcarbonyl-, phenyl-C₁-C₆-alkyl-, which optionally can be substituted once, twice or multiply with —OH, halogen, —CN, —NR⁸R⁹, —C(O)NR¹⁰R¹¹, —N(R¹⁰)C(O)NR¹⁰R¹¹, —C₁-C₆-haloalkoxy, —C₁-C₆-alkoxy, —C(O)OH, —C(O)OC₁-C₆-alkyl or —C(O)OBenzyl, and optionally hydrogen atoms can also be replaced with deuterium atoms,
R⁸ and R⁹ stand for C₁-C₆-alkyl, C₃-C₇-cycloalkyl, phenyl or benzyl, optionally substituted with halogen or deuterium,
R¹⁰ and R¹¹ stand for hydrogen or C₁-C₆-alkyl, C₃-C₇-cycloalkyl, phenyl or benzyl optionally substituted with halogen or deuterium,
Y stands for a per- or partially-fluorinated —C₁-C₄-alkyl or per- or partially-fluorinated C₃-C₈-cycloalkyl,
m stands for 4, 5, 6 or 7,
n stands for 2, 3, 4, 5 or 6,
p stands for 0, 1 or 2,
q stands for 0, 1, 2, 3, 4, 5 or 6
   and their salts, solvates or solvates of the salts, including all crystal modifications.

It was found that 6,7-dihydro-5H-benzo[7]annulene derivatives (I), which are linked in the 8-position to a fluorinated aromatic substituent and which are linked in position 9 to an optionally substituted aliphatic chain, act as SERMs. Many of the claimed 6,7-dihydro-5H-benzo[7]annulene derivatives display—in contrast to the currently known SERMs such as tamoxifen, raloxifenes or similar compounds—additionally a destabilizing action on the ERα content (residual relative ERα content less than or equal to 30%). Over the entire structural range, these compounds display a high anti-oestrogenic action in vitro (IC₅₀ values below 0.6 micromolar) and primarily even double-digit or single-digit nanomolar IC₅₀ values for the inhibition of estradiol-induced luciferase activity).

Compounds according to the invention are the compounds of formula (I) and their salts, solvates and solvates of the salts, the compounds of the formulae given below that are covered by formula (I) and their salts, solvates and solvates of the salts and the compounds presented below as examples, which are covered by formula (I), and their salts, solvates and solvates of the salts, provided that the compounds stated below that are covered by formula (I) are not already salts, solvates and solvates of the salts.

The compounds according to the invention can, depending on their structure, exist in stereoisomeric forms (enantiomers, diastereomers). In compounds of formula (I), there can be stereocentres on the sulphur atom (for p=1) and/or in the residue X. The invention therefore comprises the enantiomers and/or diastereomers and respective mixtures thereof. The stereoisomerically uniform constituents can be isolated in a known way from said mixtures of enantiomers and/or diastereomers. Within the scope of the present invention, a compound is enantiomerically pure at an enantiomeric excess of more than 90% (>90% ee).

If the compounds according to the invention can occur in tautomeric forms, the present invention comprises all tautomeric forms.

Physiologically harmless salts of the compounds according to the invention are preferred as salts within the scope of the present invention. However, salts that are not suitable in themselves for pharmaceutical uses but can be used for example for isolation or purification of the compounds according to the invention are also covered.

Physiologically harmless salts of the compounds according to the invention comprise salts of acid addition of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, acetic acid, formic acid, trifluoroacetic acid, propionic acid, lactic acid, tartaric acid, malic acid, citric acid, fumaric acid, maleic acid and benzoic acid.

Physiologically harmless salts of the compounds according to the invention also comprise salts of usual bases, such as for example and preferably alkali metal salts (e.g. sodium and potassium salts), salts of alkaline-earth metals (e.g. calcium and magnesium salts) and ammonium salts, derived from ammonia or organic amines with 1 to 16 carbon atoms, such as for example and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Those forms of the compounds that form a complex in the solid or liquid state by coordination with solvent molecules are designated as solvates within the scope of the invention. Hydrates are a special form of solvates, in which coordination takes place with water. Hydrates are preferred as solvates within the scope of the present invention.

Furthermore, the present invention also comprises prodrugs of the compounds according to the invention. The term “prodrugs” comprises compounds which may themselves be biologically active or inactive, however, during their residence time in the body they are converted to compounds according to the invention (for example metabolically or hydrolytically).

Within the scope of the present invention, the substituents have, unless stated otherwise, the following meaning:

Alkyl per se and “alk” and “alkyl” in alkoxy, alkylcarbonyl, alkylamino, alkylaminocarbonyl, alkoxycarbonyl, alkoxycarbonylamino and alkylcarbonylamino stand for a linear or branched alkyl residue with as a rule 1 to 6, preferably 1 to 4, especially preferably 1 to 3 carbon atoms, for example and preferably for methyl, ethyl, n-propyl, isopropyl, tert-butyl, n-pentyl and n-hexyl.

Alkoxy stands for example and preferably for methoxy, ethoxy, n-propoxy, isopropoxy, tert-butoxy, n-pentoxy and n-hexoxy.

Alkylcarbonyl stands for example and preferably for formyl, acetyl and propanoyl.

Alkylamino stands for an alkylamino residue with one or two (selected independently of one another) alkyl substituents. (C₁-C₃)-alkylamino stands for example for a monoalkylamino residue with 1 to 3 carbon atoms or for a dialkylamino residue with in each case 1 to 3 carbon atoms per alkyl substituent. For example and preferably, we may mention: methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Alkylaminocarbonyl stands for an alkylaminocarbonyl residue with one or two (selected independently of one another) alkyl substituents. (C₁-C₃)-alkylaminocarbonyl stands for example for a monoalkylaminocarbonyl residue with 1 to 3 carbon atoms or for a dialkylaminocarbonyl residue with in each case 1 to 3 carbon atoms per alkyl substituent. For example and preferably, we may mention: methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylamino-carbonyl and N-n-hexyl-N-methylaminocarbonyl.

Alkoxycarbonyl stands for example and preferably for methoxycarbonyl, ethoxycarbonyl, n-propoxycarbonyl, isopropoxycarbonyl, tert-butoxycarbonyl, n-pentoxycarbonyl and n-hexoxycarbonyl.

Alkoxycarbonylamino stands for example and preferably for methoxycarbonylamino, ethoxycarbonylamino, n-propoxycarbonylamino, isopropoxycarbonylamino, tert-butoxycarbonylamino, n-pentoxycarbonylamino, n-hexoxycarbonylamino, methoxycarbonyl-N-methylamino, ethoxycarbonyl-N-methylamino, n-propoxycarbonyl-N-methylamino, isopropoxycarbonyl-N-methylamino, tert-butoxycarbonyl-N-methylamino, n-pentoxycarbonyl-N-methylamino and n-hexoxycarbonyl-N-methylamino.

Alkylcarbonylamino stands for example and preferably for acetylamino, acetyl-N-methylamino, ethylcarbonylamino and ethylcarbonyl-N-methylamino

Cycloalkyl stands for a cycloalkyl group with as a rule 3 to 8, preferably 5 to 7 carbon atoms, wherein the ring can also be partially unsaturated, for example and preferably for cyclopropyl, cyclobutyl, cyclopentyl, cyclohexyl and cycloheptyl.

Aryl stands for a mono- to tricyclic aromatic, carbocyclic residue with as a rule 6 to 14 carbon atoms; for example and preferably for phenyl, naphthyl and phenanthrenyl.

Heteroaryl stands for an aromatic, mono- or bicyclic residue with as a rule 5 to 10, preferably 5 to 6 ring atoms and up to 5, preferably up to 4 heteroatoms from the series S, O and N, for example and preferably for thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

Heterocyclyl stands for a mono- or polycyclic, preferably mono- or bicyclic, non-aromatic heterocyclic residue with as a rule 4 to 10, preferably 5 to 8 ring atoms and up to 3, preferably up to 2 heteroatoms and/or hetero groups from the series N, O, S, SO, SO₂. The heterocyclyl residues can be saturated or partially unsaturated. Five-to 8-membered, monocyclic saturated heterocyclyl residues with up to two heteroatoms from the series O, N and S are preferred. For example and preferably, we may mention: tetrahydrofuranyl, pyrrolidinyl, pyrrolinyl, piperidinyl, morpholinyl, thiomorpholinyl, perhydroazepinyl.

Halogen stands for fluorine, chlorine, bromine and iodine.

Deuterium or D is used in reference to substances in which, in the respective position, the proportion of deuterium is greatly increased relative to the naturally occurring isotopic ratio, for example compounds with an isotopic purity of 10-100%, in particular with an isotopic purity of 50, 60, 70, 80, 90% or higher.

Perfluorinated —C₁-C₄-alkyl stands for a completely fluorinated linear or branched alkyl residue with as a rule 1 to 4, preferably 1 to 3 carbon atoms, for example and preferably for trifluoromethyl, pentafluoroethyl, heptafluoropropyl and heptafluoroisopropyl.

Partially fluorinated —C₁-C₄-alkyl stands for a partially fluorinated linear or branched alkyl residue with as a rule 1 to 4 carbon atoms—selected from, but not restricted to, 1,2,2,2-tetrafluoroethyl, 1,1,2,2-tetrafluoroethyl, 2,2,2-trifluoro-1-(trifluoromethyl)ethyl, 1,1,3,3,3-pentafluoropropyl, 1,1,2,3,3,3-hexafluoropropyl, 1,1,2,2,3,3,4,4-octafluorobutyl, 1,2,2,3,3,3-hexafluoro-1-methylpropyl, 1,1,3,3,3-pentafluoro-2-(trifluoromethyl)propyl, 2,2,2-trifluoro-1-methyl-1-(trifluoromethyl)ethyl, 2-fluoro-1,1-bis(fluoromethyl)ethyl. 1,2,2,2-Tetrafluoroethyl, 1,1,3,3,3-pentafluoropropyl, 1,1,2,3,3,3-hexafluoropropyl and 2,2,2-trifluoro-1-(trifluoromethyl)ethyl are preferred, and 2,2,2-trifluoro-1-(trifluoromethyl)ethyl and 1,1,3,3,3-pentafluoropropyl are especially preferred.

Perfluorinated —C₃-C₇-cycloalkyl stands for a completely fluorinated cycloalkyl group with as a rule 3-7, preferably 5-6 carbon atoms, for example and preferably for perfluorocyclopentyl and perfluorocyclohexyl.

Partially fluorinated —C₃-C₇-cycloalkyl stands for a partially fluorinated cycloalkyl group with as a rule 3 to 7 carbon atoms—selected from, but not restricted to: 2,2-difluorocycloheptyl, 2-fluorocycloheptyl, 3,3-difluorocycloheptyl, 3-fluorocycloheptyl, 4,4-difluorocycloheptyl, 4-fluorocycloheptyl, 4,4-difluorocyclohexyl, 4-fluorocyclohexyl, 3,3-difluorocyclohexyl, 3-fluorocyclohexyl, 2,2-difluorocyclohexyl, 2-difluorocyclohexyl, 3,3-difluorocyclopentyl, 3-fluorocyclopentyl, 2,2-difluorocyclopentyl, 2-fluorocyclopentyl, 3,3-difluorocyclobutyl, 3-fluorocyclobutyl, 2,2-difluorocyclobutyl, 2-fluorocyclobutyl, 2,2-difluorocyclopropyl, 2-fluorocyclopropyl. 4,4-Difluorocyclohexyl, 4-fluorocyclohexyl, 3,3-difluorocyclohexyl, 3,3-difluorocyclopentyl, 3,3-difluorocyclobutyl and 2,2-difluorocyclopropyl are preferred. 4,4-Difluorocyclohexyl is especially preferred.

A symbol * on a bond denotes the position of attachment in the molecule.

When residues in the compounds according to the invention are substituted, unless stated otherwise, the residues can be monosubstituted or polysubstituted. Within the scope of the present invention, for all residues that occur more than once, their meaning is independent of one another. A substitution with one, two or three identical or different substituents is preferred. Substitution with one substituent is quite especially preferred.

Compounds of formula (I) are preferred, in which
R¹, R², R³, R⁴, R⁵, R⁶ or R⁷ independently of one another stand for hydrogen or fluorine, wherein at least one substituent R¹, R², R³ and R⁴ stands for fluorine.
X is selected from the group comprising hydrogen, C₁-C₆-alkyl-, C₃-C₈-cycloalkyl-, C₁-C₆-alkyl-S(O)₂—, C₁-C₆-alkylcarbonyl-, phenyl-C₁-C₆-alkyl-, which optionally can be substituted once, twice or multiply with —OH, halogen, deuterium, —CN, —NR⁸R⁹, —C(O)NR¹⁰R¹¹, —N(R¹⁰)C(O)NR¹⁰R¹¹, alkoxy, —C(O)OH, —C(O)OC₁-C₆-alkyl or —C(O)OBenzyl,
R⁸ and R⁹ stand for C₁-C₆-alkyl or benzyl,
R¹⁰ and R¹¹ stand for hydrogen, C₁-C₆-alkyl or benzyl,
Y stands for —CF₃, —C₂F₅, —C₃F₇, —C₄F₉ or —C₃-C₇-cycloalkyl with 2-4 fluorine atoms,
m stands for 4, 5 or 6,
n stands for 2, 3, 4, 5 or 6,
P stands for 0, 1 or 2,
q stands for 0, 1, 2, 3, 4, 5 or 6
   and their salts, solvates or solvates of the salts, including all crystal modifications.

Compounds of formula (I) are also preferred, in which
R¹, R², R³, R⁴ independently of one another stand for hydrogen or fluorine, wherein at least one and at most two fluorine atoms should be contained,
R⁵ and R⁶ independently of one another stand for hydrogen or fluorine,
R⁷ stands for hydrogen,
X is selected from the group comprising hydrogen, —C₁-C₄-alkyl, cyclopropyl-, which can optionally be substituted singly with —OH, —CN, methoxy, —C(O)OH, —C(O)OCH₃ or —C(O)OBenzyl or singly or multiply with —F or deuterium, or X is selected from methyl-S(O)₂— or methylcarbonyl-
Y stands for —CF₃, —C₂F₅, —CF₂CF₂CF₃, —CF(CF₃)₂ or

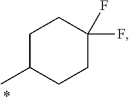

m stands for 5 or 6,
n stands for 3, 4 or 5,
P stands for 0, 1 or 2,
q stands for 0, 1, 2, 3, 4 or 5
   and their salts, solvates or solvates of the salts, including all crystal modifications.

Furthermore, compounds of formula (I) are preferred in which
R¹, R², R³ and R⁴ independently of one another stand for hydrogen or fluorine, wherein at least one and at most two fluorine atoms should be contained,
R⁵ and R⁶ independently of one another stand for hydrogen or fluorine, with the restriction that R⁵ and R⁶ do not mean fluorine simultaneously,
X stands for C₁-C₄-alkyl-, optionally substituted with deuterium,
Y stands for —CF₃, —C₂F₅, 4,4-difluorocyclohexyl,
m stands for 5 or 6,
n stands for 3 or 4,
P stands for 1 or 2,
q stands for 2, 3, 4 or 5
   or in the special case in which Y stands for 4,4-difluorocyclohexyl,
q stands for 0 or 1
   and their salts, solvates or solvates of the salts, including all crystal modifications.

Compounds of formula (II) are especially preferred, as a subset of formula (I)

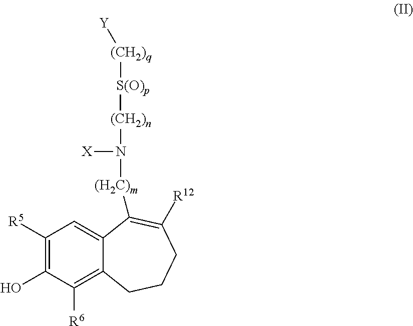

in which
R¹² stands for 3,5-difluorophenyl-, 3,4-difluorophenyl, 2,4-difluorophenyl-, 4-fluorophenyl,
R⁵ and R⁶ independently of one another stand for hydrogen or fluorine, wherein R⁵ and R⁶ do not mean fluorine simultaneously,
X stands for C₁-C₄-alkyl- optionally substituted with deuterium,
Y stands for —CF₃, —C₂F₅, 4,4-difluorocyclohexyl,
m stands for 6,
n stands for 3 or 4,
p stands for 1 or 2,
q stands for 2, 3, 4 or 5
   or in the special case in which Y stands for 4,4-difluorocyclohexyl,
q stands for 0 or 1
   and their salts, solvates or solvates of the salts, including all crystal modifications.

The invention further relates to compounds of formula (I), in which
R¹, R², R³ and R⁴ independently of one another stand for hydrogen or fluorine, wherein at least one substituent R¹, R², R³ and R⁴ stands for fluorine.

The invention further relates to compounds of formula (I), in which
R⁵, R⁶ and R⁷ independently of one another stand for hydrogen, fluorine, chlorine, bromine, methyl, ethyl, trifluoromethyl or nitrile.

The invention further relates to compounds of formula (I), in which
X is selected from the group comprising H, C₁-C₆-alkyl-, C₃-C₈-cycloalkyl-, C₂-C₆-alkenyl, C₂-C₆-alkynyl, C₁-C₆-alkyl-S(O)₂—, C₁-C₆-alkylcarbonyl-, phenyl-C₁-C₆-alkyl-, which optionally can be substituted once, twice or multiply with —OH, halogen, —CN, —NR⁸R⁹, —C(O)NR¹⁰R¹¹, —N(R¹⁰)C(O)NR¹⁰R¹¹, —C₁-C₆-haloalkoxy, —C₁-C₆-alkoxy, —C(O)OH, —C(O)OC₁-C₆-alkyl or —C(O)OBenzyl, and optionally hydrogen atoms can also be replaced with deuterium atoms.

The invention further relates to compounds of formula (I), in which

R⁸ and R⁹ stand for C₁-C₆-alkyl, C₃-C₇-cycloalkyl, phenyl or benzyl optionally substituted with halogen and/or deuterium.

The invention further relates to compounds of formula (I), in which

R¹⁰ and R¹¹ stand for hydrogen or C₁-C₆-alkyl, C₃-C₇-cycloalkyl, phenyl or benzyl optionally substituted with halogen and/or deuterium.

The invention further relates to compounds of formula (I), in which
Y stands for a per- or partially-fluorinated —C₁-C₄-alkyl or per- or partially-fluorinated C₃-C₈-cycloalkyl.

The invention further relates to compounds of formula (I), in which
m stands for 4, 5, 6 or 7.

The invention further relates to compounds of formula (I), in which
n stands for 2, 3, 4, 5 or 6.

The invention further relates to compounds of formula (I), in which
p stands for 0, 1 or 2.

The invention further relates to compounds of formula (I), in which
q stands for 0, 1, 2, 3, 4, 5 or 6.

The invention further relates to compounds of formula (I), in which
R¹, R², R³, R⁴, R⁵, R⁶ or R⁷ independently of one another stand for hydrogen or fluorine, wherein at least one substituent R¹, R², R³ and R⁴ stands for fluorine.

The invention further relates to compounds of formula (I), in which
X is selected from the group comprising H, C₁-C₆-alkyl-, C₃-C₈-cycloalkyl-, C₁-C₆-alkyl-S(O)₂—, C₁-C₆-alkylcarbonyl-, phenyl-C₁-C₆-alkyl-, which optionally can be substituted once, twice or multiply with —OH, halogen, deuterium, —CN, —NR⁸R⁹, —C(O)NR¹⁰R¹¹, —N(R¹⁰)C(O)NR¹⁰R¹¹, alkoxy, —C(O)OH, —C(O)OC₁-C₆-alkyl or —C(O)OBenzyl.

The invention further relates to compounds of formula (I), in which
R⁸ and R⁹ stand for C₁-C₆-alkyl or benzyl.

The invention further relates to compounds of formula (I), in which
R¹⁰ and R¹¹ stand for hydrogen, C₁-C₆-alkyl or benzyl.

The invention further relates to compounds of formula (I), in which
Y stands for —CF₃, —C₂F₅, —C₃F₇, —C₄F₉ or —C₃-C₇-cycloalkyl with 2-4 fluorine atoms.

The invention further relates to compounds of formula (I), in which
m stands for 4, 5 or 6.

The invention further relates to compounds of formula (I), in which
n stands for 2, 3, 4, 5 or 6.

The invention further relates to compounds of formula (I), in which

R¹, R², R³, R⁴ independently of one another stand for hydrogen or fluorine, wherein at least one and at most two fluorine atoms should be contained.

The invention further relates to compounds of formula (I), in which
R⁵ and R⁶ independently of one another stand for hydrogen or fluorine.

The invention further relates to compounds of formula (I), in which
R⁷ stands for hydrogen.

The invention further relates to compounds of formula (I), in which
X is selected from the group comprising hydrogen, —C₁-C₄-alkyl, cyclopropyl-, optionally substituted singly with —OH, —CN, methoxy, —C(O)OH, —C(O)OCH₃ or —C(O)OBn or singly or multiply with —F or deuterium, methyl-S(O)₂— or methylcarbonyl-.

The invention further relates to compounds of formula (I), in which
Y stands for —CF₃, —C₂F₅, —CF₂CF₂CF₃, —CF(CF₃)₂ or

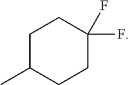

The invention further relates to compounds of formula (I), in which
m stands for 5 or 6.

The invention further relates to compounds of formula (I), in which
n stands for 3, 4 or 5.

The invention further relates to compounds of formula (I), in which
q stands for 0, 1, 2, 3, 4 or 5.

The invention further relates to compounds of formula (I), in which
R⁵ and R⁶ independently of one another stand for hydrogen or fluorine, with the restriction that R⁵ and R⁶ do not mean fluorine simultaneously.

The invention further relates to compounds of formula (I), in which
X stands for C₁-C₄-alkyl-.

The invention further relates to compounds of formula (I), in which
Y stands for —CF₃, —C₂F₅, 4,4-difluorocyclohexyl.

The invention further relates to compounds of formula (I), in which
m stands for 5 or 6.

The invention further relates to compounds of formula (I), in which
n stands for 3 or 4.

The invention further relates to compounds of formula (I), in which
p stands for 1 or 2.

The invention further relates to compounds of formula (I), in which
q stands for 2, 3, 4 or 5.

The invention further relates to compounds of formula (I), in which in the special case in which Y stands for 4,4-difluorocyclohexyl, q stands for 0 or 1.

The invention further relates to compounds of formula (II), in which
R¹² stands for 3,5-difluorophenyl-, 3,4-difluorophenyl, 2,4-difluorophenyl-, 4-fluorophenyl.

The invention further relates to compounds of formula (II), in which
R⁵ and R⁶ independently of one another stand for hydrogen or fluorine, with the restriction that R⁵ and R⁶ do not mean fluorine simultaneously.

The invention further relates to compounds of formula (II), in which
X stands for C₁-C₄-alkyl-.
   The invention further relates to compounds of formula (II), in which
Y stands for —CF₃, —C₂F₅, 4,4-difluorocyclohexyl.

The invention further relates to compounds of formula (II), in which
m stands for 6.

The invention further relates to compounds of formula (II), in which
n stands for 3 or 4.

The invention further relates to compounds of formula (II), in which
p stands for 1 or 2.

The invention further relates to compounds of formula (II), in which
q stands for 2, 3, 4 or 5.

The invention further relates to compounds of formula (II), in which in the special case in which Y stands for 4,4-difluorocyclohexyl, q stands for 0 or 1.

The definitions of residues stated individually in the particular combinations or preferred combinations of residues are if desired also replaced with definitions of residues of another combination independently of the particular stated combinations of the residues.

Combinations of two or more of the preferred ranges stated above are quite especially preferred.

The definitions of residues given above generally or in preferred ranges apply both to the end products of formula (I) and correspondingly to the raw materials or intermediates required for production in each case.

The invention further relates to a method of production of the compounds according to the invention. The production of the compounds according to the invention (I) or the compounds (II) as a subset of formula (I) can be explained by the following synthesis scheme.

Intermediates 5, which were prepared as in patent specification WO 03/033461 A1, are shown in the following general scheme (Synthesis Scheme 1), where R¹, R², R³, R⁴, R⁵, R⁶ and R⁷ have the meanings given in formula (I).

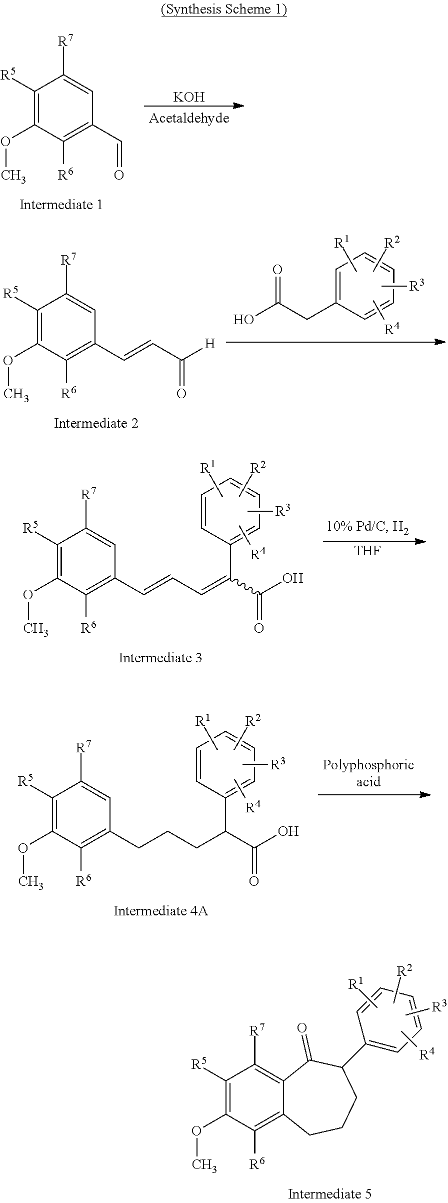

Intermediates 2 are synthesized by the condensation reactions of acetaldehyde known by a person skilled in the art with one of the intermediates 1 (commercially available e.g. from Aldrich, ABCR) under basic catalysis in water with or without addition of an organic solvent that is stable under these conditions (Organic Reactions 1968, 16, 1; Justus Liebigs Ann. Chem. 1917, 412, 322; J. Org. Chem. 1951, 16, 1519; Helv. Chim. Acta 1993, 76, 1901). Reaction with potassium hydroxide with addition of dichloromethane between 1-30° C. is especially preferred. Intermediates 3 are then reacted according to the Knoevenagel conditions known by a person skilled in the art with an arylacetic acid (commercially available from e.g. Aldrich, ABCR) (Organic Reactions 1967, 15, 204; Tetrahedron Lett. 1998, 39, 8013). Reaction with acetic anhydride and triethylamine at a temperature of 90° C. under reflux is especially preferred. Intermediates 4 are synthesized by catalytic hydrogenations, as known by a person skilled in the art (Houben Weyl, “Methoden der organischen Chemie” [Methods of organic chemistry], Vol. 4/1c Part 1, p. 14 ff. (1980), Georg Thieme Verlag Stuttgart, New York). Intermediates 5 are prepared by the Friedel-Crafts ring-closure methods that are familiar to a person skilled in the art (Chem. Rev. 1970, 70, 553; J. Org. Chem. 1958, 23, 789, J. Org. Chem. 1981, 46, 2974; J. Med. Chem. 1986, 29, 1615). The use of phosphorus pentoxide in methanesulphonic acid or trifluoromethanesulphonic acid in the temperature range 0-30° C. may be mentioned as being especially preferred.

Alternatively, intermediates 5 can be produced according to Synthesis Scheme 2, wherein R¹, R², R³, R⁴, R⁵, R⁶ and R⁷ have the meanings given in formula (I).

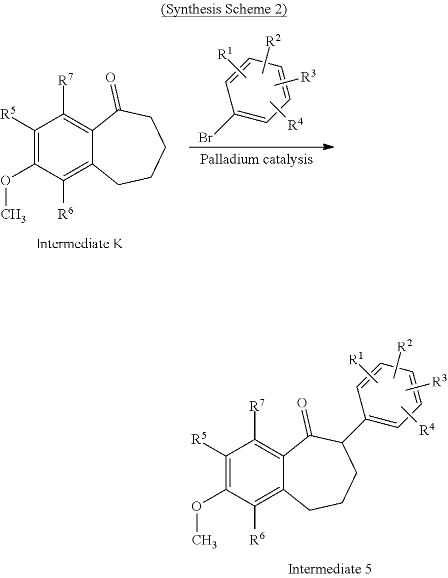

Intermediates 5 can be prepared by arylation of the intermediates K, as are known by a person skilled in the art (J. Am. Chem. Soc. 1997, 119, 11108; J. Am. Chem. Soc. 2002, 124, 15168; J. Am. Chem. Soc. 1997, 119, 12382; J. Am. Chem. Soc. 1999, 121, 1473; J. Am. Chem. Soc. 2000, 122, 1360; Tetrahedron 2001, 57, 5967; J. Org. Chem. 2001, 66, 3284; J. Org. Chem. 2006, 71, 3816; Org. Lett. 2002, 4, 4053; J. Organomet. Chem. 2005, 690, 5832; Org. Lett. 2003, 5, 1479; J. Org. Chem. 2006, 71, 685; Tetrahedron 2005, 61, 9716; Angew. Chem. 2005, 117, 2497; Angew. Chem. 2005, 117, 407; Angew. Chem. 2006, 118, 7789). For this, a palladium compound (e.g. Pd(OAc)₂, Pd₂(dba)₃) is reacted with a ligand (e.g. BINAP, 2,2′-bis(diphenylphosphino)-1,1′-binaphthyl, xantphos, triphenylphosphine, DTPF, 1,1′-bis(di-o-tolylphosphino)ferrocene, 1,3-di-tert-butyl-2-chloro-1,3,2-diazaphospholidine, 2′-(dicyclohexylphosphino)-N,N-dimethylbiphenyl-2-amine) in a solvent (e.g. toluene, xylene, tetrahydrofuran, dioxane, dimethoxyethane, tert-butyl-methyl ether) with a base (e.g. sodium tert-butanolate, potassium tert-butanolate, sodium hydride, potassium hydride, potassium hexamethyldisilazide, tripotassium phosphate, caesium carbonate) and an aromatic halide or triflate at a temperature of 40-160° C. The temperature used also depends on the solvent. The palladium compound used can also have already been joined to corresponding ligands beforehand, for example (ItBu)Pd(allyl)Cl, (IPr)Pd(acaac)Cl, Pd(dppf)Cl, [PdBrPtBu]₂. Especially preferably, palladium(II) acetate with BINAP or xantphos or allylchloro(1,3-bis(2,6-di-isopropylphenyl)imidazol-2-ylidene)palladium are used for the reactions. An alkali salt of an alcohol as base in THF at 60-80° C. is especially preferred. Reaction with palladium(II) acetate, xantphos, sodium tert-butanolate in THF under reflux is quite especially preferred. The excess of aryl halide is to be kept as low as possible, preferably just one equivalent of aryl halide and one equivalent of ketone are used.

Intermediates 10 can be synthesized according to Synthesis Scheme 3, wherein R¹, R², R³, R⁴, R⁵, R⁶ and R⁷ and m have the meanings given in formula (I).

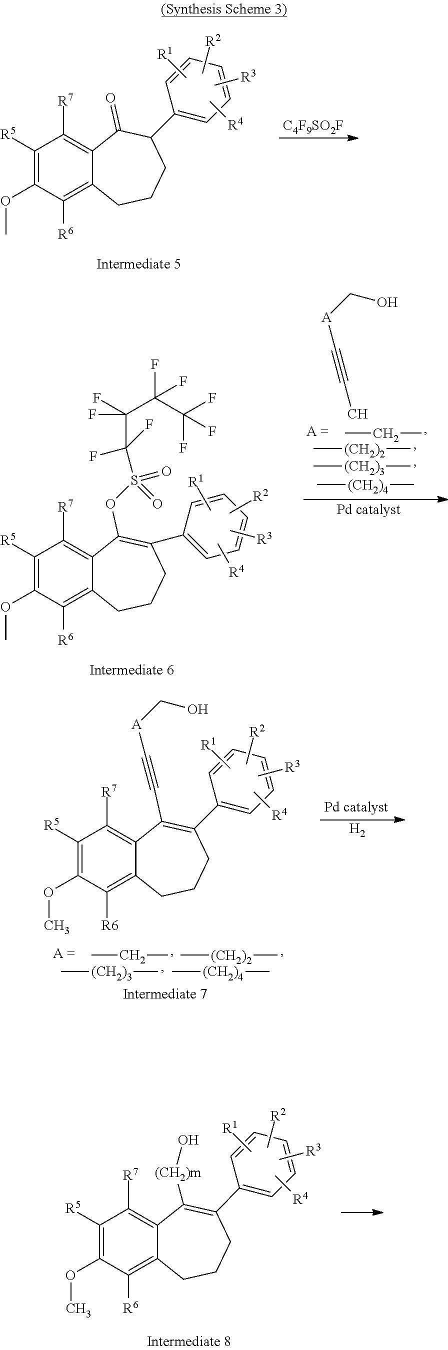

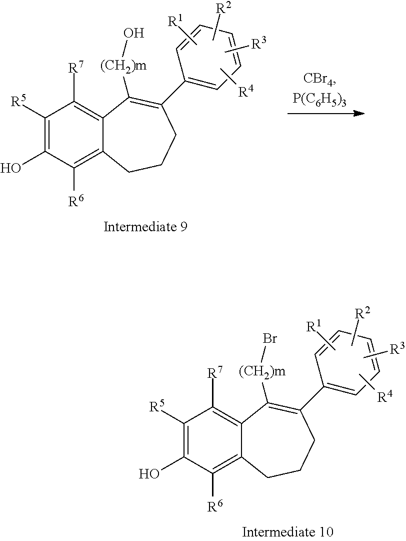

Intermediate 6 can be prepared according to the conditions known by a person skilled in the art (Tetrahedron: Asymmetry 1990, 1, 97; J. Org. Chem. 1996, 61, 8536; Synthesis 2002, 2064). Analogous perfluorinated sulphonylenol ethers can also be produced, with the nonafluorobutyl residue being replaced with e.g. trifluoromethyl. Reaction in the presence of organic amines in ethers or halogenated solvents is especially preferred for the production of intermediate 6. Reaction with nonafluorobutylsulphonyl fluoride in tetrahydrofuran/methyl tert-butyl ether with 2,3,4,5,7,8,9,10-octahydropyrido[1,2-4][1,3]diazepine as base and with cooling at 0-15° C. is quite especially preferred. Intermediates 7 can be prepared according to Sonogashira with a palladium catalyst (e.g. Pd(PPh₃)₄, Pd(Cl)₂(PPh₃)₂ and the same commercial catalysts) and an amino base in an aprotic solvent (Chem. Rev. 2007, 107, 874; Synthesis 1986, 320; Angew. Chem. 1994, 106, 1568), as known by a person skilled in the art. Reaction with palladium tetrakistriphenylphosphine and triethylamine in DMF at 60-100° C. is especially preferred. Intermediates 8 can be synthesized by the methods known by a person skilled in the art (J. Org. Chem. 1990, 55, 3484; J. Org. Chem. 1964, 29, 3660; Chem. Ber. 1959, 92, 541) with a transition catalyst and hydrogen. Hydrogenation with palladium is especially preferred. Hydrogenation in methanol with addition of base e.g. potassium hydroxide, is quite especially preferred. To obtain intermediate 9, methyl ether must be cleaved by methods that are known by a person skilled in the art (“Protective Groups in Organic Synthesis” 3rd edition, p. 250 ff. (1999), John Wiley & Sons New York). Cleavage with boron tribromide is especially preferred and methyl ether cleavage with boron tribromide with addition of a pyridine derivative (e.g. lutidine) with cooling in an inert solvent (e.g. dichloromethane) at 0-10° C. is quite especially preferred. For preparation of the example compounds, intermediate 10 in the side chain is transformed to an activated form, as is known by a person skilled in the art (J. Am. Chem. Soc. 1964, 86, 964; Tetrahedron Lett. 1973, 3937; Angew. Chem. Int. Ed. 1975, 14, 801; J. Org. Chem. 1969, 34, 212; J. Am. Chem. Soc. 1970, 92, 2139; J. Chem. Soc., Perkin Trans. 1, 1980, 2866; J. Org. Chem. 1986, 51, 5291; J. Org. Chem. 1962, 27, 349). Conversion to the bromine compound with triphenylphosphine and carbon tetrabromide in an inert solvent (e.g. tetrahydrofuran) at 0-10° C. is especially preferred.

Intermediates 11 can be prepared according to Synthesis Scheme 4, wherein halogen stands for chlorine, bromine or iodine, n has the meaning given in formula (I) and X1 is selected from the group comprising H, C₁-C₆-alkyl-, C₃-C₈-cycloalkyl-, phenyl-C₁-C₆-alkyl-, which optionally can be substituted once, twice or multiply with —OH, halogen, —CN, alkoxy.

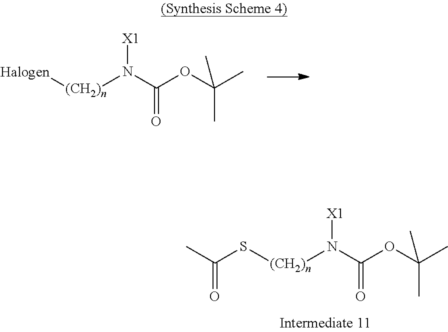

The intermediates 11 can be prepared according to the conditions known by a person skilled in the art (J. Chem. Soc. 1950, 579; J. Am. Chem. Soc. 1953, 75, 3700).

Intermediates 16 can be prepared according to Synthesis Scheme 5, where Y, q, n have the meanings given in formula (I), X2 is selected from the group comprising H, C₁-C₆-alkyl-, C₃-C₈-cycloalkyl-, phenyl-C₁-C₆-alkyl-, which optionally can be substituted once, twice or multiply with —OH, deuterium, halogen, —CN, alkoxy.

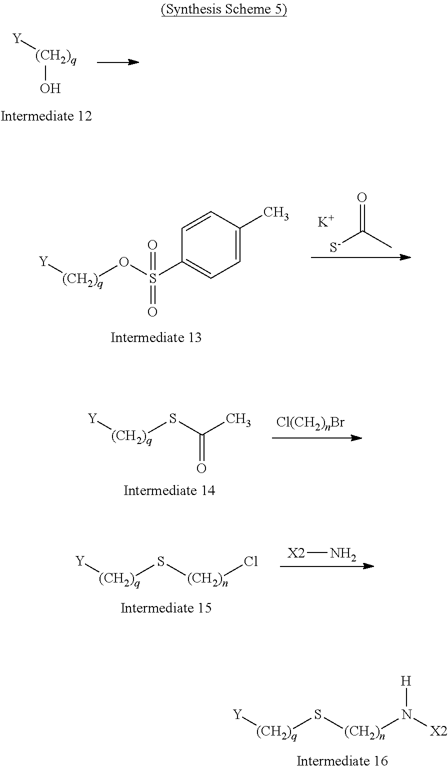

The commercial intermediates 12 (e.g. Aldrich) are converted by the methods known by a person skilled in the art to the intermediates 13 (J. Chem. Soc. 1939, 1248; Synthesis 1996, 594; Helv. Chim. Acta 1946, 29, 671). Intermediates 14 can be synthesized by the methods known by a person skilled in the art (J. Chem. Soc. 1950, 579; J. Am. Chem. Soc. 1953, 75, 3700). Intermediates 15 are prepared by the methods of synthesis known by a person skilled in the art (Pharm. Chem. J. 1989, 23, 998). Intermediates 16 are synthesized by the methods known by a person skilled in the art (Org. Synth. Coll. Vol. 1, 102, 1941; Org. Synth. Coll. Vol. 2, 290, 1943; Org. Synth. Coll. Vol. 3, 256, 1953; J. Am. Chem. Soc. 1952, 74, 5105; J. Am. Chem. Soc. 1954, 76, 658).

Intermediates 18 can be prepared according to Synthesis Scheme 6, where Y, q, n have the meanings given in formula (I), X3 is selected from the group comprising H, C₁-C₆-alkyl-, C₃-C₈-cycloalkyl-, phenyl-C₁-C₆-alkyl-, which optionally can be substituted once, twice or multiply with —OH, deuterium, halogen, —CN, alkoxy.

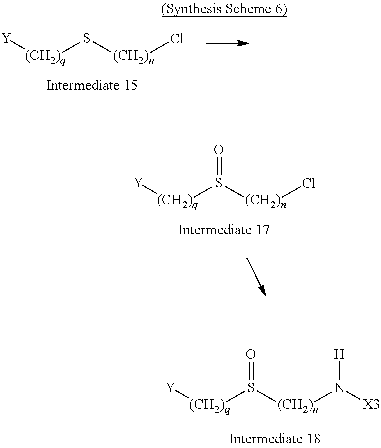

Intermediates 17 can be prepared by the methods known by a person skilled in the art (Org. Prep. Proced. Int. 1982, 14, 45; J. Org. Chem. 1962, 27, 282). Oxidation with metaperiodate is especially preferred. Oxidation with sodium metaperiodate is quite especially preferred. Intermediates 18 can be prepared as described for intermediates 16.

Intermediates 20 can be prepared according to Synthesis Scheme 7, where Y, q, n have the meanings given in formula (I), X4 is selected from the group comprising H, C₁-C₆-alkyl-, C₃-C₈-cycloalkyl-, phenyl-C₁-C₆-alkyl-, which optionally can be substituted once, twice or multiply with —OH, deuterium, halogen, —CN, alkoxy.

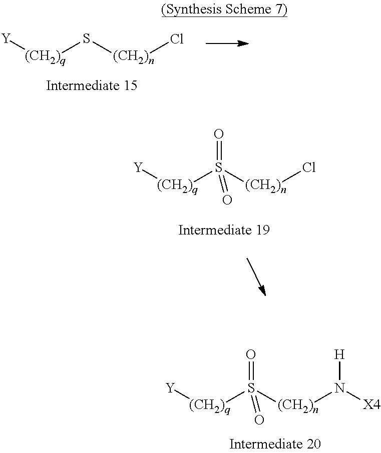

Intermediates 19 can be prepared by the methods known by a person skilled in the art (J. Org. Chem. 1957, 22, 241; J. Org. Chem. 2004, 69, 3824; J. Am. Chem. Soc. 1941, 63, 2939; Org. Lett. 1999, 1, 189). Oxidation with per acids is especially preferred. Intermediates 20 can be prepared as described for intermediates 16.

Intermediates 14 can also be prepared according to Synthesis Scheme 8, where Y and q have the meanings given in formula (I).

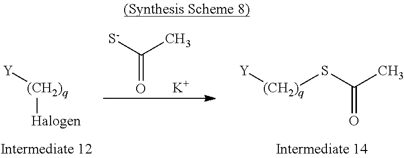

Intermediates 14 can also be prepared from the corresponding halogen compounds by the methods known by a person skilled in the art (J. Am. Chem. Soc. 1953, 75, 3700; J. Org. Chem. 1984, 49, 3231).

Intermediates 16, 18 and 20 can alternatively also be prepared via Synthesis Scheme 9, where Y, p, q, n have the meanings given in formula (I), X5 is selected from the group comprising H, C₁-C₆-alkyl-, C₃-C₈-cycloalkyl-, phenyl-C₁-C₆-alkyl-, which optionally can be substituted once, twice or multiply with —OH, deuterium, halogen, —CN, alkoxy.

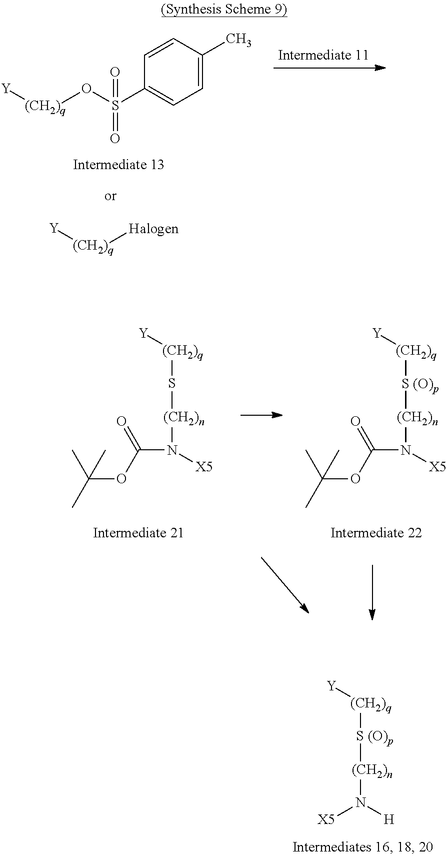

Intermediates 21 are synthesized by reacting the tosylates 13 or the corresponding halogen compounds with an intermediate 11 by the methods known by a person skilled in the art, as described for intermediate 15. Conversion to the intermediates 22 takes place similarly to the methods for production of intermediates 17 and 19. Conversion to intermediates 16, 18 and 20 starting from intermediates 21 or 22 can take place by the methods known by a person skilled in the art (e.g. “Protective Groups in Organic Synthesis” 3rd edition, p. 520 ff. (1999), John Wiley & Sons New York). Cleavage with acids is especially preferred and cleavage with trifluoroacetic acid is quite especially preferred.

The example compounds can be synthesized according to Synthesis Scheme 10 by reaction of intermediates 16, 18 or 20 with intermediate 10, where R¹, R², R³, R⁴, R⁵, R⁶, R⁷, m, n, p, q, Y have the meanings given in formula (I), X6 is selected from the group comprising H, C₁-C₆-alkyl-, C₃-C₈-cycloalkyl-, phenyl-C₁-C₆-alkyl-, which optionally can be substituted once, twice or multiply with —OH, deuterium, halogen, —CN, alkoxy.

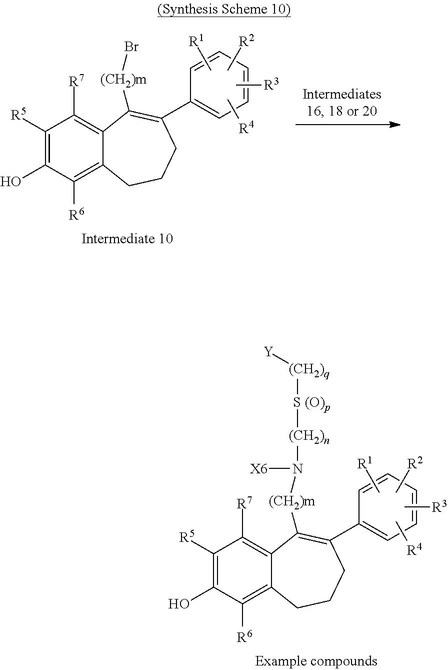

The example compounds were synthesized according to Synthesis Scheme 10 by reaction of intermediates 16, 18 or 20 with intermediate 10. The reactions can be carried out by the methods known by a person skilled in the art as described for the conversion of intermediate 15 to intermediate 16. The reaction in the presence of an alkali metal iodide and a carbonate of the alkali metals in an aprotic solvent such as DMF or NMP is especially preferred.

Further example compounds can be obtained according to Synthesis Scheme 11 by reaction of example compounds with the meaning X6=H to example compounds with X7 selected from the group comprising C₁-C₆-alkyl-, C₃-C₈-cycloalkyl-, C₁-C₆-alkyl-S(O)₂—, C₁-C₆-alkylcarbonyl-, phenyl-C₁-C₆-alkyl-, which optionally can be substituted once, twice or multiply with —OH, deuterium, halogen, —CN, NR⁷R⁸, —C(O)NR⁹R¹⁰, —N(R⁹)C(O)NR⁹R¹⁰, alkoxy or —C(O)OC₁-C₆-alkyl.

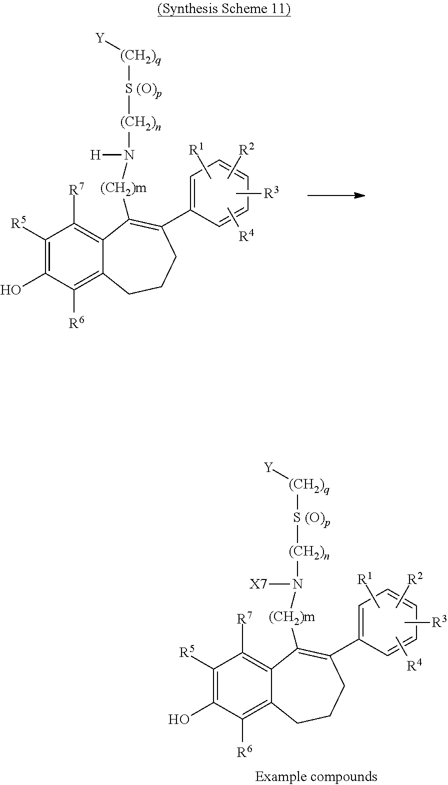

The reaction according to Synthesis Scheme 11 can be carried out by the methods as described for the conversion of intermediate 15 to intermediate 16.

Further example compounds can be obtained according to Synthesis Scheme 12 by reaction of example compounds with the meaning X7=C₁-C₆-alkyl-, C₃-C₈-cycloalkyl-, C₁-C₆-alkylcarbonyl-, phenyl-C₁-C₆-alkyl-, which have been substituted once, twice or multiply with —C(O)OC₁-C₆-alkyl, to the example compounds with the meaning X8=C₁-C₆-alkyl-, C₃-C₈-cycloalkyl-, C₁-C₆-alkylcarbonyl-, phenyl-C₁-C₆-alkyl-, which have been substituted once, twice or multiply with —C(O)OH.

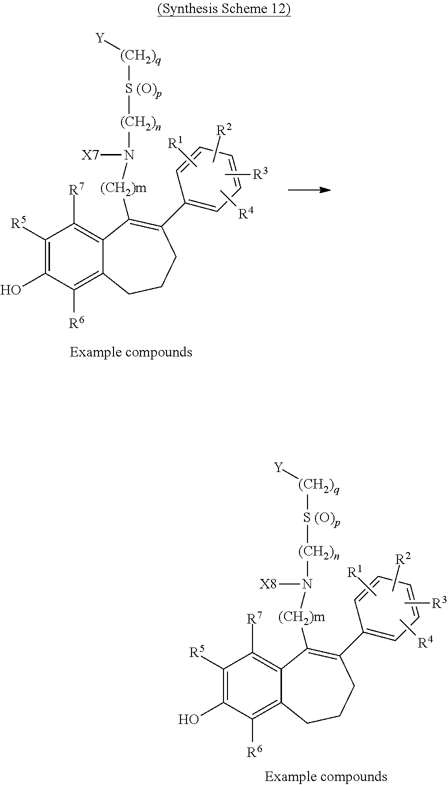

Hydrolysis of the example compounds with the meaning X7=C₁-C₆-alkyl-, C₃-C₈-cycloalkyl-, C₁-C₆-alkylcarbonyl-, phenyl-C₁-C₆-alkyl-, which have been substituted once, twice or multiply with —C(O)OC₁-C₆-alkyl, to example compounds with the meaning X8=C₁-C₆-alkyl-, C₃-C₈-cycloalkyl-, C₁-C₆-alkylcarbonyl-, phenyl-C₁-C₆-alkyl-, which have been substituted once, twice or multiply with —C(O)OH, can be effected by methods that are known by a person skilled in the art (“Protective Groups in Organic Synthesis” 3rd edition, p. 250 ff. (1999), John Wiley & Sons New York; J. Am. Chem. Soc. 1946, 68, 1855; J. Org. Chem. 1959, 24, 1367). Reactions with aqueous alkaline solution and an alcohol are especially preferred. Reactions with an alkali metal hydroxide (e.g. NaOH, KOH, LiOH) are quite especially preferred.

The compounds according to the invention display an unforeseeable, valuable pharmacological and pharmacokinetic spectrum of action. They are therefore suitable for use as medicinal products for the treatment and/or prophylaxis of diseases in humans and animals. Within the scope of the present invention, the term “treatment” includes prophylaxis. The pharmaceutical efficacy of the compounds according to the invention can be explained by their action as SERMs.

The present invention further relates to the use of the compounds according to the invention for the treatment and/or prophylaxis of diseases, preferably of gynaecological diseases, for alleviating the symptoms of the andropause and menopause, i.e. for male and female hormone replacement therapy (HRT), namely both for prevention and for treatment; for the treatment of problems accompanying dysmenorrhoea; treatment of dysfunctional uterine bleeding; treatment of acne; prevention and treatment of cardiovascular diseases; treatment of hypercholesterolaemia and hyperlipidaemia; prevention and treatment of atherosclerosis; for inhibiting proliferation of arterial smooth muscle cells; for the treatment of respiratory distress syndrome of the newborn; treatment of primary pulmonary hypertension; for prevention and treatment of osteoporosis (Black, L. J., Sato, M., Rowley, E. R., Magee, D. E., Bekele, A., Williams, D. C., Cullinan, G. J., Bendele, R., Kauffman, R. F., Bensch, W. R., Frolik, C. A., Termine, J. D. and Bryant, H. U.: Raloxifene [LY 139481 HCl] prevents bone loss and reduces serum cholesterol without causing uterine hypertrophy in ovariectomized rats; J. Clin. Invest. 93: 63-69, 1994); for preventing bone loss in postmenopausal women, in hysterectomized women or in women who have been treated with LHRH agonists or antagonists; inhibition of sperm maturation; treatment of rheumatoid arthritis; for the prevention of Alzheimer's disease; treatment of endometriosis; treatment of myomata; treatment of myomata and endometriosis in combination with LHRH analogues; treatment of hormone-dependent tumours (also in premenopausal women), e.g. of breast cancer or e.g. of endometrial carcinoma, treatment of prostatic diseases, treatment of benign diseases of the breast e.g. mastopathy. Moreover, based on their pharmacological profile, the compounds according to the invention are suitable both for male and for female contraception.

The present invention further relates to the use of the compounds according to the invention for the treatment of infertility and for induction of ovulation.

The present invention further relates to the use of the compounds according to the invention for the treatment and prophylaxis of stroke and Alzheimer's and other diseases of the central nervous system, which is accompanied by cellular death of neurons.

The present invention further relates to the use of the compounds according to the invention for the production of a medicinal product for the treatment and/or prophylaxis of diseases, in particular the aforementioned diseases.

The present invention further relates to a method of treatment and/or prophylaxis of diseases, in particular the aforementioned diseases, using an effective amount of the compounds according to the invention.

The present invention further relates to the use of the compounds according to the invention for the treatment and/or prophylaxis of diseases, in particular the aforementioned diseases.

The present invention further relates to the compounds according to the invention for use in a method of treatment and/or prophylaxis of the aforementioned diseases.

The present invention further relates to medicinal products containing at least one compound according to the invention and at least one or more other active substances, in particular for the treatment and/or prophylaxis of the aforementioned diseases. For example and preferably, the following may be mentioned as suitable combination active substances: oestrogens, gestagens and progesterone receptor antagonists.

Oestrogens are compounds (naturally occurring or synthetic, steroidal and non-steroidal compounds) that display oestrogenic efficacy. Such compounds are for example: ethinylestradiol, estradiol, estradiol sulphamate, estradiol valerate, estradiol benzoate, estrones, mestranol, estriol, estriol succinate and conjugated oestrogen, including conjugated equine oestrogens such as estrone sulphate, 17β-estradiol sulphate, 17α-estradiol sulphate, equilin sulphate, 17β-dihydroequilin sulphate, 17α-dihydroequilin sulphate, equilenin sulphate, 17β-dihydroequilenin sulphate and 17α-dihydroequilenin sulphate. Especially interesting oestrogens are ethinylestradiol, estradiol, estradiol sulphamate, estradiol valerate, estradiol-15-benzoate, estrone, mestranol and estrone sulphate. Ethinylestradiol, estradiol and mestranol are preferred as oestrogens, and ethinylestradiol is especially preferred.

Gestagens are understood in the sense of the present invention either as natural progesterone itself or synthetic (steroidal and non-steroidal) derivatives, which like progesterone itself bind to the progesterone receptor and, in dosages that are above the ovulation inhibiting dose, inhibit ovulation. The following may be mentioned as examples of gestagens: levonorgestrel, norgestimate, norethisterone, dydrogesterone, drospirenone, 3-beta-hydroxydesogestrel, 3-ketodesogestrel (=etonogestrel), 17-deacetylnorgestimate, 19-norprogesterone, acetoxypregnenolone, allylestrenol, amgestone, chloromadinone, cyproterone, demegestone, desogestrel, dienogest, dihydrogesterone, dimethisterone, ethisterone, ethynodiol diacetate, fluorogestone acetate, gastrinone, gestodene, gestrinone, hydroxymethylprogesterone, hydroxyprogesterone, lynestrenol (=lynoestrenol), mecirogestone, medroxyprogesterone, megestrol, melengestrol, nomegestrol, norethindrone (=norethisterone), norethynodrel, norgestrel (including d-norgestrel and dl-norgestrel), norgestrienone, normethisterone, progesterone, quingestanol, (17alpha)-17-hydroxy-11-methylene-19-norpregna-4,15-dien-20-yn-3-one, tibolone, trimegestone, algestone acetophenide, nestorone, promegestone, 17-hydroxyprogesterone ester, 19-nor-17hydroxyprogesterone, 17alpha-ethinyl-testosterone, 17alpha-ethinyl-19-nortestosterone, d-17beta-acetoxy-13beta-ethyl-17alpha-ethinyl-gon-4-en-3-onoxime or the compounds disclosed in WO 00/66570, in particular tanaproget. Levonorgestrel, norgestimate, norethisterone, drospirenone, dydrogesterone and dienogest are preferred. Drospirenone and dienogest are especially preferred.

Progesterone receptor antagonists are compounds which inhibit the action of progesterone on its receptor. As examples we may mention RU 486, onapristone, lonaprisan (11β-(4-acetylphenyl)-17β-hydroxy-17β-(1,1,2,2,2-pentafluoroethyl)estra-4,9-dien-3-one cf. WO 98/34947) and the compounds claimed in WO 08/58767.

The invention also relates to pharmaceutical preparations that contain at least one compound of general formula I (or physiologically compatible salts of addition with organic and inorganic acids thereof) and the use of these compounds for the production of medicinal products, in particular for the indications mentioned above.

The compounds can be used for the indications mentioned above, both by oral and parenteral administration.

The compounds can also be used in combination with the natural vitamin D3 or with calcitriol analogues for osteogenesis or as supporting therapy for therapies that cause loss of bone mass (for example therapy with glucocorticoids, chemotherapy).

The compounds of general formula I can also be used in combination with progesterone receptor antagonists or in combination with pure oestrogen, and in particular for use in hormone replacement therapy and for the treatment of gynaecological disorders and for controlling female fertility. A therapeutic product, containing an oestrogen and a pure anti-oestrogen for simultaneous, sequential or separate use for selective oestrogen therapy of perimenopausal or postmenopausal states is already described in EP-A 0 346 014.

The compounds of general formula I can also be given in combination with gestagens, substances with gestagenic action or COCs (combined oral contraceptives), in particular for use in premenopausal women for the treatment of gynaecological diseases such as endometriosis, myomata or disturbances of menstruation e.g. dysmenorrhoea or hypermenorrhoea or for the treatment of hormone-dependent tumours, e.g. breast cancer.

The compounds of general formula I can be administered both continuously (for example once daily) and in intermittent regimens. We may mention for example (but not exclusively) treatment regimes such as once weekly, once monthly, daily for a period of several days, on particular days of the female menstrual cycle (e.g. on 14 consecutive days of the secretory phase or several days in the middle of the menstrual cycle). The compounds of general formula I can also be administered continuously over a longer treatment period (e.g. 14-168 successive days) followed by a treatment pause, which is either established (e.g. 14-84 days) or is flexible and lasts until the next menstrual bleed. In these intermittent treatment regimens the compounds of general formula I can be administered alone or in combination with the aforementioned combination therapies, and these in their turn can be administered continuously or also intermittently.

The compounds according to the invention can have systemic and/or local action. For this purpose, they can be administered in a suitable way, e.g. oral, parenteral, pulmonary, nasal, sublingual, lingual, buccal, rectal, dermal, transdermal, conjunctival, otic or as implant or stent.

For these routes of application, the compounds according to the invention can be administered in suitable dosage forms.

Dosage forms that function according to the prior art, with rapid and/or modified release of the compounds according to the invention, containing the compounds according to the invention in crystalline and/or amorphized and/or dissolved form, are suitable for oral application, for example tablets (uncoated or coated tablets, for example with enteric coatings or coatings with delayed dissolution or insoluble coatings, which control the release of the compound according to the invention), tablets that disintegrate rapidly in the oral cavity or films/wafers, films/lyophilizates, capsules (for example hard-gelatin or soft-gelatin capsules), coated tablets, granules, pellets, powders, emulsions, suspensions, aerosols or solutions.

Parenteral application can take place with avoidance of an absorption step (e.g. intravenous, intraarterial, intracardial, intraspinal or intralumbar) or with inclusion of absorption (e.g. intramuscular, subcutaneous, intracutaneous, percutaneous or intraperitoneal). Suitable dosage forms for parenteral application are among others injection and infusion preparations in the form of solutions, suspensions, emulsions, lyophilizates or sterile powders.

Suitable dosage forms for the other routes of administration are e.g. inhalational pharmaceutical forms (e.g. powder inhalers, nebulizers), nasal drops, solutions, and sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powder, implants, intrauterine systems IUS for drug release (e.g. intrauterine coils), vaginal rings or stents.

The compounds according to the invention can be transformed into the stated dosage forms. This can take place in a manner that is known per se by mixing with inert, non-toxic, pharmaceutically suitable excipients. These excipients include inter alia vehicles (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, such as ascorbic acid), colorants (e.g. inorganic pigments, such as iron oxides) and taste and/or odour correctants.

The present invention further relates to medicinal products that contain at least one compound according to the invention, usually together with one or more inert, non-toxic, pharmaceutically suitable excipients, and use thereof for the purposes stated above.

In the case of oral administration, the amount per day is about 0.01 to 100 mg/kg body weight. The amount of a compound of general formula I to be administered varies over a wide range and can cover every effective amount. Depending on the condition to be treated and the method of administration, the amount of the compound administered can be 0.01-100 mg/kg body weight per day.

Nevertheless, it may optionally be necessary to deviate from the stated amounts, namely depending on body weight, route of administration, individual response to the active substance, type of preparation and time point or interval in which application takes place. Thus, in some cases it may be sufficient to use less than the aforementioned minimum amount, whereas in other cases the stated upper limit must be exceeded. In the case of administration of larger amounts, it may be advisable to divide these into several individual doses throughout the day.

The percentages in the following tests and examples are, unless stated otherwise, percentages by weight; parts are parts by weight. Proportions of solvents, dilution ratios and information on concentration for liquid/liquid solutions relate in each case to volume.

### LIST OF ABBREVIATIONS, CHEMISTRY

### Abbreviations and Acronyms

CI chemical ionization (in MS)
TLC thin layer chromatography
DMF dimethylformamide
DMSO dimethylsulphoxide
of theor. of theoretical (relating to yield)
ESI electrospray ionization (in MS)
GC-MS gas chromatography coupled with mass spectroscopy
h hour(s)
HPLC high-performance (high-pressure) liquid chromatography
LC-MS liquid chromatography coupled with mass spectroscopy
Mass found mass found in the mass spectrum
min minute(s)
MS mass spectroscopy
NMR nuclear magnetic resonance
R_{f} retention index (in TLC)
Rₜ retention time (in HPLC)
RT room temperature
TFA trifluoroacetic acid
THF tetrahydrofuran

### Purification of the Compounds According to the Invention

In some cases the compounds according to the invention could be purified by preparative HPLC for example using an autopurifier apparatus from the company Waters (detection of the compounds by UV-detection and electrospray ionization) in combination with commercially available, prepacked HPLC columns (for example XBridge column (from Waters), C18, 5 μm, 30×100 mm). Acetonitrile/water+0.1% TFA or 0.1% formic acid was used as the solvent system. Instead of acetonitrile, methanol for example could also be used.

The flow during purification was 50 mL/min.

In some cases the compounds according to the invention were purified by the following method (HPLC-Method 1):

Waters HPLC autopurification system Pump 2525, Sample Manager 2767, CFO, DAD 2996, ELSD 2424, ZQ 4000, column: XBridge C18, 5 μm, 100×30 mm, 50 mL/min, solvent: water with 0.1% formic acid-acetonitrile 99:1, 0-1 minute; 99:1→1:99, 1-7.5 minutes; 1:99, 7.5-10 minutes, detection per DAD scan range 210-400 nm, ELSD, MS ESI (+), ESI (−), scan range 160-1000 m/z.

In some cases the compounds according to the invention were purified by the following method (HPLC-Method 2):

XBridge C18, 5 μm, 100×30 mm, 50 mL/min, solvent: water with 0.1% formic acid-methanol 70:30, 0-1 minute; 70:30→1:99, 1-7.5 minutes; 1:99, 7.5-10 minutes, other conditions were similar to Method 1.

Freeze-drying or vacuum centrifugation was used for removing the HPLC solvent mixture. The compounds thus obtained could be in the form of TFA salts or formate salts and could be transformed to the respective free bases by standard laboratory procedures that are known by a person skilled in the art.

In some cases the compounds according to the invention could be purified by silica gel chromatography. For this, it would be possible for example to use prepacked silica gel cartridges (for example from the company Separtis, Isolute® Flash silica gel) in combination with the Flashmaster II chromatograph (Argonaut/Biotage) and chromatography solvents or solvent mixtures such as for example hexane, ethyl acetate and dichloromethane and methanol, and additions of aqueous ammonia solution could also be used.

### Structural Analysis of the Compounds According to the Invention:

In some cases the compounds according to the invention were analysed by LC-MS:

One method of analysis used was based on the following parameters:

System Waters Acquity UPLC-MS: Binary Solvent Manager, Sample Manager/Organizer, Column Manager, PDA, ELSD, SQD 3001, column: Acquity BEH C18, 1.7 μm, 50×2.1 mm. Water with 0.1% TFA or with 0.1% formic acid was used as solvent A. Solvent B consisted of acetonitrile. Gradient 0-1.6 min 1-99% B, 1.6-2.0 min 99% B, flow 0.8 mL/min, temperature 60° C., sample solution 1.0 mg/mL in acetonitrile/water 7:3, injection 2.0 μl, detection per DAD scan range 210-400 nm, ELSD, MS ESI (+), ESI (−), scan range 160-1000 m/z.

In some cases the compounds according to the invention were analysed by LC-MS: retention times Rₜ from LC-MS analysis: detection: UV=200-400 nm (Acquity HPLC system from the company Waters)/MS 100-800 dalton; 20 V (Micromass/Waters ZQ 4000) in ESIpos mode (for production of positively charged molecular ions); HPLC column: X Bridge (Waters), 2.1×50 mm, BEH 1.7 μm; solvent: A: water/0.05% formic acid, B: acetonitrile. Gradient: 10-90% B in 1.7 min, 90% B for 0.2 min, 98-2% B in 0.6 min; flow rate: 1.3 mL/min.

In some cases a Waters ZQ4000 instrument or a Single Quadrupol API (atomic pressure ionization) mass detector (Waters) was used for recording a mass spectrum.

The following symbols are used in the NMR data of the compounds according to the invention:

| | |
|---|---|
| s | singlet |
| d | doublet |
| t | triplet |
| q | quadruplet |
| quin | quintuplet |
| m | multiplet |
| br | broad |
| mc | centred multiplet |

### Intermediate 1-2

### (2E)-3-(2-Fluoro-3-methoxyphenyl)acrylaldehyde

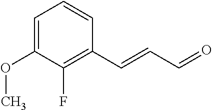

50 g of potassium hydroxide was dissolved in 250 mL water, and 50 g (0.324 mol) of 2-fluoro-3-methoxybenzaldehyde in 200 mL dichloromethane was added. 57.16 g acetaldehyde in 250 mL water was added dropwise in 3 hours. Stirring was continued overnight and for 1 day at room temperature. 15 g acetaldehyde in 60 mL water was added dropwise. It was stirred for a further 24 hours at room temperature. It was shaken three times with dichloromethane. The combined organic phases were adjusted with acetic acid-water 1:4 to a pH of 5-6, washed with water, dried over magnesium sulphate and concentrated by evaporation. It was purified on Silica Gel 60 (solvent: hexane, hexane-ethyl acetate 95:5 and 90:10). 38 g (65% of theor.) of product was obtained.

¹H-NMR (400 MHz, chloroform-d₁): δ=3.92 (s, 3H), 6.77 (dd, 1H), 7.02-7.07 (m, 1H), 7.10-7.18 (m, 2H), 7.69 (d, 1H), 9.73 (d, 1H).

### Intermediate 2-2

### (2E)-3-(4-Fluoro-3-methoxyphenyl)acrylaldehyde

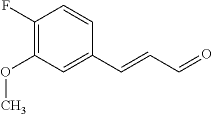

142 mL of 20% potassium hydroxide solution was added to 50 g (0.324 mol) of 4-fluoro-3-methoxybenzaldehyde in 250 mL dichloromethane. 73 mL (1.298 mol) of acetaldehyde in 210 mL water was added dropwise at below 30° C. in 2 hours. Stirring was continued overnight at room temperature. On each of four days, 1 mol-equivalent of acetaldehyde was added dropwise in 3 portions of 6 mL and stirring was continued overnight or over the weekend. The reaction mixture was shaken three times with dichloromethane. The combined organic phases were adjusted to a pH of 5-6 with acetic acid-water 1:3, washed with water, dried over magnesium sulphate and concentrated by evaporation. It was purified on Silica Gel 60 (solvent: hexane, hexane-ethyl acetate 95:5, 90:10, 85:15, 80:20 and 70:30). 17.56 g (30% of theor.) of product was obtained.

¹H-NMR (400 MHz, chloroform-d₁): δ=3.93 (s, 3H), 6.64 (dd, 1H), 7.11-7.17 (m, 3H), 7.42 (d, 1H), 9.69 (d, 1H).

### Intermediate 1-3

### (2E,4E)-5-(2-Fluoro-3-methoxyphenyl)-2-(4-fluorophenyl)penta-2,4-dienoic acid

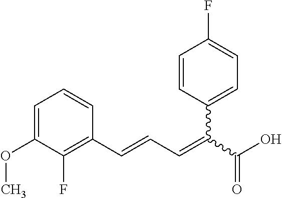

17.88 g (0.116 mol) of (4-fluorophenyl)acetic acid and 32.2 mL (0.232 mol) of triethylamine were added to 19.00 g (0.105 mol) of (2E)-3-(2-fluoro-3-methoxyphenyl)acrylaldehyde and 21.9 mL (0.232 mol) of acetic anhydride. It was stirred for 10 hours at 100° C. and overnight at room temperature. The reaction mixture was poured onto ice/water with 5 vol. % concentrated hydrochloric acid and extracted with chloroform three times. The combined organic phases were washed with water twice, dried over magnesium sulphate and concentrated by evaporation. Diisopropyl ether-hexane 1:1 was added to the residue, filtered with suction and dried in a drying cabinet. 21.0 g (63% of theor.) of product was isolated.

¹H-NMR (400 MHz, chloroform-d₁): principal isomer: δ=3.88 (s, 3H), 6.82-6.96 (m, 3H), 6.99 (d, 1H), 7.08-7.15 (m, 2H), 7.20 (d, 1H), 7.27-7.32 (m, 2H), 7.76 (d, 1H).

### Intermediate 2-3

### (2E,4E)-5-(4-Fluoro-3-methoxyphenyl)-2-(4-fluorophenyl)penta-2,4-dienoic acid

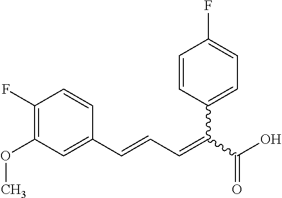

15.3 mL (162.2 mmol) of acetic anhydride and 22.5 mL (162.3 mmol) of triethylamine were added to 15.2 g (84.4 mmol) of (2E)-3-(4-fluoro-3-methoxyphenyl)acrylaldehyde and 13 g (84.3 mmol) of (4-fluorophenyl)acetic acid. It was stirred for 8 hours at 100° C. This reaction mixture, together with a second mixture (15.95 g (88.5 mmol) of (E)-3-(4-fluoro-3-methoxyphenyl)-propenal) was poured onto 800 mL ice/water with 5 vol. % concentrated hydrochloric acid, and stirred. It was extracted twice with 300 mL dichloromethane and twice with 500 mL chloroform. The combined organic phases were heated until all solids had dissolved; then washed three times with 200 mL water, dried over magnesium sulphate and concentrated by evaporation. The residue was stirred in a mixture of n-hexane and diisopropyl ether 1:1 for 1 hour under reflux. The reaction mixture was cooled, finally in an ice bath, the solid was filtered with suction, washed again with n-hexane-diisopropyl ether 1:1 and dried in a vacuum drying cabinet. 38.67 g (71% of theor.) of product was isolated.

¹H-NMR (300 MHz, chloroform-d₁): principal isomer: δ=3.87 (s, 3H), 6.68 (dd, 1H), 6.86-7.18 (m, 6H), 7.27-7.33 (m, 2H), 7.72 (d, 1H).

### Examples for Intermediate 4

General specification 4 for the preparation of 4: 1 g of dienecarboxylic acid was dissolved in 20 mL tetrahydrofuran and was hydrogenated with 0.1 g of 10 wt. % palladium on activated charcoal at normal pressure until the hydrogen had been absorbed completely. The catalyst was filtered on kieselguhr and washed with tetrahydrofuran. The filtrate was evaporated to dryness. The product was formed quantitatively.

### Intermediate 1-4

### 5-(2-Fluoro-3-methoxyphenyl)-2-(4-fluorophenyl)pentanoic acid

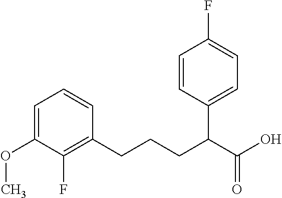 [SMILES: COc1cccc(CCCC(C(=O)O)c2ccc(F)cc2)c1F]

21.0 g (66.4 mmol) of (2E,4E)-5-(2-fluoro-3-methoxyphenyl)-2-(4-fluorophenyl)penta-2,4-dienoic acid was reacted according to general specification 4.

¹H-NMR (300 MHz, chloroform-d₁): δ=1.45-1.68 (m, 2H), 1.72-1.89 (m, 1H), 2.02-2.17 (m, 1H), 2.54-2.73 (m, 2H), 3.55 (t, 1H), 3.86 (s, 3H), 6.69 (mc, 1H), 6.79 (dt, 1H), 6.91-7.05 (m, 3H), 7.22-7.30 (m, 2H).

### Intermediate 2-4

### 5-(4-Fluoro-3-methoxyphenyl)-2-(4-fluorophenyl)pentanoic acid

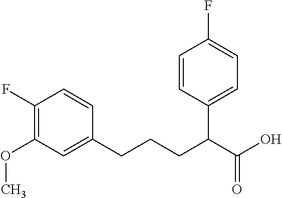 [SMILES: COc1cc(CCCC(C(=O)O)c2ccc(F)cc2)ccc1F]

38.9 g (123.0 mmol) of (2E,4E)-5-(4-fluoro-3-methoxyphenyl)-2-(4-fluorophenyl)penta-2,4-dienoic acid was reacted according to general specification 4. 39.5 g (100% of theor.) of product was obtained.

¹H-NMR (300 MHz, chloroform-d₁): δ=1.45-1.68 (m, 2H), 1.71-1.89 (m, 1H), 2.00-2.14 (m, 1H), 2.48-2.65 (m, 2H), 3.54 (t, 1H), 3.84 (s, 3H), 6.62 (ddd, 1H), 6.70 (dd, 1H), 6.90-7.05 (m, 3H), 7.22-7.29 (m, 2H).

### Examples for Intermediate 5

General specification 5 for the preparation of 5 with exclusion of humidity of the air: 1 g carboxylic acid was dissolved in 5-7.2 mL of methanesulphonic acid and 2.7-2.8 equivalents of phosphorus pentoxide were added in portions, with cooling. It was stirred for 3-16 hours at room temperature. The reaction mixture was poured into ice/water and shaken with ethyl acetate three times. The combined organic phases were washed with 2M sodium hydroxide solution until the wash water had a pH of 7-8, washed with saturated sodium chloride solution, dried over sodium sulphate and concentrated by evaporation.

General specification 5-A for the preparation of 5 with exclusion of humidity of the air: 1 g carboxylic acid was dissolved in approx. 5-10 mL of trifluoromethanesulphonic acid. 2.8 equivalents of phosphorus pentoxide were added in 3 portions at 5-20° C. Stirring was continued overnight. The reaction mixture was poured into ice/water and stirred for a further 30 min. The aqueous phase was shaken with ethyl acetate three times. The combined organic phases were washed with water, saturated sodium chloride solution and sodium carbonate solution until the wash water had a pH of 7-8, dried over magnesium sulphate and concentrated by evaporation.

### Intermediate 1-5

### 1-Fluoro-6-(4-fluorophenyl)-2-methoxy-6,7,8,9-tetrahydro-5H-benzo[7]annulen-5-one

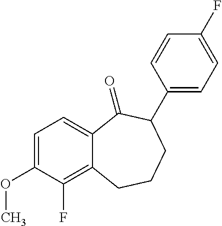 [SMILES: COc1ccc2c(c1F)CCCC(c1ccc(F)cc1)C2=O]

21.0 g (65.6 mmol) of 5-(2-fluoro-3-methoxyphenyl)-2-(4-fluorophenyl)pentanoic acid was reacted at 5-10° C. according to general specification 5-A. After stirring for a further 30 min, the precipitate was filtered with suction and washed with water four times. The residue was dried at 40° C. in a drying cabinet. 18.6 g (94% of theor.) of product was obtained.

¹H-NMR (300 MHz, DMSO-d₆): δ=1.48-1.65 (m, 1H), 1.88-2.21 (m, 3H), 2.81-2.95 (m, 1H), 3.14-3.27 (m, 1H), 3.86 (s, 3H), 4.26 (dd, 1H), 7.05-7.14 (m, 3H), 7.23-7.30 (m, 2H), 7.36 (dd, 1H).

### Intermediate 2-5

### 3-Fluoro-6-(4-fluorophenyl)-2-methoxy-6,7,8,9-tetrahydro-5H-benzo[7]annulen-5-one

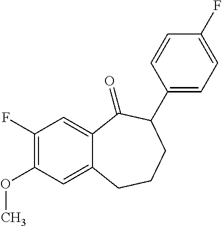 [SMILES: COc1cc2c(cc1F)C(=O)C(c1ccc(F)cc1)CCC2]

37.5 g (117 mmol) of 5-(4-fluoro-3-methoxyphenyl)-2-(4-fluorophenyl)pentanoic acid was reacted according to general specification 5. After stirring for a further 3 hours at room temperature, it was poured into ice/water and stirred again. It was extracted with 1 L dichloromethane. The organic phase was washed three times with saturated sodium hydrogen carbonate solution and three times with water, and concentrated by evaporation. The residue was dissolved in 700 mL chloroform and dried over magnesium sulphate. After filtering, activated charcoal was added, filtered on a PTFE filter and evaporated to dryness. 34.15 g (96% of theor.) of product was obtained.

¹H-NMR (300 MHz, chloroform-d₁): δ=1.72-1.88 (m, 1H), 2.03-2.28 (m, 3H), 2.96 (ddd, 1H), 3.13 (mc, 1H), 3.95 (s, 3H), 4.04 (dd, 1H), 6.81 (d, 1H), 7.03 (tt, 1H), 7.18-7.25 (m, 2H), 7.48 (d, 1H).

### Preparation of Intermediates 5 Using Palladium Catalysis

General specification 5-vPd for the preparation of 5 via palladium catalysis under argon atmosphere: 1.3 equivalents of sodium tert-butanolate, 0.05 equivalents of palladium(II) acetate and 0.024 equivalent of xantphos were put in tetrahydrofuran (20 mL/1 g ketone) under argon. 1 equivalent of 2-methoxy-6,7,8,9-tetrahydro-5H-benzo[7]annulen-5-one (ketone) dissolved in tetrahydrofuran (5 mL/1 g ketone) was added dropwise. After stirring for a further 10 minutes, 1 equivalent of aryl bromide in tetrahydrofuran (5 mL/1 g aryl bromide) was added dropwise. It was stirred under reflux for 10-25 hours. The reaction mixture was cooled and poured into potassium phosphate buffer pH 7. It was extracted with ethyl acetate four times. The combined organic phases were dried over magnesium sulphate or sodium sulphate and concentrated by evaporation. The residue was purified using Silica Gel 60.

### Intermediate 3-5

### 6-(3,4-Difluorophenyl)-2-methoxy-6,7,8,9-tetrahydro-5H-benzo[7]annulen-5-one

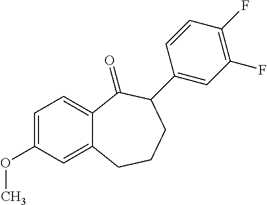 [SMILES: COc1ccc2c(c1)CCCC(c1ccc(F)c(F)c1)C2=O]

29.55 g (155.3 mmol) of 2-methoxy-6,7,8,9-tetrahydro-5H-benzo[7]annulen-5-one was reacted according to general specification 5-vPd with 29.98 g (155.4 mmol) of 4-bromo-1,2-fluorobenzene. It was stirred under reflux for 24 hours. The residue was purified using Silica Gel 60 (solvent: hexane, hexane-acetone 95:5; second column, solvent: hexane, hexane-ethyl acetate 95:5). 12.4 g (26% of theor.) of product was isolated.

¹H-NMR (400 MHz, chloroform-d₁): δ=1.75-1.88 (m, 1H), 2.04-2.24 (m, 3H), 2.96 (ddd, 1H), 3.07-3.17 (m, 1H), 3.87 (s, 3H), 4.02 (dd, 1H), 6.77 (d, 1H), 6.83 (dd, 1H), 6.93-6.98 (m, 1H), 7.07-7.15 (m, 2H), 7.71 (d, 1H).

### Intermediate 4-5

### 6-(3,5-Difluorophenyl)-2-methoxy-6,7,8,9-tetrahydro-5H-benzo[7]annulen-5-one

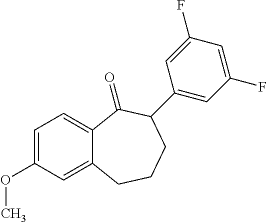 [SMILES: COc1ccc2c(c1)CCCC(c1cc(F)cc(F)c1)C2=O]

23 g (120.9 mmol) of 2-methoxy-6,7,8,9-tetrahydro-5H-benzo[7]annulen-5-one was reacted according to general specification 5-vPd with 23.33 g (120.9 mmol) of 1-bromo-3,5-fluorobenzene. It was stirred under reflux for 16 hours. The residue was purified using Silica Gel 60 (solvent: hexane, hexane-acetone 95:5). 21 g (57% of theor.) of product was isolated.

¹H-NMR (400 MHz, chloroform-d₁): δ=1.76-1.91 (m, 1H), 2.06-2.24 (m, 3H), 2.90-2.99 (m, 1H), 3.06-3.15 (m, 1H), 3.86 (s, 3H), 4.02 (dd, 1H), 6.68-6.85 (m, 5H), 7.72 (d, 1H).

### Intermediate 5-5

### 6-(2,5-Difluorophenyl)-2-methoxy-6,7,8,9-tetrahydro-5H-benzo[7]annulen-5-one

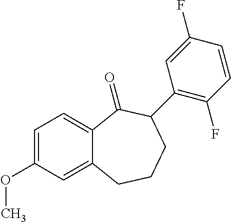 [SMILES: COc1ccc2c(c1)CCCC(c1cc(F)ccc1F)C2=O]

24.63 g (129.5 mmol) of 2-methoxy-6,7,8,9-tetrahydro-5H-benzo[7]annulen-5-one was reacted according to general specification 5-vPd with 25 g (129.5 mmol) of 1-bromo-2,5-fluorobenzene. It was stirred for 30 hours under reflux, overnight at room temperature and again for 3 hours under reflux. The residue was purified using Silica Gel 60 (solvent: hexane, hexane-ethyl acetate 95:5, 94:6, 93:7, 92:8, 90:10 and 80:20). 9.53 g (24% of theor.) of product was isolated. The intermediate fractions were purified again using Silica Gel 60 (solvent: hexane-ethyl acetate 95:5, 93:7 and 90:10). A further 7.55 g (19% of theor.) of product was obtained.

¹H-NMR (300 MHz, chloroform-d₁): δ=1.76-1.92 (m, 1H), 1.99-2.27 (m, 3H), 2.94 (dt, 1H), 3.15 (mc, 1H), 3.86 (s, 3H), 4.23 (dd, 1H), 6.76 (d, 1H), 6.84 (dd, 1H), 6.87-7.08 (m, 3H), 7.76 (d, 1H).

The following intermediates were prepared similarly by reaction of 2-methoxy-6,7,8,9-tetrahydro-5H-benzo[7]annulen-5-one with aryl halides.

| | | Name of | Example of | |
|---|---|---|---|---|
| Intermediate | Aryl halide | intermediate | structure | Analytical data |
| 6-5 | 1-bromo- 4-fluorobenzene | 6-(4- fluorophenyl)-2- methoxy- 6,7,8,9- tetrahydro-5H- benzo[7]annulen- 5-one | 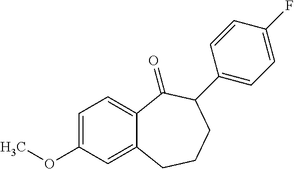 [SMILES: COc1ccc2c(c1)CCCC(c1ccc(F)cc1)C2=O] | C₁₈H₁₇FO₂ (284.3). ¹H- NMR (300 MHz, chloroform-d₁): δ = 1.74-1.91 (m, 1H), 2.01-2.25 (m, 3H), 2.89-3.01 (m, 1H), 3.06-3.14 (m, 1H), 3.86 (s, 3H), 4.04 (dd, 1H), 6.74-6.78 (m, 1H), 6.78-6.85 (m, 1H), 6.98-7.07 (m, 2H), 7.19-7.26 (m, 2H), 7.71 (d, 1H). |
| 7-5 | 1-bromo- 3-fluorobenzene | 6-(3- fluorophenyl)-2- methoxy- 6,7,8,9- tetrahydro-5H- benzo[7]annulen- 5-one | 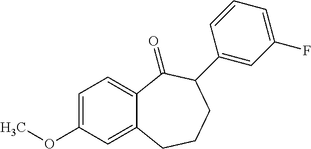 [SMILES: COc1ccc2c(c1)CCCC(c1cccc(F)c1)C2=O] | C₁₈H₁₇FO₂ (284.3). ¹H- NMR (raw product, selected signals, 400 MHz, DMSO-d₆): δ = 1.55-1.68 (m, 1H), 2.87-2.95 (m, 1H), 3.09-3.20 (m, 1H), 3.79 (s, 3H), 4.22 (dd, 1H), 6.84-6.91 (m, 2H), 7.27-7.34 (m, 1H), 7.54 (d, 1H). |
| 8-5 | 1-bromo-2- fluorobenzene | 6-(2- fluorophenyl)-2- methoxy- 6,7,8,9- tetrahydro-5H- benzo[7]annulen- 5-one | 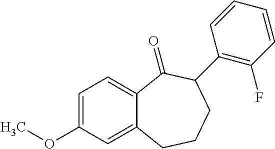 [SMILES: COc1ccc2c(c1)CCCC(c1ccccc1F)C2=O] | C₁₈H₁₇FO₂ (284.3). ¹H- NMR (raw product, selected signals, 300 MHz, DMSO-d₆): δ = 3.79 (s, 3H), 4.21 (dd, 1H). |
| 9-5 | 1-bromo- 2,4- difluorobenzene | 6-(2,4- difluorophenyl)- 2-methoxy- 6,7,8,9- tetrahydro-5H- benzo[7]annulen- 5-one | 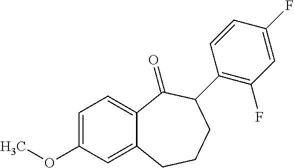 [SMILES: COc1ccc2c(c1)CCCC(c1ccc(F)cc1F)C2=O] | ¹H-NMR (300 MHz, DMSO-d₆): δ = 1.60- 1.70 (m, 1H), 1.87- 1.95 (m, 1H), 2.00- 2.14 (m, 2H), 2.87- 2.92 (m, 1H), 3.11- 3.19 (m, 1H), 3.79 (s, 3H), 4.20-4.24 (m, 1H), 6.86-6.88 (m, 2H), 7.00-7.05 (m, 1H), 7.10-7.16 (m, 1H), 7.34-7.40 (m, 1H), 7.55-7.58 (m, 1H) |

### Examples for Intermediate 6

General specification 6-1 for the preparation of 6 under argon atmosphere: 1 g ketone was dissolved in 4.5-12.5 mL of anhydrous tetrahydrofuran and 1.2 equivalent of 2,3,4,6,7,8,9,10-octahydropyrimido[1,2-a]azepine was added at 3° C. At this temperature, 1.2 equivalent of 1,1,2,2,3,3,4,4,4-nonafluorobutane-1-sulphonyl fluoride in anhydrous tetrahydrofuran (1 g in 0.6-4.5 mL) was added dropwise. It was stirred for a further 2 hours at 3° C. and overnight at room temperature. Then it was poured into saturated sodium hydrogen carbonate solution (10-20 mL solution per 1 g ketone), and extracted three times with methyl tert-butyl ether (approx. 10-20 mL per 1 g ketone). The combined organic phases were washed twice with saturated sodium chloride solution (approx. 5-20 mL per 1 g ketone), dried over magnesium sulphate and evaporated to dryness. Pentane was added to the residue and it was stirred for one hour at room temperature. It was filtered with suction, washed again with pentane and dried in a drying cabinet at room temperature.

General specification 6-2 for the preparation of 6 under argon atmosphere: 1 g ketone was dissolved in 5-7.5 mL of anhydrous tetrahydrofuran/tert-butyl methyl ether (1:1 to 4:3) and 2.4 equivalents of 2,3,4,6,7,8,9,10-octahydropyrimido[1.2-a]azepine were added. At this temperature, 2.4 equivalents of 1,1,2,2,3,3,4,4,4-nonafluorobutane-1-sulphonyl fluoride in anhydrous tetrahydrofuran (1 g in 1 mL) were added dropwise. It was stirred for a further 3 hours at 3° C. It was allowed to reach room temperature, saturated potassium carbonate solution was added, the phases were separated and the aqueous phase was shaken twice with tert-butyl methyl ether. The combined organic phases were dried over sodium sulphate and evaporated to dryness.

### Intermediate 1-6

### 8-(3,4-Difluorophenyl)-3-methoxy-6,7-dihydro-5H-benzo[7]annulen-9-yl-1,1,2,2,3,3,4,4,4-nonafluorobutane-1-sulphonate

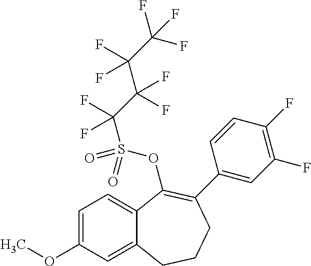 [SMILES: COc1ccc2c(c1)CCCC(c1ccc(F)c(F)c1)=C2OS(=O)(=O)C(F)(F)C(F)(F)C(F)(F)C(F)(F)F]

12.40 g (41.0 mmol) of 6-(3,4-difluorophenyl)-2-methoxy-6,7,8,9-tetrahydro-5H-benzo[7]annulen-5-one was reacted according to general specification 6-2. 23.80 g (99% of theor.) of raw product was isolated.

¹H-NMR (300 MHz, chloroform-d₁): δ=2.23 (t, 2H), 2.39 (quin, 2H), 2.84 (t, 2H), 3.86 (s, 3H), 6.83 (d, 1H), 6.88 (dd, 1H), 7.15-7.30 (m, 3H), 7.44 (d, 1H).

### Intermediate 2-6

### 8-(3,5-Difluorophenyl)-3-methoxy-6,7-dihydro-5H-benzo[7]annulen-9-yl-1,1,2,2,3,3,4,4,4-nonafluorobutane-1-sulphonate

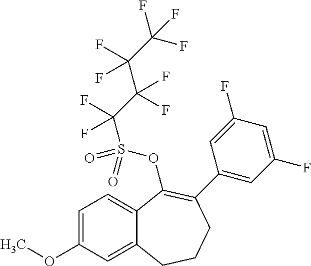 [SMILES: COc1ccc2c(c1)CCCC(c1cc(F)cc(F)c1)=C2OS(=O)(=O)C(F)(F)C(F)(F)C(F)(F)C(F)(F)F]

12.50 g (41.3 mmol) of 6-(3,5-difluorophenyl)-2-methoxy-6,7,8,9-tetrahydro-5H-benzo[7]annulen-5-one was reacted according to general specification 6-2. 24.00 g (99% of theor.) of raw product was isolated.

¹H-NMR (400 MHz, chloroform-d₁): δ=2.23 (t, 2H), 2.40 (quin, 2H), 2.84 (t, 2H), 3.86 (s, 3H), 6.75-6.85 (m, 2H), 6.89 (dd, 1H), 6.93-7.00 (m, 2H), 7.45 (d, 1H).

### Intermediate 3-6

### 4-Fluoro-8-(4-fluorophenyl)-3-methoxy-6,7-dihydro-5H-benzo[7]annulen-9-yl-1,1,2,2,3,3,4,4,4-nonafluorobutane-1-sulphonate

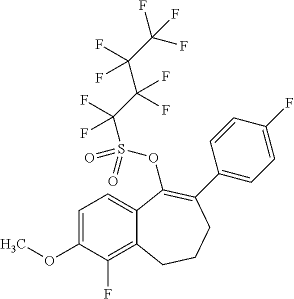 [SMILES: COc1ccc2c(c1F)CCCC(c1ccc(F)cc1)=C2OS(=O)(=O)C(F)(F)C(F)(F)C(F)(F)C(F)(F)F]

19.00 g (62.8 mmol) of 1-fluoro-6-(4-fluorophenyl)-2-methoxy-6,7,8,9-tetrahydro-5H-benzo[7]annulen-5-one was reacted according to general specification 6-1. 36.00 g (98% of theor.) of raw product was isolated.

¹H-NMR (400 MHz, chloroform-d₁): δ=2.24 (t, 2H), 2.37 (quin, 2H), 2.94 (dt, 2H), 3.94 (s, 3H), 6.93 (t, 1H), 7.07-7.13 (m, 2H), 7.25-7.30 (m, 1H), 7.37-7.44 (m, 2H).

### Intermediate 4-6

### 8-(2,5-Difluorophenyl)-3-methoxy-6,7-dihydro-5H-benzo[7]annulen-9-yl-1,1,2,2,3,3,4,4,4-nonafluorobutane-1-sulphonate

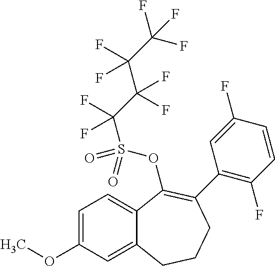 [SMILES: COc1ccc2c(c1)CCCC(c1cc(F)ccc1F)=C2OS(=O)(=O)C(F)(F)C(F)(F)C(F)(F)C(F)(F)F]

15.5 g (51.3 mmol) of 6-(2,5-difluorophenyl)-2-methoxy-6,7,8,9-tetrahydro-5H-benzo[7]annulen-5-one was reacted according to general specification 6-1, but without treating with pentane. 33.81 g (113% of theor.) of raw product was isolated.

¹H-NMR (300 MHz, chloroform-d₁): δ=2.20 (t, 2H), 2.39 (quin, 2H), 2.86 (t, 2H), 3.86 (s, 3H), 6.84 (d, 1H), 6.88 (dd, 1H), 6.97-7.14 (m, 3H), 7.46 (d, 1H).

### Intermediate 5-6

### 2-Fluoro-8-(4-fluorophenyl)-3-methoxy-6,7-dihydro-5H-benzo[7]annulen-9-yl-1,1,2,2,3,3,4,4,4-nonafluorobutane-1-sulphonate

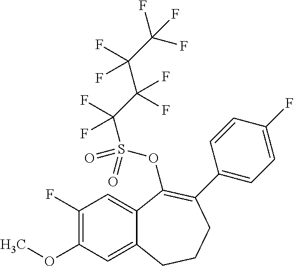 [SMILES: COc1cc2c(cc1F)C(OS(=O)(=O)C(F)(F)C(F)(F)C(F)(F)C(F)(F)F)=C(c1ccc(F)cc1)CCC2]

32.1 g (106.2 mmol) of 3-fluoro-6-(4-fluorophenyl)-2-methoxy-6,7,8,9-tetrahydro-5H-benzo[7]annulen-5-one was reacted according to general specification 6-1. It was stirred for a further 3 days at room temperature. A further 0.42 equivalent of 2,3,4,6,7,8,9,10-octahydropyrimido[1,2-a]azepine and 0.40 equivalent of 1,1,2,2,3,3,4,4,4-nonafluorobutane-1-sulphonyl fluoride were added and it was stirred for a further 2 hours at room temperature. It was worked up as described in specification 6-1, but without treating with pentane. 71.5 g (115% of theor.) of raw product was isolated.

¹H-NMR (400 MHz, chloroform-d₁): δ=2.24 (t, 2H), 2.40 (quin, 2H), 2.83 (t, 2H), 3.95 (s, 3H), 6.87 (d, 1H), 7.10 (tt, 2H), 7.22 (d, 1H), 7.40 (mc, 2H).

### Intermediate 6-6

### 8-(4-Fluorophenyl)-3-methoxy-6,7-dihydro-5H-benzo[7]annulen-9-yl-1,1,2,2,3,3,4,4,4-nonafluorobutane-1-sulphonate

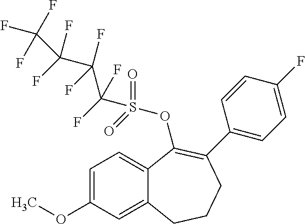 [SMILES: COc1ccc2c(c1)CCCC(c1ccc(F)cc1)=C2OS(=O)(=O)C(F)(F)C(F)(F)C(F)(F)C(F)(F)F]

13.5 g (47 mmol) of 6-(4-fluoro-phenyl)-2-methoxy-6,7,8,9-tetrahydro-benzocyclohepten-5-one was put in 100 mL THF, and 10.6 mL DBU (1,8-diazabicyclo[5.4.0]undec-7-ene) and 12.8 mL perfluorobutane-1-sulphonic acid fluoride diluted with 20 mL THF were added dropwise, with cooling on an ice bath. It was stirred for 2 hours with cooling on an ice bath and for 19 hours at room temperature. Saturated sodium hydrogen carbonate solution was added, the phases were separated and the aqueous phase was extracted twice with ethyl acetate. The combined organic phases were washed with water and saturated sodium chloride solution. It was dried over sodium sulphate, filtered, concentrated by evaporation and dried in vacuum. 37 g of a residue was obtained, which was reacted further without analysis.

The following intermediates were prepared similarly:

| | | Structure of |
|---|---|---|
| Intermediate | Name of intermediate | intermediate |
| 7-6 | 8-(3-fluorophenyl)-3-methoxy-6,7- dihydro-5H-benzo[7]annulen-9-yl- 1,1,2,2,3,3,4,4,4-nonafluorobutane- 1-sulphonate | 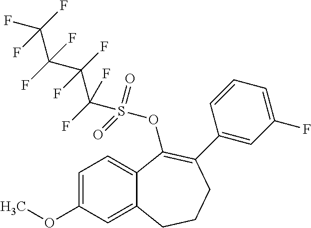 [SMILES: COc1ccc2c(c1)CCCC(c1cccc(F)c1)=C2OS(=O)(=O)C(F)(F)C(F)(F)C(F)(F)C(F)(F)F] |
| 8-6 | 8-(2-fluorophenyl)-3-methoxy-6,7- dihydro-5H-benzo[7]annulen-9-yl- 1,1,2,2,3,3,4,4,4-nonafluorobutane- 1-sulphonate | 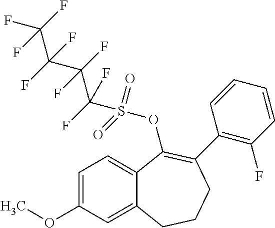 [SMILES: COc1ccc2c(c1)CCCC(c1ccccc1F)=C2OS(=O)(=O)C(F)(F)C(F)(F)C(F)(F)C(F)(F)F] |
| 9-6 | 8-(2,4-difluorophenyl)-3-methoxy- 6,7-dihydro-5H-benzo[7]annulen-9- yl-1,1,2,2,3,3,4,4,4- nonafluorobutane-1-sulphonate | 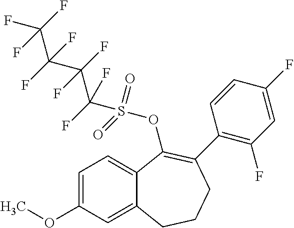 [SMILES: COc1ccc2c(c1)CCCC(c1ccc(F)cc1F)=C2OS(=O)(=O)C(F)(F)C(F)(F)C(F)(F)C(F)(F)F] |

### Intermediates for 7

General specification 7 for the preparation of 7 under argon atmosphere and with exclusion of moisture: 1 g nonaflatenol ether was dissolved in approx. 8-13 mL of anhydrous N,N-dimethylformamide. 2.5-2.6 equivalents of alkynol, 4.1 equivalents of triethylamine and 0.033 equivalent of tetrakis-(triphenylphosphine)-palladium(0) were added. It was stirred for 0.5-1.5 hours at 80° C. The reaction mixture was cooled and the volatile constituents were removed in oil pump vacuum on a rotary evaporator. The residue was taken up in ethyl acetate and washed three times with water. It was dried over magnesium sulphate or sodium sulphate and evaporated to dryness. The residue was purified using Silica Gel 60.

### Intermediate 1-7

### 6-[8-(3,4-Difluorophenyl)-3-methoxy-6,7-dihydro-5H-benzo[7]annulen-9-yl]hex-5-yn-1-ol

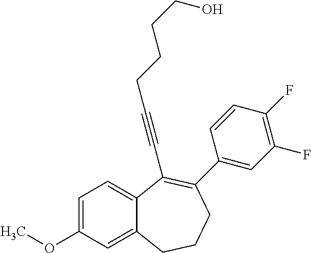 [SMILES: COc1ccc2c(c1)CCCC(c1ccc(F)c(F)c1)=C2C#CCCCCO]

23.8 g (40.7 mmol) of 8-(3,4-difluorophenyl)-3-methoxy-6,7-dihydro-5H-benzo[7]annulen-9-yl-1,1,2,2,3,3,4,4,4-nonafluorobutane-1-sulphonate and 11.3 mL (102.5 mmol) hex-5-yn-1-ol was reacted according to general specification 7. The residue was purified using Silica Gel 60 (solvent: hexane, hexane-ethyl acetate 8:2, 6:4 and 1:1). 12.9 g (83% of theor.) of product was isolated.

¹H-NMR (300 MHz, chloroform-d₁): δ=1.47-1.65 (m, 4H), 2.15-2.37 (m, 6H), 2.66 (t, 2H), 3.54-3.67 (m, 2H), 3.84 (s, 3H), 6.75 (d, 1H), 6.84 (dd, 1H), 7.13 (mc, 1H), 7.27-7.34 (m, 1H), 7.46-7.57 (m, 2H).

### Intermediate 2-7

### 6-[8-(3,5-Difluorophenyl)-3-methoxy-6,7-dihydro-5H-benzo[7]annulen-9-yl]hex-5-yn-1-ol

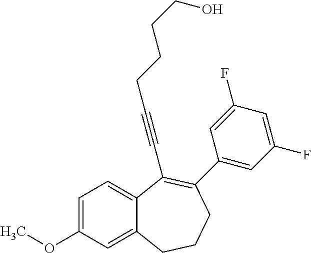 [SMILES: COc1ccc2c(c1)CCCC(c1cc(F)cc(F)c1)=C2C#CCCCCO]

24.0 g (41.1 mmol) of 8-(3,5-difluorophenyl)-3-methoxy-6,7-dihydro-5H-benzo[7]annulen-9-yl-1,1,2,2,3,3,4,4,4-nonafluorobutane-1-sulphonate and 10.15 g (103.4 mmol) hex-5-yn-1-ol was reacted according to general specification 7. The residue was purified using Silica Gel 60 (solvent: hexane, hexane-ethyl acetate 8:2, 6:4 and 1:1). 10.6 g (67% of theor.) of product was isolated.

¹H-NMR (400 MHz, chloroform-d₁): δ=1.52-1.64 (m, 4H), 2.18-2.39 (m, 6H), 2.67 (t, 2H), 3.62 (mc, 2H), 3.84 (s, 3H), 6.69-6.77 (m, 2H), 6.84 (dd, 1H), 7.18 (mc, 2H), 7.49 (d, 1H).

### Intermediate 3-7

### 6-[4-Fluoro-8-(4-fluorophenyl)-3-methoxy-6,7-dihydro-5H-benzo[7]annulen-9-yl]hex-5-yn-1-ol

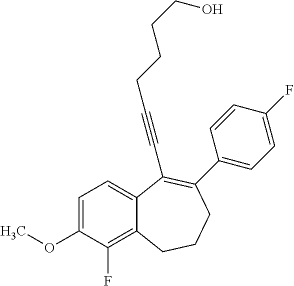 [SMILES: COc1ccc2c(c1F)CCCC(c1ccc(F)cc1)=C2C#CCCCCO]

36.00 g (61.6 mmol) of 4-fluoro-8-(4-fluorophenyl)-3-methoxy-6,7-dihydro-5H-benzo[7]annulen-9-yl-1,1,2,2,3,3,4,4,4-nonafluorobutane-1-sulphonate and 15.22 g (155.1 mmol) hex-5-yn-1-ol was reacted according to general specification 7. The residue was purified using Silica Gel 60 (solvent: hexane, hexane-ethyl acetate 8:2, 6:4 and 1:1). 10.1 g (43% of theor.) of product was isolated.

¹H-NMR (400 MHz, chloroform-d₁): δ=1.17 (mc, 1H), 1.48-1.60 (m, 4H), 2.20 (quin, 2H), 2.26-2.35 (m, 4H), 2.78 (dt, 2H), 3.60 (mc, 2H), 3.91 (s, 3H), 6.88 (t, 1H), 7.02-7.08 (m, 2H), 7.30 (dd, 1H), 7.55-7.61 (m, 2H).

### Intermediate 4-7

### 6-[8-(2,5-Difluorophenyl)-3-methoxy-6,7-dihydro-5H-benzo[7]annulen-9-yl]hex-5-yn-1-ol

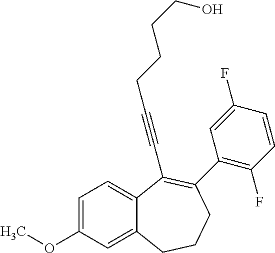 [SMILES: COc1ccc2c(c1)CCCC(c1cc(F)ccc1F)=C2C#CCCCCO]

33.0 g (56.5 mmol) of 8-(2,5-difluorophenyl)-3-methoxy-6,7-dihydro-5H-benzo[7]annulen-9-yl-1,1,2,2,3,3,4,4,4-nonafluorobutane-1-sulphonate and 14.21 g (144.8 mmol) hex-5-yn-1-ol was reacted according to general specification 7. The residue was purified using Silica Gel 60 (solvent: hexane, hexane-ethyl acetate 9:1, 8:2 and 1:1). 12.55 g (58% of theor.) of product was isolated.

¹H-NMR (300 MHz, chloroform-d₁): δ=1.49 (mc, 4H), 2.17-2.32 (m, 6H), 2.70 (mc, 2H), 3.58 (mc, 2H), 3.84 (s, 3H), 6.77 (d, 1H), 6.84 (dd, 1H), 6.90-6.99 (m, 1H), 7.04 (dt, 1H), 7.21-7.28 (m, 1H), 7.49 (d, 1H).

### Intermediate 5-7

### 6-[2-Fluoro-8-(4-fluorophenyl)-3-methoxy-6,7-dihydro-5H-benzo[7]annulen-9-yl]hex-5-yn-1-ol

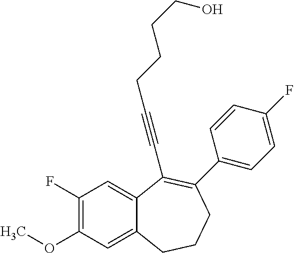 [SMILES: COc1cc2c(cc1F)C(C#CCCCCO)=C(c1ccc(F)cc1)CCC2]

71.5 g (122.3 mmol) of 2-fluoro-8-(4-fluorophenyl)-3-methoxy-6,7-dihydro-5H-benzo[7]annulen-9-yl-1,1,2,2,3,3,4,4,4-nonafluorobutane-1-sulphonate and 30.86 g (314.4 mmol) hex-5-yn-1-ol was reacted according to general specification 7. The residue was purified using Silica Gel 60 (solvent: hexane, hexane-ethyl acetate 9:1, 8:2 and 1:1). 13.94 g (30% of theor.) of product was isolated.

¹H-NMR (300 MHz, chloroform-d₁): δ=1.46-1.63 (m, 4H), 2.16-2.37 (m, 6H), 2.64 (t, 2H), 3.60 (mc, 2H), 3.92 (s, 3H), 6.79 (d, 1H), 7.05 (mc, 2H), 7.30 (d, 1H), 7.58 (mc, 2H).

The following intermediates were prepared similarly to general specification 7, optionally with addition of 0.4 equivalent of copper(I) iodide:

| | Name of | Structure of | |
|---|---|---|---|
| Intermediate | intermediate | intermediate | Analytical data |
| 6-7 | 6-[8-(4- fluorophenyl)-3- methoxy-6,7- dihydro-5H- benzo[7]annulen- 9-yl]hex-5-yn-1- ol | 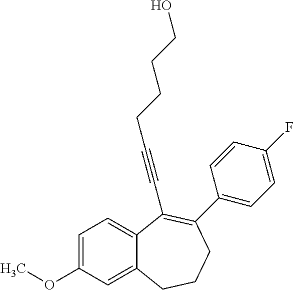 [SMILES: COc1ccc2c(c1)CCCC(c1ccc(F)cc1)=C2C#CCCCCO] | C₂₄H₂₅FO₂ (364.5). 1H- NMR (selected signals, 400 MHz, chloroform-d₁): δ = 1.58-1.76 (m), 2.17- 2.36 (m), 2.67 (t, 3H), 3.56- 3.61 (m, 2H), 3.68 (t, 1H), 3.38 (s, 3H), 6.76 (d, 1H), 6.83 (dd, 1H), 7.01- 7.09 (m, 2H), 7.01-7.09 (m, 2H), 7.49 (d, 1H), 7.56- 7.63 (m, 2H). |
| 7-7 | 6-[8-(3- fluorophenyl)-3- methoxy-6,7- dihydro-5H- benzo[7]annulen- 9-yl]hex-5-yn-1- ol | 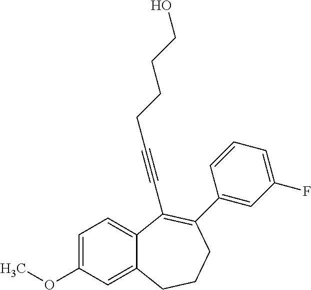 [SMILES: COc1ccc2c(c1)CCCC(c1cccc(F)c1)=C2C#CCCCCO] | MS (ESIpos): Mass found = 364.00 |
| 8-7 | 6-[8-(2- fluorophenyl)-3- methoxy-6,7- dihydro-5H- benzo[7]annulen- 9-yl]hex-5-yn-1- ol | 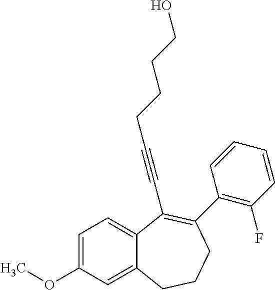 [SMILES: COc1ccc2c(c1)CCCC(c1ccccc1F)=C2C#CCCCCO] | C₂₄H₂₅FO₂ (364.5). ¹H- NMR (selected signals, 300 MHz, DMSO-d₆): δ = 1.21-1.35 (m, 4H), 2.06- 2.20 (m, 6H), 2.55-2.66 (m, 2H), 3.74 (s, 3H), 4.28 (t, 1H), 6.80-6.88 (2H), 7.15-7.23 (m, 2H), 7.27- 7.37 (m, 2H), 7.45 (mc, 1H). |
| 9-7 | 5-[8-(4- fluorophenyl)-3- methoxy-6,7- dihydro-5H- benzo[7]annulen- 9-yl]pent-4-yn-1- ol | 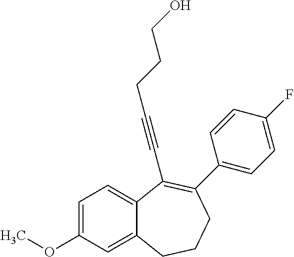 [SMILES: COc1ccc2c(c1)CCCC(c1ccc(F)cc1)=C2C#CCCCO] | C₂₃H₂₃FO₂ (350.4). MS (ESIpos): Mass found = 350.0. |
| 10-7 | 5-[8-(2,4- difluorophenyl)- 3-methoxy-6,7- dihydro-5H- benzo[7]annulen- 9-yl]hex-5-yn-1- ol | 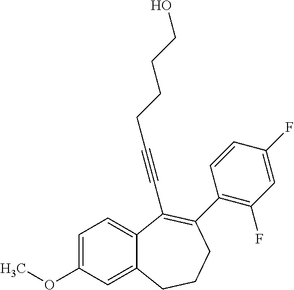 [SMILES: COc1ccc2c(c1)CCCC(c1ccc(F)cc1F)=C2C#CCCCCO] | ¹H-NMR (300 MHz, DMSO-d₆): δ = 1.22-1.33 (m, 4H), 2.09-2.17 (m, 6H), 2.58-2.61 (m, 2H), 3.24-3.29 (m, 2H), 3.74 (s, 3H), 4.31 (t, 1H), 6.82- 6.85 (m, 2H), 7.04-7.08 (m, 1H), 7.19-7.25 (m, 1H), 7.34 (d, 1H), 7.46- 7.52 (m, 1H). |

### Intermediates for 8

### Intermediate 1-8

### 6-[8-(3,4-Difluorophenyl)-3-methoxy-6,7-dihydro-5H-benzo[7]annulen-9-yl]hexan-1-ol

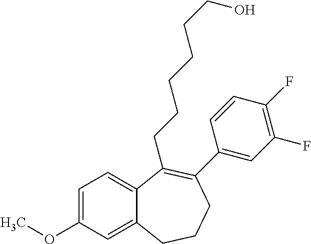 [SMILES: COc1ccc2c(c1)CCCC(c1ccc(F)c(F)c1)=C2CCCCCCO]

11.8 g (30.9 mmol) of 6-[8-(3,4-difluorophenyl)-3-methoxy-6,7-dihydro-5H-benzo[7]annulen-9-yl]hex-5-yn-1-ol and 1.41 g of 5 wt. % palladium on activated charcoal in 300 mL of 0.2% methanolic solution of potassium hydroxide were hydrogenated at room temperature and normal pressure. It was filtered on Celite with suction, washed with methanol again and concentrated by evaporation. The residue was taken up in dichloromethane and washed three times with water, dried over magnesium sulphate and concentrated by evaporation. 11.3 g was obtained (83% of theor.)

¹H-NMR (300 MHz, chloroform-d₁): δ=1.09-1.29 (m, 6H), 1.43 (quin, 2H), 2.01-2.18 (m, 4H), 2.37 (t, 2H), 2.64 (t, 2H), 3.54 (mc, 2H), 3.84 (s, 3H), 6.77 (d, 1H), 6.82 (dd, 1H), 6.92-6.98 (m, 1H), 7.05 (ddd, 1H), 7.13 (mc, 1H), 7.22 (d, 1H).

### Intermediate 2-8

### 6-[8-(3,5-Difluorophenyl)-3-methoxy-6,7-dihydro-5H-benzo[7]annulen-9-yl]hexan-1-ol

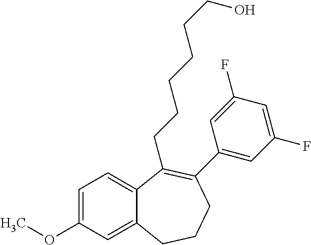 [SMILES: COc1ccc2c(c1)CCCC(c1cc(F)cc(F)c1)=C2CCCCCCO]

10.0 g (26.1 mmol) of 6-[8-(3,5-difluorophenyl)-3-methoxy-6,7-dihydro-5H-benzo[7]annulen-9-yl]hex-5-yn-1-ol and 1.195 g of 5 wt. % palladium on activated charcoal in 300 mL of 0.2% methanolic solution of potassium hydroxide were hydrogenated at room temperature and normal pressure. It was filtered on Celite with suction, washed with methanol again and concentrated by evaporation. The residue was taken up in dichloromethane and washed three times with water, dried over magnesium sulphate and concentrated by evaporation. 10.1 g (100% of theor.) of product was obtained.

¹H-NMR (300 MHz, chloroform-d₁): δ=1.10-1.31 (m, 6H), 1.44 (quin, 2H), 2.01-2.18 (m, 4H), 2.38 (t, 2H), 2.64 (t, 2H), 3.55 (mc, 2H), 3.84 (s, 3H), 6.66-6.85 (m, 5H), 7.22 (d, 1H).

### Intermediate 3-8

### 6-[4-Fluoro-8-(4-fluorophenyl)-3-methoxy-6,7-dihydro-5H-benzo[7]annulen-9-yl]hexan-1-ol

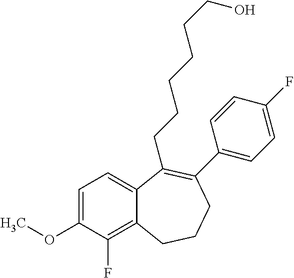 [SMILES: COc1ccc2c(c1F)CCCC(c1ccc(F)cc1)=C2CCCCCCO]

10.0 g (26.1 mmol) of 6-[4-fluoro-8-(4-fluorophenyl)-3-methoxy-6,7-dihydro-5H-benzo[7]annulen-9-yl]hex-5-yn-1-ol and 1.2 g of 5 wt. % palladium on activated charcoal in 300 mL of 0.2% methanolic solution of potassium hydroxide were hydrogenated at room temperature and normal pressure. It was filtered on Celite with suction, washed with methanol again and concentrated by evaporation. The residue was taken up in dichloromethane and washed three times with water, dried over magnesium sulphate and concentrated by evaporation. 10.1 g (99% of theor.) of product was obtained.

¹H-NMR (300 MHz, chloroform-d₁): δ=1.07-1.29 (m, 6H), 1.37-1.50 (m, 2H), 2.02-2.17 (m, 4H), 2.31-2.41 (m, 2H), 2.70-2.81 (m, 2H), 3.49-3.60 (m, 2H), 3.91 (s, 3H), 6.86 (t, 1H), 6.99-7.10 (m, 3H), 7.14-7.23 (m, 2H).

### Intermediate 4-8

### 6-[8-(2,5-Difluorophenyl)-3-methoxy-6,7-dihydro-5H-benzo[7]annulen-9-yl]hexan-1-ol

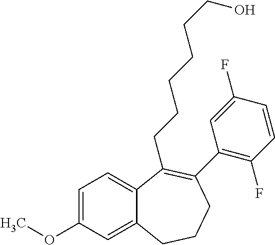 [SMILES: COc1ccc2c(c1)CCCC(c1cc(F)ccc1F)=C2CCCCCCO]

12.5 g (36.7 mmol) of 6-[8-(2,5-difluorophenyl)-3-methoxy-6,7-dihydro-5H-benzo[7]annulen-9-yl]hex-5-yn-1-ol and 1.2 g of 5 wt. % palladium on activated charcoal in 250 mL of 0.2% methanolic solution of potassium hydroxide were hydrogenated at room temperature and normal pressure. It was filtered on Celite with suction, washed with methanol again and concentrated by evaporation. The residue was taken up in dichloromethane and washed three times with water, dried over magnesium sulphate and concentrated by evaporation. 10.62 g (84% of theor.) of product was obtained.

¹H-NMR (300 MHz, chloroform-d₁): δ=1.08-1.25 (m, 6H), 1.42 (m, 2H), 2.00-2.21 (m, 4H), 2.32 (t, 2H), 2.68 (t, 2H), 3.53 (t, 2H), 3.84 (s, 3H), 6.77-6.84 (m, 2H), 6.87-6.97 (m, 2H), 6.99-7.08 (m, 1H), 7.23 (d, 1H).

### Intermediate 5-8

### 6-[2-Fluoro-8-(4-fluorophenyl)-3-methoxy-6,7-dihydro-5H-benzo[7]annulen-9-yl]hexan-1-ol

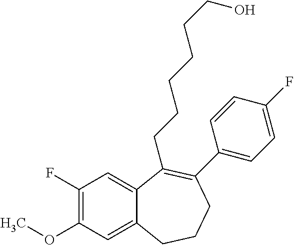 [SMILES: COc1cc2c(cc1F)C(CCCCCCO)=C(c1ccc(F)cc1)CCC2]

13.8 g (36.1 mmol) of 6-[2-fluoro-8-(4-fluorophenyl)-3-methoxy-6,7-dihydro-5H-benzo[7]annulen-9-yl]hex-5-yn-1-ol and 1.38 g of 5 wt. % palladium on activated charcoal in 275 mL of 0.2% methanolic solution of potassium hydroxide were hydrogenated at room temperature and normal pressure. It was filtered on Celite with suction and hydrogenated further with 0.5 g of 5 wt. % palladium on activated charcoal. It was filtered on Celite with suction, washed with methanol again and evaporated to dryness. The residue was taken up in dichloromethane, washed three times with water, dried over magnesium sulphate and concentrated by evaporation. 17.22 g (124% of theor.) of product was obtained.

¹H-NMR (400 MHz, chloroform-d₁): δ=1.08-1.28 (m, 6H), 1.43 (mc, 2H), 2.04-2.18 (m, 4H), 2.32 (m, 2H), 2.62 (t, 2H), 3.54 (t, 2H), 3.93 (s, 3H), 6.82 (d, 1H), 7.01-7.08 (m, 3H), 7.19 (mc, 2H).

### Intermediate 6-8

### 6-[8-(4-Fluorophenyl)-3-methoxy-6,7-dihydro-5H-benzo[7]annulen-9-yl]hexan-1-ol

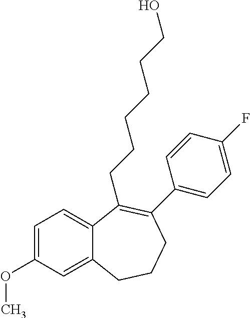 [SMILES: COc1ccc2c(c1)CCCC(c1ccc(F)cc1)=C2CCCCCCO]

90 mg palladium on calcium carbonate (10%) was added to 870 mg of 6-[8-(4-fluorophenyl)-3-methoxy-6,7-dihydro-5H-benzo[7]annulen-9-yl]hex-5-yn-1-ol in 30 mL THF and stirred under a hydrogen atmosphere. Then it was filtered on Celite, concentrated by evaporation and THF and 87 mg palladium on activated charcoal (10%) were added. Hydrogen was supplied. After filtration on Celite and removal of the solvent, the title compound was isolated as raw product. C₂₄H₂₉FO₂ (368.5). MS (ESIpos): m/z=369. ¹H-NMR (selected signals, 300 MHz, DMSO-d₆): δ 1.89-2.09 (m, 4H), 2.25-2.34 (m, 2H), 2.54-2.63 (m, 2H), 3.18-3.25 (m, 2H), 3.73 (s, 3H), 4.22 (t, 1H), 6.77-6.83 (m, 2H), 7.12-7.28 (m, 5H).

The following intermediates were prepared similarly:

| | Name of | Structure of | |
|---|---|---|---|
| Intermediate | intermediate | intermediate | Analytical data |
| 7-8 | 6-[8-(3- fluorophenyl)-3- methoxy-6,7- dihydro-5H- benzo[7]annulen- 9-yl]hexan-1-ol | 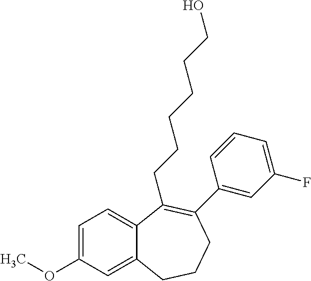 [SMILES: COc1ccc2c(c1)CCCC(c1cccc(F)c1)=C2CCCCCCO] | C₂₄H₂₉FO₂ (368.5). MS ESIpos = 368.0 (mass found). |
| 8-8 | 6-[8-(2- fluorophenyl)-3- methoxy-6,7- dihydro-5H- benzo[7]annulen- 9-yl]hexan-1-ol | 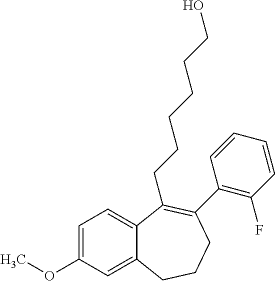 [SMILES: COc1ccc2c(c1)CCCC(c1ccccc1F)=C2CCCCCCO] | ¹H-NMR (selected signals, 300 MHz, DMSO-d₆): δ = 1.87-1.97 (m, 2H), 1.97- 2.10 (m, 2H), 2.17-2.28 (m, 2H), 2.57-2.66 (m, 2H), 3.16-3.25 (m, 2H), 3.74 (s, 3H), 4.20 (t, 1H), 6.78-6.85 (m, 2H), 7.14- 7.35 (m, 5H). |
| 9-8 | 5-[8-(4- fluorophenyl)-3- methoxy-6,7- dihydro-5H- benzo[7]annulen- 9-yl]pentan-1-ol | 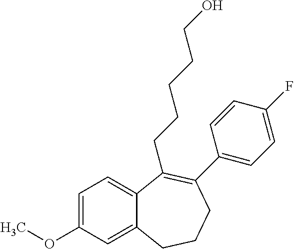 [SMILES: COc1ccc2c(c1)CCCC(c1ccc(F)cc1)=C2CCCCCO] | ¹H-NMR (selected signals, 300 MHz, DMSO-d₆): δ = 2.58 (t, 2H), 3.15-3.23 (m, 2H), 3.73 (s, 3H), 4.20 (t, 1H), 6.77-6.82 (m, 2H), 7.12-7.28 (m, 5H). |
| 10-8 | 6-[8-(2,4- difluorophenyl)- 3-methoxy-6,7- dihydro-5H- benzo[7]annulen- 9-yl]hexan-1-ol | 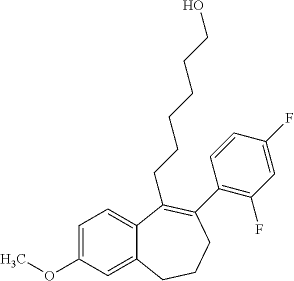 [SMILES: COc1ccc2c(c1)CCCC(c1ccc(F)cc1F)=C2CCCCCCO] | ¹H-NMR (300 MHz, DMSO-d₆): δ = 1.03-1.11 (m, 6H), 1.20-1.26 (m, 2H), 1.91-1.95 (m, 2H), 2.03-2.10 (m, 2H), 2.23- 2.26 (m, 2H), 2.62-2.64 (m, 2H), 3.23-3.27 (m, 2H), 3.77 (s, 3H), 4.25 (t, 1H), 6.83-6.85 (m, 2H), 7.08-7.13 (m, 1H), 7.24- 7.35 (m, 3H) |

### Intermediates for 9

General specification 9 for the preparation of 9 under protective gas atmosphere and with exclusion of moisture: 3.5 equivalents of 2,6-dimethylpyridine in dichloromethane (approx. 4.4-5.5 mL/g) were added at 3-5° C. to 3.5 equivalents of boron tribromide (1 mmol boron tribromide in 1.5-4 mL dichloromethane). 1 equivalent of methyl ether dissolved in dichloromethane (4.3-6.1 mL/g) was added dropwise at 3-5° C. and stirred overnight at room temperature. It was poured onto ice water, the phases were separated and the aqueous phase was shaken three times with dichloromethane. The combined organic phases were washed with water, dried over magnesium sulphate and concentrated by evaporation.

### Intermediate 1-9

### 8-(3,4-Difluorophenyl)-9-(6-hydroxyhexyl)-6,7-dihydro-5H-benzo[7]annulen-3-ol

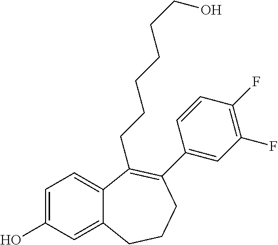 [SMILES: OCCCCCCC1=C(c2ccc(F)c(F)c2)CCCc2cc(O)ccc21]

11.5 g (29.76 mmol) of 6-[8-(3,4-difluorophenyl)-3-methoxy-6,7-dihydro-5H-benzo[7]annulen-9-yl]hexan-1-ol was reacted according to general specification 9. 11.16 g (99% of theor.) of product was obtained.

¹H-NMR (300 MHz, chloroform-d₁): δ=1.07-1.29 (m, 6H), 1.44 (quin, 2H), 2.00-2.17 (m, 4H), 2.35 (t, 2H), 2.60 (t, 2H), 3.56 (t, 2H), 6.71 (d, 1H), 6.74 (dd, 1H), 6.91-6.98 (m, 1H), 7.04 (ddd, 1H), 7.08-7.18 (m, 2H).

### Intermediate 2-9

### 8-(3,5-Difluorophenyl)-9-(6-hydroxyhexyl)-6,7-dihydro-5H-benzo[7]annulen-3-ol

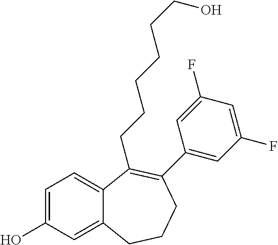 [SMILES: OCCCCCCC1=C(c2cc(F)cc(F)c2)CCCc2cc(O)ccc21]

10.0 g (25.87 mmol) of 6-[8-(3,5-difluorophenyl)-3-methoxy-6,7-dihydro-5H-benzo[7]annulen-9-yl]hexan-1-ol was reacted according to general specification 9. Hexane was added to the residue and it was filtered with suction. 9.3 g (97% of theor.) of product was obtained.

¹H-NMR (300 MHz, chloroform-d₁): δ=1.09-1.26 (m, 6H), 1.44 (mc, 2H), 2.02-2.18 (m, 4H), 2.37 (t, 2H), 2.61 (t, 2H), 3.55 (t, 2H), 6.66-6.80 (m, 5H), 7.16 (d, 1H).

### Intermediate 3-9

### 4-Fluoro-8-(4-fluorophenyl)-9-(6-hydroxyhexyl)-6,7-dihydro-5H-benzo[7]annulen-3-ol

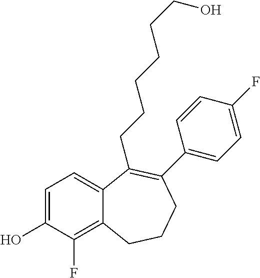 [SMILES: OCCCCCCC1=C(c2ccc(F)cc2)CCCc2c1ccc(O)c2F]

10.0 g (25.87 mmol) of 6-[4-fluoro-8-(4-fluorophenyl)-3-methoxy-6,7-dihydro-5H-benzo[7]annulen-9-yl]hexan-1-ol was reacted according to general specification 9. The precipitate was filtered with suction and washed with water. It was dried at 40° C. in a drying cabinet. The filtrate was shaken three times with dichloromethane. The combined organic phases were washed with water twice, dried over magnesium sulphate and concentrated by evaporation. Diisopropyl ether was added to the residue and it was filtered with suction. A total of 6.1 g (62% of theor.) of product was obtained.

¹H-NMR (300 MHz, chloroform-d₁): δ=1.06-1.27 (m, 6H), 1.43 (mc, 2H), 2.01-2.17 (m, 4H), 2.28-2.41 (m, 2H), 2.65-2.79 (m, 2H), 3.55 (t, 2H), 5.22 (s, 1H), 6.88 (t, 1H), 6.95-7.09 (m, 3H), 7.14-7.23 (m, 2H).

### Intermediate 4-9

### 8-(2,5-Difluorophenyl)-9-(6-hydroxyhexyl)-6,7-dihydro-5H-benzo[7]annulen-3-ol

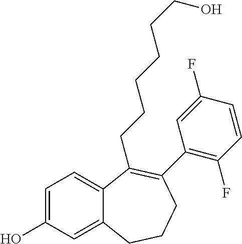 [SMILES: OCCCCCCC1=C(c2cc(F)ccc2F)CCCc2cc(O)ccc21]

10.6 g (27.4 mmol) of 6-[8-(2,5-difluorophenyl)-3-methoxy-6,7-dihydro-5H-benzo[7]annulen-9-yl]hexan-1-ol was reacted according to general specification 9. It was stirred overnight at room temperature, poured into ice/water and stirred for a further 1 hour. It was filtered with suction, and washed with a little dichloromethane and with water five times. It was dried at 40° C. in a drying cabinet. 9.55 g (93% of theor.) of product was obtained.

¹H-NMR (300 MHz, chloroform-d₁): δ=1.06-1.30 (m, 6H), 1.43 (mc, 2H), 1.99-2.19 (m, 4H), 2.31 (mc, 2H), 2.64 (t, 2H), 3.54 (t, 2H), 6.69-6.77 (m, 2H), 6.86-6.97 (m, 2H), 7.04 (dt, 1H), 7.17 (d, 1H).

### Intermediate 5-9

### 2-Fluoro-8-(4-fluorophenyl)-9-(6-hydroxyhexyl)-6,7-dihydro-5H-benzo[7]annulen-3-ol

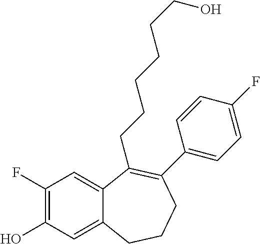 [SMILES: OCCCCCCC1=C(c2ccc(F)cc2)CCCc2cc(O)c(F)cc21]

12.38 g (32.0 mmol) of 6-[2-fluoro-8-(4-fluorophenyl)-3-methoxy-6,7-dihydro-5H-benzo[7]annulen-9-yl]hexan-1-ol was reacted with 4.0 equivalents according to general specification 9. It was stirred overnight at room temperature, poured into ice/water, stirred for a further 2 hours, filtered with suction and taken up in 1 litre of dichloromethane. It was washed three times with water, dried over magnesium sulphate and concentrated by evaporation. 12.75 g (107% of theor.) of product was obtained.

¹H-NMR (300 MHz, chloroform-d₁): δ=1.07-1.27 (m, 6H), 1.44 (mc, 2H), 2.02-2.16 (m, 4H), 2.31 (m, 2H), 2.58 (m, 2H), 3.55 (t, 2H), 5.38 (s, 1H), 6.84 (d, 1H), 6.98-7.09 (m, 3H), 7.14-7.22 (m, 2H).

The following intermediates were prepared similarly

| | Name of | Structure of | |
|---|---|---|---|
| Intermediate | intermediate | intermediate | Analytical data |
| 6-9 | 8-(4- fluorophenyl)-9- (6-hydroxyhexyl)- 6,7-dihydro-5H- benzo[7]annulen- 3-ol | 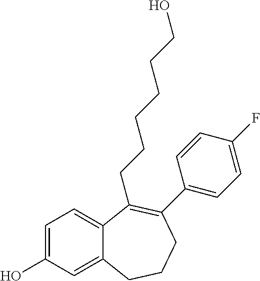 [SMILES: OCCCCCCC1=C(c2ccc(F)cc2)CCCc2cc(O)ccc21] | C₂₃H₂₇FO₂ (M = 354.5). ¹H- NMR (raw product, selected signals, 400 MHz, chloroform-d₁): δ = 2.57- 2.67 (m), 2.30-2.40 (m), 6.69-6.77 (m, 2H), 7.00- 7.08 (m, 2H), 7.15-7.23 (m, 3H). |
| 7-9 | 8-(3- fluorophenyl)-9- (6-hydroxyhexyl)- 6,7-dihydro-5H- benzo[7]annulen- 3-ol | 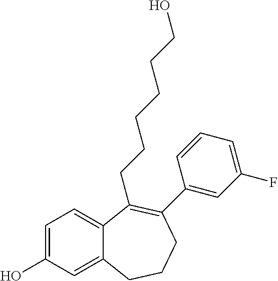 [SMILES: OCCCCCCC1=C(c2cccc(F)c2)CCCc2cc(O)ccc21] | C₂₃H₂₇FO₂ (M = 354.5). ¹H- NMR (600 MHz, DMSO-d₆): δ = 1.02-1.10 (4H), 1.10- 1.18 (2H), 1.21-1.29 (2H), 1.97-2.01 (2H), 2.01-2.08 (2H), 2.32 (t, 2H), 2.55 (t, 2H), 3.26 (t, 2H), 4.24 (t, 1H), 6.64-6.69 (2H), 7.01-7.05 (1H), 7.05-7.12 (2H), 7.12- 7.15 (1H), 7.39-7.44 (1H), 9.33 (s, 1H). |
| 8- | 8-(2- fluorophenyl)-9- (6-hydroxyhexyl)- 6,7-dihydro-5H- benzo[7]annulen- 3-ol | 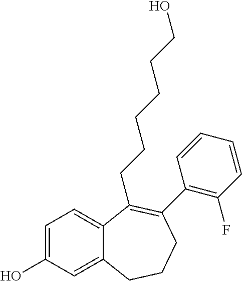 [SMILES: OCCCCCCC1=C(c2ccccc2F)CCCc2cc(O)ccc21] | C₂₃H₂₇FO₂ (M = 354.5). ¹H- NMR (selected signals, 300 MHz. DMSO-d₆): δ = 2.15- 2.25 (m, 2H), 2.50-2.59 (m, 2H), 3.17-3.25 (m, 2H), 4.20 (t, 1H), 6.60-6.67 (m, 2H), 7.08-7.13 (m, 1H), 7.13-7.34 (m, 4H), 9.30 (s, 1H). |
| 9-9 | 8-(4- fluorophenyl)-9- (5- hydroxypentyl)- 6,7-dihydro-5H- benzo[7]annulen- 3-ol | 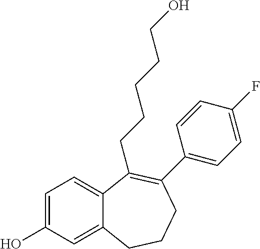 [SMILES: OCCCCCC1=C(c2ccc(F)cc2)CCCc2cc(O)ccc21] | ¹H-NMR (500 MHz, DMSO- d₆): δ = 1.04-1.24 (m, 6H), 1.96-2.06 (m, 4H), 2.30 (dd, 2H), 2.55 (t, 2H), 3.20- 3.26 (m, 2H), 4.24 (t, 1H), 6.63-6.68 (m, 2H), 7.13 (d, 1H), 7.16-7.23 (m, 2H), 7.24-7.29 (m, 2H), 9.31 (s, 1H). |
| 10-9 | 8- (2,4- difluorophenyl)- 9-(6- hydroxyhexyl)- 6,7-dihydro-5H- benzo[7]annulen- 3-ol | 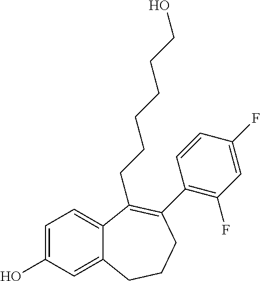 [SMILES: OCCCCCCC1=C(c2ccc(F)cc2F)CCCc2cc(O)ccc21] | ¹H-NMR (300 MHz, DMSO- d₆): δ = 1.03-1.12 (m, 6H), 1.20-1.25 (m, 2H), 1.93 (t, 2H), 2.01-2.07 (m, 2H), 2.22 (t, 2H), 2.57 (t, 1H), 3.25 (q, 2H), 4.24 (t, 1H), 6.66-6.68 (m, 2H), 7.07-7.12 (m, 1H), 7.14 (d, 1H), 7.23-7.28 (m, 1H), 7.29-7.33 (m, 1H), 9.35 (br, 1H). |

### Intermediates for 10

General specification 10 for the preparation of 10 under protective gas atmosphere and with exclusion of moisture: 1 g alcohol was dissolved in approx. 13-33 mL dichloromethane, a mixture of dichloromethane and tetrahydrofuran or pure tetrahydrofuran. 1.5-1.6 equivalents of triphenylphosphine and 1.5-1.6 equivalents of carbon tetrabromide were added in portions at 0-5° C. It was stirred at 3-5° C. for a further 2-3 hours, unless described otherwise. The reaction mixture was diluted with dichloromethane or methyl tert-butyl ether, washed with saturated sodium hydrogen carbonate solution and saturated sodium chloride solution, dried over magnesium sulphate or sodium sulphate and concentrated by evaporation. Then it was chromatographed using Silica Gel 60.

### Intermediate 1-10

### 9-(6-Bromohexyl)-8-(3,4-difluorophenyl)-6,7-dihydro-5H-benzo[7]annulen-3-ol

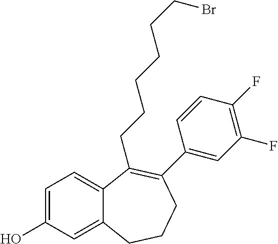 [SMILES: Oc1ccc2c(c1)CCCC(c1ccc(F)c(F)c1)=C2CCCCCCBr]

11.0 g (29.53 mmol) of 6-[8-(3,4-difluorophenyl)-3-methoxy-6,7-dihydro-5H-benzo[7]annulen-9-yl]hexan-1-01 was reacted with 11.85 g triphenylphosphine and 14.99 g carbon tetrabromide according to general specification 10. The residue was chromatographed using Silica Gel 60 (solvent: hexane, hexane-ethyl acetate 95:5, 9:1 and 8:2). 11.2 g (78% of theor.) of product was obtained.

¹H-NMR (300 MHz, chloroform-d₁): δ=1.06-1.32 (m, 6H), 1.71 (quin, 2H), 2.00-2.17 (m, 4H), 2.35 (t, 2H), 2.61 (t, 2H), 3.30 (t, 2H), 6.71 (d, 1H), 6.74 (dd, 1H), 6.90-6.98 (m, 1H), 7.04 (ddd, 1H), 7.11-7.20 (m, 2H).

### Intermediate 2-10

### 9-(6-Bromohexyl)-8-(3,5-difluorophenyl)-6,7-dihydro-5H-benzo[7]annulen-3-ol

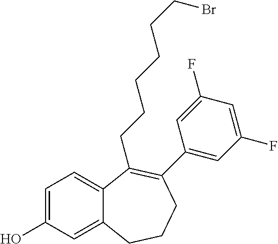 [SMILES: Oc1ccc2c(c1)CCCC(c1cc(F)cc(F)c1)=C2CCCCCCBr]

9.20 g (24.70 mmol) of 6-[8-(3,5-difluorophenyl)-3-methoxy-6,7-dihydro-5H-benzo[7]annulen-9-yl]hexan-1-ol was reacted with 9.91 g triphenylphosphine and 12.53 g carbon tetrabromide according to general specification 10. The residue was chromatographed using Silica Gel 60 (solvent: hexane, hexane-ethyl acetate 95:5, 9:1 and 8:2). 9.2 g (77% of theor.) of product was obtained.

¹H-NMR (400 MHz, chloroform-d₁): δ=1.10-1.30 (m, 6H), 1.72 (quin, 2H), 2.03-2.16 (m, 4H), 2.37 (t, 2H), 2.61 (t, 2H), 3.31 (t, 2H), 4.78 (s, 1H), 6.68-6.79 (m, 5H), 7.17 (d, 1H).

### Intermediate 3-10

### 9-(6-Bromohexyl)-4-fluoro-8-(4-fluorophenyl)-6,7-dihydro-5H-benzo[7]annulen-3-ol

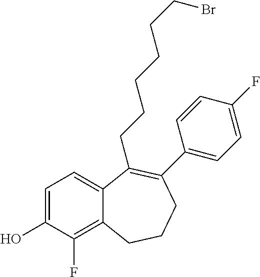 [SMILES: Oc1ccc2c(c1F)CCCC(c1ccc(F)cc1)=C2CCCCCCBr]

4.30 g (11.54 mmol) of 4-fluoro-8-(4-fluorophenyl)-9-(6-hydroxyhexyl)-6,7-dihydro-5H-benzo[7]annulen-3-ol was reacted with 4.33 g triphenylphosphine and 5.86 g carbon tetrabromide according to general specification 10. The residue was chromatographed using Silica Gel 60 (solvent: hexane, hexane-ethyl acetate 95:5, 9:1 and 8:2). 4.2 g (79% of theor.) of product was obtained.

¹H-NMR (300 MHz, chloroform-d₁): δ=1.06-1.31 (m, 6H), 1.71 (quin, 2H), 2.04-2.18 (m, 4H), 2.35 (t, 2H), 2.68-2.78 (m, 2H), 3.30 (t, 2H), 5.09 (d, 1H), 6.89 (t, 1H), 6.96-7.10 (m, 3H), 7.15-7.23 (m, 2H).

### Intermediate 4-10

### 9-(6-Bromohexyl)-8-(2,5-difluorophenyl)-6,7-dihydro-5H-benzo[7]annulen-3-ol

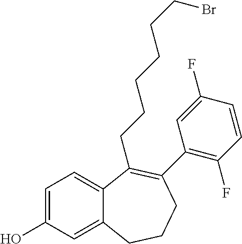 [SMILES: Oc1ccc2c(c1)CCCC(c1cc(F)ccc1F)=C2CCCCCCBr]

6.28 g (16.9 mmol) of 6-[8-(2,5-difluorophenyl)-3-methoxy-6,7-dihydro-5H-benzo[7]annulen-9-yl]hexan-1-01 was reacted with 6.77 g triphenylphosphine and 8.56 g carbon tetrabromide according to general specification 10. The residue was chromatographed using Silica Gel 60 (solvent: hexane, hexane-ethyl acetate 95:5, 9:1 and 8:2). 6.29 g (86% of theor.) of product was obtained.

¹H-NMR (400 MHz, chloroform-d₁): δ=1.08-1.31 (m, 6H), 1.70 (quin, 2H), 2.01-2.20 (m, 4H), 2.31 (t, 2H), 2.65 (mc, 2H), 3.29 (t, 2H), 6.71-6.79 (m, 2H), 6.87-6.98 (m, 2H), 7.04 (dt, 1H), 7.18 (d, 1H).

### Intermediate 5-10

### 9-(6-Bromohexyl)-2-fluoro-8-(4-fluorophenyl)-6,7-dihydro-5H-benzo[7]annulen-3-ol

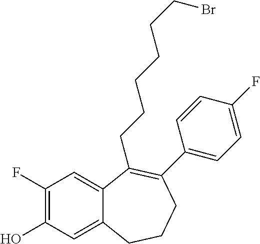 [SMILES: Oc1cc2c(cc1F)C(CCCCCCBr)=C(c1ccc(F)cc1)CCC2]

12.75 g (34.2 mmol) of 2-fluoro-8-(4-fluorophenyl)-9-(6-hydroxyhexyl)-6,7-dihydro-5H-benzo[7]annulen-3-ol was reacted with 13.74 g triphenylphosphine and 17.37 g carbon tetrabromide according to general specification 10. It was stirred further overnight at room temperature and was worked up according to specification 10. The residue was chromatographed using Silica Gel 60 (solvent: hexane, hexane-ethyl acetate 95:5, 9:1 and 8:2). 10.2 g (68% of theor.) of product was obtained.

¹H-NMR (300 MHz, chloroform-d₁): δ=1.07-1.32 (m, 6H), 1.65-1.78 (m, 2H), 2.02-2.18 (m, 4H), 2.32 (m, 2H), 2.54-2.64 (m, 2H), 3.30 (t, 2H), 5.13 (d, 1H), 6.86 (d, 1H), 6.99-7.11 (m, 3H), 7.15-7.23 (m, 2H).

The following intermediates were prepared similarly

| | Name of | Structure of | |
|---|---|---|---|
| Intermediate | intermediate | intermediate | Analytical data |
| 6-10 | 9-(6- bromohexyl)-8- (4-fluorophenyl)- 6,7-dihydro-5H- benzo[7]annulen- 3-ol | 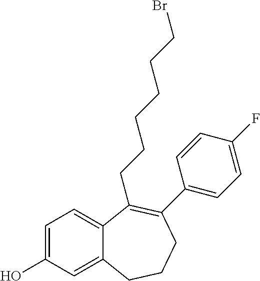 [SMILES: Oc1ccc2c(c1)CCCC(c1ccc(F)cc1)=C2CCCCCCBr] | C₂₃H₂₆BrFO (417.4). ¹H-NMR (600 MHz, chloroform-d₁): δ = 1.09-1.16 (quin., 2H), 1.16-1.28 (4H), 1.70 (quin., 2H), 2.04-2.14 (m, 4H), 2.35 (t, 2H), 2.62 (t, 2H), 3.29 (t, 2H), 6.70-6.72 (1H), 6.73- 6.76 (1H), 7.02-7.07 (2H), 7.15-7.22 (3H). |
| 7-10 | 9-(6- bromohexyl)-8- (3-fluorophenyl)- 6,7-dihydro-5H- benzo[7]annulen- 3-ol | 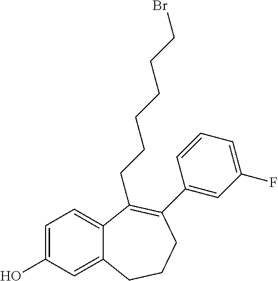 [SMILES: Oc1ccc2c(c1)CCCC(c1cccc(F)c1)=C2CCCCCCBr] | C₂₃H₂₆BrFO (417.4). ¹H-NMR (300 MHz, DMSO-d₆) δ = 0.98-1.21 (m, 6H), 1.59 (quin, 2H), 1.90-2.10 (m, 4H), 2.24-2.34 (m, 2H), 2.48-2.58 (m, partly superimposed with solvent signal), 3.36 (t, 2H), 6.59- 6.67 (m, 2H), 6.96-7.14 (m, 4H), 7.32-7.43 (m, 1H), 9.29 (s, 1H). |
| 8-10 | 9-(6- bromohexyl)-8- (2-fluorophenyl)- 6,7-dihydro-5H- benzo[7]annulen- 3-ol | 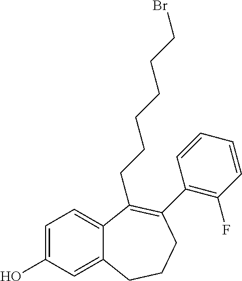 [SMILES: Oc1ccc2c(c1)CCCC(c1ccccc1F)=C2CCCCCCBr] | C₂₃H₂₆BrFO (417.4). ¹H-NMR (300 MHz, DMSO-d₆, selected signals) δ = 1.0- 1.2 (6H), 1.5-1.6 (2H), 1.9- 2.1 (4H), 2.1-2.3 (2H), 2.6 (t), 6.6-6.7 (2H), 7.1-7.3 (5H), 9.3 (s). |
| 9-10 | 9-(5- bromopentyl)-8- (4-fluorophenyl)- 6,7-dihydro-5H- benzo[7]annulen- 3-ol | 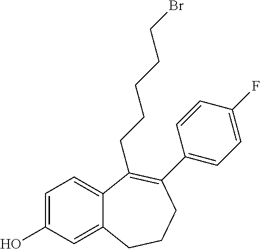 [SMILES: Oc1ccc2c(c1)CCCC(c1ccc(F)cc1)=C2CCCCCBr] | C₂₂H₂₄BrFO (404.3). ¹H-NMR (300 MHz, DMSO-d₆) δ = 1.06-1.21 (m, 4H), 1.48- 1.60 (m, 2H), 1.90-2.06 (m, 4H), 2.23-2.32 (m, 2H), 2.49-2.56 (m), 3.33 (t, 2H), 6.60-6.66 (m, 2H), 7.08- 7.27 (m, 5H), 9.28 (s). |
| 10-10 | 9-(6- bromohexyl)-8- (2,4- difluorophenyl)- 6,7-dihydro-5H- benzo[7]annulen- 3-ol | 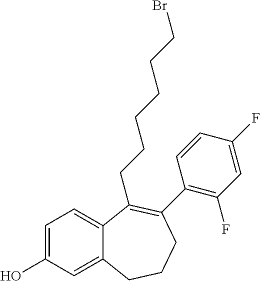 [SMILES: Oc1ccc2c(c1)CCCC(c1ccc(F)cc1F)=C2CCCCCCBr] | ¹H-NMR (300 MHz, DMSO- d₆): δ = 1.02-1.15 (m, 6H), 1.57-1.64 (m, 2H), 1.93 (t, 1H), 2.01-2.07 (m, 2H), 2.21- 2.24 (m, 2H), 2.57 (t, 2H), 3.39 (t, 2H), 6.66-6.68 (m. 2H), 7.07-7.12 (m, 1H), 7.14 (d, 1H), 7.24-7.34 (m, 2H), 9.36 (s, 1H) |

### Intermediates for 11

### Intermediate 1-11

### S-{4-[(tert-Butoxycarbonyl)(methyl)amino]butyl}ethanethioate

### Stage a: Preparation of 4-[(tert-butoxycarbonyl)(methyl)amino]butyl-4-methylbenzene sulphonate

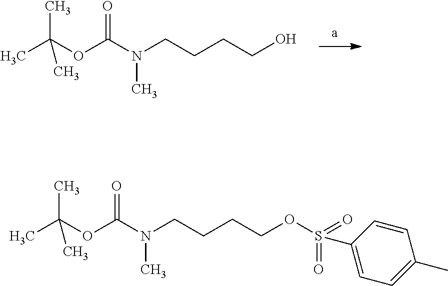 [SMILES: CN(CCCCO)C(=O)OC(C)(C)C ; Cc1ccc(S(=O)(=O)OCCCCN(C)C(=O)OC(C)(C)C)cc1]

4 mL pyridine, 2.44 g of 4-methylbenzene sulphonyl chloride and a spatula tip of N,N-dimethylpyridin-4-amine were added to an ice-cold solution of 2.00 g tert-butyl-(4-hydroxybutyl)methylcarbamate in 20 mL dichloromethane and the mixture was stirred at room temperature for 18 hours. It was poured onto 1M aqueous hydrochloric acid solution, the organic phases were separated and were extracted twice with dichloromethane. The combined organic phases were washed with saturated sodium hydrogen carbonate solution and saturated sodium chloride solution, dried over sodium sulphate, filtered and concentrated by evaporation. After purification by column chromatography on silica gel (hexane/ethyl acetate), 2.7 g of the title compound was obtained.

¹H-NMR (300 MHz, chloroform-d₁): δ 1.42 (s, 9H), 1.45-1.69 (m), 2.45 (s, 3H), 2.79 (s, 3H), 3.17 (t, 2H), 4.04 (t, 2H), 7.34 (d, 2H), 7.79 (d, 2H).

### Stage b: Preparation of S-{4-[(tert-butoxycarbonyl)(methyl)amino]butyl}ethanethioate

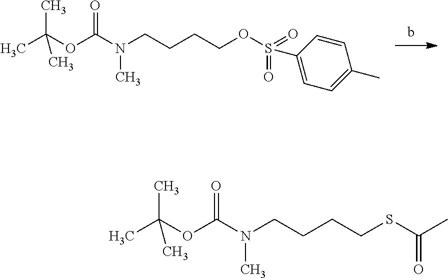

5.66 g sodium iodide and 4.31 g of potassium thioacetate were added to 2.70 g of 4-[(tert-butoxycarbonyl)(methyl)amino]butyl-4-methylbenzene sulphonate in 60 mL 2-butanone and the mixture was heated under reflux overnight. It was poured onto water, extracted three times with tert-butyl methyl ether, washed with saturated sodium chloride solution, dried over sodium sulphate and concentrated by evaporation. 2.1 g of the title compound was obtained.

¹H-NMR (400 MHz, chloroform-d₁): δ 1.45 (s, 9H), 1.50-1.63 (m), 2.33 (s, 3H), 2.82 (s, 3H), 2.86-2.93 (m, 2H), 3.17-3.25 (m, 2H).

### Intermediate 2-11

### S-{4-[(tert-Butoxycarbonyl)(methyl)amino]propyl}ethanethioate

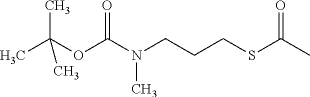 [SMILES: CC(=O)SCCCN(C)C(=O)OC(C)(C)C]

It is prepared similarly to intermediate 1-11 stage b from 2.0 g tert-butyl-(3-chloropropyl)methylcarbamate and potassium thioacetate. 2.6 g of a raw product was obtained.

MS (CI) mass found: 248 [48], 209 [100].

### Intermediate 13

General specification 13 for the preparation of 13 with exclusion of moisture: 1 mol-equivalent of alcohol was dissolved in 5 mol-equivalents of pyridine, and 1.1 mol-equivalent of tosyl chloride was added at 0-5° C. Then it was stirred for a further 2.5 hours at 0° C. and 1-2 hours or overnight at room temperature. The reaction mixture was stirred into a mixture of ice water and concentrated sulphuric acid (10 mL: 1 mL). 29-53 mL water per 10 mL pyridine was taken as the basis. It was shaken three times with diethyl ether, the combined organic phases were washed once with water and with saturated sodium chloride solution, dried over sodium sulphate or magnesium sulphate and concentrated by evaporation.

### Intermediate 1-13

### 4,4,5,5,5-Pentafluoropentyl-4-methylbenzene sulphonate

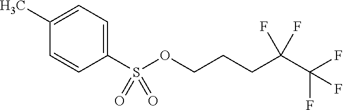 [SMILES: Cc1ccc(S(=O)(=O)OCCCC(F)(F)C(F)(F)F)cc1]

40 g (224.6 mmol) of 4,4,5,5,5-pentafluoropentan-1-ol was reacted with 47.04 g tosyl chloride according to general specification 13. 39.5 g (53% of theor.) of product was obtained.

¹H-NMR (400 MHz, chloroform-d₁): δ=1.90-2.00 (m, 2H), 2.01-2.17 (m, 2H), 2.46 (s, 3H), 4.10 (t, 2H), 7.37 (d, 2H), 7.80 (d, 2H).

### Intermediate 2-13

### 3,3,4,4,4-Pentafluorobutyl-4-methylbenzene sulphonate

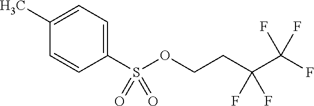 [SMILES: Cc1ccc(S(=O)(=O)OCCC(F)(F)C(F)(F)F)cc1]

19.82 g (120.8 mmol) of 3,3,4,4,4-pentafluorobutan-1-ol was reacted with 25.33 g tosyl chloride according to general specification 13. 27.5 g (72% of theor.) of product was obtained.

¹H-NMR (400 MHz, chloroform-d₁): δ=2.40-2.54 (m, 5H), 4.28 (t, 2H), 7.38 (d, 2H), 7.80 (dt, 2H).

### Intermediate 3-13

### 5,5,5-Trifluoropentyl-4-methylbenzene sulphonate

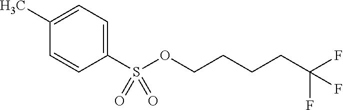 [SMILES: Cc1ccc(S(=O)(=O)OCCCCC(F)(F)F)cc1]

4.3 g (30.3 mmol) of 5,5,5-trifluoropentan-1-ol was reacted with 6.43 g tosyl chloride according to general specification 13. 8.5 g (95% of theor.) of product was obtained.

¹H-NMR (300 MHz, chloroform-d₁): δ=1.58-1.71 (m, 2H), 1.72-1.84 (m, 2H), 1.99-2.17 (m, 2H), 2.50 (s, 3H), 4.09 (t, 2H), 7.40 (d, 2H), 7.84 (d, 2H).

### Intermediate 4-13

### 3,3,3-Trifluoropropyl-4-methylbenzene sulphonate

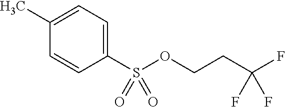 [SMILES: Cc1ccc(S(=O)(=O)OCCC(F)(F)F)cc1]

25.5 g (223.5 mmol) of 3,3,3-trifluoropropan-1-ol was reacted with 45.93 g tosyl chloride according to general specification 13. 47.26 g (80% of theor.) of product was obtained.

¹H-NMR (300 MHz, chloroform-d₁): δ=2.43-2.59 (m, 5H), 4.22 (t, 2H), 7.37 (d, 2H), 7.80 (dt, 2H).

### Intermediates 14

General specification 14 for the preparation of 14: 1 mol-equivalent of tosylate/iodide/chloride was stirred with 1.63 mol-equivalent of potassium thioacetate in acetone (5.1-8.1 mL acetone per g of substance) for 3-3.5 hours under reflux. After cooling, the solvent was drawn off and the residue was added to water. It was shaken three times with diethyl ether. The combined organic phases were washed once with water and once or twice with saturated sodium chloride solution, dried over sodium sulphate or magnesium sulphate and concentrated by evaporation. General specification 14a for the preparation of 14: 1 mol-equivalent of halide was stirred with 1.63 mol-equivalents of potassium thioacetate in acetone (5.1-8.1 mL acetone per g of substance) for 3-3.5 hours under reflux. After cooling, it was filtered with suction and the filtrate was concentrated by evaporation. Water was added and it was shaken three times with diethyl ether. The combined organic phases were dried over magnesium sulphate and concentrated by evaporation.

### Intermediate 1-14

### S-(4,4,5,5,5-Pentafluoropentyl)ethanethioate

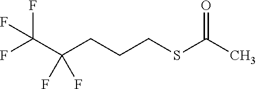

155 g (466.5 mmol) of 4,4,5,5,5-pentafluoropentyl-4-methylbenzene sulphonate was reacted with 86.92 g of potassium thioacetate according to general specification 14. The residue was distilled at normal pressure in a small Vigreux column (10 cm). At 170° C., 84.3 g (77% of theor.) of product was obtained.

¹H-NMR (300 MHz, chloroform-d₁): δ=1.82-1.95 (m, 2H), 2.00-2.20 (m, 2H), 2.35 (s, 3H), 2.95 (t, 2H).

### Intermediate 2-14

### S-(3,3,4,4,4-Pentafluorobutyl)ethanethioate

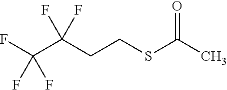

35.6 g (111.9 mmol) of 3,3,4,4,4-pentafluorobutyl-4-methylbenzene sulphonate was reacted with 20.82 g of potassium thioacetate according to general specification 14. The residue was distilled at normal pressure in a small Vigreux column (10 cm). At 70° C., 16.6 g (67% of theor.) of product was obtained.

¹H-NMR (300 MHz, chloroform-d₁): δ=2.24-2.44 (m, 5H), 3.07 (mc, 2H).

### Intermediate 3-14

### S-(5,5,5-Trifluoropentyl)ethanethioate

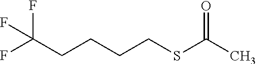

8.5 g (28.7 mmol) of 5,5,5-trifluoropentyl-4-methylbenzene sulphonate was reacted with 5.35 g of potassium thioacetate according to general specification 14. The residue was distilled in vacuum in a small Vigreux column (10 cm). At 48-50° C. (0.7 mbar), 2.74 g (48% of theor.) of product was obtained. A second fraction of 0.34 g (6% of theor.) was obtained at 50-52° C. (0.4 mbar).

¹H-NMR (300 MHz, chloroform-d₁): δ=1.57-1.72 (m, 4H), 2.00-2.18 (m, 2H), 2.34 (s, 3H), 2.85-2.92 (m, 2H).

### Intermediate 4-14

### S-(3,3,3-Trifluoropropyl)ethanethioate

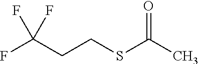

44.88 g (167.3 mmol) of 3,3,3-trifluoropropyl-4-methylbenzene sulphonate was reacted with 31.18 g of potassium thioacetate according to general specification 14. The residue was distilled at normal pressure in a small Vigreux column (10 cm). At 135-137° C., 20.71 g (72% of theor.) of product was obtained.

¹H-NMR (400 MHz, chloroform-d₁): δ=2.33-2.45 (m, 5H), 3.03 (mc, 2H).

### Intermediate 5-14

### S-(5,5,6,6,6-Pentafluorohexyl)ethanethioate

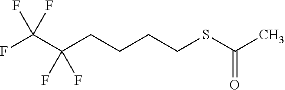

25 g (82.8 mmol) of 1,1,1,2,2-pentafluoro-6-iodohexane was reacted with 15.4 g of potassium thioacetate according to general specification 14. 21.35 g (103% of theor.) of product was obtained.

¹H-NMR (300 MHz, chloroform-d₁): δ=1.59-1.74 (m, 4H), 1.93-2.14 (m, 2H), 2.34 (s, 3H), 2.89 (mc, 2H).

### Intermediate 6-14

### S-(4,4,4-Trifluorobutyl)ethanethioate

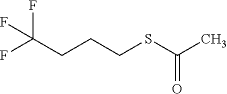

125 g (0.525 mol) of 1,1,1-trifluoro-4-iodobutane was reacted with 97.8 g of potassium thioacetate according to general specification 14a. It was distilled at 95 mbar. The first fraction contained 36.57 g (37% of theor.; 35-95° C.) and the second fraction 48.02 g (49% of theor.; 95-98° C.).

¹H-NMR (400 MHz, chloroform-d₁): δ=1.81-1.90 (m, 2H), 2.09-2.23 (m, 2H), 2.35 (s, 3H), 2.93 (t, 2H).

### Intermediate 7-14

### S-[3,4,4,4-Tetrafluoro-3-(trifluoromethyl)butyl]ethanethioate

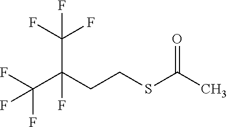 [SMILES: CC(=O)SCCC(F)(C(F)(F)F)C(F)(F)F]

25 g (90.3 mmol) of 4-bromo-1,1,1,2-tetrafluoro-2-(trifluoromethyl)butane was reacted with 16.82 g of potassium thioacetate according to general specification 14a. 22.0 g (90% of theor.) of product was obtained.

¹H-NMR (400 MHz, chloroform-d₁): δ=2.31-2.43 (m, 5H), 3.05 (mc, 2H).

### Intermediate 8-14

### S-(6,6,6-Trifluorohexyl)ethanethioate

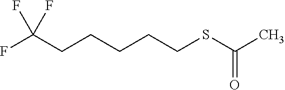

5 g (22.8 mmol) of 6-bromo-1,1,1-trifluorohexane was reacted with 4.25 g of potassium thioacetate according to general specification 14. The acetone was drawn off only at 200 mbar and 40° C. bath temperature. 4.7 g (96% of theor.) of product was obtained.

¹H-NMR (300 MHz, chloroform-d₁): δ=1.37-1.49 (m, 2H), 1.51-1.66 (m, 4H), 1.98-2.16 (m, 2H), 2.33 (s, 3H), 2.87 (t, 2H).

### Intermediates 15

General specification 15 for the preparation of 15: 1 mol-equivalent of thioacetate was added dropwise to 1.1-2.0 mol-equivalent of 30% sodium methylate solution in methanol with cooling on an ice bath. It was stirred for a further 30 minutes at room temperature. This solution was added dropwise at room temperature to 1.3-2 mol-equivalent of 1-bromo-ω-chloroalkane in methanol (1.2-1.7 mL per g of halide). It was stirred for a further 2-4 hours at room temperature. Diethyl ether or methyl tert-butyl ether was added, the phases were separated and the organic phase was washed with water, with saturated sodium chloride solution if necessary, dried over sodium sulphate or magnesium sulphate and concentrated by evaporation. The residue was submitted to fractional distillation in a small Vigreux column (10 cm).

### Intermediate 1-15

### 3-Chloropropyl-4,4,5,5,5-pentafluoropentyl)sulphide

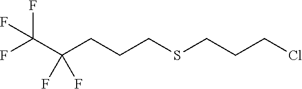 [SMILES: FC(F)(F)C(F)(F)CCCSCCCCl]

132 g (558.54 mmol) of S-(4,4,5,5,5-pentafluoropentyl)ethanethioate was reacted with 131.97 g (558.84 mmol) of 1-bromo-3-chloropropane according to general specification 15.126 g (83% of theor.) of product was obtained. BP_{18 mbar}=117° C.

¹H-NMR (400 MHz, chloroform-d₁): δ=1.85-1.94 (m, 2H), 2.04 (quin, 2H), 2.10-2.25 (m, 2H), 2.61 (t, 2H), 2.68 (t, 2H), 3.66 (t, 2H).

### Intermediate 2-15

### 4-Chlorobutyl-4,4,5,5,5-pentafluoropentylsulphide

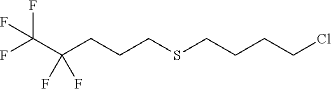 [SMILES: FC(F)(F)C(F)(F)CCCSCCCCCl]

30 g (127.01 mmol) of S-(4,4,5,5,5-pentafluoropentyl)ethanethioate was reacted with 32.67 g (190.51 mmol) of 1-bromo-4-chlorobutane according to general specification 15. 32.28 g (89% of theor.) of product was obtained. BP_{3.6 mbar}=110-112° C.

¹H-NMR (300 MHz, chloroform-d₁): δ=1.74-1.86 (m, 2H), 1.88-2.00 (m, 4H), 2.12-2.32 (m, 2H), 2.55-2.68 (m, 4H), 3.61 (t, 2H).

### Intermediate 3-15

### 3-Chloropropyl-3,3,4,4,4-pentafluorobutylsulphide

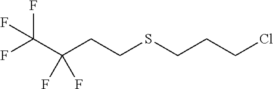 [SMILES: FC(F)(F)C(F)(F)CCSCCCCl]

16.6 g (74.72 mmol) of S-(3,3,4,4,4-pentafluorobutyl)ethanethioate in 10 mL methanol was reacted with 14.7 mL (149.43 mmol) of 1-bromo-3-chloropropane according to general specification 15. 17.6 g (92% of theor.) of product was obtained. BP_{55 mbar}=70° C.

¹H-NMR (300 MHz, chloroform-d₁): δ=2.05 (quin, 2H), 2.24-2.44 (m, 2H), 2.69-2.77 (m, 4H), 3.66 (t, 2H).

### Intermediate 4-15

### 3-[(3-Chloropropyl)sulphanyl]-1,1,1-trifluoropropane

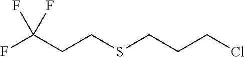 [SMILES: FC(F)(F)CCSCCCCl]

40 g (232.33 mmol) of S-(3,3,3-trifluoropropyl)ethanethioate in 60 mL methanol was reacted with 47.55 g (302.03 mmol) of 1-bromo-3-chloropropane according to general specification 15. The raw product was submitted to fractional distillation in vacuum with a Vigreux column. 36.5 g (76% of theor.) of product was obtained. BP_{10 mbar}=75° C.

¹H-NMR (400 MHz, chloroform-d₁): δ=2.05 (quin, 2H), 2.32-2.46 (m, 2H), 2.67-2.75 (m, 4H), 3.66 (t, 2H).

### Intermediate 5-15

### 3-Chloropropyl-4,4,4-trifluorobutylsulphide

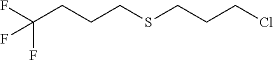 [SMILES: FC(F)(F)CCCSCCCCl]

3.0 g (16.11 mmol) of S-(4,4,4-trifluorobutyl)ethanethioate in 10 mL methanol was reacted with 5.07 g (32.22 mmol) of 1-bromo-3-chloropropane according to general specification 15. All highly volatile constituents were pumped off. 3.7 g (104% of theor.) of product was obtained.

¹H-NMR (400 MHz, chloroform-d₁): δ=1.82-1.91 (m, 2H), 2.04 (quin, 2H), 2.16-2.33 (m, 2H), 2.59 (t, 2H), 2.68 (t, 2H), 3.66 (t, 2H).

### Intermediate 6-15

### 1-Chloro-4-[(3,3,3-trifluoropropyl)sulphanyl]butane

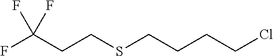 [SMILES: FC(F)(F)CCSCCCCCl]

19.3 g (0.112 mol) of S-(3,3,3-trifluoropropyl)ethanethioate in 30 mL methanol was reacted with 24.99 g (0.146 mol) of 1-bromo-4-chlorobutanane according to general specification 15. The solvent was drawn off at 150 mbar and 40° C. The raw product was submitted to fractional distillation with a Vigreux column. 18.5 g (75% of theor.) of product was obtained. BP_{3 mbar}=85° C.

¹H-NMR (400 MHz, chloroform-d₁): δ=1.72-1.82 (m, 2H), 1.85-1.94 (m, 2H), 2.31-2.45 (m, 2H), 2.59 (t, 2H), 2.66-2.72 (m, 2H), 3.57 (t, 2H).

### Intermediate 7-15

### 3-Chloropropyl-5,5,6,6,6-pentafluorohexylsulphide

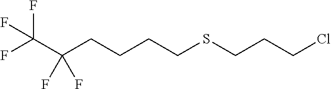 [SMILES: FC(F)(F)C(F)(F)CCCCSCCCCl]

21.3 g (85.1 mmol) of S-(5,5,6,6,6-pentafluorohexyl)ethanethioate in 34 mL methanol was reacted with 26.8 g (170.2 mmol) of 1-bromo-3-chloropropane according to general specification 15. All volatile constituents of the residue were distilled off in a small Vigreux column at 60 mbar and a bath temperature of 90-110° C. 20.34 g (84% of theor.) of product remained.

¹H-NMR (300 MHz, chloroform-d₁): δ=1.62-1.78 (m, 4H), 1.94-2.15 (m, 4H), 2.55 (m, 2H), 2.67 (t, 2H), 3.66 (t, 2H).

### Intermediate 8-15

### 1-Chloro-5-[(3,3,3-trifluoropropyl)sulphanyl]pentane

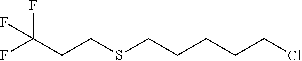 [SMILES: FC(F)(F)CCSCCCCCCl]

4.0 g (23.2 mmol) of S-(3,3,3-trifluoropropyl)ethanethioate in 20 mL methanol was reacted with 4.74 g (25.6 mmol) of 1-bromo-5-chloropentane in 20 mL methanol according to general specification 15, stirring overnight at room temperature. All highly volatile constituents were pumped off. 5.4 g (99% of theor.) of product remained.

¹H-NMR (400 MHz, chloroform-d₁): δ=1.51-1.67 (m, 4H), 1.80 (quin, 2H), 2.31-2.44 (m, 2H), 2.56 (t, 2H), 2.65-2.71 (m, 2H), 3.54 (t, 2H).

### Intermediate 9-15

### 4-[(4-Chlorobutyl)sulphanyl]-1,1,1,2,2-pentafluorobutane

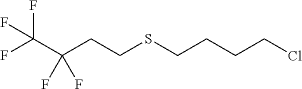 [SMILES: FC(F)(F)C(F)(F)CCSCCCCCl]

4.0 g (18.0 mmol) of S-(3,3,4,4,4-pentafluorobutyl)ethanethioate in 20 mL methanol was reacted with 3.40 g (18.8 mmol) of 1-bromo-4-chlorobutane in 20 mL methanol according to general specification 15, stirring overnight at room temperature. All highly volatile constituents were pumped off. 4.2 g (86% of theor.) of product remained.

¹H-NMR (300 MHz, chloroform-d₁): δ=1.71-1.83 (m, 2H), 1.84-1.95 (m, 2H), 2.23-2.43 (m, 2H), 2.59 (t, 2H), 2.68-2.76 (m, 2H), 3.57 (t, 2H).

### Intermediate 10-15

### 4-[(4-Chlorobutyl)sulphanyl]-1,1,1-trifluorobutane

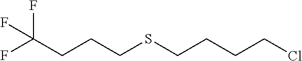 [SMILES: FC(F)(F)CCCSCCCCCl]

6.0 g (32.2 mmol) of S-(4,4,4-trifluorobutyl)ethanethioate in 20 mL methanol was reacted with 6.08 g (35.4 mmol) of 1-bromo-4-chlorobutane in 20 mL methanol according to general specification 15, stirring overnight at room temperature. All highly volatile constituents were pumped off. 7.0 g (93% of theor.) of product remained.

¹H-NMR (400 MHz, chloroform-d₁): δ=1.71-1.80 (m, 2H), 1.81-1.93 (m, 4H), 2.16-2.29 (m, 2H), 2.52-2.61 (m, 4H), 3.56 (t, 2H).

### Intermediate 11-15

### 3-Chloropropyl-6,6,6-trifluorohexylsulphide

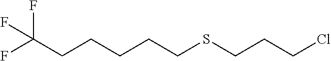 [SMILES: FC(F)(F)CCCCCSCCCCl]

4.7 g (21.9 mmol) of S-(6,6,6-trifluorohexyl)ethanethioate in 10 mL methanol was reacted with 3.8 g (24.1 mmol) of 1-bromo-3-chloropropane according to general specification 15. 4.46 g (82% of theor.) of product was obtained.

¹H-NMR (300 MHz, chloroform-d₁): δ=1.41-1.69 (m, 6H), 1.98-2.17 (m, 4H), 2.53 (t, 2H), 2.67 (t, 2H), 3.66 (t, 2H).

### Intermediate 12-15

### 3-Chloropropyl-5,5,5-trifluoropentylsulphide

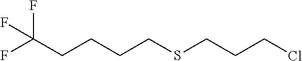 [SMILES: FC(F)(F)CCCCSCCCCl]

9.67 g (48.3 mmol) of S-(5,5,5-trifluoropentyl)ethanethioate in 19.3 mL methanol was reacted with 15.2 g (96.6 mmol) of 1-bromo-3-chloropropane in 19.3 mL methanol according to general specification 15. At 15 mbar and 115° C., 7.92 g (70% of theor.) of product was obtained.

¹H-NMR (300 MHz, chloroform-d₁): δ=1.60-1.76 (m, 4H), 1.98-2.20 (m, 4H), 2.54 (mc, 2H), 2.67 (t, 2H), 3.66 (t, 2H).

### Intermediate 13-15

### 4-[(4-Chlorobutyl)sulphanyl]-1,1,1,2-tetrafluoro-2-(trifluoromethyl)butane

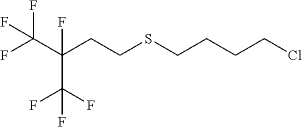 [SMILES: FC(F)(F)C(F)(CCSCCCCCl)C(F)(F)F]

11.0 g (40.4 mmol) of S-[3,4,4,4-tetrafluoro-3-(trifluoromethyl)butyl]ethanethioate in 40 mL methanol was reacted with 7.6 g (44.3 mmol) of 1-bromo-4-chlorobutane in 40 mL methanol according to general specification 15. 10.0 g (73% of theor.) of product was obtained.

¹H-NMR (300 MHz, chloroform-d₁): δ=1.71-1.83 (m, 2H), 1.84-1.96 (m, 2H), 2.30-2.46 (m, 2H), 2.59 (t, 2H), 2.66-2.74 (mc, 2H), 3.57 (t, 2H).

### Intermediate 14-15

### 3-Chloropropyl-3,4,4,4-tetrafluoro-3-(trifluoromethyl)butylsulphide

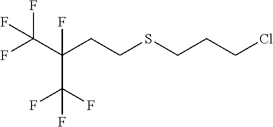 [SMILES: FC(F)(F)C(F)(CCSCCCCl)C(F)(F)F]

11.0 g (40.4 mmol) of S-[3,4,4,4-tetrafluoro-3-(trifluoromethyl)butyl]ethanethioate in 40 mL methanol was reacted with 7.0 g (44.5 mmol) of 1-bromo-3-chloropropane in 40 mL methanol according to general specification 15. 9.8 g (75% of theor.) of product was obtained.

¹H-NMR (300 MHz, chloroform-d₁): δ=2.05 (quin, 2H), 2.31-2.46 (m, 2H), 2.65-2.76 (m, 4H), 3.66 (t, 2H).

### Intermediates 17

General specification 17 for the preparation of 17: 1 mol-equivalent of thioether was dissolved in acetone (1 g of substance in 7.3-11.2 mL), methanol (1 g of substance in 4.3-6.7 mL) and water (2 mL water per 1 g sodium metaperiodate) and 1.1 mol-equivalent of sodium metaperiodate was added. It was stirred at room temperature for 24-60 hours. The precipitate was filtered with suction and washed again thoroughly with acetone. The filtrate was evaporated to dryness, the residue was dissolved in methyl tert-butyl ether, washed with water, dried over sodium sulphate or magnesium sulphate and concentrated by evaporation.

### Intermediate 1-17

### 3-Chloropropyl-4,4,5,5,5-pentafluoropentylsulphoxide

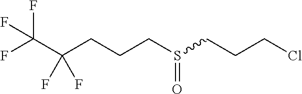 [SMILES: O=S(CCCCl)CCCC(F)(F)C(F)(F)F]

18 g (66.5 mmol) of 3-chloropropyl-4,4,5,5,5-pentafluoropentylsulphide was reacted according to general specification 17. The raw product was digested in hot hexane, filtered with suction and dried. 17.3 g (91% of theor.) of white crystals was obtained.

¹H-NMR (300 MHz, chloroform-d₁): δ=2.15-2.41 (m, 6H), 2.75-3.01 (m, 4H), 3.69-3.83 (m, 2H).

### Intermediate 2-17

### 4-Chlorobutyl-4,4,5,5,5-pentafluoropentylsulphoxide

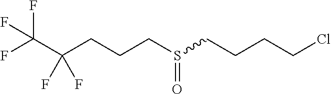 [SMILES: O=S(CCCCCl)CCCC(F)(F)C(F)(F)F]

13 g (45.66 mmol) of 4-chlorobutyl-4,4,5,5,5-pentafluoropentylsulphide was reacted according to general specification 17. The raw product was digested in hot hexane, filtered with suction and dried. 12.77 g (93% of theor.) of white crystals was obtained.

¹H-NMR (300 MHz, chloroform-d₁): δ=1.90-2.12 (m, 4H), 2.15-2.41 (m, 4H), 2.68-2.90 (m, 4H), 3.62 (t, 2H).

### Intermediate 3-17

### 3-Chloropropyl-3,3,4,4,4-pentafluorobutylsulphoxide

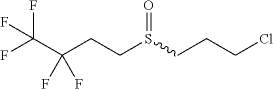 [SMILES: O=S(CCCCl)CCC(F)(F)C(F)(F)F]

5.02 g (19.56 mmol) of 3-chloropropyl-3,3,4,4,4-pentafluorobutylsulphide was reacted according to general specification 17. 4.8 g (90% of theor.) of product was obtained.

¹H-NMR (400 MHz, chloroform-d₁): δ=2.31 (quin, 2H), 2.50-2.66 (m, 2H), 2.83-3.01 (m, 4H), 3.66-3.78 (m, 2H).

### Intermediate 4-17

### 3-[(3-Chloropropyl)sulphinyl]-1,1,1-trifluoropropane

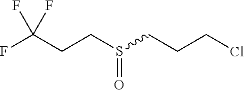 [SMILES: O=S(CCCCl)CCC(F)(F)F]

18 g (87.1 mmol) of 3-[(3-chloropropyl)sulphanyl]-1,1,1-trifluoropropane was reacted according to general specification 17. 17.5 g (90% of theor.) of product was obtained.

¹H-NMR (300 MHz, chloroform-d₁): δ=2.25-2.36 (m, 2H), 2.54-2.71 (m, 2H), 2.80-2.99 (m, 4H), 3.64-3.78 (m, 2H).

### Intermediates 19

General specification 19 for the preparation of 19: 1 mol-equivalent of thioether was dissolved in chloroform. On an ice bath, meta-chloroperbenzoic acid (approx. 80-90%) was added in portions, in such a way that the temperature did not rise above 10° C. It was stirred for a further 1.5-3 hours at room temperature, before being diluted with dichloromethane. Excess per-acid was reduced by washing with 39% sodium hydrogen sulphite solution. The organic phase was washed with saturated sodium hydrogen carbonate solution and/or with saturated sodium carbonate solution and/or with 2M NaOH and optionally with water, dried over sodium sulphate or magnesium sulphate and concentrated by evaporation.

### Intermediate 1-19

### 3-Chloropropyl-4,4,5,5,5-pentafluoropentylsulphone

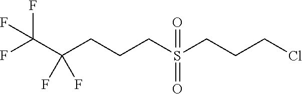 [SMILES: O=S(=O)(CCCCl)CCCC(F)(F)C(F)(F)F]

2.7 g (9.97 mmol) of 3-chloropropyl-4,4,5,5,5-pentafluoropentylsulphide was reacted in 27 mL chloroform with 3.44 g (19.95 mmol) of meta-chloroperbenzoic acid according to general specification 19. 2.81 g (93% of theor.) of product was obtained.

¹H-NMR (300 MHz, chloroform-d₁): δ=2.15-2.40 (m, 6H), 3.09 (t, 2H), 3.19 (mc, 2H), 3.71 (t, 2H).

### Intermediate 2-19

### 4-Chlorobutyl-4,4,5,5,5-pentafluoropentylsulphone

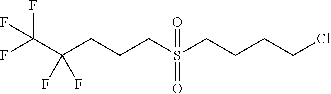 [SMILES: O=S(=O)(CCCCCl)CCCC(F)(F)C(F)(F)F]

15 g (52.68 mmol) of 4-chlorobutyl-4,4,5,5,5-pentafluoropentylsulphide in 143 mL chloroform was reacted with 27.27 g (158.05 mmol) according to general specification 19. 16.25 g (97% of theor.) of product was obtained.

¹H-NMR (300 MHz, chloroform-d₁): δ=1.91-2.12 (m, 4H), 2.14-2.38 (m, 4H), 2.99-3.11 (m, 4H), 3.59 (t, 2H).

### Intermediate 3-19

### 3-Chloropropyl-3,3,4,4,4-pentafluorobutylsulphone

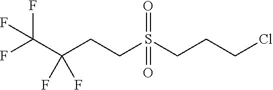 [SMILES: O=S(=O)(CCCCl)CCC(F)(F)C(F)(F)F]

7 g (27.27 mmol) of 3-chloropropyl-3,3,4,4,4-pentafluorobutylsulphide in 75 mL chloroform was reacted with 15.06 g (87.27 mmol) of meta-chloroperbenzoic acid according to general specification 19. 7.28 g (92% of theor.) of product was obtained.

¹H-NMR (300 MHz, chloroform-d₁): δ=2.38 (mc, 2H), 2.54-2.75 (m, 2H), 3.21-3.31 (m, 4H), 3.72 (t, 2H).

### Intermediate 4-19

### 3-[(3-Chloropropyl)sulphonyl]-1,1,1-trifluoropropane

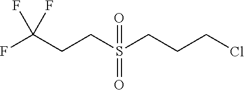 [SMILES: O=S(=O)(CCCCl)CCC(F)(F)F]

18.2 g (88.07 mmol) of 3-[(3-chloropropyl)sulphanyl]-1,1,1-trifluoropropane in 300 mL chloroform was reacted with 45.59 g (264.2 mmol) of meta-chloroperbenzoic acid according to general specification 19. The raw product was stirred with hexane, filtered with suction and dried in a drying cabinet. 20.6 g (98% of theor.) of product was obtained.

¹H-NMR (400 MHz, chloroform-d₁): δ=2.32-2.40 (m, 2H), 2.63-2.76 (m, 2H), 3.19-3.27 (m, 4H), 3.72 (t, 2H).

### Intermediate 5-19

### 1-Chloro-4-[(3,3,3-trifluoropropyl)sulphonyl]butane

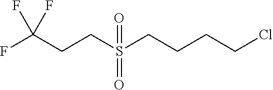 [SMILES: O=S(=O)(CCCCCl)CCC(F)(F)F]

20.0 g (0.091 mol) of 1-chloro-4-[(3,3,3-trifluoropropyl)sulphanyl]butane in 200 mL chloroform was reacted with 46.92 g (0.272 mol) of meta-chloroperbenzoic acid according to general specification 19. The raw product was stirred with pentane, filtered with suction and dried in a drying cabinet. 22.5 g (98% of theor.) of product was obtained.

¹H-NMR (300 MHz, chloroform-d₁): δ=1.91-2.14 (m, 4H), 2.60-2.78 (m, 2H), 3.08 (t, 2H), 3.15-3.24 (mc, 2H), 3.60 (t, 2H).

### Intermediate 6-19

### 4-[(4-Chlorobutyl)sulphonyl]-1,1,1-trifluorobutane

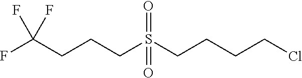 [SMILES: O=S(=O)(CCCCCl)CCCC(F)(F)F]

1 g (4.26 mmol) of 4-[(4-chlorobutyl)sulphanyl]-1,1,1-trifluorobutane in 10 mL chloroform was reacted with 3 g (17.38 mmol) of meta-chloroperbenzoic acid according to general specification 19. 1.1 g (97% of theor.) of product was obtained.

¹H-NMR (300 MHz, chloroform-d₁): δ=1.90-2.22 (m, 6H), 2.25-2.43 (m, 2H), 2.98-3.10 (m, 4H), 3.59 (t, 2H).

### Intermediate 7-19

### 1-Chloro-5-[(3,3,3-trifluoropropyl)sulphonyl]pentane

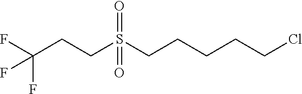 [SMILES: O=S(=O)(CCCCCCl)CCC(F)(F)F]

5.4 g (23.0 mmol) of 1-chloro-5-[(3,3,3-trifluoropropyl)sulphanyl]pentane in 100 mL chloroform was reacted overnight with 11.91 g (69.0 mmol) of meta-chloroperbenzoic acid according to general specification 19. 6.1 g (99% of theor.) of product was obtained.

¹H-NMR (300 MHz, chloroform-d₁): δ=1.58-1.70 (m, 2H), 1.78-1.97 (m, 4H), 2.60-2.76 (m, 2H), 3.05 (mc, 2H), 3.18 (mc, 2H), 3.56 (t, 2H).

### Intermediate 8-19

### 4-[(4-Chlorobutyl)sulphonyl]-1,1,1,2,2-pentafluorobutane

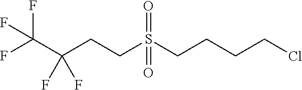 [SMILES: O=S(=O)(CCCCCl)CCC(F)(F)C(F)(F)F]

4.2 g (15.5 mmol) of 4-[(4-chlorobutyl)sulphanyl]-1,1,1,2,2-pentafluorobutane in 100 mL chloroform was reacted overnight with 8.03 g (46.5 mmol) of meta-chloroperbenzoic acid according to general specification 19. 4.5 g (96% of theor.) of product was obtained.

¹H-NMR (300 MHz, chloroform-d₁): δ=1.92-2.14 (m, 4H), 2.63 (mc, 2H), 3.10 (mc, 2H), 3.22 (mc, 2H), 3.60 (t, 2H).

### Intermediate 9-19

### 3-Chloropropyl-5,5,6,6,6-pentafluorohexylsulphone

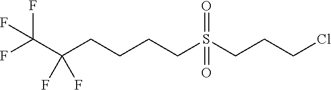 [SMILES: O=S(=O)(CCCCl)CCCCC(F)(F)C(F)(F)F]

10 g (35.1 mmol) of 3-chloropropyl-5,5,6,6,6-pentafluorohexylsulphide in 95 mL chloroform was reacted with 19.4 g (112.4 mmol) of meta-chloroperbenzoic acid according to general specification 19. 10.33 g (93% of theor.) of product was obtained.

¹H-NMR (300 MHz, chloroform-d₁): δ=1.72-1.85 (m, 2H), 1.91-2.19 (m, 4H), 2.28-2.39 (m, 2H), 3.03 (mc, 2H), 3.16 (mc, 2H), 3.71 (t, 2H).

### Intermediate 10-19

### 3-Chloropropyl-5,5,5-trifluoropentylsulphone

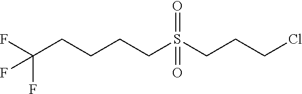 [SMILES: O=S(=O)(CCCCl)CCCCC(F)(F)F]

7.9 g (33.7 mmol) of 3-chloropropyl-5,5,5-trifluoropentylsulphide in 90 mL chloroform was reacted with 18.36 g (106.4 mmol) of meta-chloroperbenzoic acid according to general specification 19, but it was stirred for 3 hours at 0° C. and overnight at room temperature. 8.74 g (99% of theor.) of product was obtained.

¹H-NMR (300 MHz, chloroform-d₁): δ=1.69-1.82 (m, 2H), 1.96 (mc, 2H), 2.07-2.24 (m, 2H), 2.28-2.38 (m, 2H), 3.02 (mc, 2H), 3.16 (mc, 2H), 3.70 (t, 2H).

### Intermediate 11-19

### 3-Chloropropyl-4,4,4-trifluorobutylsulphone

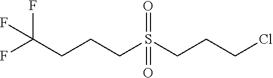 [SMILES: O=S(=O)(CCCCl)CCCC(F)(F)F]

5 g (22.7 mmol) of 3-chloropropyl-4,4,4-trifluorobutylsulphide in 53 mL chloroform was reacted with 14.66 g (85.0 mmol) of meta-chloroperbenzoic acid according to general specification 19, but it was stirred overnight at room temperature. Pentane was added to the residue and it was filtered with suction. 4.9 g (86% of theor.) of product was obtained.

¹H-NMR (300 MHz, chloroform-d₁): δ=2.11-2.24 (m, 2H), 2.26-2.43 (m, 4H), 3.08 (mc, 2H), 3.16 (mc, 2H), 3.71 (t, 2H).

### Intermediate 12-19

### 3-Chloropropyl-6,6,6-trifluorohexylsulphone

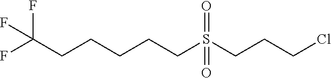 [SMILES: O=S(=O)(CCCCl)CCCCCC(F)(F)F]

4.4 g (17.7 mmol) of 3-chloropropyl-6,6,6-trifluorohexylsulphide in 50 mL chloroform was reacted overnight with 11.45 g (66.3 mmol) of meta-chloroperbenzoic acid according to general specification 19. The residue was digested with pentane, filtered with suction and dried in a drying cabinet. 4.4 g (89% of theor.) of product was obtained.

¹H-NMR (400 MHz, chloroform-d₁): δ=1.51-1.68 (m, 4H), 1.91 (mc, 2H), 2.04-2.18 (m, 2H), 2.34 (mc, 2H), 3.01 (mc, 2H), 3.16 (mc, 2H), 3.71 (t, 2H).

### Intermediate 13-19

### 4-[(4-Chlorobutyl)sulphonyl]-1,1,1,2-tetrafluoro-2-(trifluoromethyl)butane

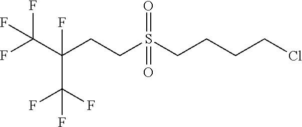 [SMILES: O=S(=O)(CCCCCl)CCC(F)(C(F)(F)F)C(F)(F)F]

10.0 g (31.2 mmol) of 4-[(4-chlorobutyl)sulphanyl]-1,1,1,2-tetrafluoro-2-(trifluoromethyl)butane in 200 mL chloroform was reacted overnight with 20.18 g (116.9 mmol) of meta-chloroperbenzoic acid according to general specification 19.

10.0 g (86% of theor.) of product was obtained.

¹H-NMR (300 MHz, chloroform-d₁): δ=1.91-2.14 (m, 4H), 2.60-2.75 (m, 2H), 3.10 (mc, 2H), 3.20 (mc, 2H), 3.60 (t, 2H).

### Intermediate 14-19

### 3-Chloropropyl-3,4,4,4-tetrafluoro-3-(trifluoromethyl)butylsulphone

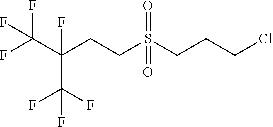 [SMILES: O=S(=O)(CCCCl)CCC(F)(C(F)(F)F)C(F)(F)F]

9.8 g (32.0 mmol) of 4-[(4-chlorobutyl)sulphanyl]-1,1,1,2-tetrafluoro-2-(trifluoromethyl)butane in 200 mL chloroform was reacted overnight with 20.68 g (119.8 mmol) of meta-chloroperbenzoic acid according to general specification 19.

9.6 g (84% of theor.) of product was obtained.

¹H-NMR (300 MHz, chloroform-d₁): δ=2.37 (mc, 2H), 2.61-2.77 (m, 2H), 3.19-3.29 (m, 4H), 3.72 (t, 2H).

### Intermediates 16, 18, 20

General specification 16-18-20-A for the preparation of 16-18-20:1 mol-equivalent of chloride was dissolved in ethanol (1.7-5.5 mL per g chloride) and 40% aqueous methylamine solution (12-18 mL per g chloride) was added. It was stirred for 4 hours at 40° C. in an autoclave. After cooling, it was extracted three times with methyl tert-butyl ether. The combined organic phases were washed with 1M NaOH, dried over sodium sulphate and concentrated by evaporation.

General specification 16-18-20-B for the preparation of 16-18-20: 1 g of chloride was dissolved in 10-25 mL of 33% ethanolic methylamine solution and stirred at 40° C. in an autoclave. After cooling, it was concentrated by evaporation.

General specification 16-18-20-C for the preparation of 16-18-20: 1 g of chloride was dissolved in 7-14 mL methanol and stirred with 1.05 mol-equivalents triethylamine and 2-5 mol-equivalents amine at 60° C. Alternatively it could also be stirred in a microwave. The reaction mixture was concentrated in a rotary evaporator, saturated sodium carbonate solution or water and 2M sodium hydroxide solution were added and it was extracted with dichloromethane or chloroform three or four times. The combined organic phases were, if necessary, washed with water, dried over magnesium sulphate and concentrated by evaporation.

General specification 16-18-20-D for the preparation of 16-18-20 :1 g of chloride was dissolved in 10-67 mL of 33% ethanolic methylamine solution and stirred at 40° C. in an autoclave. After cooling, it was concentrated by evaporation. The residue was taken up in water and shaken twice with dichloromethane. The aqueous phase was adjusted with 2M sodium hydroxide solution to pH>10 and extracted three times with dichloromethane. The combined organic phases were dried over magnesium sulphate and concentrated by evaporation.

### Intermediate 1-16

### Stage A

### 1,1,1,2,2-Pentafluoro-5-[(3-iodopropyl)sulphanyl]pentane

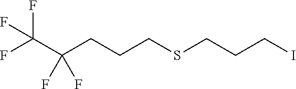 [SMILES: FC(F)(F)C(F)(F)CCCSCCCI]

10 g (36.94 mmol) of 3-chloropropyl-4,4,5,5,5-pentafluoropentylsulphide was dissolved in 220 mL methyl ethyl ketone and 17.6 g (117.4 mmol) of sodium iodide was added. It was stirred for 5 hours at 100° C. bath temperature. After cooling, water was added, it was extracted with ethyl acetate and dried over sodium sulphate and concentrated by evaporation. 13.32 g (99% of theor.) of product was obtained.

¹H-NMR (300 MHz, chloroform-d₁): δ=1.84-1.96 (m, 2H), 2.01-2.31 (m, 4H), 2.57-2.67 (m, 4H), 3.29 (t, 2H).

### Stage B

### N-Methyl-3-[(4,4,5,5,5-pentafluoropentyl)sulphanyl]propan-1-amine

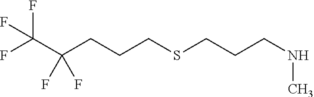 [SMILES: CNCCCSCCCC(F)(F)C(F)(F)F]

13.2 g (36.45 mmol) of 1,1,1,2,2-pentafluoro-5-[(3-iodopropyl)sulphanyl]pentane was dissolved in 20 mL ethanol and 140 mL of 40% aqueous methylamine solution. It was stirred for 4 hours at 40° C. in an autoclave. After cooling, it was extracted three times with methyl tert-butyl ether. The combined organic phases were washed once with 1M sodium hydroxide, dried over sodium sulphate and concentrated by evaporation. It was purified on Silica Gel 60 (solvent: dichloromethane, dichloromethane-methanol 4:1, 3:1, 2:1, 1:1 and methanol). 5.15 g (53% of theor.) of product was obtained.

¹H-NMR (300 MHz, chloroform-d₁): δ=1.78-1.93 (m, 4H), 2.05-2.26 (m, 2H), 2.47 (s, 3H), 2.58 (t, 2H), 2.59 (t, 2H), 2.74 (t, 2H).

### Intermediate 1-18

### N-Methyl-3-[(4,4,5,5,5-pentafluoropentyl)sulphinyl]propan-1-amine

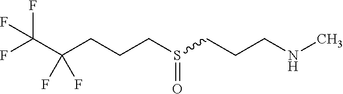 [SMILES: CNCCCS(=O)CCCC(F)(F)C(F)(F)F]

30 g (104.6 mmol) of 3-chloropropyl-4,4,5,5,5-pentafluoropentylsulphoxide was reacted according to general specification 16-18-20-A for 24 hours at 40° C. It was purified on Silica Gel 60 (solvent: dichloromethane, dichloromethane-methanol 1:1 and methanol). 12.84 g (44% of theor.) of product was obtained.

¹H-NMR (300 MHz, chloroform-d₁): δ=1.12 (s-br, 1H), 1.90-2.05 (m, 2H), 2.08-2.34 (m, 4H), 2.43 (s, 3H), 2.70-2.81 (m, 6H).

### Intermediate 2-18

### N-Methyl-4-[(4,4,5,5,5-pentafluoropentyl)sulphinyl]butan-1-amine

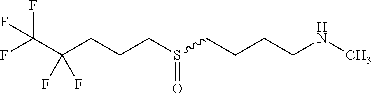 [SMILES: CNCCCCS(=O)CCCC(F)(F)C(F)(F)F]

14 g (46.56 mmol) of 4-chlorobutyl-4,4,5,5,5-pentafluoropentylsulphoxide was reacted according to general specification 16-18-20-A. It was purified on Silica Gel 60 (solvent: dichloromethane, dichloromethane-methanol 4:1, 3:1, 2:1, 1:1 and methanol with 1 vol. % and 10 vol. % of 33% ammonia solution). 12.09 g (88% of theor.) of product was obtained.

¹H-NMR (300 MHz, chloroform-d₁): δ=1.56-1.93 (m, 4H), 1.96-2.36 (m, 5H), 2.44 (s, 3H), 2.60-2.83 (m, 6H).

### Intermediate 3-18

### N-Methyl-3-[(3,3,3-trifluoropropyl)sulphinyl]propan-1-amine

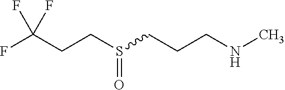

4.2 g (18.86 mmol) of 3-[(3-chloropropyl)sulphinyl]-1,1,1-trifluoropropane was reacted according to general specification 16-18-20-B for 20 hours. It was purified on Silica Gel 60 (solvent: dichloromethane, dichloromethane-methanol 2:1, 1:1 and methanol with 2 vol. % and 5 vol. % of 33% ammonia solution). 1.86 g (45% of theor.) of product was obtained.

¹H-NMR (400 MHz, DMSO-d₆): δ=1.72-1.88 (m, 2H), 2.25-2.33 (m, 3H), 2.54-2.92 (m, 7H), 2.96-3.06 (m, 1H).

### Intermediate 4-18

### 2-Methyl-1-({3-[(3,3,3-trifluoropropyl)sulphinyl]propyl}amino)propan-2-ol

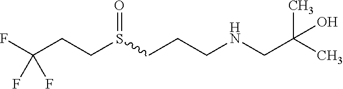 [SMILES: CC(C)(O)CNCCCS(=O)CCC(F)(F)F]

4 g (17.96 mmol) of 3-[(3-chloropropyl)sulphinyl]-1,1,1-trifluoropropane and 5.61 mL of 1-amino-2-methylpropan-2-ol were stirred and worked up according to general specification 16-18-20-C for 30 hours. It was purified on Silica Gel 60 (solvent: dichloromethane, dichloromethane-methanol 4:1, 1:1 and methanol). 2.2 g (44% of theor.) of product was obtained.

¹H-NMR (300 MHz, methanol-d₄): δ=1.23 (s, 6H), 2.09 (quin, 2H), 2.58-2.78 (m, 4H), 2.84-3.06 (m, 5H), 3.12 (ddd, 1H).

### Intermediate 5-18

### 2-Methyl-1-({3-[(4,4,5,5,5-pentafluoropentyl)sulphinyl]propyl}amino)propan-2-ol

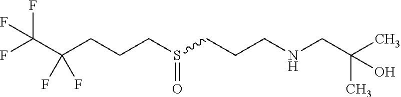 [SMILES: CC(C)(O)CNCCCS(=O)CCCC(F)(F)C(F)(F)F]

6.126 g (21.4 mmol) of 3-chloropropyl-4,4,5,5,5-pentafluoropentylsulphoxide and 4.84 g (54.3 mmol) of 1-amino-2-methylpropan-2-ol were stirred and worked up according to general specification 16-18-20-C for 5 days at 60° C. It was purified on Silica Gel 60 (solvent: dichloromethane, dichloromethane-methanol 2:1, 1:1 and methanol with 5 vol. % and 10 vol. % of 33% ammonia solution). 2.3 g (31% of theor.) of product was obtained.

¹H-NMR (400 MHz, chloroform-d₁): δ=1.18 (s, 6H), 1.95-2.06 (m, 2H), 2.11-2.32 (m, 4H), 2.56 (AB, 2H), 2.69-2.88 (m, 6H).

### Intermediate 6-18

### N-Methyl-3-[(3,3,4,4,4-pentafluorobutyl)sulphinyl]propan-1-amine

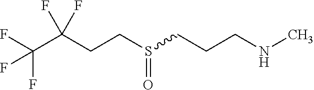 [SMILES: CNCCCS(=O)CCC(F)(F)C(F)(F)F]

4.75 g (17.4 mmol) of 3-chloropropyl-3,3,4,4,4-pentafluorobutylsulphoxide was stirred and worked up in 100 mL of 33% ethanolic methylamine solution for 20 hours. It was purified on Silica Gel 60 (solvent: dichloromethane, dichloromethane-methanol 2:1, 1:1 and methanol with 5 vol. % and 10 vol. % of 33% ammonia solution). 4.45 g (96% of theor.) of product was obtained.

¹H-NMR (300 MHz, methanol-d₄): δ=1.74 (mc, 2H), 2.25 (s, 3H), 2.44-2.91 (m, 7H), 3.06 (ddd, 1H).

### Intermediate 1-20

### N-Methyl-3-[(4,4,5,5,5-pentafluoropentyl)sulphonyl]propan-1-amine

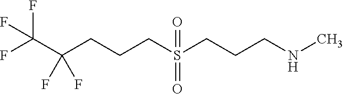 [SMILES: CNCCCS(=O)(=O)CCCC(F)(F)C(F)(F)F]

30 g (99.1 mmol) of 3-chloropropyl-4,4,5,5,5-pentafluoropentylsulphone was reacted and worked up according to general specification 16-18-20-A for 24 hours at 40° C. 27.8 g (94% of theor.) of product was obtained.

¹H-NMR (400 MHz, chloroform-d₁): δ=1.22 (s-br, 1H), 2.00 (mc, 2H), 2.13-2.34 (m, 4H), 2.42 (s, 3H), 2.73 (t, 2H), 3.06 (t, 2H) 3.11 (mc, 2H).

### Intermediate 2-20

### N-Methyl-4-[(4,4,5,5,5-pentafluoropentyl)sulphonyl]butan-1-amine

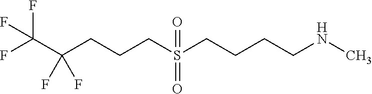 [SMILES: CNCCCCS(=O)(=O)CCCC(F)(F)C(F)(F)F]

16.2 g (51.15 mmol) of 4-chlorobutyl-4,4,5,5,5-pentafluoropentylsulphone was reacted and worked up according to general specification 16-18-20-B for 20 hours at 40° C. It was purified on Silica Gel 60 (solvent: dichloromethane, dichloromethane-methanol 2:1, 1:1 and methanol with 1 vol. % and 10 vol. % of 33% ammonia solution). 14.2 g (89% of theor.) of product was obtained.

¹H-NMR (600 MHz, chloroform-d₁): δ=1.49 (s-br, 1H), 1.66 (quin, 2H), 1.92 (mc, 2H), 2.16-2.34 (m, 4H), 2.44 (s, 3H), 2.64 (t, 2H), 3.01-3.08 (m, 4H).

### Intermediate 3-20

### N-Methyl-3-[(3,3,3-trifluoropropyl)sulphonyl]propan-1-amine

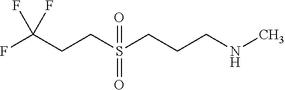

5.8 g (24.2 mmol) of 3-[(3-chloropropyl)sulphonyl]-1,1,1-trifluoropropane was stirred and worked up according to general specification 16-18-20-B for 20 hours. It was purified on Silica Gel 60 (solvent: dichloromethane, dichloromethane-methanol 2:1, 1:1 and methanol with 1.5 vol. % of 33% ammonia solution). 3.92 g (69% of theor.) of product was obtained.

¹H-NMR (400 MHz, DMSO-d₆): δ=2.03 (quin, 2H), 2.49 (s, 3H), 2.66-2.81 (m, 2H), 2.94 (t, 2H), 3.33-3.45 (m, 4H).

### Intermediate 4-20

### N-Ethyl-3-[(3,3,3-trifluoropropyl)sulphonyl]propan-1-amine

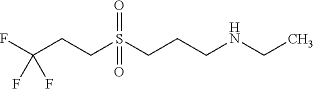

4 g (16.76 mmol) of 3-[(3-chloropropyl)sulphonyl]-1,1,1-trifluoropropane was stirred with 25 mL of 30-40% methanolic ethylamine solution for 30 hours at 60° C. After it had cooled, the reaction solution was concentrated by evaporation, saturated sodium carbonate solution was added and it was extracted three times with dichloromethane. The combined organic phases were washed once with water, dried over magnesium sulphate and concentrated by evaporation. 3.6 g (87% of theor.) of product was isolated.

¹H-NMR (300 MHz, chloroform-d₁): δ=1.05 (s-br, 1H), 1.09 (t, 3H), 1.96-2.07 (m, 2H), 2.59-2.81 (m, 6H), 3.13-3.25 (m, 4H).

### Intermediate 5-20

### 2-({3-[(3,3,3-Trifluoropropyl)sulphonyl]propyl}amino)ethanol

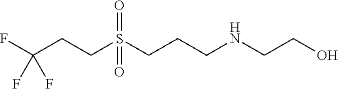 [SMILES: O=S(=O)(CCCNCCO)CCC(F)(F)F]

4 g (16.76 mmol) of 3-[(3-chloropropyl)sulphonyl]-1,1,1-trifluoropropane and 5.98 mL 2-aminoethan-1-ol were stirred and worked up according to general specification 16-18-20-C for 30 hours. It was purified on Silica Gel 60 (solvent: dichloromethane, dichloromethane-methanol 4:1, 1:1 and methanol). 2.3 g (52% of theor.) of product was obtained.

¹H-NMR (400 MHz, chloroform-d₁): δ=1.82 (s-br, 2H), 2.04 (mc, 2H), 2.62-2.74 (m, 2H), 2.75-2.84 (m, 4H), 3.14-3.23 (m, 4H), 3.66 (t, 2H).

### Intermediate 6-20

### 3-({3-[(3,3,3-Trifluoropropyl)sulphonyl]propyl}amino)propan-1-ol

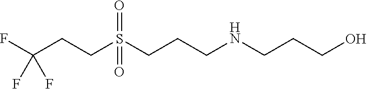 [SMILES: O=S(=O)(CCCNCCCO)CCC(F)(F)F]

4 g (16.76 mmol) of 3-[(3-chloropropyl)sulphonyl]-1,1,1-trifluoropropane and 5.88 mL 3-aminopropan-1-ol were stirred and worked up according to general specification 16-18-20-C for 30 hours. It was purified on Silica Gel 60 (solvent: dichloromethane, dichloromethane-methanol 4:1, 1:1 and methanol). 2.7 g (58% of theor.) of product was obtained.

¹H-NMR (400 MHz, chloroform-d₁): δ=1.70 (quin, 2H), 2.04 (mc, 2H), 2.61-2.74 (m, 2H), 2.79 (t, 2H), 2.86 (t, 2H), 3.13 (mc, 2H), 3.19 (mc, 2H), 3.79 (t, 2H).

### Intermediate 7-20

### 2-Methyl-1-({3-[(3,3,3-trifluoropropyl)sulphonyl]propyl}amino)propan-2-ol

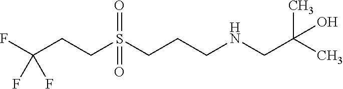 [SMILES: CC(C)(O)CNCCCS(=O)(=O)CCC(F)(F)F]

4 g (16.76 mmol) of 3-[(3-chloropropyl)sulphonyl]-1,1,1-trifluoropropane and 5.24 mL of 1-amino-2-methylpropan-2-ol were stirred and worked up according to general specification 16-18-20-C for 30 hours. It was purified on Silica Gel 60 (solvent: dichloromethane, dichloromethane-methanol 4:1 and 1:1). 2.1 g (43% of theor.) of product was obtained.

¹H-NMR (300 MHz, methanol-d₄): δ=1.19 (s, 6H), 1.93-2.05 (m, 2H), 2.53 (s, 2H), 2.62-2.79 (m, 4H), 3.24 (mc, 2H), 3.30-3.42 (m, 2H).

### Intermediate 8-20

### N-Methyl-3-[(3,3,4,4,4-pentafluorobutyl)sulphonyl]propan-1-amine

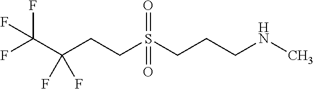 [SMILES: CNCCCS(=O)(=O)CCC(F)(F)C(F)(F)F]

7.7 g (26.67 mmol) of 3-chloropropyl-3,3,4,4,4-pentafluorobutylsulphone was stirred and worked up according to general specification 16-18-20-B for 20 hours. It was purified on Silica Gel 60 (solvent: dichloromethane, dichloromethane-methanol 2:1, 1:1 and methanol with 1.5 vol. % of 33% ammonia solution). 5.21 g (69% of theor.) of product was obtained.

¹H-NMR (400 MHz, DMSO-d₆): δ=2.03 (quin, 2H), 2.50 (s, 3H), 2.57-2.77 (m, 2H), 2.94 (t, 2H), 3.39 (t, 2H), 3.45 (mc, 2H).

### Intermediate 9-20

### 2-({3-[(4,4,5,5,5-Pentafluoropentyl)sulphonyl]propyl}amino)ethanol

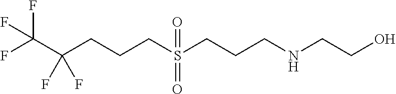 [SMILES: O=S(=O)(CCCNCCO)CCCC(F)(F)C(F)(F)F]

7.39 g (24.4 mmol) of 3-chloropropyl-4,4,5,5,5-pentafluoropentylsulphone and 5.97 g (97.7 mmol) of 3-aminopropan-1-ol were stirred according to general specification 16-18-20-C for 30 minutes at 120 watt in a microwave and extracted four times with chloroform. After extraction, a white precipitate from the combined organic phases was filtered with suction and dried. 385 mg (5% of theor.) of product was obtained. From the aqueous phase, a precipitate was filtered with suction, taken up in chloroform, washed once with water, dried over magnesium sulphate and concentrated by evaporation. 0.92 g (12% of theor.) of white product was obtained. The combined organic phases were dried over magnesium sulphate and concentrated by evaporation. It was purified on Silica Gel 60 (solvent: dichloromethane, dichloromethane-methanol 4:1, 1:1 and methanol with 4 vol. % of 33% ammonia solution). 1.36 g (17% of theor.) of product was obtained.

¹H-NMR (300 MHz, chloroform-d₁): δ=1.98-2.09 (m, 2H), 2.14-2.38 (m, 4H), 2.75-2.85 (m, 4H), 3.03-3.16 (m, 4H) 3.66 (mc, 2H).

### Intermediate 10-20

### 3-({3-[(4,4,5,5,5-Pentafluoropentyl)sulphonyl]propyl}amino)propan-1-ol

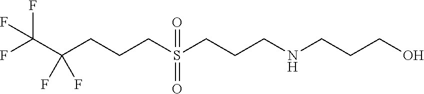 [SMILES: O=S(=O)(CCCNCCCO)CCCC(F)(F)C(F)(F)F]

7 g (23.1 mmol) of 3-chloropropyl-4,4,5,5,5-pentafluoropentylsulphone and 6.95 g (92.5 mmol) of 3-aminopropan-1-ol were stirred and worked up according to general specification 16-18-20-C for 7 days at 60° C. It was purified on Silica Gel 60 (solvent: dichloromethane, dichloromethane-methanol 4:1, 1:1 and methanol with 2 vol. % and 5 vol. % of 33% ammonia solution). 4.18 g (53% of theor.) of product was obtained.

¹H-NMR (400 MHz, chloroform-d₁): δ=1.71 (quin, 2H), 1.98-2.08 (m, 2H), 2.14-2.35 (m, 4H), 2.71 (br s, 2H), 2.79 (t, 2H), 2.87 (t, 2H), 3.03-3.11 (m, 4H) 3.79 (t, 2H).

### Intermediate 11-20

### 2-Methyl-1-({3-[(4,4,5,5,5-pentafluoropentyl)sulphonyl]propyl}amino)propan-2-ol

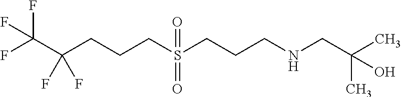 [SMILES: CC(C)(O)CNCCCS(=O)(=O)CCCC(F)(F)C(F)(F)F]

6.5 g (21.5 mmol) of 3-chloropropyl-4,4,5,5,5-pentafluoropentylsulphone and 4.86 g (54.6 mmol) of 1-amino-2-methylpropan-2-ol were stirred and worked up according to general specification 16-18-20-C for 8 days at 60° C. It was purified on Silica Gel 60 (solvent: dichloromethane, dichloromethane-methanol 4:1, 1:1 and methanol with 4 vol. % and 5 vol. % of 33% ammonia solution). 1.45 g (19% of theor.) of product was obtained.

¹H-NMR (400 MHz, chloroform-d₁): δ=1.19 (s, 6H), 2.03 (mc, 2H), 2.15-2.38 (m, 4H), 2.55 (s, 2H), 2.84 (t, 2H), 3.07 (t, 2H) 3.12 (mc, 2H).

### Intermediate 12-20

### N-(2-Methoxyethyl)-3-[(4,4,5,5,5-pentafluoropentyl)sulphonyl]propan-1-amine

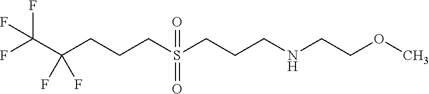 [SMILES: COCCNCCCS(=O)(=O)CCCC(F)(F)C(F)(F)F]

8.00 g (26.4 mmol) of 3-chloropropyl-4,4,5,5,5-pentafluoropentylsulphone and 5.96 g (79.3 mmol) of 2-methoxyethylamine were reacted according to general specification 16-18-20-C for 7 days at 60° C. It was purified on Silica Gel 60 (solvent: dichloromethane, dichloromethane-methanol 95:5, 90:10, 80:20, 50:50 and methanol with 4 vol. % of 33% ammonia solution). 3.36 g (37% of theor.) of product was obtained.

¹H-NMR (300 MHz, chloroform-d₁): δ=2.02 (mc, 2H), 2.12-2.38 (m, 4H), 2.75-2.83 (m, 4H), 3.06 (t, 2H), 3.13 (mc, 2H), 3.36 (s, 3H), 3.48 (t, 2H).

### Intermediate 13-20

### 3-Methoxy-N-{3-[(4,4,5,5,5-pentafluoropentyl)sulphonyl]propyl}propan-1-amine

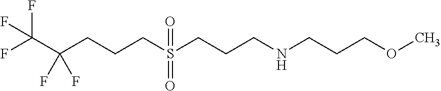 [SMILES: COCCCNCCCS(=O)(=O)CCCC(F)(F)C(F)(F)F]

8.00 g (26.4 mmol) of 3-chloropropyl-4,4,5,5,5-pentafluoropentylsulphone and 5.89 g (66.1 mmol) of 3-methoxypropylamine were reacted according to general specification 16-18-20-C for 7 days at 60° C. It was purified on Silica Gel 60 (solvent: dichloromethane, dichloromethane-methanol 95:5, 90:10, 70:30, 50:50 and methanol with 4 vol. % of 33% ammonia solution). 3.99 g (42% of theor.) of product was obtained.

¹H-NMR (300 MHz, chloroform-d₁): δ=1.74 (quin, 2H), 2.00 (mc, 2H), 2.12-2.37 (m, 4H), 2.68 (t, 2H), 2.76 (t, 2H), 3.06 (t, 2H), 3.12 (mc, 2H), 3.32 (s, 3H), 3.44 (t, 2H).

### Intermediate 14-20

### N-(2-Fluoroethyl)-3-[(4,4,5,5,5-pentafluoropentyl)sulphonyl]propan-1-amine

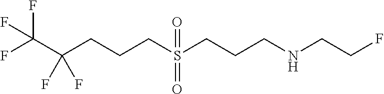 [SMILES: O=S(=O)(CCCNCCF)CCCC(F)(F)C(F)(F)F]

2.00 g (6.61 mmol) of 3-chloropropyl-4,4,5,5,5-pentafluoropentylsulphone, 1.97 g (19.79 mmol) of 2-fluoroethylamine hydrochloride and 2.01 g (19.86 mmol) of triethylamine were stirred in 20 mL ethanol for 3 days at 60° C. in a pressure tube. After cooling, it was concentrated by evaporation, the residue was taken up in 30 mL water (pH 6) and washed twice with dichloromethane. The aqueous phase was adjusted to a pH of 14 with 2M sodium hydroxide solution and was extracted three times with dichloromethane. These combined organic phases were dried over magnesium sulphate and concentrated by evaporation. 0.6 g (28% of theor.) of product was isolated.

¹H-NMR (400 MHz, chloroform-d₁): δ=2.02 (mc, 2H), 2.15-2.35 (m, 4H), 2.83 (t, 2H), 2.91 (dt, 2H), 3.07 (t, 2H), 3.14 (mc, 2H), 4.52 (dt, 2H).

### Intermediate 15-20

### N-{3-[(4,4,5,5,5-Pentafluoropentyl)sulphonyl]propyl}cyclopropanamine

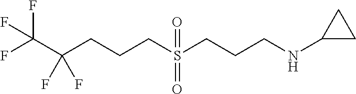 [SMILES: O=S(=O)(CCCNC1CC1)CCCC(F)(F)C(F)(F)F]

4.00 g (13.2 mmol) of 3-chloropropyl-4,4,5,5,5-pentafluoropentylsulphone and 3.02 g (52.9 mmol) of cyclopropylamine were stirred in 24 mL ethanol for 2 days at 60° C. in a pressure tube. After cooling, it was concentrated by evaporation, the residue was taken up in water and was washed three times with dichloromethane. The aqueous phase was adjusted with 2M sodium hydroxide solution to a pH of 14 and was extracted three times with dichloromethane. These combined organic phases were dried over magnesium sulphate and concentrated by evaporation. 0.5 g (12% of theor.) of product was isolated.

¹H-NMR (400 MHz, chloroform-d₁): δ=0.27-0.31 (m, 2H), 0.45 (mc, 2H), 2.01 (mc, 2H), 2.11 (mc, 1H), 2.14-2.35 (m, 4H), 2.85 (t, 2H), 3.02-3.11 (m, 4H).

### Intermediate 16-20

### N-Methyl-4-[(3,3,3-trifluoropropyl)sulphonyl]butan-1-amine

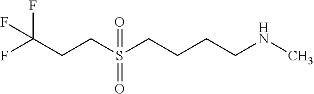

5.0 g (19.8 mmol) of 1-chloro-4-[(3,3,3-trifluoropropyl)sulphonyl]butane was stirred in 80 mL of 33% ethanolic methylamine solution for 24 hours at 40° C. The volatile constituents were drawn off, 50 mL water was added and it was washed twice with dichloromethane. The pH was adjusted to 14 with 2M sodium hydroxide solution and it was extracted three times with dichloromethane. These combined organic phases were dried over magnesium sulphate and concentrated by evaporation. 4.4 g (90% of theor.) of product was obtained.

¹H-NMR (400 MHz, chloroform-d₁): δ=1.65 (quin, 2H), 1.88-1.98 (m, 2H), 2.43 (s, 3H), 2.66-2.75 (m, 4H), 3.08 (mc, 2H), 3.15-3.21 (m, 2H).

### Intermediate 17-20

### N-tert-Butyl-3-[(4,4,5,5,5-pentafluoropentyl)sulphonyl]propan-1-amine

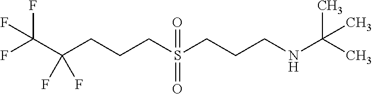 [SMILES: CC(C)(C)NCCCS(=O)(=O)CCCC(F)(F)C(F)(F)F]

2.70 g (8.92 mmol) of 3-chloropropyl-4,4,5,5,5-pentafluoropentylsulphone and 4.57 g (62.5 mmol) of tert-butylamine were stirred in 20 mL of DMF for 3 days at 75° C. in a pressure tube. After cooling, it was concentrated by evaporation, the residue was taken up in 50 mL water and washed three times with dichloromethane. The aqueous phase was adjusted with 2M sodium hydroxide solution to a pH of 14 and extracted three times with dichloromethane. These combined organic phases were dried over magnesium sulphate and concentrated by evaporation. 1.8 g (59% of theor.) of product was isolated.

¹H-NMR (400 MHz, chloroform-d₁): δ=1.08 (s, 9H), 1.95 (mc, 2H), 2.15-2.34 (m, 4H), 2.70 (t, 2H), 3.06 (t, 2H), 3.14 (mc, 2H).

### Intermediate 18-20

### 3-[(4,4,5,5,5-Pentafluoropentyl)sulphonyl]-N-(2,2,2-trifluoroethyl)propan-1-amine

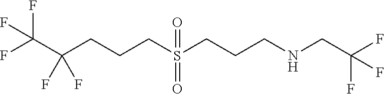 [SMILES: O=S(=O)(CCCNCC(F)(F)F)CCCC(F)(F)C(F)(F)F]

1.00 g (3.30 mmol) of 3-chloropropyl-4,4,5,5,5-pentafluoropentylsulphone and 1.636 g (16.52 mmol) 2,2,2-trifluoroethylamine were stirred in 3 mL of DMF for 6 days at 100° C. in a pressure tube. After cooling, it was concentrated by evaporation, the residue was taken up in water and shaken three times with dichloromethane. The combined organic phases were dried over magnesium sulphate and concentrated by evaporation. It was purified on Silica Gel 60 (solvent: dichloromethane, dichloromethane-methanol 99:1). 0.8 g (66% of theor.) of product was isolated.

¹H-NMR (400 MHz, chloroform-d₁): δ=2.01 (mc, 2H), 2.15-2.35 (m, 4H), 2.91 (t, 2H), 3.08 (t, 2H), 3.11-3.23 (m, 4H).

### Intermediate 19-20

### N-(2,2-Difluoroethyl)-3-[(4,4,5,5,5-pentafluoropentyl)sulphonyl]propan-1-amine

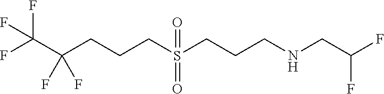 [SMILES: O=S(=O)(CCCNCC(F)F)CCCC(F)(F)C(F)(F)F]

2.50 g (8.26 mmol) of 3-chloropropyl-4,4,5,5,5-pentafluoropentylsulphone and 2.01 g (24.8 mmol) of 2,2-difluoroethylamine were stirred in 20 mL ethanol for 3 days at 60° C. in a pressure tube. It was concentrated by evaporation, the residue was taken up in water and washed twice with dichloromethane. The aqueous phase was adjusted with 2M sodium hydroxide solution to pH 14 and shaken three times with dichloromethane. These combined organic phases were dried over magnesium sulphate and concentrated by evaporation. 0.5 g (17% of theor.) of product was isolated.

¹H-NMR (400 MHz, chloroform-d₁): δ=2.01 (mc, 2H), 2.15-2.35 (m, 4H), 2.86 (t, 2H), 2.97 (dt, 2H), 3.07 (t, 2H), 3.13 (mc, 2H), 5.82 (tt, 1H).

### Intermediate 20-20

### N-(4-Fluorobenzyl)-3-[(4,4,5,5,5-pentafluoropentyl)sulphonyl]propan-1-amine

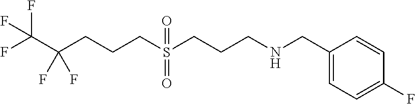 [SMILES: O=S(=O)(CCCNCc1ccc(F)cc1)CCCC(F)(F)C(F)(F)F]

2.50 g (8.26 mmol) of 3-chloropropyl-4,4,5,5,5-pentafluoropentylsulphone, 4.134 g (33.04 mmol) of 4-fluorobenzylamine, 1.751 g (16.52 mmol) of sodium carbonate and 2.476 g (16.52 mmol) of sodium iodide were stirred in 20 mL acetonitrile for 15 hours at 80° C. The volatile constituents were drawn off and the residue was taken up in dichloromethane. It was washed three times with water, dried over magnesium sulphate and concentrated by evaporation. Pentane was added to the residue and it was filtered with suction. 2.8 g (87% of theor.) of product was isolated.

¹H-NMR (400 MHz, chloroform-d₁): δ=2.01 (mc, 2H), 2.13-2.34 (m, 4H), 2.77 (t, 2H), 3.04 (t, 2H), 3.13 (mc, 2H), 3.75 (s, 2H), 7.01 (mc, 2H), 7.23-7.30 (m, 2H).

### Intermediate 21-20

### N-Methyl-5-[(3,3,3-trifluoropropyl)sulphonyl]pentan-1-amine

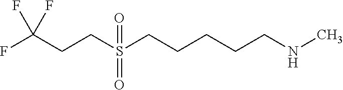 [SMILES: CNCCCCCS(=O)(=O)CCC(F)(F)F]

6.1 g (22.9 mmol) of 1-chloro-5-[(3,3,3-trifluoropropyl)sulphonyl]pentane was stirred and worked up according to general specification 16-18-20-D for 24 hours. 3.53 g (59% of theor.) of product was obtained.

¹H-NMR (400 MHz, chloroform-d₁): δ=1.47-1.60 (m, 4H), 1.89 (mc, 2H), 2.43 (s, 3H), 2.57-2.74 (m, 4H), 3.04 (mc, 2H), 3.17 (mc, 2H).

### Intermediate 22-20

### N-Methyl-4-[(3,3,4,4,4-pentafluorobutyl)sulphonyl]butan-1-amine

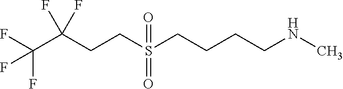 [SMILES: CNCCCCS(=O)(=O)CCC(F)(F)C(F)(F)F]

4.5 g (14.9 mmol) of 4-[(4-chlorobutyl)sulphonyl]-1,1,1,2,2-pentafluorobutane was stirred and worked up in 150 mL of 33% methylamine solution in ethanol according to general specification 16-18-20-D for 24 hours. 3.67 g (83% of theor.) of product was obtained.

¹H-NMR (400 MHz, chloroform-d₁): δ=1.66 (quin, 2H), 1.95 (mc, 2H), 2.43 (s, 3H), 2.56-2.70 (m, 4H), 3.10 (mc, 2H), 3.20 (mc, 2H).

### Intermediate 23-20

### Benzyl-N-{4-[(4,4,4-trifluorobutyl)sulphonyl]butyl}glycinate

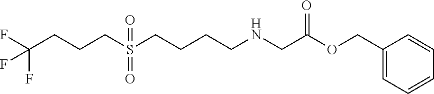 [SMILES: O=C(CNCCCCS(=O)(=O)CCCC(F)(F)F)OCc1ccccc1]

1 g (3.45 mmol) of 4-[(4-chlorobutyl)sulphonyl]-1,1,1-trifluorobutane was stirred with 3.024 g (15.00 mmol) of aminoacetic acid benzyl ester hydrochloride, 1.987 g (18.75 mmol) of sodium carbonate and 843.0 mg (5.62 mmol) of sodium iodide in 25 mL acetonitrile for 24 hours under reflux. The volatile constituents were drawn off and water was added to the residue. It was extracted with dichloromethane four times. The combined organic phases were dried over magnesium sulphate and concentrated by evaporation. The residue was purified using Silica Gel 60 (solvent: dichloromethane, dichloromethane-methanol 98:2, 95:5 and 90:10). Diisopropyl ether was added to the raw product, it was sonicated in an ultrasonic bath, filtered with suction and dried at 40° C. in a drying cabinet. 455.5 mg (29% of theor.) of product was obtained.

¹H-NMR (300 MHz, chloroform-d₁): δ=1.65 (quin, 2H), 1.92 (mc, 2H), 2.09-2.20 (m, 2H), 2.24-2.41 (m, 2H), 2.67 (t, 2H), 2.98-3.07 (m, 4H), 3.45 (s, 2H), 5.17 (s, 2H), 7.30-7.42 (m, 5H).

### Intermediate 24-20

### N-Methyl-3-[(5,5,6,6,6-pentafluorohexyl)sulphonyl]propan-1-amine

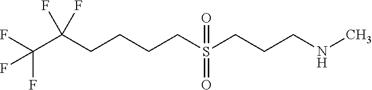 [SMILES: CNCCCS(=O)(=O)CCCCC(F)(F)C(F)(F)F]

5 g (15.79 mmol) of 3-chloropropyl-5,5,6,6,6-pentafluorohexylsulphone was stirred and worked up in 100 mL of 33% methylamine solution in ethanol according to general specification 16-18-20-D for 24 hours. 4.18 g (85% of theor.) of product was obtained.

¹H-NMR (300 MHz, chloroform-d₁): δ=1.69-1.84 (m, 2H), 1.87-2.21 (m, 6H), 2.41 (s, 3H), 2.72 (t, 2H), 2.99 (t, 2H), 3.07 (mc, 2H).

### Intermediate 25-20

### N-Methyl-3-[(5,5,5-trifluoropentyl)sulphonyl]propan-1-amine

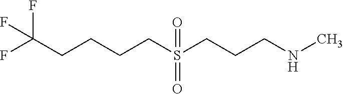 [SMILES: CNCCCS(=O)(=O)CCCCC(F)(F)F]

4.3 g (16.12 mmol) of 3-chloropropyl-5,5,5-trifluoropentylsulphone was stirred and worked up in 100 mL of 33% methylamine solution in ethanol according to general specification 16-18-20-D for 24 hours. 3.49 g (83% of theor.) of product was obtained.

¹H-NMR (300 MHz, chloroform-d₁): δ=1.67-1.81 (m, 2H), 1.88-2.24 (m, 6H), 2.43 (s, 3H), 2.73 (t, 2H), 2.99 (mc, 2H), 3.08 (mc, 2H).

### Intermediate 26-20

### 2-({4-[(4,4,4-Trifluorobutyl)sulphonyl]butyl}amino)ethanol

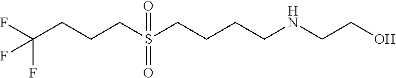 [SMILES: O=S(=O)(CCCCNCCO)CCCC(F)(F)F]

1.5 g (5.62 mmol) of 4-[(4-chlorobutyl)sulphonyl]-1,1,1-trifluorobutane and 1.72 g (28.12 mmol) of 2-aminoethanol was reacted according to general specification 16-18-20-C for 30 hours at 55° C. Pentane was added to the product and it was filtered with suction. 0.96 g (53% of theor.) of product was obtained.

¹H-NMR (300 MHz, chloroform-d₁): δ=1.66 (quin, 2H), 1.93 (mc, 2H), 2.09-2.21 (m, 2H), 2.25-2.42 (m, 2H), 2.69 (t, 2H), 2.78 (t, 2H), 2.97-3.10 (m, 4H), 3.64 (t, 2H).

### Intermediate 27-20

### (2S)-1-({4-[(4,4,4-Trifluorobutyl)sulphonyl]butyl}amino)propan-2-ol

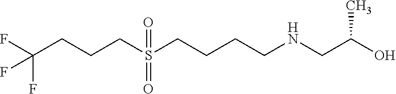 [SMILES: C[C@H](O)CNCCCCS(=O)(=O)CCCC(F)(F)F]

1.5 g (5.62 mmol) of 4-[(4-chlorobutyl)sulphonyl]-1,1,1-trifluorobutane and 2.11 g (28.12 mmol) of (2S)-1-aminopropan-2-ol were reacted according to general specification 16-18-20-C for 30 hours at 55° C. Pentane was added to the product and it was filtered with suction. 1.5 g (87% of theor.) of product was obtained.

¹H-NMR (300 MHz, chloroform-d₁): δ=1.15 (d, 3H), 1.64 (quin, 2H), 1.92 (mc, 2H), 2.08-2.20 (m, 2H), 2.24-2.45 (m, 3H), 2.59-2.76 (m, 3H), 2.96-3.08 (m, 4H), 3.75 (mc, 1H).

### Intermediate 28-20

### (2R)-1-({4-[(4,4,4-Trifluorobutyl)sulphonyl]butyl}amino)propan-2-ol

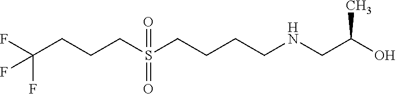 [SMILES: C[C@@H](O)CNCCCCS(=O)(=O)CCCC(F)(F)F]

1.5 g (5.62 mmol) of 4-[(4-chlorobutyl)sulphonyl]-1,1,1-trifluorobutane and 2.11 g (28.12 mmol) of (2R)-1-aminopropan-2-ol were reacted according to general specification 16-18-20-C for 30 hours at 55° C. Pentane was added to the product and it was filtered with suction. Because this time the product still contained a large amount of the starting material, it was stirred with 2.1 g of (2R)-1-aminopropan-2-ol in 20 mL methanol for 30 hours at 60° C. It was evaporated to dryness. Water was added to the residue and it was acidified with dilute hydrochloric acid. It was extracted twice with dichloromethane. The aqueous phase was made alkaline with 2M sodium hydroxide solution and was shaken three times with dichloromethane. The combined organic phases were dried over magnesium sulphate and concentrated by evaporation. Pentane was added to the raw product and it was filtered with suction. 1.3 g (76% of theor.) of product was obtained.

¹H-NMR (400 MHz, chloroform-d₁): δ=1.15 (d, 3H), 1.65 (quin, 2H), 1.92 (mc, 2H), 2.10-2.19 (m, 2H), 2.27-2.43 (m, 3H), 2.61-2.75 (m, 3H), 2.98-3.07 (m, 4H), 3.76 (mc, 1H).

### Intermediate 29-20

### 2-({3-[(4,4,4-Trifluorobutyl)sulphonyl]propyl}amino)ethanol

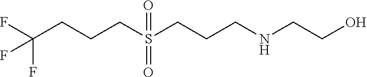 [SMILES: O=S(=O)(CCCNCCO)CCCC(F)(F)F]

1.5 g (5.94 mmol) of 3-chloropropyl-4,4,4-trifluorobutylsulphone and 1.81 g (29.68 mmol) of 2-aminoethanol were reacted according to general specification 16-18-20-C for 30 hours at 60° C. Water was added to the residue and it was acidified with dilute hydrochloric acid. It was extracted twice with dichloromethane. The aqueous phase was made alkaline with 2M sodium hydroxide solution, sodium chloride was added and it was shaken with chloroform five times. The combined organic phases were dried over magnesium sulphate and concentrated by evaporation. Pentane was added to the raw product and it was filtered with suction. 0.8 g (44% of theor.) of product was obtained.

¹H-NMR (400 MHz, chloroform-d₁): δ=2.03 (mc, 2H), 2.11-2.20 (m, 2H), 2.27-2.40 (m, 2H), 2.76-2.84 (m, 4H), 3.06 (t, 2H), 3.12 (mc, 2H), 3.66 (t, 2H).

### Intermediate 30-20

### 3-[(5,5,5-Trifluoropentyl)sulphonyl]propan-1-amine

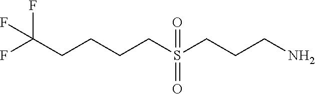 [SMILES: NCCCS(=O)(=O)CCCCC(F)(F)F]

3.2 g (12.0 mmol) of 3-chloropropyl-5,5,5-trifluoropentylsulphone was stirred in 260 mL of 7M ammonia solution in methanol for 48 hours at 80° C. It was concentrated by evaporation, dissolved in water, extracted twice with dichloromethane, made basic with 2M NaOH and shaken three times with dichloromethane. The combined organic phases were dried over magnesium sulphate and concentrated by evaporation. 2.0 g (67% of theor.) of product was obtained.

¹H-NMR (300 MHz, chloroform-d₁): δ=1.69-1.81 (m, 2H), 1.89-2.03 (m, 4H), 2.07-2.24 (m, 2H), 2.88 (t, 2H), 3.00 (mc, 2H), 3.09 (mc, 2H).

### Intermediate 31-20

### N-Methyl-3-[(4,4,4-trifluorobutyl)sulphonyl]propan-1-amine

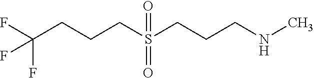

1.0 g (3.96 mmol) of 3-chloropropyl-4,4,4-trifluorobutylsulphone was stirred and worked up in 50 mL of 33% methylamine solution in ethanol according to general specification 16-18-20-D for 24 hours. 0.56 g (57% of theor.) of product was obtained.

¹H-NMR (400 MHz, chloroform-d₁): δ=2.00 (mc, 2H), 2.10-2.19 (m, 2H), 2.25-2.38 (m, 2H), 2.42 (s, 3H), 2.73 (t, 2H), 3.04 (mc, 2H), 3.10 (mc, 2H).

### Intermediate 32-20

### N-Methyl-3-[(6,6,6-trifluorohexyl)sulphonyl]propan-1-amine

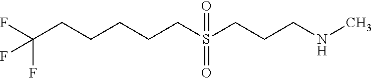 [SMILES: CNCCCS(=O)(=O)CCCCCC(F)(F)F]

1.5 g (5.34 mmol) of 3-chloropropyl-6,6,6-trifluorohexylsulphone was stirred and worked up in 100 mL of 33% methylamine solution in ethanol according to general specification 16-18-20-D for 24 hours. 0.75 g (51% of theor.) of product was obtained.

¹H-NMR (300 MHz, chloroform-d₁): δ=1.47-1.68 (m, 4H), 1.88 (mc, 2H), 1.94-2.21 (m, 4H), 2.42 (s, 3H), 2.73 (t, 2H), 2.97 (mc, 2H), 3.07 (mc, 2H).

### Intermediate 33-20

### N-Methyl-4-[(4,4,4-trifluorobutyl)sulphonyl]butan-1-amine

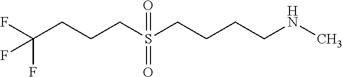 [SMILES: CNCCCCS(=O)(=O)CCCC(F)(F)F]

15.0 g (56.2 mmol) of 4-[(4-chlorobutyl)sulphonyl]-1,1,1-trifluorobutane was stirred and worked up in 300 mL of 33% methylamine solution in ethanol according to general specification 16-18-20-D for 36 hours. 12.8 g (87% of theor.) of product was obtained.

¹H-NMR (400 MHz, chloroform-d₁): δ=1.65 (quin, 2H), 1.87-1.97 (m, 2H), 2.10-2.20 (m, 2H), 2.26-2.41 (m, 2H), 2.43 (s, 3H), 2.64 (t, 2H), 3.00-3.07 (mc, 4H).

### Intermediate 34-20

### 4-[(4,4,4-Trifluorobutyl)sulphonyl]butan-1-amine

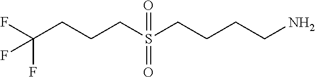 [SMILES: NCCCCS(=O)(=O)CCCC(F)(F)F]

0.5 g (1.87 mmol) of 4-[(4-chlorobutyl)sulphonyl]-1,1,1-trifluorobutane was stirred in 40 mL of 7M ammonia solution in methanol for 48 hours at 80° C. in an autoclave. It was evaporated to dryness. The residue was taken up in 25 mL water and washed twice with dichloromethane. The aqueous phase was made basic with 2M sodium hydroxide solution. It was shaken three times with dichloromethane, dried over magnesium sulphate and concentrated by evaporation. 330 mg (71% of theor.) of product was isolated.

¹H-NMR (300 MHz, chloroform-d₁): δ=1.60 (quin, 2H), 1.85-1.97 (m, 2H), 2.09-2.21 (m, 2H), 2.25-2.42 (m, 2H), 2.76 (t, 2H), 2.98-3.08 (m, 4H).

### Intermediate 35-20

### N-Methyl-4-{[3,4,4,4-tetrafluoro-3-(trifluoromethyl)butyl]sulphonyl}butan-1-amine

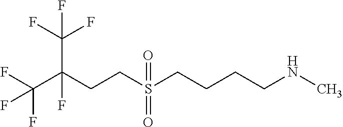 [SMILES: CNCCCCS(=O)(=O)CCC(F)(C(F)(F)F)C(F)(F)F]

4 g (11.34 mmol) of 4-[(4-chlorobutyl)sulphonyl]-1,1,1,2-tetrafluoro-2-(trifluoromethyl)butane was stirred in 150 mL of 33% methylamine solution in ethanol according to general specification 16-18-20-D for 23 hours and concentrated by evaporation. It was taken up in 100 mL water, adjusted to a pH of 1 with 4M hydrochloric acid and extracted twice with dichloromethane. The aqueous phase was adjusted with 2M sodium hydroxide solution to pH 14 and was extracted three times with dichloromethane. The combined organic phases were dried over magnesium sulphate and concentrated by evaporation. 1.94 g (48% of theor.) of product was obtained.

¹H-NMR (400 MHz, chloroform-d₁): δ=1.66 (quin, 2H), 1.94 (mc, 2H), 2.43 (s, 3H), 2.61-2.73 (m, 4H), 3.09 (mc, 2H), 3.18 (mc, 2H).

### Intermediate 36-20

### N-Methyl-3-{[3,4,4,4-tetrafluoro-3-(trifluoromethyl)butyl]sulphonyl}propan-1-amine

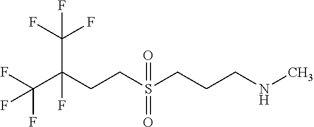 [SMILES: CNCCCS(=O)(=O)CCC(F)(C(F)(F)F)C(F)(F)F]

4 g (11.81 mmol) of 3-chloropropyl-3,4,4,4-tetrafluoro-3-(trifluoromethyl)butylsulphone was stirred in 150 mL of 33% methylamine solution in ethanol according to general specification 16-18-20-D for 23 hours and was concentrated by evaporation. It was taken up in 100 mL water, adjusted to a pH of 1 with 4M hydrochloric acid and extracted twice with dichloromethane. The aqueous phase was adjusted with 2M sodium hydroxide solution to pH 14 and extracted three times with dichloromethane. The combined organic phases were dried over magnesium sulphate and concentrated by evaporation. 2.0 g (46% of theor.) of product was obtained.

¹H-NMR (400 MHz, chloroform-d₁): δ=2.03 (mc, 2H), 2.43 (s, 3H), 2.61-2.72 (m, 2H), 2.75 (t, 2H), 3.16-3.24 (m, 4H).

### Intermediate 37-20

### 2-[(3-{[3,4,4,4-Tetrafluoro-3-(trifluoromethyl)butyl]sulphonyl}propyl)amino]ethanol

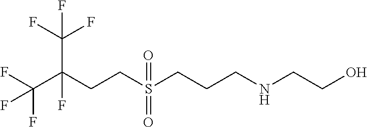 [SMILES: O=S(=O)(CCCNCCO)CCC(F)(C(F)(F)F)C(F)(F)F]

1.8 g (5.31 mmol) of 3-chloropropyl-3,4,4,4-tetrafluoro-3-(trifluoromethyl)butylsulphone and 2.27 g (37.20 mmol) of 2-aminoethanol were reacted according to general specification 16-18-20-C for 30 hours at 60° C. and concentrated by evaporation. Water was added to the residue and it was adjusted to pH 1 with dilute hydrochloric acid. It was shaken twice with dichloromethane. The aqueous phase was adjusted with 2M sodium hydroxide solution to pH 14 and was extracted five times with dichloromethane. The combined organic phases were dried over magnesium sulphate and concentrated by evaporation. 1.1 g (57% of theor.) of product was obtained.

¹H-NMR (300 MHz, chloroform-d₁): δ=2.04 (mc, 2H), 2.59-2.74 (m, 2H), 2.75-2.85 (m, 4H), 3.15-3.25 (m, 4H), 3.66 (t, 2H).

### Intermediate 38-20

### 2-[(4-{[3,4,4,4-Tetrafluoro-3-(trifluoromethyl)butyl]sulphonyl}butyl)amino]ethanol

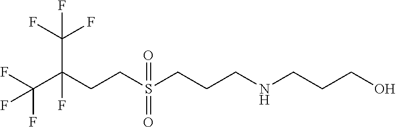 [SMILES: O=S(=O)(CCCNCCCO)CCC(F)(C(F)(F)F)C(F)(F)F]

1.8 g (5.10 mmol) of 4-[(4-chlorobutyl)sulphonyl]-1,1,1,2-tetrafluoro-2-(trifluoromethyl)butane and 2.18 g (35.72 mmol) of 2-aminoethanol were reacted according to general specification 16-18-20-C for 30 hours at 60° C. and concentrated by evaporation. Water was added to the residue and it was adjusted to pH 1 with dilute hydrochloric acid. It was shaken twice with dichloromethane. The aqueous phase was adjusted with 2M sodium hydroxide solution to pH 14 and extracted with chloroform five times. The combined organic phases were dried over magnesium sulphate and concentrated by evaporation. 0.52 g (27% of theor.) of product was obtained.

¹H-NMR (300 MHz, chloroform-d₁): δ=1.66 (quin, 2H), 1.95 (mc, 2H), 2.59-2.74 (m, 4H), 2.77 (t, 2H), 3.08 (mc, 2H), 3.18 (mc, 2H), 3.65 (t, 2H).

### Intermediate 39-20

### N-(²H₃)methyl-3-[(5,5,5-trifluoropentyl)sulphonyl]propan-1-amine

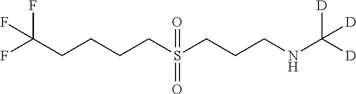 [SMILES: [2H]C([2H])([2H])NCCCS(=O)(=O)CCCCC(F)(F)F]

2.5 g (9.37 mmol) of 3-chloropropyl-5,5,5-trifluoropentylsulphone and 6.0 g (176.1 mmol) of (²H₃)methanamine were reacted in 30 mL ethanol for 24 hours at 40° C. and concentrated by evaporation. Water was added to the residue and it was shaken twice with dichloromethane. The aqueous phase was adjusted with 2M sodium hydroxide solution to pH 10 and extracted with dichloromethane four times. The combined organic phases were dried over magnesium sulphate and concentrated by evaporation. 1.3 g (52% of theor.) of product was obtained.

¹H-NMR (300 MHz, chloroform-d₁): δ=1.67-1.80 (m, 2H), 1.88-2.07 (m, 4H), 2.08-2.23 (m, 2H), 2.73 (t, 2H), 2.98 (mc, 2H), 3.08 (m, 2H).

### Intermediate 40-20

### 3-[(4,4-Difluorocyclohexyl)sulphonyl]-N-methylpropan-1-amine (trifluoroacetic acid salt)

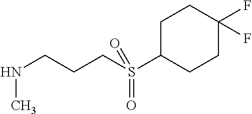 [SMILES: CNCCCS(=O)(=O)C1CCC(F)(F)CC1]

186 mg of tert-butyl-{3-[(4,4-difluorocyclohexyl)sulphonyl]propyl}methylcarbamate was put in 8 mL dichloromethane and 0.40 mL trifluoroacetic acid was added. After 18 hours of stirring at room temperature it was concentrated by evaporation, toluene was added several times and it was dried in vacuum. 238 mg of the title compound was obtained as trifluoroacetic acid salt.

MS (CI): Mass found=256 [100]

### Intermediate 41-20

### 4-[(4,4-Difluorocyclohexyl)sulphonyl]-N-methylbutan-1-amine

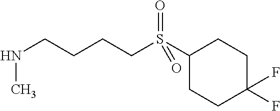 [SMILES: CNCCCCS(=O)(=O)C1CCC(F)(F)CC1]

Preparation was carried out similarly to intermediate 40-20 starting from tert-butyl-{4-[(4,4-difluorocyclohexyl)sulphonyl]butyl}methylcarbamate.

¹H-NMR (300 MHz, chloroform-di, selected signals): δ 2.16-2.39 (m, 4H), 2.45 (s, 3H), 2.65 (t, 2H), 2.84-3.04 (m, 3H), MS (CI): Mass found=270 [100].

### Intermediate 42-20

### 3-{[(4,4-Difluorocyclohexyl)methyl]sulphonyl}-N-methylpropan-1-amine

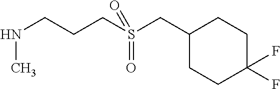 [SMILES: CNCCCS(=O)(=O)CC1CCC(F)(F)CC1]

Preparation was carried out similarly to intermediate 40-20 starting from tert-butyl-(3-{[(4,4-difluorocyclohexyl)methyl]sulphonyl}propyl)methylcarbamate.

¹H-NMR (300 MHz, chloroform-di, selected signals): δ 2.44 (s, 3H), 2.75 (t, 2H), 2.91 (d, 2H), 3.06-3.14 (t, 2H).

### Intermediates 21

### Intermediate 1-21

### tert-Butyl-{3-[(4,4-difluorocyclohexyl)sulphanyl]propyl}methylcarbamate

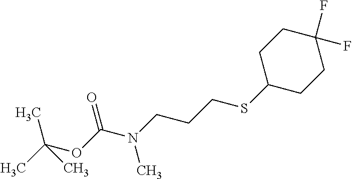 [SMILES: CN(CCCSC1CCC(F)(F)CC1)C(=O)OC(C)(C)C]

558 mg sodium methanolate was added to a solution of 1.28 g of S-{3-[(tert-butoxycarbonyl)(methyl)amino]propyl}ethanethioate in 13 mL methanol and it was stirred at room temperature for 30 min. 1.00 g of 4,4-difluorocyclohexyl-4-methylbenzene sulphonate was added and it was heated in a microwave (100° C./100 watt/60 min). The reaction mixture was diluted with tert-butyl methyl ether and water, the phases were separated, extracted twice with tert-butyl methyl ether and the combined organic phases were washed with sodium chloride solution and dried over sodium sulphate. After purification by column chromatography on silica gel (hexane/ethyl acetate), 464 mg of the title compound was obtained.

¹H-NMR (300 MHz, chloroform-d₁): δ 1.45 (s, 9H), 1.65-1.91 (m, 6H), 1.94-2.24 (m, 4H), 2.52 (t, 2H), 2.74-2.84 (m, 1H), 2.85 (s, 3H), 3.29 (t, 2H). MS (CI): m/z=324, 268, 224 [100].

### Intermediate 2-21

### tert-Butyl-{4-[(4,4-difluorocyclohexyl)sulphanyl]butyl}methylcarbamate

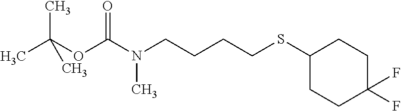 [SMILES: CN(CCCCSC1CCC(F)(F)CC1)C(=O)OC(C)(C)C]

Preparation was carried out similarly to intermediate 1-21 starting from S-{4-[(tert-butoxycarbonyl)(methyl)amino]butyl}ethanethioate. The title compound was obtained as raw product.

MS (CI): Mass found=338, 282, 238.

### Intermediate 3-21

### tert-Butyl-(3-{[(4,4-difluorocyclohexyl)methyl]sulphanyl}propyl)methylcarbamate

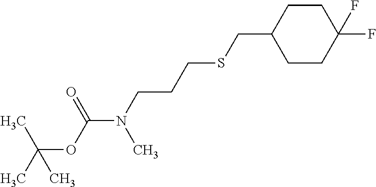 [SMILES: CN(CCCSCC1CCC(F)(F)CC1)C(=O)OC(C)(C)C]

S-{4-[(tert-butoxycarbonyl)(methyl)amino]propyl}ethanethioate by reaction with 4-(bromomethyl)-1,1-difluorocyclohexane. The title compound was obtained as raw product.

MS (CI): Mass found=338, 282 [100], 238

### Intermediates 22

### Intermediate 1-22

### tert-Butyl-{3-[(4,4-difluorocyclohexyl)sulphonyl]propyl}methylcarbamate

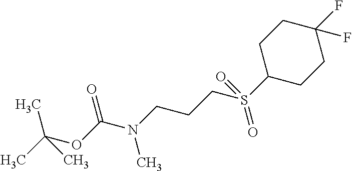 [SMILES: CN(CCCS(=O)(=O)C1CCC(F)(F)CC1)C(=O)OC(C)(C)C]

460 mg of tert-butyl-{3-[(4,4-difluorocyclohexyl)sulphanyl]propyl}methylcarbamate was reacted with meta-chloroperbenzoic acid, similarly to general specification 19. 140 mg of the title compound was obtained by purification by column chromatography on silica gel (hexane/ethyl acetate)

¹H-NMR (300 MHz, chloroform-d₁): δ 1.45 (s, 9H), 1.65-2.15 (m, 6H), 2.17-2.38 (m, 4H), 2.82-3.00 (m, 6H, contains s at 2.87 ppm), 3.38 (t, 2H). MS (CI): Mass found=356, 300, 256.

### Intermediate 2-22

### tert-Butyl-{4-[(4,4-difluorocyclohexyl)sulphonyl]butyl}methylcarbamate

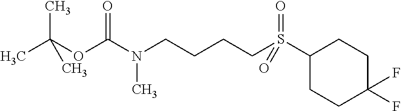 [SMILES: CN(CCCCS(=O)(=O)C1CCC(F)(F)CC1)C(=O)OC(C)(C)C]

tert-Butyl-{3-[(4,4-difluorocyclohexyl)sulphanyl]butyl}methylcarbamate was reacted with meta-chloroperbenzoic acid to the title compound, similarly to general specification 19.

¹H-NMR (300 MHz, chloroform-d₁): δ 1.45 (s, 9H), 1.62-2.03 (m, 8H), 2.18-2.38 (m, 4H), 2.78-3.11 (m, 6H), 3.27 (t, 2H). MS (CI): m/z=370, 314 [100], 270

### Intermediate 3-22

### tert-Butyl-(3-{[(4,4-difluorocyclohexyl)methyl]sulphonyl}propyl)methyl-carbamate

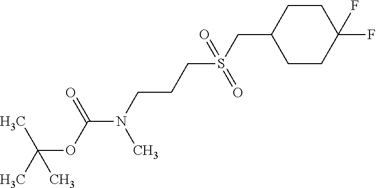 [SMILES: CN(CCCS(=O)(=O)CC1CCC(F)(F)CC1)C(=O)OC(C)(C)C]

Preparation of the title compound was carried out similarly to general specification 19 with meta-chloroperbenzoic acid starting from tert-butyl-(3-{[(4,4-difluorocyclohexyl)methyl]sulphanyl}propyl)methylcarbamate.

¹H-NMR (300 MHz, chloroform-di, selected signals): δ 1.46 (s, 9H), 2.86 (s, 3H), 2.90 (d, 2H), 2.93-3.02 (m, 2H), 3.04 (t, 2H). MS (CI): m/z=370, 314, 270

### EXAMPLES

General specification 11 for preparation of the examples under protective gas atmosphere and with exclusion of moisture: 1 g bromide was dissolved in approx. 30-55 mL DMF. 1.2-1.4 equivalents of amine (relative to the bromide), 0.5 equivalent of sodium iodide (relative to the bromide) and 1.0 equivalent of sodium carbonate (relative to the bromide) were added. It was stirred for 10-20 hours at 85° C. bath temperature. After cooling to room temperature, the solution was concentrated in the oil pump vacuum in a rotary evaporator. The residue was taken up in ethyl acetate or dichloromethane, washed two or three times (water, optionally saturated sodium chloride solution), dried over magnesium sulphate and concentrated by evaporation. Then it was chromatographed using Silica Gel 60 or by HPLC.

### Example 1

### 8-(3,5-Difluorophenyl)-9-[6-(methyl{3-[(4,4,5,5,5-pentafluoropentyl)sulphonyl]propyl}amino)hexyl]-6,7-dihydro-5H-benzo[7]annulen-3-ol

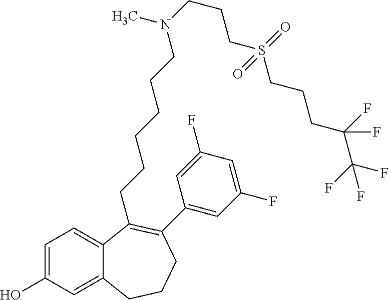 [SMILES: CN(CCCCCCC1=C(c2cc(F)cc(F)c2)CCCc2cc(O)ccc21)CCCS(=O)(=O)CCCC(F)(F)C(F)(F)F]

160 mg (0.37 mmol) of 9-(6-bromohexyl)-8-(3,5-difluorophenyl)-6,7-dihydro-5H-benzo[7]annulen-3-ol was reacted with 153 mg (0.52 mmol) of N-methyl-3-[(4,4,5,5,5-pentafluoropentyl)sulphonyl]propan-1-amine according to general specification 11. It was purified using HPLC-Method 2. 129.1 mg (54% of theor.) of product was isolated.

¹H-NMR (400 MHz, chloroform-d₁): δ=1.02-1.26 (m, 6H), 1.30-1.39 (m, 2H), 2.04-2.41 (m, 12H), 2.43-2.49 (m, 5H), 2.57-2.64 (m, 2H), 2.85 (t, 2H), 3.14 (mc, 4H), 6.67-6.80 (m, 5H), 7.14 (d, 1H).

### Example 2

### 8-(3,5-Difluorophenyl)-9-[6-(methyl{3-[(3,3,4,4,4-pentafluorobutyl)sulphinyl]propyl}amino)hexyl]-6,7-dihydro-5H-benzo[7]annulen-3-ol

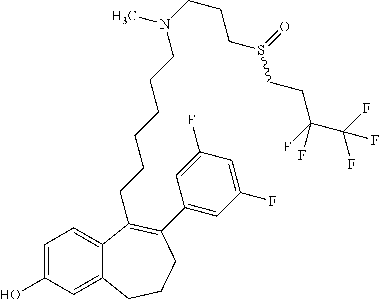 [SMILES: CN(CCCCCCC1=C(c2cc(F)cc(F)c2)CCCc2cc(O)ccc21)CCCS(=O)CCC(F)(F)C(F)(F)F]

200 mg (0.46 mmol) of 9-(6-bromohexyl)-8-(3,5-difluorophenyl)-6,7-dihydro-5H-benzo[7]annulen-3-ol was reacted with 147.3 mg (0.55 mmol) of N-methyl-3-[(3,3,4,4,4-pentafluorobutyl)sulphinyl]propan-1-amine according to general specification 11. It was purified using HPLC-Method 2. 132.8 mg (46% of theor.) of product was isolated.

¹H-NMR (600 MHz, chloroform-d₁): δ=1.04-1.10 (m, 2H), 1.10-1.17 (m, 2H), 1.18-1.28 (m, 4H), 2.02 (mc, 2H), 2.06-2.15 (m, 4H), 2.19-2.28 (m, 5H), 2.38 (t, 2H), 2.50-2.66 (m, 6H), 2.83 (t, 2H), 2.90-3.02 (m, 2H), 6.70 (tt, 1H), 6.73-6.79 (m, 4H), 7.15 (d, 1H).

### Example 3

### 8-(3,5-Difluorophenyl)-9-[6-(methyl{3-[(3,3,4,4,4-pentafluorobutyl)sulphonyl]propyl}amino)hexyl]-6,7-dihydro-5H-benzo[7]annulen-3-ol

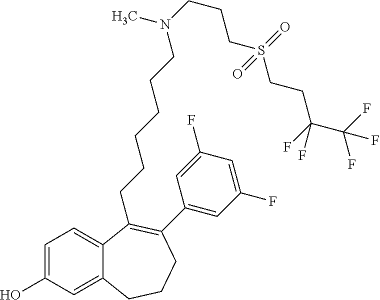 [SMILES: CN(CCCCCCC1=C(c2cc(F)cc(F)c2)CCCc2cc(O)ccc21)CCCS(=O)(=O)CCC(F)(F)C(F)(F)F]

200 mg (0.46 mmol) of 9-(6-bromohexyl)-8-(3,5-difluorophenyl)-6,7-dihydro-5H-benzo[7]annulen-3-ol was reacted with 156.2 mg (0.55 mmol) of N-methyl-3-[(3,3,4,4,4-pentafluorobutyl)sulphonyl]propan-1-amine according to general specification 11. It was purified using HPLC-Method 2. 131.3 mg (45% of theor.) of product was isolated.

¹H-NMR (600 MHz, chloroform-d₁): δ=1.06-1.16 (m, 4H), 1.21 (quin, 2H), 1.31 (mc, 2H), 2.05-2.16 (m, 6H), 2.29-2.35 (m, 5H), 2.38 (t, 2H), 2.59-2.69 (m, 6H), 3.18 (t, 2H), 3.26 (mc, 2H), 6.71 (tt, 1H), 6.73-6.79 (m, 4H), 7.16 (d, 1H).

### Example 4

### 8-(3,5-Difluorophenyl)-9-[6-(methyl{4-[(4,4,5,5,5-pentafluoropentyl)sulphinyl]butyl}amino)hexyl]-6,7-dihydro-5H-benzo[7]annulen-3-ol

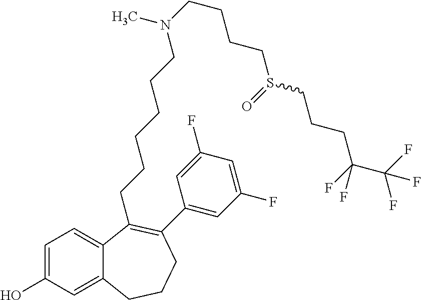 [SMILES: CN(CCCCCCC1=C(c2cc(F)cc(F)c2)CCCc2cc(O)ccc21)CCCCS(=O)CCCC(F)(F)C(F)(F)F]

200 mg (0.46 mmol) of 9-(6-bromohexyl)-8-(3,5-difluorophenyl)-6,7-dihydro-5H-benzo[7]annulen-3-ol was reacted with 162.8 mg (0.55 mmol) of N-methyl-4-[(4,4,5,5,5-pentafluoropentyl)sulphinyl]butan-1-amine according to general specification 11. It was purified using HPLC-Method 2. 114.6 mg (37% of theor.) of product was isolated.

¹H-NMR (400 MHz, chloroform-d₁): δ=1.07-1.14 (m, 2H), 1.15-1.35 (m, 8H), 1.86 (mc, 2H), 2.05-2.14 (m, 4H), 2.15-2.54 (m, 13H), 2.61 (t, 2H), 2.61 (t, 2H), 2.71-2.90 (m, 4H), 6.70 (tt, 1H), 6.73-6.80 (m, 4H), 7.14 (d, 1H).

### Example 5

### 8-(3,5-Difluorophenyl)-9-[6-(methyl{3-[(3,3,3-trifluoropropyl)sulphinyl]propyl}amino)hexyl]-6,7-dihydro-5H-benzo[7]annulen-3-ol

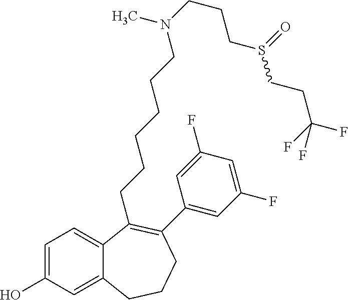 [SMILES: CN(CCCCCCC1=C(c2cc(F)cc(F)c2)CCCc2cc(O)ccc21)CCCS(=O)CCC(F)(F)F]

200 mg (0.46 mmol) of 9-(6-bromohexyl)-8-(3,5-difluorophenyl)-6,7-dihydro-5H-benzo[7]annulen-3-ol was reacted with 119.8 mg (0.55 mmol) of N-methyl-3-[(3,3,3-trifluoropropyl)sulphinyl]propan-1-amine according to general specification 11. It was purified using HPLC-Method 2. 144.6 mg (55% of theor.) of product was isolated.

¹H-NMR (600 MHz, chloroform-d₁): δ=1.05-1.10 (m, 2H), 1.11-1.17 (m, 2H), 1.19-1.32 (m, 4H), 1.96-2.15 (m, 6H), 2.16-2.29 (m, 5H), 2.39 (t, 2H), 2.47-2.70 (m, 6H), 2.83 (mc, 2H), 2.89-2.98 (m, 2H), 6.70 (tt, 1H), 6.73-6.79 (m, 4H), 7.15 (d, 1H).

### Example 6

### 8-(3,5-Difluorophenyl)-9-[6-(methyl{3-[(3,3,3-trifluoropropyl)sulphonyl]propyl}amino)hexyl]-6,7-dihydro-5H-benzo[7]annulen-3-ol

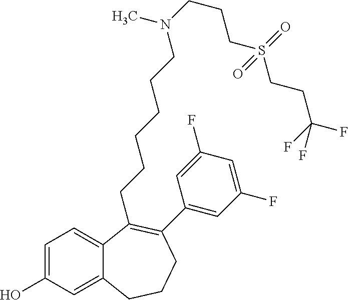 [SMILES: CN(CCCCCCC1=C(c2cc(F)cc(F)c2)CCCc2cc(O)ccc21)CCCS(=O)(=O)CCC(F)(F)F]

200 mg (0.46 mmol) of 9-(6-bromohexyl)-8-(3,5-difluorophenyl)-6,7-dihydro-5H-benzo[7]annulen-3-ol was reacted with 128.6 mg (0.55 mmol) of N-methyl-3-[(3,3,3-trifluoropropyl)sulphonyl]propan-1-amine according to general specification 11. It was purified using HPLC-Method 2. 111.6 mg (41% of theor.) of product was isolated.

¹H-NMR (600 MHz, chloroform-d₁): δ=1.07-1.15 (m, 4H), 1.20 (mc, 2H), 1.24-1.33 (m, 2H), 2.02 (mc, 2H), 2.05-2.15 (m, 4H), 2.19 (s, 3H), 2.23 (mc, 2H), 2.37 (t, 2H), 2.47 (mc, 2H), 2.62 (t, 2H), 2.64-2.73 (m, 2H), 3.12 (t, 2H), 3.20 (mc, 2H), 6.68-6.74 (m, 2H), 6.74-6.78 (m, 3H), 7.17 (d, 1H).

### Example 7

### 8-(3,5-Difluorophenyl)-9-[6-(methyl{3-[(4,4,4-trifluorobutyl)sulphonyl]propyl}amino)hexyl]-6,7-dihydro-5H-benzo[7]annulen-3-ol

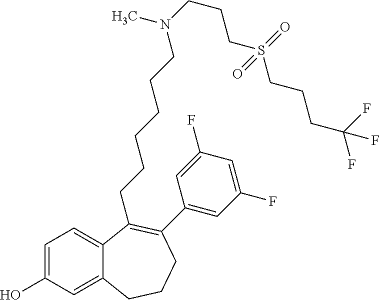 [SMILES: CN(CCCCCCC1=C(c2cc(F)cc(F)c2)CCCc2cc(O)ccc21)CCCS(=O)(=O)CCCC(F)(F)F]

200 mg (0.46 mmol) of 9-(6-bromohexyl)-8-(3,5-difluorophenyl)-6,7-dihydro-5H-benzo[7]annulen-3-ol was reacted with 136.3 mg (0.55 mmol) of N-methyl-3-[(4,4,4-trifluorobutyl)sulphonyl]propan-1-amine according to general specification 11. It was purified using HPLC-Method 2. 144.7 mg (52% of theor.) of product was isolated.

¹H-NMR (600 MHz, chloroform-d₁): δ=1.06-1.16 (m, 4H), 1.21 (quin, 2H), 1.29-1.35 (m, 2H), 2.05-2.20 (m, 8H), 2.26-2.40 (m, 9H), 2.57-2.68 (m, 4H), 3.07-3.13 (m, 4H), 6.71 (tt, 1H), 6.74-6.79 (m, 4H), 7.16 (d, 1H).

### Example 8

### 8-(3,5-Difluorophenyl)-9-[6-(methyl{3-[(4,4,5,5,5-pentafluoropentyl)sulphinyl]propyl}amino)hexyl]-6,7-dihydro-5H-benzo[7]annulen-3-ol

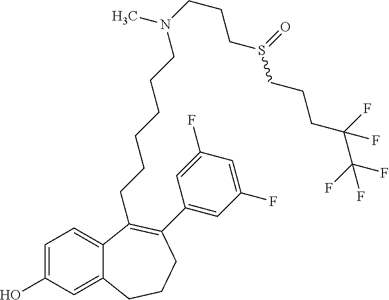 [SMILES: CN(CCCCCCC1=C(c2cc(F)cc(F)c2)CCCc2cc(O)ccc21)CCCS(=O)CCCC(F)(F)C(F)(F)F]

200 mg (0.46 mmol) of 9-(6-bromohexyl)-8-(3,5-difluorophenyl)-6,7-dihydro-5H-benzo[7]annulen-3-ol was reacted with 155.1 mg (0.55 mmol) of N-methyl-3-[(4,4,5,5,5-pentafluoropentyl)sulphinyl]propan-1-amine according to general specification 11. It was purified using HPLC-Method 2. 231.3 mg (78% of theor.) of product was isolated.

¹H-NMR (600 MHz, chloroform-d₁): δ=1.00-1.07 (m, 2H), 1.17 (mc, 2H), 1.20-1.26 (m, 2H), 1.31 (mc, 2H), 2.06-2.38 (m, 12H), 2.40 (t, 2H), 2.44 (s, 3H), 2.61 (t, 2H), 2.74-2.92 (m, 6H), 6.70 (tt, 1H), 6.73-6.77 (m, 3H), 6.79 (dd, 1H), 7.14 (d, 1H).

### Example 9

### 8-(3,5-Difluorophenyl)-9-[6-(methyl{4-[(4,4,5,5,5-pentafluoropentyl)sulphonyl]butyl}amino)hexyl]-6,7-dihydro-5H-benzo[7]annulen-3-ol

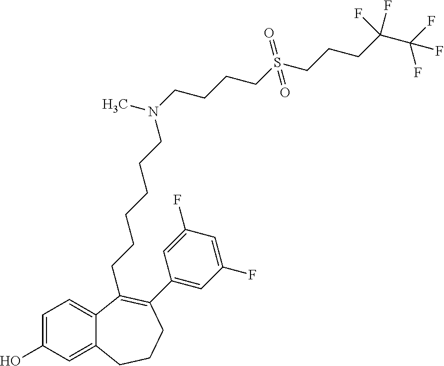 [SMILES: CN(CCCCCCC1=C(c2cc(F)cc(F)c2)CCCc2cc(O)ccc21)CCCCS(=O)(=O)CCCC(F)(F)C(F)(F)F]

200 mg (0.46 mmol) of 9-(6-bromohexyl)-8-(3,5-difluorophenyl)-6,7-dihydro-5H-benzo[7]annulen-3-ol was reacted with 171.6 mg (0.55 mmol) of N-methyl-4-[(4,4,5,5,5-pentafluoropentyl)sulphonyl]butan-1-amine according to general specification 11. It was purified using HPLC-Method 2. 222 mg (71% of theor.) of product was isolated.

¹H-NMR (600 MHz, chloroform-d₁): δ=1.07-1.16 (m, 4H), 1.20 (mc, 2H), 1.29-1.36 (m, 2H), 1.70 (mc, 2H), 1.90 (quin, 2H), 2.05-2.15 (m, 4H), 2.17-2.35 (m, 9H), 2.38 (t, 2H), 2.47 (mc, 2H), 2.61 (t, 2H), 3.02-3.11 (m, 4H), 6.71 (tt, 1H), 6.73-6.78 (m, 4H), 7.16 (d, 1H).

### Example 10

### 8-(3,5-Difluorophenyl)-9-{6-[(2-hydroxy-2-methylpropyl){3-[(3,3,3-trifluoropropyl)sulphinyl]propyl}amino]hexyl}-6,7-dihydro-5H-benzo[7]annulen-3-ol

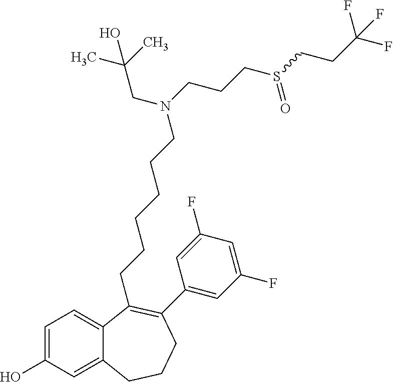 [SMILES: CC(C)(O)CN(CCCCCCC1=C(c2cc(F)cc(F)c2)CCCc2cc(O)ccc21)CCCS(=O)CCC(F)(F)F]

200 mg (0.46 mmol) of 9-(6-bromohexyl)-8-(3,5-difluorophenyl)-6,7-dihydro-5H-benzo[7]annulen-3-ol was reacted with 151.8 mg (0.55 mmol) of 2-methyl-1-({3-[(3,3,3-trifluoropropyl)sulphinyl]propyl}amino)propan-2-ol according to general specification 11. It was purified using HPLC-Method 2. 53.4 mg (18% of theor.) of product was isolated.

¹H-NMR (400 MHz, chloroform-d₁): δ=1.00-1.28 (m, 14H), 1.83-2.00 (m, 2H), 2.02-2.15 (m, 4H), 2.31-2.43 (m, 6H), 2.51-2.85 (m, 8H), 2.87-2.95 (m, 2H), 6.66-6.79 (m, 5H), 7.14 (d, 1H).

### Example 11

### 8-(3,5-Difluorophenyl)-9-{6-[(2-hydroxy-2-methylpropyl){3-[(3,3,3-trifluoropropyl)sulphonyl]propyl}amino]hexyl}-6,7-dihydro-5H-benzo[7]annulen-3-ol

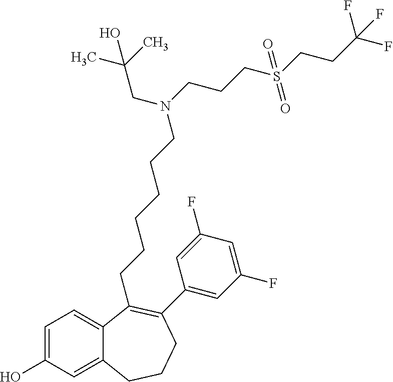 [SMILES: CC(C)(O)CN(CCCCCCC1=C(c2cc(F)cc(F)c2)CCCc2cc(O)ccc21)CCCS(=O)(=O)CCC(F)(F)F]

200 mg (0.46 mmol) of 9-(6-bromohexyl)-8-(3,5-difluorophenyl)-6,7-dihydro-5H-benzo[7]annulen-3-ol was reacted with 160.6 mg (0.55 mmol) of 2-methyl-1-({3-[(3,3,3-trifluoropropyl)sulphonyl]propyl}amino)propan-2-ol according to general specification 11. It was purified using HPLC-Method 2. 51.4 mg (17% of theor.) of product was isolated.

¹H-NMR (400 MHz, chloroform-d₁): δ=1.01-1.34 (m, 14H), 1.98 (mc, 2H), 2.03-2.16 (m, 4H), 2.32-2.45 (m, 6H), 2.56-2.76 (m, 6H), 3.09 (mc, 2H), 3.16-3.23 (m, 2H), 6.66-6.80 (m, 5H), 7.16 (d, 1H).

### Example 12

### 8-(3,5-Difluorophenyl)-9-{6-[(2-hydroxy-2-methylpropyl){3-[(4,4,5,5,5-pentafluoropentyl)sulphinyl]propyl}amino]hexyl}-6,7-dihydro-5H-benzo[7]annulen-3-ol

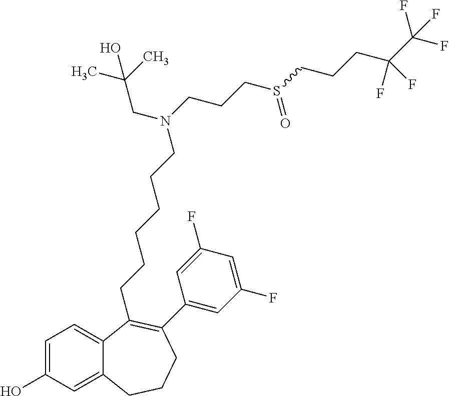 [SMILES: CC(C)(O)CN(CCCCCCC1=C(c2cc(F)cc(F)c2)CCCc2cc(O)ccc21)CCCS(=O)CCCC(F)(F)C(F)(F)F]

200 mg (0.46 mmol) of 9-(6-bromohexyl)-8-(3,5-difluorophenyl)-6,7-dihydro-5H-benzo[7]annulen-3-ol was reacted with 187.1 mg (0.55 mmol) of 2-methyl-1-({3-[(4,4,5,5,5-pentafluoropentyl)sulphinyl]propyl}amino)propan-2-ol according to general specification 11. It was purified using HPLC-Method 2. 74.7 mg (23% of theor.) of product was isolated.

¹H-NMR (400 MHz, chloroform-d₁): δ=1.00-1.30 (m, 14H), 1.82-1.99 (m, 2H), 2.02-2.42 (m, 14H), 2.50-2.90 (m, 8H), 6.65-6.80 (m, 5H), 7.13 (d, 1H).

### Example 13

### 8-(3,5-Difluorophenyl)-9-{6-[(2-hydroxy-2-methylpropyl){3-[(4,4,5,5,5-pentafluoropentyl)sulphonyl]propyl}amino]hexyl}-6,7-dihydro-5H-benzo[7]annulen-3-ol

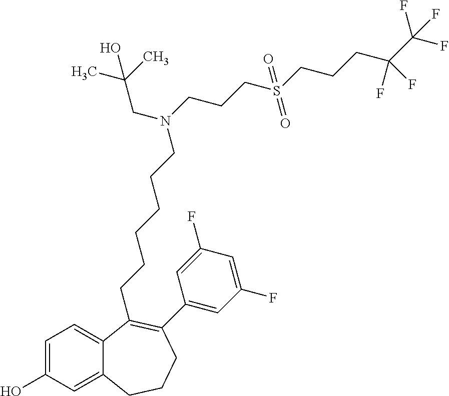 [SMILES: CC(C)(O)CN(CCCCCCC1=C(c2cc(F)cc(F)c2)CCCc2cc(O)ccc21)CCCS(=O)(=O)CCCC(F)(F)C(F)(F)F]

200 mg (0.46 mmol) of 9-(6-bromohexyl)-8-(3,5-difluorophenyl)-6,7-dihydro-5H-benzo[7]annulen-3-ol was reacted with 195.9 mg (0.55 mmol) of 2-methyl-1-({3-[(4,4,5,5,5-pentafluoropentyl)sulphonyl]propyl}amino)propan-2-ol according to general specification 11. It was purified using HPLC-Method 2. 40.2 mg (12% of theor.) of product was isolated.

¹H-NMR (400 MHz, chloroform-d₁): δ=1.02-1.33 (m, 14H), 1.97 (mc, 2H), 2.03-2.45 (m, 14H), 2.58-2.69 (m, 4H), 3.00-3.11 (m, 4H), 6.66-6.80 (m, 5H), 7.16 (d, 1H).

### Example 14

### 8-(3,5-Difluorophenyl)-9-[6-(ethyl{3-[(3,3,3-trifluoropropyl)sulphonyl]propyl}amino)hexyl]-6,7-dihydro-5H-benzo[7]annulen-3-ol

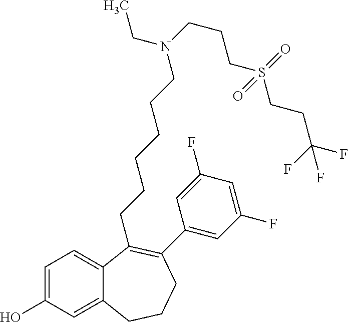 [SMILES: CCN(CCCCCCC1=C(c2cc(F)cc(F)c2)CCCc2cc(O)ccc21)CCCS(=O)(=O)CCC(F)(F)F]

200 mg (0.46 mmol) of 9-(6-bromohexyl)-8-(3,5-difluorophenyl)-6,7-dihydro-5H-benzo[7]annulen-3-ol was reacted with 136.3 mg (0.55 mmol) of N-ethyl-3-[(3,3,3-trifluoropropyl)sulphonyl]propan-1-amine according to general specification 11. It was purified using HPLC-Method 2. 115.1 mg (42% of theor.) of product was isolated.

¹H-NMR (600 MHz, chloroform-d₁): δ=1.04-1.15 (m, 7H), 1.19-1.29 (m, 4H), 2.05-2.15 (m, 6H), 2.36-2.42 (m, 4H), 2.61 (t, 2H), 2.66-2.76 (m, 6H), 3.17 (mc, 2H), 3.24 (mc, 2H), 6.71 (tt, 1H), 6.73-6.79 (m, 4H), 7.16 (d, 1H).

### Example 15

### 8-(3,5-Difluorophenyl)-9-{6-[(2-methoxyethyl){3-[(4,4,5,5,5-pentafluoropentyl)sulphonyl]propyl}amino]hexyl}-6,7-dihydro-5H-benzo[7]annulen-3-ol

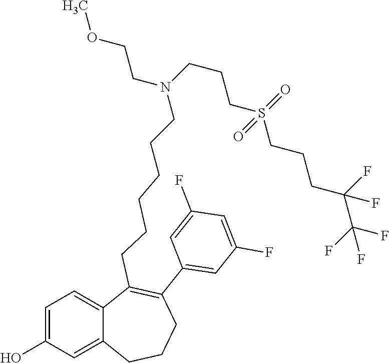 [SMILES: COCCN(CCCCCCC1=C(c2cc(F)cc(F)c2)CCCc2cc(O)ccc21)CCCS(=O)(=O)CCCC(F)(F)C(F)(F)F]

100 mg (0.23 mmol) of 9-(6-bromohexyl)-8-(3,5-difluorophenyl)-6,7-dihydro-5H-benzo[7]annulen-3-ol was reacted with 94.1 mg (0.28 mmol) of N-(2-methoxyethyl)-3-[(4,4,5,5,5-pentafluoropentyl)sulphonyl]propan-1-amine according to general specification 11. It was purified using HPLC-Method 1. 56 mg (35% of theor.) of product was isolated.

¹H-NMR (400 MHz, chloroform-d₁): δ=1.03-1.14 (m, 4H), 1.15-1.32 (m, 4H), 1.99-2.40 (m, 12H), 2.47 (mc, 2H), 2.60 (t, 2H), 2.72-2.81 (m, 4H), 3.05-3.14 (m, 4H), 3.32 (s, 3H), 3.49 (t, 2H), 6.67-6.79 (m, 5H), 7.14 (d, 1H).

### Example 16

### 8-(3,5-Difluorophenyl)-9-{6-[(3-methoxypropyl){3-[(4,4,5,5,5-pentafluoropentyl)sulphonyl]propyl}amino]hexyl}-6,7-dihydro-5H-benzo[7]annulen-3-ol

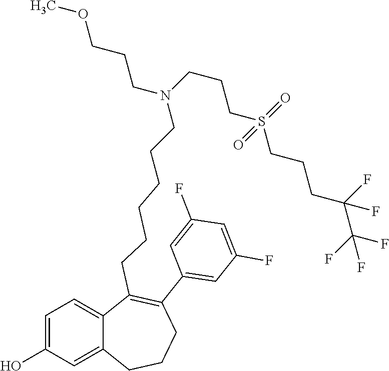 [SMILES: COCCCN(CCCCCCC1=C(c2cc(F)cc(F)c2)CCCc2cc(O)ccc21)CCCS(=O)(=O)CCCC(F)(F)C(F)(F)F]

100 mg (0.23 mmol) of 9-(6-bromohexyl)-8-(3,5-difluorophenyl)-6,7-dihydro-5H-benzo[7]annulen-3-ol was reacted with 98 mg (0.28 mmol) of 3-methoxy-N-{3-[(4,4,5,5,5-pentafluoropentyl)sulphonyl]propyl}propan-1-amine according to general specification 11. It was purified using HPLC-Method 1. 62 mg (38% of theor.) of product was isolated.

¹H-NMR (400 MHz, chloroform-d₁): δ=1.01-1.15 (m, 4H), 1.17-1.30 (m, 4H), 1.70-1.80 (m, 2H), 2.02-2.43 (m, 14H), 2.61 (t, 2H), 2.64-2.76 (m, 4H), 3.05-3.14 (m, 4H), 3.32 (s, 3H), 3.40 (t, 2H), 6.67-6.79 (m, 5H), 7.14 (d, 1H).

### Example 17

### 8-(3,4-Difluorophenyl)-9-[6-(methyl{3-[(4,4,5,5,5-pentafluoropentyl)sulphonyl]propyl}amino)hexyl]-6,7-dihydro-5H-benzo[7]annulen-3-ol

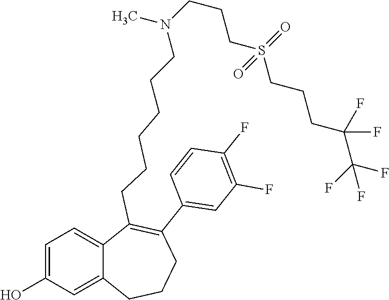 [SMILES: CN(CCCCCCC1=C(c2ccc(F)c(F)c2)CCCc2cc(O)ccc21)CCCS(=O)(=O)CCCC(F)(F)C(F)(F)F]

150 mg (0.35 mmol) of 9-(6-bromohexyl)-8-(3,4-difluorophenyl)-6,7-dihydro-5H-benzo[7]annulen-3-ol was reacted with 143.3 mg (0.52 mmol) of N-methyl-3-[(4,4,5,5,5-pentafluoropentyl)sulphonyl]propan-1-amine according to general specification 11. It was purified using HPLC-Method 2. 162.5 mg (72% of theor.) of product was isolated.

¹H-NMR (400 MHz, chloroform-d₁): δ=1.02-1.26 (m, 6H), 1.30-1.40 (m, 2H), 2.03-2.41 (m, 12H), 2.42-2.49 (m, 5H), 2.61 (t, 2H), 2.84 (t, 2H), 3.14 (mc, 4H), 6.74 (d, 1H), 6.78 (dd, 1H), 6.94 (ddd, 1H), 7.03 (ddd, 1H), 7.09-7.18 (m, 2H).

### Example 18

### 8-(3,4-Difluorophenyl)-9-[6-(methyl{3-[(3,3,4,4,4-pentafluorobutyl)sulphinyl]propyl}amino)hexyl]-6,7-dihydro-5H-benzo[7]annulen-3-ol

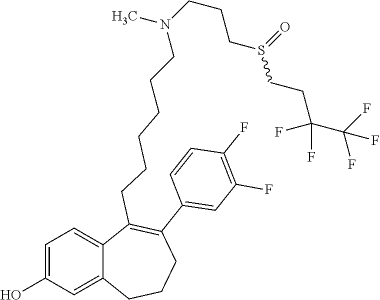 [SMILES: CN(CCCCCCC1=C(c2ccc(F)c(F)c2)CCCc2cc(O)ccc21)CCCS(=O)CCC(F)(F)C(F)(F)F]

200 mg (0.46 mmol) of 9-(6-bromohexyl)-8-(3,4-difluorophenyl)-6,7-dihydro-5H-benzo[7]annulen-3-ol was reacted with 147.3 mg (0.55 mmol) of N-methyl-3-[(3,3,4,4,4-pentafluorobutyl)sulphinyl]propan-1-amine according to general specification 11. It was purified using HPLC-Method 2. 133 mg (46% of theor.) of product was isolated.

¹H-NMR (600 MHz, DMSO-d₆): δ=1.00-1.10 (m, 4H), 1.11-1.17 (m, 2H), 1.21-1.28 (m, 2H), 1.76 (mc, 2H), 1.98 (t, 2H), 2.05 (mc, 2H), 2.12 (s, 3H), 2.22 (mc, 2H), 2.31 (t, 2H), 2.34-2.45 (m, 2H), 2.52-2.69 (m, 4H), 2.70-2.76 (m, 1H), 2.77-2.89 (m, 2H), 3.08 (ddd, 1H), 6.64 (d, 1H), 6.66 (dd, 1H), 7.06 (mc, 1H), 7.12 (d, 1H), 7.25 (ddd, 1H), 7.40 (mc, 1H), 9.33 (s, 1H).

### Example 19

### 8-(3,4-Difluorophenyl)-9-[6-(methyl{3-[(3,3,4,4,4-pentafluorobutyl)sulphonyl]propyl}amino)hexyl]-6,7-dihydro-5H-benzo[7]annulen-3-ol

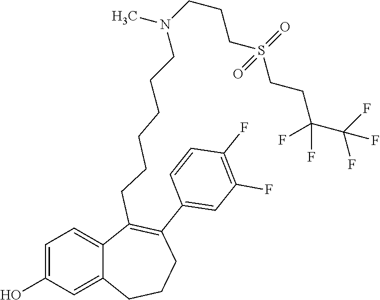 [SMILES: CN(CCCCCCC1=C(c2ccc(F)c(F)c2)CCCc2cc(O)ccc21)CCCS(=O)(=O)CCC(F)(F)C(F)(F)F]

200 mg (0.46 mmol) of 9-(6-bromohexyl)-8-(3,4-difluorophenyl)-6,7-dihydro-5H-benzo[7]annulen-3-ol was reacted with 156.2 mg (0.55 mmol) of N-methyl-3-[(3,3,4,4,4-pentafluorobutyl)sulphonyl]propan-1-amine according to general specification 11. It was purified using HPLC-Method 2. 174.6 mg (60% of theor.) of product was isolated.

¹H-NMR (600 MHz, DMSO-d₆): δ=1.00-1.17 (m, 6H), 1.23 (mc, 2H), 1.80 (mc, 2H), 1.97 (t, 2H), 2.01-2.11 (m, 5H), 2.17 (mc, 2H), 2.28-2.38 (m, 4H), 2.54 (t, 2H), 2.59-2.69 (m, 2H), 3.21 (mc, 2H), 3.44 (mc, 2H), 6.64 (d, 1H), 6.66 (dd, 1H), 7.07 (mc, 1H), 7.13 (d, 1H), 7.27 (ddd, 1H), 7.41 (mc, 1H), 9.34 (s, 1H).

### Example 20

### 8-(3,4-Difluorophenyl)-9-[6-(methyl{4-[(4,4,5,5,5-pentafluoropentyl)sulphinyl]butyl}amino)hexyl]-6,7-dihydro-5H-benzo[7]annulen-3-ol

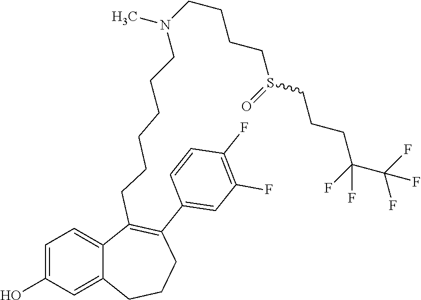 [SMILES: CN(CCCCCCC1=C(c2ccc(F)c(F)c2)CCCc2cc(O)ccc21)CCCCS(=O)CCCC(F)(F)C(F)(F)F]

200 mg (0.46 mmol) of 9-(6-bromohexyl)-8-(3,4-difluorophenyl)-6,7-dihydro-5H-benzo[7]annulen-3-ol was reacted with 162.8 mg (0.55 mmol) of N-methyl-4-[(4,4,5,5,5-pentafluoropentyl)sulphinyl]butan-1-amine according to general specification 11. It was purified using HPLC-Method 2. 207.2 mg (66% of theor.) of product was isolated.

¹H-NMR (500 MHz, DMSO-d₆): δ=0.99-1.17 (m, 6H), 1.19-1.28 (m, 2H), 1.44-1.54 (m, 2H), 1.56-1.66 (m, 2H), 1.91 (quin, 2H), 1.97 (t, 2H), 2.01-2.11 (m, 5H), 2.16 (mc, 2H), 2.22-2.44 (m, 6H), 2.54 (t, 2H), 2.65-2.77 (m, 3H), 2.80-2.88 (mc, 1H), 6.63-6.68 (m, 2H), 7.05-7.09 (m, 1H), 7.12 (d, 1H), 7.26 (ddd, 1H), 7.41 (mc, 1H), 9.32 (s, 1H).

### Example 21

### 8-(3,4-Difluorophenyl)-9-[6-(methyl{3-[(3,3,3-trifluoropropyl)sulphinyl]propyl}amino)hexyl]-6,7-dihydro-5H-benzo[7]annulen-3-ol

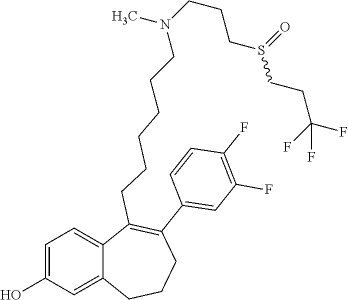 [SMILES: CN(CCCCCCC1=C(c2ccc(F)c(F)c2)CCCc2cc(O)ccc21)CCCS(=O)CCC(F)(F)F]

200 mg (0.46 mmol) of 9-(6-bromohexyl)-8-(3,4-difluorophenyl)-6,7-dihydro-5H-benzo[7]annulen-3-ol was reacted with 119.8 mg (0.55 mmol) of N-methyl-3-[(3,3,3-trifluoropropyl)sulphinyl]propan-1-amine according to general specification 11. It was purified using HPLC-Method 2. 171.1 mg (65% of theor.) of product was isolated.

¹H-NMR (500 MHz, DMSO-d₆): δ=1.00-1.18 (m, 6H), 1.19-1.28 (m, 2H), 1.70-1.80 (m, 2H), 1.95-2.00 (m, 2H), 2.01-2.25 (m, 7H), 2.38-2.43 (m, 4H), 2.55 (t, 2H), 2.60-2.73 (m, 3H), 2.75-2.86 (m, 2H), 3.02 (ddd, 1H), 6.63-6.68 (m, 2H), 7.04-7.09 (m, 1H), 7.13 (d, 1H), 7.26 (ddd, 1H), 7.41 (mc, 1H), 9.33 (s, 1H).

### Example 22

### 8-(3,4-Difluorophenyl)-9-[6-(methyl{3-[(3,3,3-trifluoropropyl)sulphonyl]propyl}amino)hexyl]-6,7-dihydro-5H-benzo[7]annulen-3-ol

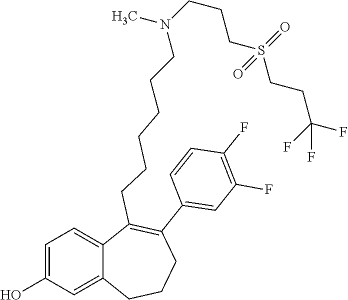 [SMILES: CN(CCCCCCC1=C(c2ccc(F)c(F)c2)CCCc2cc(O)ccc21)CCCS(=O)(=O)CCC(F)(F)F]

200 mg (0.46 mmol) of 9-(6-bromohexyl)-8-(3,4-difluorophenyl)-6,7-dihydro-5H-benzo[7]annulen-3-ol was reacted with 128.6 mg (0.55 mmol) of N-methyl-3-[(3,3,3-trifluoropropyl)sulphonyl]propan-1-amine according to general specification 11. It was purified using HPLC-Method 2. 135.1 mg (50% of theor.) of product was isolated.

¹H-NMR (500 MHz, DMSO-d₆): δ=0.99-1.18 (m, 6H), 1.20-1.29 (m, 2H), 1.81 (mc, 2H), 1.97 (t, 2H), 2.05 (mc, 2H), 2.11 (s, 3H), 2.20 (mc, 2H), 2.31 (t, 2H), 2.35-2.42 (m, 2H), 2.55 (t, 2H), 2.67-2.78 (m, 2H), 3.18 (mc, 2H), 3.40 (mc, 2H), 6.63-6.69 (m, 2H), 7.05-7.09 (m, 1H), 7.13 (d, 1H), 7.26 (ddd, 1H), 7.41 (mc, 1H), 9.33 (s, 1H).

### Example 23

### 8-(3,4-Difluorophenyl)-9-[6-(methyl{3-[(4,4,4-trifluorobutyl)sulphonyl]propyl}amino)hexyl]-6,7-dihydro-5H-benzo[7]annulen-3-ol

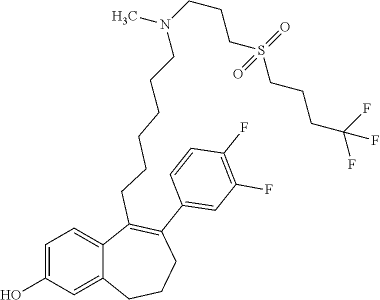 [SMILES: CN(CCCCCCC1=C(c2ccc(F)c(F)c2)CCCc2cc(O)ccc21)CCCS(=O)(=O)CCCC(F)(F)F]

200 mg (0.46 mmol) of 9-(6-bromohexyl)-8-(3,4-difluorophenyl)-6,7-dihydro-5H-benzo[7]annulen-3-ol was reacted with 136.3 mg (0.55 mmol) of N-methyl-3-[(4,4,4-trifluorobutyl)sulphonyl]propan-1-amine according to general specification 11. It was purified using HPLC-Method 2. 140.1 mg (51% of theor.) of product was isolated.

¹H-NMR (600 MHz, DMSO-d₆): δ=0.99-1.17 (m, 6H), 1.19-1.27 (m, 2H), 1.76 (mc, 2H), 1.84-1.92 (m, 2H), 1.97 (t, 2H), 2.01-2.11 (m, 5H), 2.16 (mc, 2H), 2.28-2.46 (m, 6H), 2.54 (t, 2H), 3.08 (mc, 2H), 3.19 (t, 2H), 6.64 (d, 1H), 6.66 (dd, 1H), 7.05-7.09 (m, 1H), 7.13 (d, 1H), 7.27 (ddd, 1H), 7.41 (mc, 1H), 9.35 (s, 1H).

### Example 24

### 8-(3,4-Difluorophenyl)-9-[6-(methyl{3-[(4,4,5,5,5-pentafluoropentyl)sulphinyl]propyl}amino)hexyl]-6,7-dihydro-5H-benzo[7]annulen-3-ol

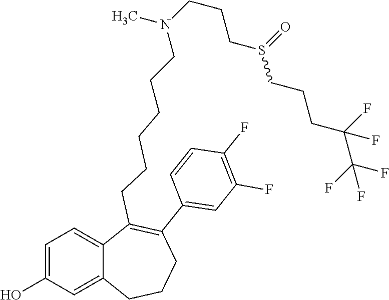 [SMILES: CN(CCCCCCC1=C(c2ccc(F)c(F)c2)CCCc2cc(O)ccc21)CCCS(=O)CCCC(F)(F)C(F)(F)F]

200 mg (0.46 mmol) of 9-(6-bromohexyl)-8-(3,4-difluorophenyl)-6,7-dihydro-5H-benzo[7]annulen-3-ol was reacted with 155.1 mg (0.55 mmol) of N-methyl-3-[(4,4,5,5,5-pentafluoropentyl)sulphinyl]propan-1-amine according to general specification 11. It was purified using HPLC-Method 2. 270 mg (92% of theor.) of product was isolated.

¹H-NMR (600 MHz, DMSO-d₆): δ=1.00-1.17 (m, 6H), 1.27 (mc, 2H), 1.78 (mc, 2H), 1.90 (quin, 2H), 1.97 (t, 2H), 2.05 (quin, 2H), 2.08-2.45 (m, 8H), 2.54 (t, 2H), 2.56-2.69 (m, 2H), 2.71-2.79 (m, 2H), 2.82-2.88 (m, 1H), 6.64 (d, 1H), 6.66 (dd, 1H), 7.05-7.09 (m, 1H), 7.13 (d, 1H), 7.28 (ddd, 1H), 7.42 (mc, 1H), 9.35 (s, 1H).

### Example 25

### 8-(3,4-Difluorophenyl)-9-[6-(methyl{4-[(4,4,5,5,5-pentafluoropentyl)sulphonyl]butyl}amino)hexyl]-6,7-dihydro-5H-benzo[7]annulen-3-ol

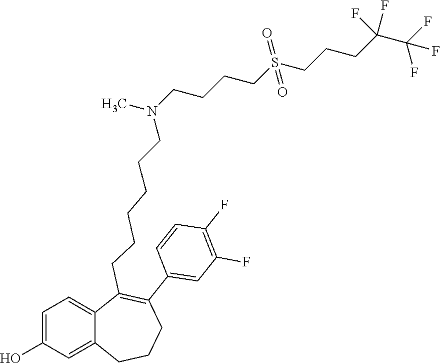 [SMILES: CN(CCCCCCC1=C(c2ccc(F)c(F)c2)CCCc2cc(O)ccc21)CCCCS(=O)(=O)CCCC(F)(F)C(F)(F)F]

200 mg (0.46 mmol) of 9-(6-bromohexyl)-8-(3,4-difluorophenyl)-6,7-dihydro-5H-benzo[7]annulen-3-ol was reacted with 171.6 mg (0.55 mmol) of N-methyl-4-[(4,4,5,5,5-pentafluoropentyl)sulphonyl]butan-1-amine according to general specification 11. It was purified using HPLC-Method 2. 301 mg (98% of theor.) of product was isolated.

¹H-NMR (500 MHz, DMSO-d₆): δ=1.00-1.18 (m, 6H), 1.28 (mc, 2H), 1.53 (mc, 2H), 1.63-1.71 (m, 2H), 1.89-2.00 (m, 4H), 2.01-2.08 (m, 2H), 2.19 (s, 3H), 2.35-2.46 (m, 8H), 2.54 (t, 2H), 3.13 (mc, 2H), 3.21 (t, 2H), 6.63-6.68 (m, 2H), 7.05-7.09 (m, 1H), 7.13 (d, 1H), 7.27 (ddd, 1H), 7.41 (mc, 1H), 9.33 (s, 1H).

### Example 26

### 8-(3,4-Difluorophenyl)-9-{6-[(2-hydroxy-2-methylpropyl){3-[(3,3,3-trifluoropropyl)sulphinyl]propyl}amino]hexyl}-6,7-dihydro-5H-benzo[7]annulen-3-ol

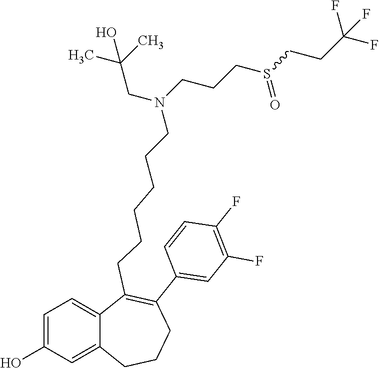 [SMILES: CC(C)(O)CN(CCCCCCC1=C(c2ccc(F)c(F)c2)CCCc2cc(O)ccc21)CCCS(=O)CCC(F)(F)F]

200 mg (0.46 mmol) of 9-(6-bromohexyl)-8-(3,4-difluorophenyl)-6,7-dihydro-5H-benzo[7]annulen-3-ol was reacted with 151.8 mg (0.55 mmol) of 2-methyl-1-({3-[(3,3,3-trifluoropropyl)sulphinyl]propyl}amino)propan-2-ol according to general specification 11. It was purified using HPLC-Method 2. 62.9 mg (22% of theor.) of product was isolated.

¹H-NMR (300 MHz, chloroform-d₁): δ=0.98-1.28 (m, 14H), 1.87-2.00 (m, 2H), 2.02-2.17 (m, 4H), 2.27-2.45 (m, 6H), 2.53-2.86 (m, 8H), 2.86-2.95 (m, 2H), 6.73 (d, 1H), 6.76 (dd, 1H), 6.90-6.97 (m, 1H), 7.04 (ddd, 1H), 7.08-7.18 (m, 2H).

### Example 27

### 8-(3,4-Difluorophenyl)-9-{6-[(2-hydroxy-2-methylpropyl){3-[(3,3,3-trifluoropropyl)sulphonyl]propyl}amino]hexyl}-6,7-dihydro-5H-benzo[7]annulen-3-ol

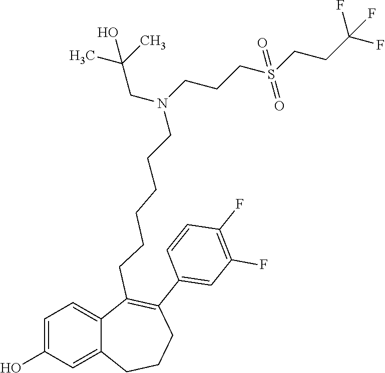 [SMILES: CC(C)(O)CN(CCCCCCC1=C(c2ccc(F)c(F)c2)CCCc2cc(O)ccc21)CCCS(=O)(=O)CCC(F)(F)F]

200 mg (0.46 mmol) of 9-(6-bromohexyl)-8-(3,4-difluorophenyl)-6,7-dihydro-5H-benzo[7]annulen-3-ol was reacted with 160.6 mg (0.55 mmol) of 2-methyl-1-({3-[(3,3,3-trifluoropropyl)sulphonyl]propyl}amino)propan-2-ol according to general specification 11. It was purified using HPLC-Method 2. 41.4 mg (14% of theor.) of product was isolated.

¹H-NMR (300 MHz, chloroform-d₁): δ=0.97-1.38 (m, 14H), 1.91-2.18 (m, 6H), 2.28-2.47 (m, 6H), 2.54-2.80 (m, 6H), 3.04-3.13 (m, 2H), 3.16-3.24 (m, 2H), 6.70-6.79 (m, 2H), 6.91-6.97 (m, 1H), 7.04 (ddd, 1H), 7.08-7.19 (m, 2H).

### Example 28

### 8-(3,4-Difluorophenyl)-9-{6-[(2-hydroxy-2-methylpropyl){3-[(4,4,5,5,5-pentafluoropentyl)sulphinyl]propyl}amino]hexyl}-6,7-dihydro-5H-benzo[7]annulen-3-ol

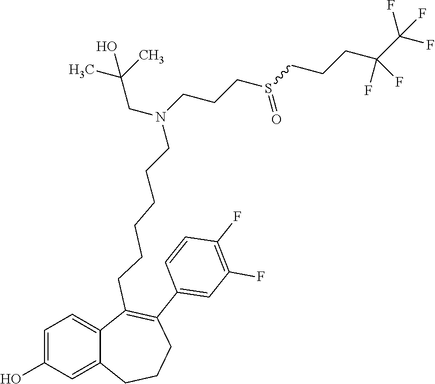 [SMILES: CC(C)(O)CN(CCCCCCC1=C(c2ccc(F)c(F)c2)CCCc2cc(O)ccc21)CCCS(=O)CCCC(F)(F)C(F)(F)F]

200 mg (0.46 mmol) of 9-(6-bromohexyl)-8-(3,4-difluorophenyl)-6,7-dihydro-5H-benzo[7]annulen-3-ol was reacted with 187.1 mg (0.55 mmol) of 2-methyl-1-({3-[(4,4,5,5,5-pentafluoropentyl)sulphinyl]propyl}amino)propan-2-ol according to general specification 11. It was purified using HPLC-Method 2. 64.7 mg (20% of theor.) of product was isolated.

¹H-NMR (300 MHz, chloroform-d₁): δ=1.00-1.29 (m, 14H), 1.82-1.99 (m, 2H), 2.02-2.42 (m, 14H), 2.53-2.99 (m, 8H), 6.73 (d, 1H), 6.77 (dd, 1H), 6.91-6.97 (m, 1H), 7.04 (ddd, 1H), 7.08-7.18 (m, 2H).

### Example 29

### 8-(3,4-Difluorophenyl)-9-{6-[(2-hydroxy-2-methylpropyl){3-[(4,4,5,5,5-pentafluoropentyl)sulphonyl]propyl}amino]hexyl}-6,7-dihydro-5H-benzo[7]annulen-3-ol

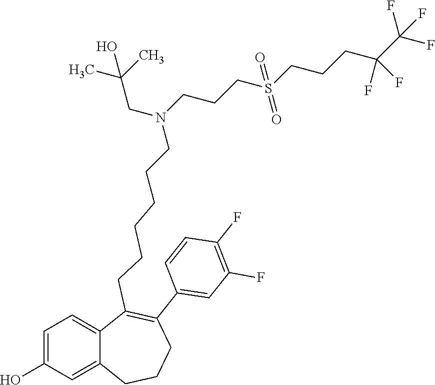 [SMILES: CC(C)(O)CN(CCCCCCC1=C(c2ccc(F)c(F)c2)CCCc2cc(O)ccc21)CCCS(=O)(=O)CCCC(F)(F)C(F)(F)F]

200 mg (0.46 mmol) of 9-(6-bromohexyl)-8-(3,4-difluorophenyl)-6,7-dihydro-5H-benzo[7]annulen-3-ol was reacted with 195.9 mg (0.55 mmol) of 2-methyl-1-({3-[(4,4,5,5,5-pentafluoropentyl)sulphonyl]propyl}amino)propan-2-ol according to general specification 11. It was purified using HPLC-Method 2. 61 mg (18% of theor.) of product was isolated.

¹H-NMR (300 MHz, chloroform-d₁): δ=0.97-1.32 (m, 14H), 1.89-2.45 (m, 16H), 2.53-2.69 (m, 4H), 2.98-3.11 (m, 4H), 6.70-6.79 (m, 2H), 6.91-6.98 (m, 1H), 7.04 (ddd, 1H), 7.08-7.19 (m, 2H).

### Example 30

### 8-(3,4-Difluorophenyl)-9-[6-(ethyl{3-[(3,3,3-trifluoropropyl)sulphonyl]propyl}amino)hexyl]-6,7-dihydro-5H-benzo[7]annulen-3-ol

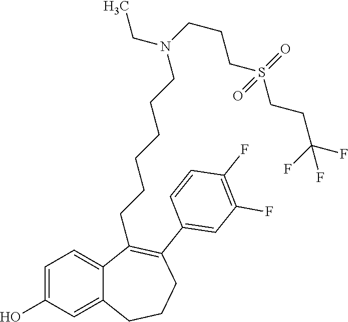 [SMILES: CCN(CCCCCCC1=C(c2ccc(F)c(F)c2)CCCc2cc(O)ccc21)CCCS(=O)(=O)CCC(F)(F)F]

200 mg (0.46 mmol) of 9-(6-bromohexyl)-8-(3,4-difluorophenyl)-6,7-dihydro-5H-benzo[7]annulen-3-ol was reacted with 136.3 mg (0.55 mmol) of N-ethyl-3-[(3,3,3-trifluoropropyl)sulphonyl]propan-1-amine according to general specification 11. It was purified using HPLC-Method 2. 144 mg (51% of theor.) of product was isolated.

¹H-NMR (300 MHz, chloroform-d₁): δ=1.01 (t, 3H), 1.05-1.34 (m, 8H), 1.93-2.17 (m, 6H), 2.29-2.41 (m, 4H), 2.48-2.76 (m, 8H), 3.12 (mc, 2H), 3.20 (mc, 2H), 6.69-6.78 (m, 2H), 6.90-6.98 (m, 1H), 7.04 (ddd, 1H), 7.10-7.19 (m, 2H).

### Example 31

### 8-(3,4-Difluorophenyl)-9-{6-[(2-methoxyethyl){3-[(4,4,5,5,5-pentafluoropentyl)sulphonyl]propyl}amino]hexyl}-6,7-dihydro-5H-benzo[7]annulen-3-ol

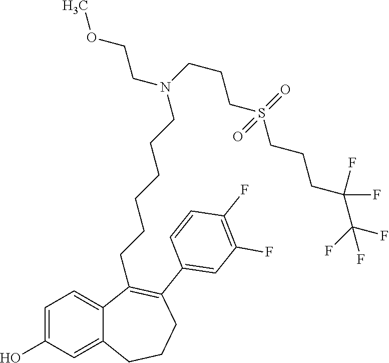 [SMILES: COCCN(CCCCCCC1=C(c2ccc(F)c(F)c2)CCCc2cc(O)ccc21)CCCS(=O)(=O)CCCC(F)(F)C(F)(F)F]

100 mg (0.23 mmol) of 9-(6-bromohexyl)-8-(3,4-difluorophenyl)-6,7-dihydro-5H-benzo[7]annulen-3-ol was reacted with 94.1 mg (0.28 mmol) of N-(2-methoxyethyl)-3-[(4,4,5,5,5-pentafluoropentyl)sulphonyl]propan-1-amine according to general specification 11. It was purified using HPLC-Method 1. 50 mg (31% of theor.) of product was isolated.

¹H-NMR (300 MHz, chloroform-d₁): δ=1.00-1.33 (m, 8H), 1.96-2.40 (m, 12H), 2.46 (mc, 2H), 2.56-2.65 (m, 2H), 2.70-2.82 (m, 4H), 3.03-3.16 (m, 4H), 3.32 (s, 3H), 3.49 (t, 2H), 6.71-6.79 (m, 2H), 6.90-6.98 (m, 1H), 7.04 (ddd, 1H), 7.11-7.19 (m, 2H).

### Example 32

### 8-(3,4-Difluorophenyl)-9-{6-[(3-methoxypropyl){3-[(4,4,5,5,5-pentafluoropentyl)sulphonyl]propyl}amino]hexyl}-6,7-dihydro-5H-benzo[7]annulen-3-ol

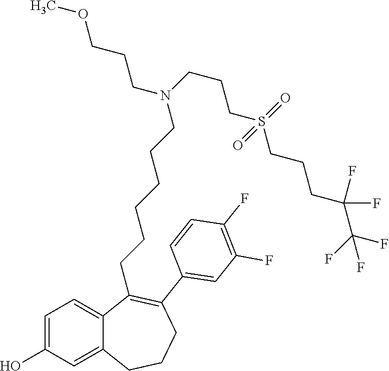 [SMILES: COCCCN(CCCCCCC1=C(c2ccc(F)c(F)c2)CCCc2cc(O)ccc21)CCCS(=O)(=O)CCCC(F)(F)C(F)(F)F]

100 mg (0.23 mmol) of 9-(6-bromohexyl)-8-(3,4-difluorophenyl)-6,7-dihydro-5H-benzo[7]annulen-3-ol was reacted with 98 mg (0.28 mmol) of 3-methoxy-N-{3-[(4,4,5,5,5-pentafluoropentyl)sulphonyl]propyl}propan-1-amine according to general specification 11. It was purified using HPLC-Method 1. 54 mg (33% of theor.) of product was isolated.

¹H-NMR (300 MHz, chloroform-d₁): δ=0.98-1.33 (m, 8H), 1.68-1.81 (m, 2H), 1.99-2.45 (m, 14H), 2.54-2.77 (m, 6H), 3.09 (mc, 4H), 3.32 (s, 3H), 3.40 (t, 2H), 6.70-6.80 (m, 2H), 6.90-6.97 (m, 1H), 7.04 (ddd, 1H), 7.08-7.19 (m, 2H).

### Example 33

### 4-Fluoro-8-(4-fluorophenyl)-9-[6-(methyl{3-[(4,4,5,5,5-pentafluoropentyl)sulphonyl]propyl}amino)hexyl]-6,7-dihydro-5H-benzo[7]annulen-3-ol

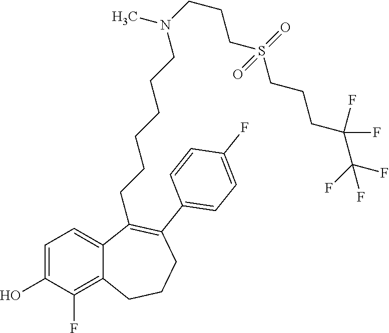 [SMILES: CN(CCCCCCC1=C(c2ccc(F)cc2)CCCc2c1ccc(O)c2F)CCCS(=O)(=O)CCCC(F)(F)C(F)(F)F]

200 mg (0.46 mmol) of 9-(6-bromohexyl)-4-fluoro-8-(4-fluorophenyl)-6,7-dihydro-5H-benzo[7]annulen-3-ol was reacted with 163.9 mg (0.55 mmol) of N-methyl-3-[(4,4,5,5,5-pentafluoropentyl)sulphonyl]propan-1-amine according to general specification 11. It was purified using Kiesel 60 (solvent: dichloromethane, dichloromethane-methanol 95:5). 156 mg (51% of theor.) of product was isolated.

¹H-NMR (300 MHz, chloroform-d₁): δ=1.01-1.37 (m, 8H), 1.91-2.03 (m, 2H), 2.04-2.38 (m, 15H), 2.42 (t, 2H), 2.68-2.78 (m, 2H), 2.99-3.10 (m, 4H), 6.88 (t, 1H), 6.95-7.09 (m, 3H), 7.14-7.23 (m, 2H).

### Example 34

### 4-Fluoro-8-(4-fluorophenyl)-9-[6-(methyl{3-[(3,3,4,4,4-pentafluorobutyl)sulphonyl]propyl}amino)hexyl]-6,7-dihydro-5H-benzo[7]annulen-3-ol

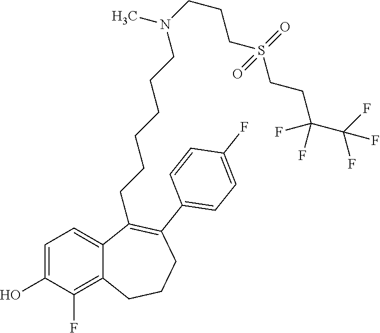 [SMILES: CN(CCCCCCC1=C(c2ccc(F)cc2)CCCc2c1ccc(O)c2F)CCCS(=O)(=O)CCC(F)(F)C(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 78.1 mg (0.28 mmol) of N-methyl-3-[(3,3,4,4,4-pentafluorobutyl)sulphonyl]propan-1-amine according to general specification 11. It was purified using HPLC-Method 1. 60 mg (41% of theor.) of product was isolated.

¹H-NMR (300 MHz, chloroform-d₁): δ=1.02-1.24 (m, 6H), 1.34 (mc, 2H), 2.02-2.17 (m, 6H), 2.27-2.44 (m, 7H), 2.52-2.77 (m, 6H), 3.17 (mc, 2H), 3.24 (mc, 2H), 6.87 (t, 1H), 6.96 (d, 1H), 7.00-7.09 (m, 2H), 7.15-7.22 (m, 2H).

### Example 35

### 4-Fluoro-8-(4-fluorophenyl)-9-[6-(methyl{4-[(4,4,5,5,5-pentafluoropentyl)sulphinyl]butyl}amino)hexyl]-6,7-dihydro-5H-benzo[7]annulen-3-ol

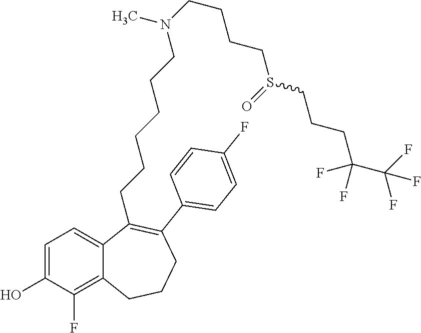 [SMILES: CN(CCCCCCC1=C(c2ccc(F)cc2)CCCc2c1ccc(O)c2F)CCCCS(=O)CCCC(F)(F)C(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 81.4 mg (0.28 mmol) of N-methyl-4-[(4,4,5,5,5-pentafluoropentyl)sulphinyl]butan-1-amine according to general specification 11. It was purified using HPLC-Method 1. 46 mg (31% of theor.) of product was isolated.

¹H-NMR (300 MHz, chloroform-d₁): δ=1.02-1.25 (m, 6H), 1.28-1.42 (m, 2H), 1.64-1.91 (m, 4H), 2.02-2.49 (m, 15H), 2.50-2.87 (m, 8H), 6.82-6.96 (m, 2H), 6.99-7.08 (m, 2H), 7.13-7.22 (m, 2H).

### Example 36

### 4-Fluoro-8-(4-fluorophenyl)-9-[6-(methyl{3-[(3,3,3-trifluoropropyl)sulphinyl]propyl}amino)hexyl]-6,7-dihydro-5H-benzo[7]annulen-3-ol

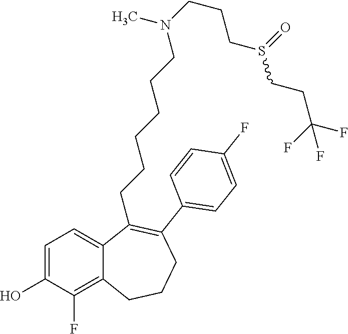 [SMILES: CN(CCCCCCC1=C(c2ccc(F)cc2)CCCc2c1ccc(O)c2F)CCCS(=O)CCC(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 59.9 mg (0.28 mmol) of N-methyl-3-[(3,3,3-trifluoropropyl)sulphinyl]propan-1-amine according to general specification 11. It was purified using HPLC-Method 1. 46 mg (35% of theor.) of product was isolated.

¹H-NMR (300 MHz, chloroform-d₁): δ=1.01-1.25 (m, 6H), 1.27-1.40 (m, 2H), 1.98-2.16 (m, 6H), 2.27-2.44 (m, 7H), 2.54-2.99 (m, 10H), 6.87 (t, 1H), 6.94 (d, 1H), 6.99-7.09 (m, 2H), 7.14-7.22 (m, 2H).

### Example 37

### 4-Fluoro-8-(4-fluorophenyl)-9-[6-(methyl{3-[(3,3,3-trifluoropropyl)sulphonyl]propyl}amino)hexyl]-6,7-dihydro-5H-benzo[7]annulen-3-ol

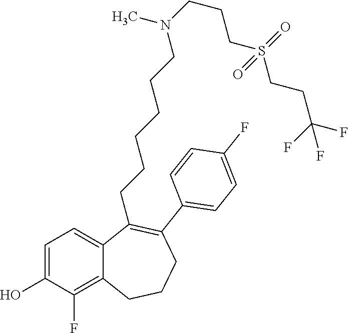 [SMILES: CN(CCCCCCC1=C(c2ccc(F)cc2)CCCc2c1ccc(O)c2F)CCCS(=O)(=O)CCC(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 64.3 mg (0.28 mmol) of N-methyl-3-[(3,3,3-trifluoropropyl)sulphonyl]propan-1-amine according to general specification 11. It was purified using HPLC-Method 1. 44 mg (33% of theor.) of product was isolated.

¹H-NMR (300 MHz, chloroform-d₁): δ=1.02-1.24 (m, 6H), 1.26-1.41 (m, 2H), 2.01-2.18 (m, 6H), 2.26-2.45 (m, 7H), 2.59-2.78 (m, 6H), 3.10-3.26 (m, 4H), 6.87 (t, 1H), 6.96 (d, 1H), 6.99-7.09 (m, 2H), 7.14-7.22 (m, 2H).

### Example 38

### 4-Fluoro-8-(4-fluorophenyl)-9-[6-(methyl{3-[(4,4,4-trifluorobutyl)sulphonyl]propyl}amino)hexyl]-6,7-dihydro-5H-benzo[7]annulen-3-ol

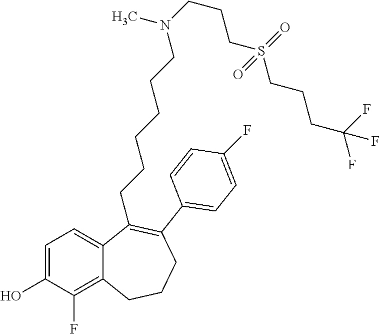 [SMILES: CN(CCCCCCC1=C(c2ccc(F)cc2)CCCc2c1ccc(O)c2F)CCCS(=O)(=O)CCCC(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 68.2 mg (0.28 mmol) of N-methyl-3-[(4,4,4-trifluorobutyl)sulphonyl]propan-1-amine according to general specification 11. It was purified using HPLC-Method 1. 41 mg (30% of theor.) of product was isolated.

¹H-NMR (300 MHz, chloroform-d₁): δ=1.01-1.24 (m, 6H), 1.28-1.43 (m, 2H), 2.02-2.21 (m, 8H), 2.23-2.48 (m, 9H), 2.72 (t, 4H), 3.09 (q, 4H), 6.87 (t, 1H), 6.95 (d, 1H), 7.00-7.09 (m, 2H), 7.14-7.23 (m, 2H).

### Example 39

### 4-Fluoro-8-(4-fluorophenyl)-9-[6-(methyl{4-[(4,4,5,5,5-pentafluoropentyl)sulphonyl]butyl}amino)hexyl]-6,7-dihydro-5H-benzo[7]annulen-3-ol

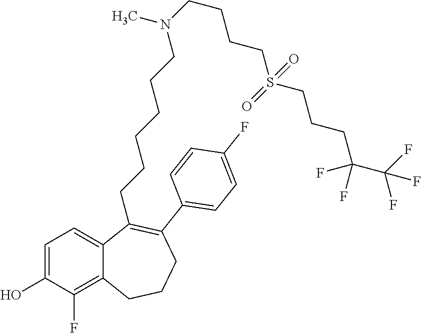 [SMILES: CN(CCCCCCC1=C(c2ccc(F)cc2)CCCc2c1ccc(O)c2F)CCCCS(=O)(=O)CCCC(F)(F)C(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 72 mg (0.28 mmol) of N-methyl-4-[(4,4,5,5,5-pentafluoropentyl)sulphonyl]butan-1-amine according to general specification 11. It was purified using HPLC-Method 1. 18.3 mg (13% of theor.) of product was isolated.

¹H-NMR (300 MHz, chloroform-d₁): δ=1.02-1.24 (m, 6H), 1.28-1.41 (m, 2H), 1.65-1.79 (m, 2H), 1.82-1.95 (m, 2H), 2.02-2.21 (m, 6H), 2.24-2.44 (m, 9H), 2.55 (mc, 2H), 2.66-2.77 (m, 2H), 2.98-3.10 (m, 4H), 6.87 (t, 1H), 6.96 (d, 1H), 7.00-7.09 (m, 2H), 7.14-7.22 (m, 2H).

### Example 40

### 4-Fluoro-8-(4-fluorophenyl)-9-{6-[(2-methoxyethyl){3-[(4,4,5,5,5-pentafluoropentyl)sulphonyl]propyl}amino]hexyl}-6,7-dihydro-5H-benzo[7]annulen-3-ol

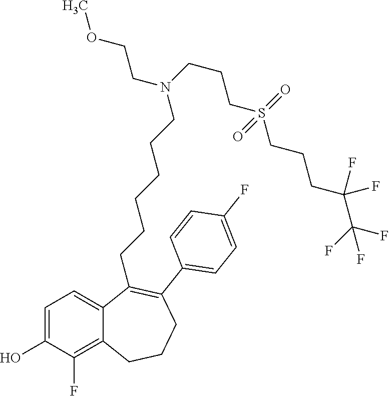 [SMILES: COCCN(CCCCCCC1=C(c2ccc(F)cc2)CCCc2c1ccc(O)c2F)CCCS(=O)(=O)CCCC(F)(F)C(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 94.1 mg (0.28 mmol) of N-(2-methoxyethyl)-3-[(4,4,5,5,5-pentafluoropentyl)sulphonyl]propan-1-amine according to general specification 11. It was purified using HPLC-Method 2. 14.2 mg (9% of theor.) of product was isolated.

¹H-NMR (400 MHz, chloroform-d₁): δ=1.01-1.29 (m, 8H), 1.93 (mc, 2H), 2.04-2.38 (m, 12H), 2.52-2.63 (m, 4H), 2.69-2.77 (m, 2H), 3.01-3.10 (m, 4H), 3.31 (s, 3H), 3.39 (t, 2H), 6.89 (t, 1H), 6.98 (d, 1H), 7.01-7.08 (m, 2H), 7.16-7.23 (m, 2H).

### Example 41

### 4-Fluoro-8-(4-fluorophenyl)-9-{6-[(3-methoxypropyl){3-[(4,4,5,5,5-pentafluoropentyl)sulphonyl]propyl}amino]hexyl}-6,7-dihydro-5H-benzo[7]annulen-3-ol

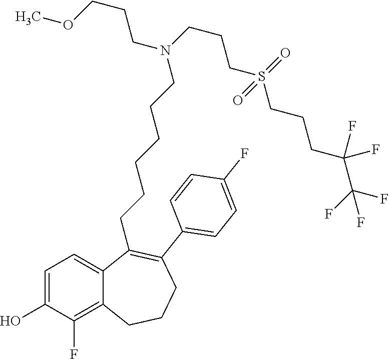 [SMILES: COCCCN(CCCCCCC1=C(c2ccc(F)cc2)CCCc2c1ccc(O)c2F)CCCS(=O)(=O)CCCC(F)(F)C(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 98 mg (0.28 mmol) of 3-methoxy-N-{3-[(4,4,5,5,5-pentafluoropentyl)sulphonyl]propyl}propan-1-amine according to general specification 11. It was purified using HPLC-Method 2. 33.5 mg (21% of theor.) of product was isolated.

¹H-NMR (400 MHz, chloroform-d₁): δ=1.01-1.31 (m, 8H), 1.68 (quin, 2H), 1.98 (quin, 2H), 2.05-2.38 (m, 12H), 2.51 (t, 2H), 2.57 (t, 2H), 2.68-2.78 (m, 2H), 3.02-3.10 (m, 4H), 3.31 (s, 3H), 3.38 (t, 2H), 6.88 (t, 1H), 6.97 (d, 1H), 7.01-7.08 (m, 2H), 7.16-7.23 (m, 2H).

### Example 42

### 4-Fluoro-8-(4-fluorophenyl)-9-[6-(methyl{3-[(4,4,5,5,5-pentafluoropentyl)sulphinyl]propyl}amino)hexyl]-6,7-dihydro-5H-benzo[7]annulen-3-ol

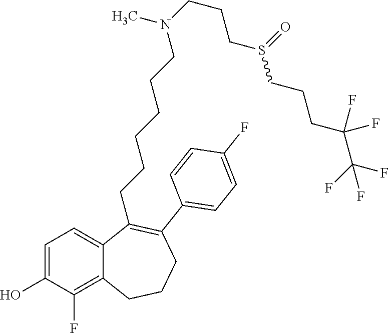 [SMILES: CN(CCCCCCC1=C(c2ccc(F)cc2)CCCc2c1ccc(O)c2F)CCCS(=O)CCCC(F)(F)C(F)(F)F]

150 mg (0.34 mmol) of 9-(6-bromohexyl)-4-fluoro-8-(4-fluorophenyl)-6,7-dihydro-5H-benzo[7]annulen-3-ol was reacted with 116.3 mg (0.41 mmol) of N-methyl-3-[(4,4,5,5,5-pentafluoropentyl)sulphinyl]propan-1-amine according to general specification 11. It was purified using HPLC-Method 1. 96 mg (44% of theor.) of product was isolated.

¹H-NMR (400 MHz, chloroform-d₁): δ=1.01-1.23 (m, 6H), 1.28-1.40 (m, 2H), 2.00-2.46 (m, 17H), 2.62-2.86 (m, 8H), 6.86 (t, 1H), 6.93 (d, 1H), 7.00-7.07 (m, 2H), 7.14-7.21 (m, 2H).

### Example 43

### 4-Fluoro-8-(4-fluorophenyl)-9-[6-(methyl{4-[(4,4,5,5,5-pentafluoropentyl)sulphonyl]butyl}amino)hexyl]-6,7-dihydro-5H-benzo[7]annulen-3-ol

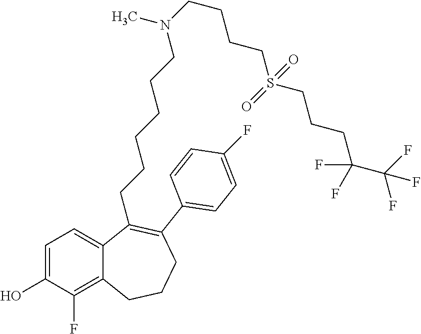 [SMILES: CN(CCCCCCC1=C(c2ccc(F)cc2)CCCc2c1ccc(O)c2F)CCCCS(=O)(=O)CCCC(F)(F)C(F)(F)F]

150 mg (0.34 mmol) of 9-(6-bromohexyl)-4-fluoro-8-(4-fluorophenyl)-6,7-dihydro-5H-benzo[7]annulen-3-ol was reacted with 128.7 mg (0.41 mmol) of N-methyl-4-[(4,4,5,5,5-pentafluoropentyl)sulphonyl]butan-1-amine according to general specification 11. It was purified using HPLC-Method 1. 110.4 mg (48% of theor.) of product was isolated.

¹H-NMR (400 MHz, chloroform-d₁): δ=1.02-1.22 (m, 6H), 1.39 (mc, 2H), 1.72-1.82 (m, 2H), 1.85-1.95 (m, 2H), 2.02-2.37 (m, 10H), 2.41 (s, 3H), 2.49 (mc, 2H), 2.60-2.75 (m, 4H), 3.06 (q, 4H), 6.85 (t, 1H), 6.92 (d, 1H), 7.00-7.07 (m, 2H), 7.14-7.21 (m, 2H).

### Example 44

### 8-(3,5-Difluorophenyl)-9-[6-(methyl{4-[(4,4,4-trifluorobutyl)sulphonyl]butyl}amino)hexyl]-6,7-dihydro-5H-benzo[7]annulen-3-ol

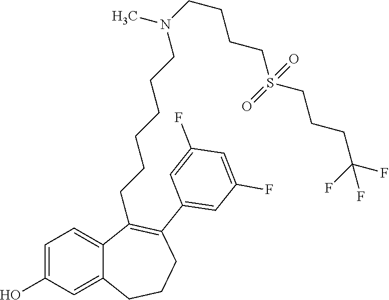 [SMILES: CN(CCCCCCC1=C(c2cc(F)cc(F)c2)CCCc2cc(O)ccc21)CCCCS(=O)(=O)CCCC(F)(F)F]

200 mg (0.46 mmol) of 9-(6-bromohexyl)-8-(3,5-difluorophenyl)-6,7-dihydro-5H-benzo[7]annulen-3-ol was reacted with 144.1 mg (0.55 mmol) of N-methyl-4-[(4,4,4-trifluorobutyl)sulphonyl]butan-1-amine according to general specification 11. It was purified using HPLC (HPLC-Method 2, then with XBridge C18, 5μ, 150×19 mm, 25 mL/min, solvent: water with 0.2% ammonia-acetonitrile 40:60, 0-1 minute; 40:60→0:100, 1-11 minutes; 0:100, 11-15 minutes). 52 mg (18% of theor.) of product was isolated.

¹H-NMR (400 MHz, chloroform-d₁): δ=1.06-1.34 (m, 8H), 1.59 (quin, 2H), 1.85 (mc, 2H), 2.03-2.24 (m, 11H), 2.26-2.40 (m, 6H), 2.61 (t, 2H), 2.98-3.06 (m, 4H), 6.66-6.79 (m, 5H), 7.15 (d, 1H).

### Example 44a

### Napthalene-1,5-disulphonic acid salt (2:1) of 8-(3,5-difluorophenyl)-9-[6-(methyl{4-[(4,4,4-trifluorobutyl)sulphonyl]butyl}amino)hexyl]-6,7-dihydro-5H-benzo[7]annulen-3-ol

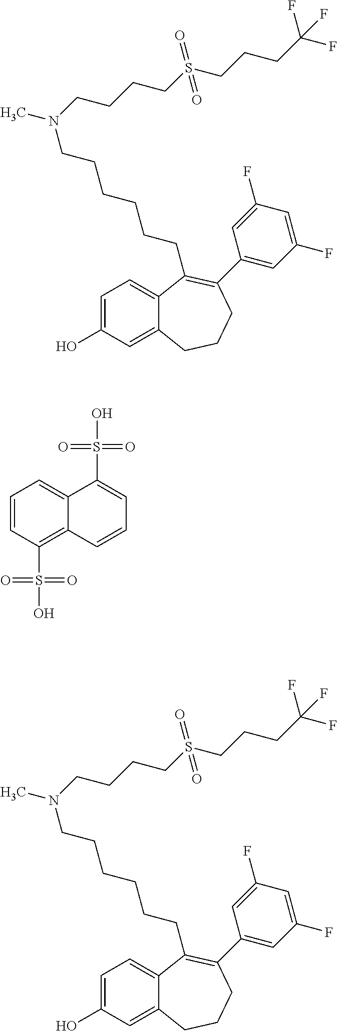 [SMILES: CN(CCCCCCC1=C(c2cc(F)cc(F)c2)CCCc2cc(O)ccc21)CCCCS(=O)(=O)CCCC(F)(F)F ; O=S(=O)(O)c1cccc2c(S(=O)(=O)O)cccc12 ; CN(CCCCCCC1=C(c2cc(F)cc(F)c2)CCCc2cc(O)ccc21)CCCCS(=O)(=O)CCCC(F)(F)F]

8-(3,5-difluorophenyl)-9-[6-(methyl{4-[(4,4,4-trifluorobutyl)sulphonyl]butyl}amino)hexyl]-6,7-dihydro-5H-benzo[7]annulen-3-ol (500 mg, 0.81 mmol) was dissolved in ethanol (10 mL), and toluene (10 mL) and then a solution of naphthalene-1,5-disulphonic acid (234 mg, 0.812 mmol) in water (1 mL) were added. The solution was stirred in an open round-bottomed flask at room temperature and left to evaporate slowly. At a residual amount of solution of about 20%, the crystalline compound that had formed was filtered off, washed with a small amount of toluene/ethanol solution (1/1) and then dried for some days in air and then briefly under high-vacuum. 470 mg (38%) of the 2:1-napthalene-1-5-disulphonic acid salt was obtained.

The data from NMR, LCMS, IR, DSC, TGA, PLM and elemental analysis are presented below. According to elemental analysis the compound contains 1 mol equivalent water. The salt melts at 186° C. (ΔH=60 J/g), which might offer potential processing advantages (grinding, tableting) compared with the example compound 44, which melts at 71° C. (ΔH=65 J/g).

¹H-NMR (400 MHz, DMSO-d₆) δ: 9.4 (bs, 2H), 8.95 (bs, 2H), 8.84 (d, 2H), 7.92 (d, 2H), 7.40 (t, 2H), 7.15 (m, 4H), 6.96 (d, 4H), 6.67 (m, 4H), 3.20 (m, 8H), 3.07 (bm, 2H), 2.95 (bm, 4H), 2.86 (bm, 2H), 2.68 (d, 6H), 2.55 (t, 4H), 2.45 (m, 8H), 2.34 (t, 4H), 2.05 (m, 4H), 1.95 (t, 4H), 1.91 (m, 4H), 1.70 (m, 8H), 1.47 (m, 4H), 1.16-1.10 (m, 8H).

¹³C-NMR (100 MHz, DMSO-d₆) δ: 162.2 (dd), 156.1, 147.6 (t), 143.8, 142.0, 137.9, 133.7, 130.6, 129.5, 129.0, 127.3, 127.1 (q), 123.9, 123.8, 115.3, 113.0, 111.3 (dd), 101.8 (t), 55.0, 54.2, 50.7, 50.2, 39.6 (signal hidden by DMSO-d₆ signal), 33.4, 32.3, 32.1, 31.4, 31.2, 31.0, 28.5, 25.6, 23.1, 22.1, 18.4, 14.9 (q).

LC-MS: Rₜ=1.30 min

MS (ESI pos): m/z 616 (M+H)⁺

LC-MS method: MHZ-QP-GO-1

Instrument: Micromass Quattro Premier with Waters UPLC Acquity; column: Thermo Hypersil GOLD 1.9μ 50×1 mm; eluent A: 1 l water+0.5 ml 50% formic acid, eluent B: 1 l acetonitrile+0.5 ml 50% formic acid; gradient: 0.0 min 90% A→0.1 min 90% A→1.5 min 10% A→2.2 min 10% A Furnace: 50° C.; flow: 0.33 ml/min; UV-detection: 210 nm.

IR

IR (ATR): 3127, 2932, 2858, 1615, 1583, 1570, 1499, 1455, 1428, 1398, 1329, 1298, 1256, 1219, 1200, 1180, 1148, 1117, 1060, 1030, 985, 964, 872, 858, 826, 806, 763, 732, 713, 676, 666, 655, 609 cm⁻¹

Differential Scanning Calorimetry (DSC)

M.p. 186° C., ΔH=60 J/g

Melting points were determined by differential scanning calorimetry, which was carried out on the Mettler-Toledo 823^{e} DSC apparatus with a TSO801RO autosampler and STAR^{e} software. The analyses were performed in 40-μL-aluminium crucibles with closed covers with a small hole (about 0.2 mm). The sample weight was as a rule 1.5-3 mg. The heat flux was measured in the temperature range 30° C. to 400° C. at a heating rate of 10° C. per minute under an argon stream of 30 mL/min.

Thermogravimetric Analysis (TGA)

no weight loss before endothermic fusion

The thermogravimetric analyses were performed on the Mettler-Toledo TGA/SDTA851^{e} TGA apparatus with a TSO801RO autosampler and STAR^{e} software. The analyses were carried out in open 100-μL-aluminium crucibles. The sample weight at the start of the experiment was as a rule 1.5-3 mg. The weight of the sample was measured over a temperature range from 30° C. to 400° C. at a heating rate of 10° C. per minute under an argon stream of 30 mL/min.

Polarization Light Microscopy (PLM)

PLM (100×): crystalline

Polarization light microscopy was carried out on a polarization light microscopy imaging system for determination of particle size distribution of the Clemex PS3 type with a microscope of the Leica DM type with 50×-, 100×-, 200×- and 500×-lenses, a high-resolution monochromatic digital camera with 1600×1200 pixels and a motorized X-Y stage from Marzhauzer, controlled by a Clemex-ST-2000 control system. For sample measurement, a small amount of crystalline material suspended in a drop of oil was placed on a slide (76×26 mm), then the suspension was covered with a cover glass (22×40 mm).

Elemental Analysis:

Analysis. Calculated for 2 (C₃₂H₄₂F₅NO₃S)+C₁₀H₈O₆S₂+H₂O: % C 57.80, % H 6.16, % N 1.82.

Found: % C 57.7, % H 6.0, % N 1.9.

The elemental analyses were carried out by Currenta according to DIN-ISO 17025.

### Example 45

### 8-(3,4-Difluorophenyl)-9-[6-(methyl{4-[(4,4,4-trifluorobutyl)sulphonyl]butyl}amino)hexyl]-6,7-dihydro-5H-benzo[7]annulen-3-ol

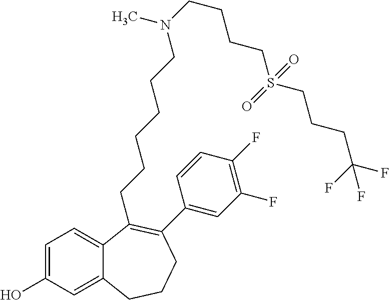 [SMILES: CN(CCCCCCC1=C(c2ccc(F)c(F)c2)CCCc2cc(O)ccc21)CCCCS(=O)(=O)CCCC(F)(F)F]

200 mg (0.46 mmol) of 9-(6-bromohexyl)-8-(3,4-difluorophenyl)-6,7-dihydro-5H-benzo[7]annulen-3-ol was reacted with 144.1 mg (0.55 mmol) of N-methyl-4-[(4,4,4-trifluorobutyl)sulphonyl]butan-1-amine according to general specification 11. It was purified using HPLC (HPLC-Method 2, XBridge C18, 5μ, 150×19 mm, 25 mL/min, solvent: water with 0.2% ammonia-acetonitrile 40:60, 0-1 minute; 40:60→0:100, 1-11 minutes; 0:100, 11-15 minutes). 48 mg (17% of theor.) of product was isolated.

¹H-NMR (400 MHz, chloroform-d₁): δ=1.05-1.35 (m, 8H), 1.60 (quin, 2H), 1.85 (mc, 2H), 2.02-2.25 (m, 11H), 2.26-2.39 (m, 6H), 2.60 (t, 2H), 2.98-3.07 (m, 4H), 6.68-6.75 (m, 2H), 6.92-6.97 (m, 1H), 7.04 (ddd, 1H), 7.09-7.18 (m, 2H).

### Example 46

### 8-(3,5-Difluorophenyl)-9-[6-(methyl{4-[(3,3,3-trifluoropropyl)sulphonyl]butyl}amino)hexyl]-6,7-dihydro-5H-benzo[7]annulen-3-ol

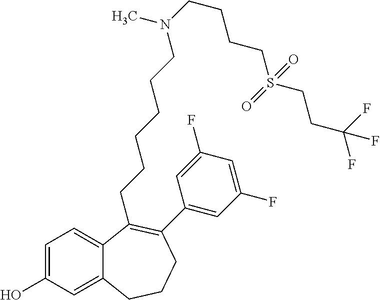 [SMILES: CN(CCCCCCC1=C(c2cc(F)cc(F)c2)CCCc2cc(O)ccc21)CCCCS(=O)(=O)CCC(F)(F)F]

130 mg (0.30 mmol) of 9-(6-bromohexyl)-8-(3,5-difluorophenyl)-6,7-dihydro-5H-benzo[7]annulen-3-ol was reacted with 88.6 mg (0.36 mmol) of N-methyl-4-[(3,3,3-trifluoropropyl)sulphonyl]butan-1-amine according to general specification 11. It was purified using HPLC-Method 1. 113 mg (62% of theor.) of product was isolated.

¹H-NMR (300 MHz, chloroform-d₁): δ=1.02-1.27 (m, 6H), 1.32-1.48 (m, 2H), 1.74-1.99 (m, 4H), 2.00-2.15 (m, 4H), 2.36 (t, 2H), 2.41-2.77 (m, 11H), 3.11 (t, 2H), 3.16-3.25 (m, 2H), 6.63-6.80 (m, 5H), 7.11 (d, 1H).

### Example 47

### 8-(3,4-Difluorophenyl)-9-[6-(methyl{4-[(3,3,3-trifluoropropyl)sulphonyl]butyl}amino)hexyl]-6,7-dihydro-5H-benzo[7]annulen-3-ol

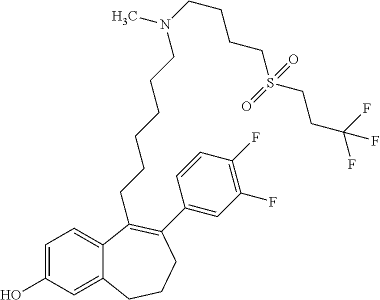 [SMILES: CN(CCCCCCC1=C(c2ccc(F)c(F)c2)CCCc2cc(O)ccc21)CCCCS(=O)(=O)CCC(F)(F)F]

130 mg (0.30 mmol) of 9-(6-bromohexyl)-8-(3,4-difluorophenyl)-6,7-dihydro-5H-benzo[7]annulen-3-ol was reacted with 88.6 mg (0.36 mmol) of N-methyl-4-[(3,3,3-trifluoropropyl)sulphonyl]butan-1-amine according to general specification 11. It was purified using HPLC-Method 1. 130 mg (68% of theor.) of product was isolated.

¹H-NMR (400 MHz, chloroform-d₁): δ=1.03-1.25 (m, 6H), 1.31-1.42 (m, 2H), 1.77 (quin, 2H), 1.91 (quin, 2H), 2.01-2.14 (m, 4H), 2.34 (t, 2H), 2.39 (s, 3H), 2.44 (mc, 2H), 2.55-2.74 (m, 6H), 3.09 (mc, 2H), 3.20 (mc, 2H), 6.70-6.77 (m, 2H), 6.91-6.96 (m, 1H), 7.03 (ddd, 1H), 7.09-7.17 (m, 2H).

### Example 48

### 8-(4-Fluorophenyl)-9-[6-(methyl{4-[(4,4,4-trifluorobutyl)sulphonyl]butyl}amino)hexyl]-6,7-dihydro-5H-benzo[7]annulen-3-ol

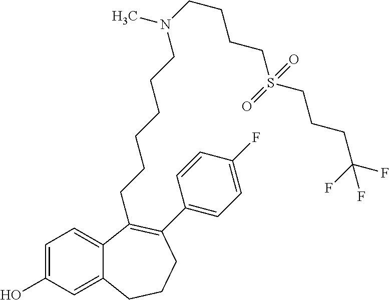 [SMILES: CN(CCCCCCC1=C(c2ccc(F)cc2)CCCc2cc(O)ccc21)CCCCS(=O)(=O)CCCC(F)(F)F]

126 mg (0.30 mmol) of 9-(6-bromohexyl)-8-(4-fluorophenyl)-6,7-dihydro-5H-benzo[7]annulen-3-ol was reacted with 94.7 mg (0.36 mmol) of N-methyl-4-[(4,4,4-trifluorobutyl)sulphonyl]butan-1-amine according to general specification 11. It was purified using HPLC-Method 1. The product was dissolved in dichloromethane, washed once with saturated sodium hydrogen carbonate solution and three times with water, dried over magnesium sulphate and concentrated by evaporation. 105 mg (58% of theor.) of product was isolated.

¹H-NMR (300 MHz, chloroform-d₁): δ=1.02-1.35 (m, 8H), 1.58 (quin, 2H), 1.78-1.91 (m, 2H), 2.02-2.41 (m, 17H), 2.61 (t, 2H), 2.96-3.06 (m, 4H), 6.66-6.74 (m, 2H), 6.99-7.08 (m, 2H), 7.12-7.23 (m, 3H).

### Example 49

### 8-(4-Fluorophenyl)-9-[5-(methyl{4-[(4,4,4-trifluorobutyl)sulphonyl]butyl}amino)pentyl]-6,7-dihydro-5H-benzo[7]annulen-3-ol

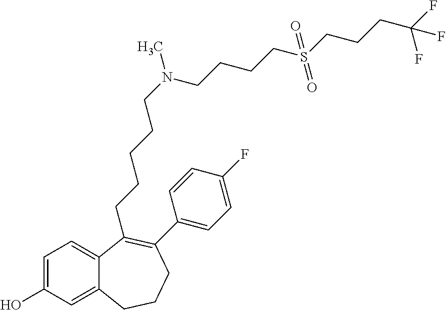 [SMILES: CN(CCCCCC1=C(c2ccc(F)cc2)CCCc2cc(O)ccc21)CCCCS(=O)(=O)CCCC(F)(F)F]

130.4 mg (0.32 mmol) of 9-(6-bromohexyl)-8-(4-fluorophenyl)-6,7-dihydro-5H-benzo[7]annulen-3-ol was reacted with 101.4 mg (0.39 mmol) of N-methyl-4-[(4,4,4-trifluorobutyl)sulphonyl]butan-1-amine according to general specification 11. It was purified using HPLC-Method 1. The product was dissolved in dichloromethane, washed once with saturated sodium hydrogen carbonate solution and three times with water, dried over magnesium sulphate and concentrated by evaporation. 98 mg (52% of theor.) of product was isolated.

¹H-NMR (300 MHz, chloroform-d₁): δ=1.02-1.35 (m, 6H), 1.57 (quin, 2H), 1.83 (mc, 2H), 2.01-2.41 (m, 17H), 2.60 (mc, 2H), 2.94-3.05 (m, 4H), 6.65-6.71 (m, 2H), 6.99-7.08 (m, 2H), 7.12-7.23 (m, 3H).

### Example 50

### 4-Fluoro-8-(4-fluorophenyl)-9-[6-(methyl{4-[(3,3,3-trifluoropropyl)sulphonyl]butyl}amino)hexyl]-6,7-dihydro-5H-benzo[7]annulen-3-ol

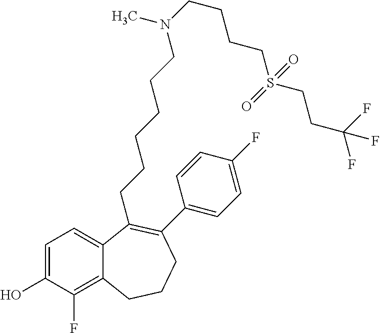 [SMILES: CN(CCCCCCC1=C(c2ccc(F)cc2)CCCc2c1ccc(O)c2F)CCCCS(=O)(=O)CCC(F)(F)F]

130 mg (0.30 mmol) of 9-(6-bromohexyl)-4-fluoro-8-(4-fluorophenyl)-6,7-dihydro-5H-benzo[7]annulen-3-ol was reacted with 88.6 mg (0.36 mmol) of N-methyl-4-[(3,3,3-trifluoropropyl)sulphonyl]butan-1-amine according to general specification 11. It was purified using HPLC-Method 1. 115 mg (63% of theor.) of product was isolated.

¹H-NMR (300 MHz, chloroform-d₁): δ=1.01-1.24 (m, 6H), 1.30-1.45 (m, 2H), 1.68-1.82 (m, 2H), 1.91 (quin, 2H), 2.01-2.16 (m, 4H), 2.27-2.39 (m, 5H), 2.44 (mc, 2H), 2.54-2.77 (m, 6H), 3.10 (mc, 2H), 3.19 (m, 2H), 6.86 (t, 1H), 6.94 (d, 1H), 7.04 (tt, 2H), 7.14-7.22 (m, 2H).

### Example 51

### 8-(4-Fluorophenyl)-9-[6-(methyl{4-[(3,3,3-trifluoropropyl)sulphonyl]butyl}amino)hexyl]-6,7-dihydro-5H-benzo[7]annulen-3-ol

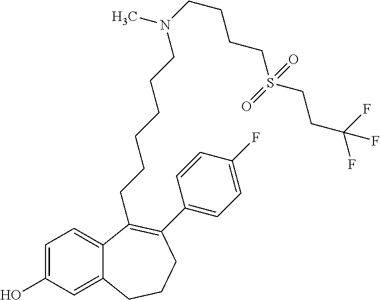 [SMILES: CN(CCCCCCC1=C(c2ccc(F)cc2)CCCc2cc(O)ccc21)CCCCS(=O)(=O)CCC(F)(F)F]

122 mg (0.29 mmol) of 9-(6-bromohexyl)-8-(4-fluorophenyl)-6,7-dihydro-5H-benzo[7]annulen-3-ol was reacted with 86.7 mg (0.35 mmol) of N-methyl-4-[(3,3,3-trifluoropropyl)sulphonyl]butan-1-amine according to general specification 11. It was purified using HPLC-Method 1. 110 mg (64% of theor.) of product was isolated.

¹H-NMR (300 MHz, chloroform-d₁): δ=1.01-1.25 (m, 6H), 1.28-1.43 (m, 2H), 1.76 (quin, 2H), 1.91 (quin, 2H), 2.01-2.17 (m, 4H), 2.29-2.48 (m, 7H), 2.53-2.76 (m, 6H), 3.09 (mc, 2H), 3.20 (mc, 2H), 6.68-6.77 (m, 2H), 7.03 (tt, 2H), 7.09-7.22 (m, 3H).

### Example 52

### 8-(4-Fluorophenyl)-9-[5-(methyl{4-[(3,3,3-trifluoropropyl)sulphonyl]butyl}amino)pentyl]-6,7-dihydro-5H-benzo[7]annulen-3-ol

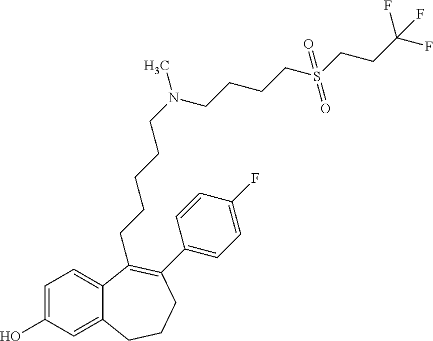 [SMILES: CN(CCCCCC1=C(c2ccc(F)cc2)CCCc2cc(O)ccc21)CCCCS(=O)(=O)CCC(F)(F)F]

122 mg (0.30 mmol) of 9-(6-bromohexyl)-8-(4-fluorophenyl)-6,7-dihydro-5H-benzo[7]annulen-3-ol was reacted with 89.8 mg (0.36 mmol) of N-methyl-4-[(3,3,3-trifluoropropyl)sulphonyl]butan-1-amine according to general specification 11. It was purified using HPLC-Method 1. 118 mg (68% of theor.) of product was isolated.

¹H-NMR (300 MHz, chloroform-d₁): δ=1.02-1.27 (m, 4H), 1.41 (mc, 2H), 1.69-1.95 (m, 4H), 1.99-2.17 (m, 4H), 2.34 (mc, 2H), 2.41 (s, 3H), 2.48 (mc, 2H), 2.53-2.75 (m, 6H), 3.07 (mc, 2H), 3.15-3.22 (m, 2H), 6.68-6.76 (m, 2H), 6.99-7.08 (m, 2H), 7.12 (d, 1H), 7.14-7.20 (m, 2H).

### Example 53

### 8-(4-Fluorophenyl)-9-[6-(methyl{3-[(3,3,3-trifluoropropyl)sulphonyl]propyl}amino)hexyl]-6,7-dihydro-5H-benzo[7]annulen-3-ol

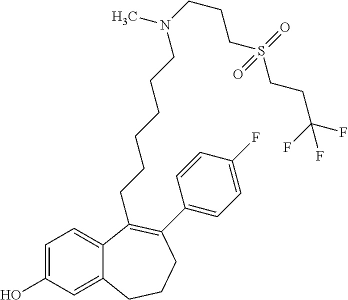 [SMILES: CN(CCCCCCC1=C(c2ccc(F)cc2)CCCc2cc(O)ccc21)CCCS(=O)(=O)CCC(F)(F)F]

130 mg (0.31 mmol) of 9-(6-bromohexyl)-8-(4-fluorophenyl)-6,7-dihydro-5H-benzo[7]annulen-3-ol was reacted with 87.2 mg (0.37 mmol) of N-methyl-3-[(3,3,3-trifluoropropyl)sulphonyl]propan-1-amine according to general specification 11. It was purified using HPLC-Method 1. 93.2 mg (53% of theor.) of product was isolated.

¹H-NMR (300 MHz, chloroform-d₁): δ=1.00-1.25 (m, 6H), 1.34 (mc, 2H), 2.01-2.20 (m, 6H), 2.29-2.44 (m, 7H), 2.56-2.77 (m, 6H), 3.11-3.27 (m, 4H), 6.71-6.79 (m, 2H), 6.98-7.08 (m, 2H), 7.10-7.23 (m, 3H).

### Example 54

### 8-(4-Fluorophenyl)-9-{6-[(2-hydroxyethyl){3-[(4,4,5,5,5-pentafluoropentyl)sulphonyl]propyl}amino]hexyl}-6,7-dihydro-5H-benzo[7]annulen-3-ol

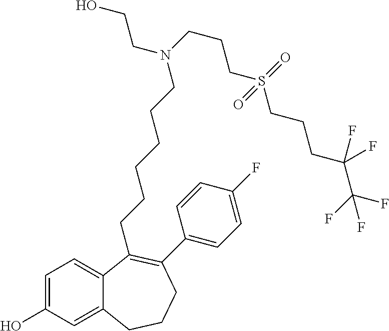 [SMILES: O=S(=O)(CCCN(CCO)CCCCCCC1=C(c2ccc(F)cc2)CCCc2cc(O)ccc21)CCCC(F)(F)C(F)(F)F]

130 mg (0.31 mmol) of 9-(6-bromohexyl)-8-(4-fluorophenyl)-6,7-dihydro-5H-benzo[7]annulen-3-ol was reacted with 122.3 mg (0.37 mmol) of 2-({3-[(4,4,5,5,5-pentafluoropentyl)sulphonyl]propyl}amino)ethanol according to general specification 11. It was purified using HPLC-Method 1. 78.4 mg (38% of theor.) of product was isolated.

¹H-NMR (300 MHz, chloroform-d₁): δ=1.00-1.38 (m, 8H), 2.01-2.41 (m, 12H), 2.52 (mc, 2H), 2.62 (t, 2H), 2.79 (mc, 2H), 2.86 (t, 2H), 3.06-3.16 (m, 4H), 3.71 (mc, 2H), 6.71-6.80 (m, 2H), 6.99-7.08 (m, 2H), 7.11-7.22 (m, 3H).

### Example 55

### 8-(4-Fluorophenyl)-9-{6-[(3-hydroxypropyl){3-[(4,4,5,5,5-pentafluoropentyl)sulphonyl]propyl}amino]hexyl}-6,7-dihydro-5H-benzo[7]annulen-3-ol

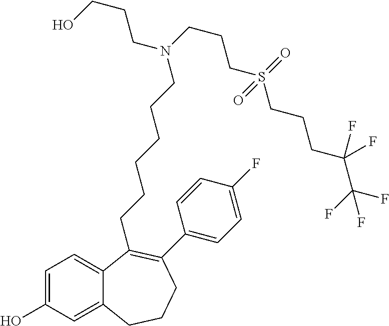 [SMILES: O=S(=O)(CCCN(CCCO)CCCCCCC1=C(c2ccc(F)cc2)CCCc2cc(O)ccc21)CCCC(F)(F)C(F)(F)F]

130 mg (0.31 mmol) of 9-(6-bromohexyl)-8-(4-fluorophenyl)-6,7-dihydro-5H-benzo[7]annulen-3-ol was reacted with 127.6 mg (0.37 mmol) of 3-({3-[(4,4,5,5,5-pentafluoropentyl)sulphonyl]propyl}amino)propan-1-ol according to general specification 11. It was purified using HPLC-Method 1. 85.8 mg (41% of theor.) of product was isolated.

¹H-NMR (300 MHz, chloroform-d₁): δ=1.00-1.39 (m, 8H), 1.77 (mc, 2H), 2.02-2.41 (m, 12H), 2.50 (mc, 2H), 2.60 (mc, 2H), 2.77-2.90 (m, 4H), 3.12 (mc, 4H), 3.74 (t, 2H), 6.72-6.80 (m, 2H), 7.03 (mc, 2H), 7.10-7.22 (m, 3H).

### Example 56

### 8-(4-Fluorophenyl)-9-{6-[(2-hydroxyethyl){3-[(3,3,3-trifluoropropyl)sulphonyl]propyl}amino]hexyl}-6,7-dihydro-5H-benzo[7]annulen-3-ol

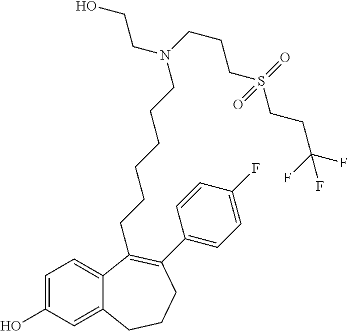 [SMILES: O=S(=O)(CCCN(CCO)CCCCCCC1=C(c2ccc(F)cc2)CCCc2cc(O)ccc21)CCC(F)(F)F]

130 mg (0.31 mmol) of 9-(6-bromohexyl)-8-(4-fluorophenyl)-6,7-dihydro-5H-benzo[7]annulen-3-ol was reacted with 98.4 mg (0.37 mmol) of 2-({3-[(3,3,3-trifluoropropyl)sulphonyl]propyl}amino)ethanol according to general specification 11. It was purified using HPLC-Method 1. 75.6 mg (40% of theor.) of product was isolated.

¹H-NMR (300 MHz, chloroform-d₁): δ=1.00-1.39 (m, 8H), 2.00-2.23 (m, 6H), 2.35 (t, 2H), 2.51-2.77 (m, 6H), 2.85 (t, 2H), 2.94 (t, 2H), 3.13-3.31 (m, 4H), 3.75 (t, 2H), 6.72-6.80 (m, 2H), 7.03 (mc, 2H), 7.10-7.21 (m, 3H).

### Example 57

### 8-(4-Fluorophenyl)-9-{6-[(3-hydroxypropyl){3-[(3,3,3-trifluoropropyl)sulphonyl]propyl}amino]hexyl}-6,7-dihydro-5H-benzo[7]annulen-3-ol

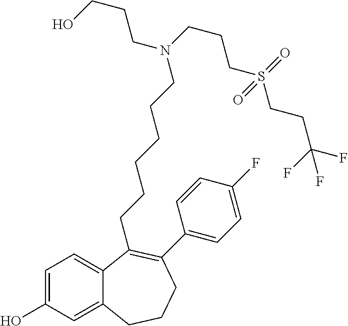 [SMILES: O=S(=O)(CCCN(CCCO)CCCCCCC1=C(c2ccc(F)cc2)CCCc2cc(O)ccc21)CCC(F)(F)F]

130 mg (0.31 mmol) of 9-(6-bromohexyl)-8-(4-fluorophenyl)-6,7-dihydro-5H-benzo[7]annulen-3-ol was reacted with 103.6 mg (0.37 mmol) of 3-({3-[(3,3,3-trifluoropropyl)sulphonyl]propyl}amino)propan-1-ol according to general specification 11. It was purified using HPLC-Method 1. 79.7 mg (42% of theor.) of product was isolated.

¹H-NMR (300 MHz, chloroform-d₁): δ=1.01-1.38 (m, 8H), 1.70-1.83 (m, 2H) 2.02-2.21 (m, 6H), 2.35 (t, 2H), 2.47 (mc, 2H), 2.55-2.90 (m, 8H), 3.15 (t, 2H), 3.25 (mc, 2H), 3.74 (t, 2H), 6.71-6.81 (m, 2H), 7.03 (mc, 2H), 7.11-7.23 (m, 3H).

### Example 58

### 9-{6-[(4-Fluorobenzyl){3-[(4,4,5,5,5-pentafluoropentyl)sulphonyl]propyl}amino]hexyl}-8-(4-fluorophenyl)-6,7-dihydro-5H-benzo[7]annulen-3-ol

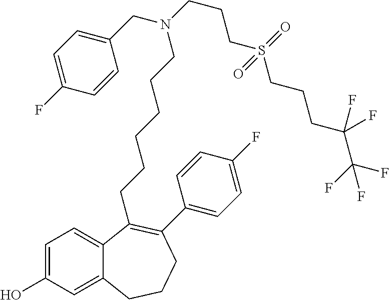 [SMILES: O=S(=O)(CCCN(CCCCCCC1=C(c2ccc(F)cc2)CCCc2cc(O)ccc21)Cc1ccc(F)cc1)CCCC(F)(F)C(F)(F)F]

130 mg (0.31 mmol) of 9-(6-bromohexyl)-8-(4-fluorophenyl)-6,7-dihydro-5H-benzo[7]annulen-3-ol was reacted with 146.3 mg (0.37 mmol) of N-(4-fluorobenzyl)-3-[(4,4,5,5,5-pentafluoropentyl)sulphonyl]propan-1-amine according to general specification 11. It was purified using HPLC-Method 1. 15.6 mg (7% of theor.) of product was isolated.

¹H-NMR (300 MHz, chloroform-d₁): δ=0.99-1.33 (m, 8H), 1.90 (mc, 2H), 2.03-2.20 (m, 6H), 2.21-2.42 (m, 6H), 2.48 (t, 2H), 2.57-2.66 (m, 2H), 2.90-3.00 (m, 4H), 3.46 (s, 2H), 6.71-6.79 (m, 2H), 6.95-7.07 (m, 4H), 7.14-7.24 (m, 5H).

### Example 59

### 4-Fluoro-8-(4-fluorophenyl)-9-{6-[(2-hydroxyethyl){3-[(4,4,5,5,5-pentafluoropentyl)sulphonyl]propyl}amino]hexyl}-6,7-dihydro-5H-benzo[7]annulen-3-ol

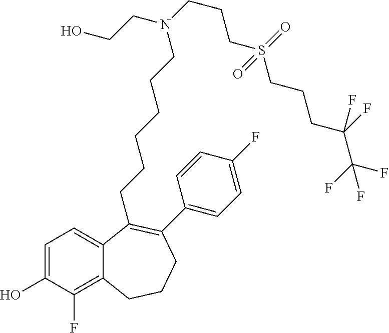 [SMILES: O=S(=O)(CCCN(CCO)CCCCCCC1=C(c2ccc(F)cc2)CCCc2c1ccc(O)c2F)CCCC(F)(F)C(F)(F)F]

130 mg (0.30 mmol) of 9-(6-bromohexyl)-4-fluoro-8-(4-fluorophenyl)-6,7-dihydro-5H-benzo[7]annulen-3-ol was reacted with 117.3 mg (0.36 mmol) of 2-({3-[(4,4,5,5,5-pentafluoropentyl)sulphonyl]propyl}amino)ethanol according to general specification 11. It was purified using HPLC-Method 1. 48.1 mg (24% of theor.) of product was isolated.

¹H-NMR (300 MHz, chloroform-d₁): δ=1.03-1.24 (m, 6H), 1.33 (m, 2H), 2.03-2.39 (m, 12H), 2.54 (mc, 2H), 2.67-2.79 (m, 4H), 2.83 (t, 2H), 3.05-3.14 (m, 4H), 3.69 (t, 2H), 6.88 (t, 1H), 6.96 (d, 1H), 7.04 (tt, 2H), 7.19 (m, 2H).

### Example 60

### 4-Fluoro-8-(4-fluorophenyl)-9-{6-[(3-hydroxypropyl){3-[(4,4,5,5,5-pentafluoropentyl)sulphonyl]propyl}amino]hexyl}-6,7-dihydro-5H-benzo[7]annulen-3-ol

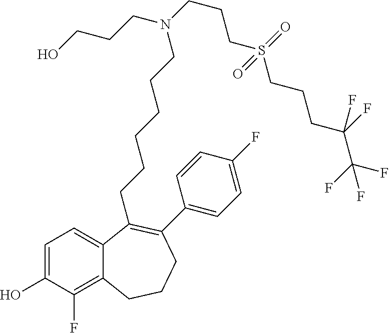 [SMILES: O=S(=O)(CCCN(CCCO)CCCCCCC1=C(c2ccc(F)cc2)CCCc2c1ccc(O)c2F)CCCC(F)(F)C(F)(F)F]

130 mg (0.30 mmol) of 9-(6-bromohexyl)-4-fluoro-8-(4-fluorophenyl)-6,7-dihydro-5H-benzo[7]annulen-3-ol was reacted with 122.3 mg (0.36 mmol) of 3-({3-[(4,4,5,5,5-pentafluoropentyl)sulphonyl]propyl}amino)propan-1-ol according to general specification 11. It was purified using HPLC-Method 1. 56.6 mg (27% of theor.) of product was isolated.

¹H-NMR (300 MHz, chloroform-d₁): δ=1.02-1.24 (m, 6H), 1.32 (m, 2H), 1.75 (mc, 2H), 2.01-2.39 (m, 12H), 2.50 (mc, 2H), 2.66-2.85 (m, 6H), 3.10 (mc, 4H), 3.75 (t, 2H), 6.87 (t, 1H), 6.95 (d, 1H), 7.04 (tt, 2H), 7.18 (m, 2H).

### Example 61

### 4-Fluoro-8-(4-fluorophenyl)-9-{6-[(2-hydroxyethyl){3-[(3,3,3-trifluoropropyl)sulphonyl]propyl}amino]hexyl}-6,7-dihydro-5H-benzo[7]annulen-3-ol

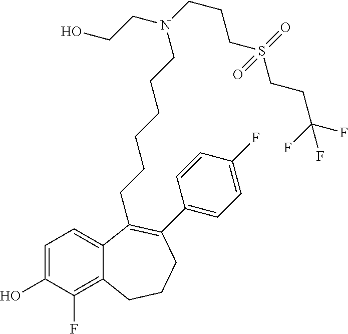 [SMILES: O=S(=O)(CCCN(CCO)CCCCCCC1=C(c2ccc(F)cc2)CCCc2c1ccc(O)c2F)CCC(F)(F)F]

130 mg (0.30 mmol) of 9-(6-bromohexyl)-4-fluoro-8-(4-fluorophenyl)-6,7-dihydro-5H-benzo[7]annulen-3-ol was reacted with 94.3 mg (0.36 mmol) of 2-({3-[(3,3,3-trifluoropropyl)sulphonyl]propyl}amino)ethanol according to general specification 11. It was purified using HPLC-Method 1. 22.7 mg (12% of theor.) of product was isolated.

¹H-NMR (300 MHz, chloroform-d₁): δ=1.03-1.25 (m, 6H), 1.34 (mc, 2H), 2.03-2.19 (m, 6H), 2.34 (t, 2H), 2.55 (mc, 2H), 2.60-2.80 (m, 6H), 2.84 (t, 2H), 3.15 (t, 2H), 3.23 (mc, 2H), 3.69 (t, 2H), 6.88 (t, 1H), 6.97 (d, 1H), 7.05 (tt, 2H), 7.19 (m, 2H).

### Example 62

### 4-Fluoro-8-(4-fluorophenyl)-9-{6-[(3-hydroxypropyl){3-[(3,3,3-trifluoropropyl)sulphonyl]propyl}amino]hexyl}-6,7-dihydro-5H-benzo[7]annulen-3-ol

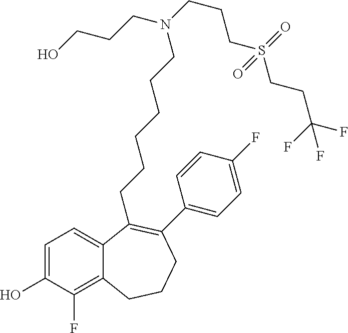 [SMILES: O=S(=O)(CCCN(CCCO)CCCCCCC1=C(c2ccc(F)cc2)CCCc2c1ccc(O)c2F)CCC(F)(F)F]

130 mg (0.30 mmol) of 9-(6-bromohexyl)-4-fluoro-8-(4-fluorophenyl)-6,7-dihydro-5H-benzo[7]annulen-3-ol was reacted with 99.4 mg (0.36 mmol) of 3-({3-[(3,3,3-trifluoropropyl)sulphonyl]propyl}amino)propan-1-ol according to general specification 11. It was purified using HPLC-Method 1. 33 mg (17% of theor.) of product was isolated.

¹H-NMR (300 MHz, chloroform-d₁): δ=1.02-1.37 (m, 8H), 1.73 (mc, 2H), 2.02-2.17 (m, 6H), 2.34 (t, 2H), 2.44 (mc, 2H), 2.60-2.80 (m, 8H), 3.12 (t, 2H), 3.23 (mc, 2H), 3.75 (t, 2H), 6.88 (t, 1H), 6.96 (d, 1H), 7.00-7.09 (m, 2H), 7.14-7.23 (m, 2H).

### Example 63

### 9-[6-(tert-Butyl{3-[(4,4,5,5,5-pentafluoropentyl)sulphonyl]propyl}amino)hexyl]-4-fluoro-8-(4-fluorophenyl)-6,7-dihydro-5H-benzo[7]annulen-3-ol

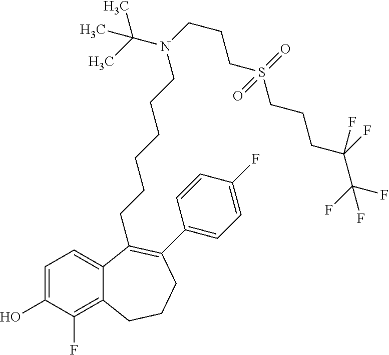 [SMILES: CC(C)(C)N(CCCCCCC1=C(c2ccc(F)cc2)CCCc2c1ccc(O)c2F)CCCS(=O)(=O)CCCC(F)(F)C(F)(F)F]

130 mg (0.30 mmol) of 9-(6-bromohexyl)-4-fluoro-8-(4-fluorophenyl)-6,7-dihydro-5H-benzo[7]annulen-3-ol was reacted with 121.6 mg (0.36 mmol) of N-tert-butyl-3-[(4,4,5,5,5-pentafluoropentyl)sulphonyl]propan-1-amine in 6.7 mL acetonitrile according to general specification 11, except that it was not stirred at 85° C., but was treated at 180° C. for 15 minutes at 250 watt in a microwave. It was purified using HPLC-Method 1. 50 mg (24% of theor.) of product was isolated.

¹H-NMR (400 MHz, chloroform-d₁): δ=1.02-1.23 (m, 15H), 1.35 (m, 2H), 1.99-2.38 (m, 12H), 2.47 (mc, 2H), 2.69-2.79 (m, 4H), 3.09 (mc, 4H), 6.90 (t, 1H), 6.97 (d, 1H), 7.05 (tt, 2H), 7.16-7.23 (m, 2H).

### Example 64

### 9-{6-[(2,2-Difluoroethyl){3-[(4,4,5,5,5-pentafluoropentyl)sulphonyl]propyl}amino]hexyl}-4-fluoro-8-(4-fluorophenyl)-6,7-dihydro-5H-benzo[7]annulen-3-ol

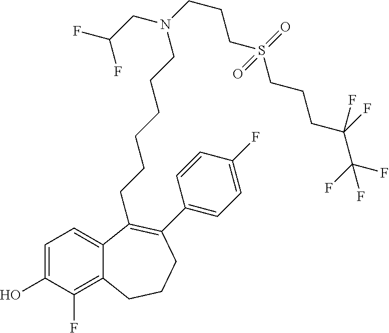 [SMILES: O=S(=O)(CCCN(CCCCCCC1=C(c2ccc(F)cc2)CCCc2c1ccc(O)c2F)CC(F)F)CCCC(F)(F)C(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 95.7 mg (0.28 mmol) of N-(2,2-difluoroethyl)-3-[(4,4,5,5,5-pentafluoropentyl)sulphonyl]propan-1-amine in 4 mL acetonitrile according to general specification 11 except that it was not stirred at 85° C., but was treated at 200° C. for 15 minutes at 250 watt in a microwave. It was purified using HPLC-Method 1. 25.4 mg (16% of theor.) of product was isolated.

¹H-NMR (300 MHz, chloroform-d₁): δ=1.01-1.31 (m, 8H), 1.91 (mc, 2H), 2.04-2.44 (m, 12H), 2.62 (mc, 2H), 2.66-2.81 (m, 4H), 3.00-3.09 (m, 4H), 5.70 (tt, 1H), 6.89 (t, 1H), 6.99 (dd, 1H), 7.04 (tt, 2H), 7.16-7.23 (m, 2H).

### Example 65

### 4-Fluoro-9-{6-[(4-fluorobenzyl){3-[(4,4,5,5,5-pentafluoropentyl)sulphonyl]propyl}amino]hexyl}-8-(4-fluorophenyl)-6,7-dihydro-5H-benzo[7]annulen-3-ol

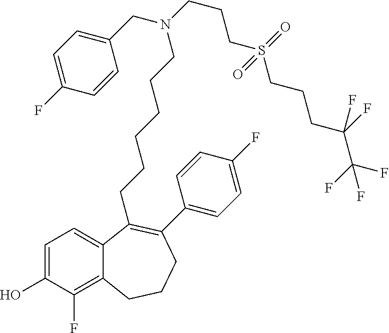 [SMILES: O=S(=O)(CCCN(CCCCCCC1=C(c2ccc(F)cc2)CCCc2c1ccc(O)c2F)Cc1ccc(F)cc1)CCCC(F)(F)C(F)(F)F]

100 mg (0.23 mmol) of 9-(6-bromohexyl)-4-fluoro-8-(4-fluorophenyl)-6,7-dihydro-5H-benzo[7]annulen-3-ol was reacted with 107.9 mg (0.28 mmol) of N-(4-fluorobenzyl)-3-[(4,4,5,5,5-pentafluoropentyl)sulphonyl]propan-1-amine in 4 mL acetonitrile according to general specification 11 except that it was not stirred at 85° C., but was treated at 200° C. for 15 minutes at 250 watt in a microwave. It was purified using HPLC-Method 1. 34.2 mg (20% of theor.) of product was isolated.

¹H-NMR (300 MHz, chloroform-d₁): δ=1.01-1.23 (m, 6H), 1.31 (m, 2H), 1.94 (mc, 2H), 2.03-2.38 (m, 12H), 2.52 (t, 2H), 2.68-2.77 (m, 2H), 2.91-3.01 (m, 4H), 3.50 (s, 2H), 6.89 (t, 1H), 6.95-7.08 (m, 5H), 7.14-7.25 (m, 4H).

### Example 66

### 9-[6-(Cyclopropyl{3-[(4,4,5,5,5-pentafluoropentyl)sulphonyl]propyl}amino)hexyl]-8-(3,4-difluorophenyl)-6,7-dihydro-5H-benzo[7]annulen-3-ol

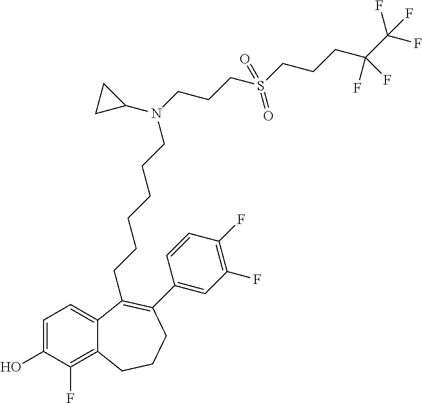 [SMILES: O=S(=O)(CCCN(CCCCCCC1=C(c2ccc(F)c(F)c2)CCCc2c1ccc(O)c2F)C1CC1)CCCC(F)(F)C(F)(F)F]

130 mg (0.30 mmol) of 9-(6-bromohexyl)-8-(3,4-difluorophenyl)-6,7-dihydro-5H-benzo[7]annulen-3-ol was reacted with 115.9 mg (0.36 mmol) of N-{3-[(4,4,5,5,5-pentafluoropentyl)sulphonyl]propyl}cyclopropanamine according to general specification 11 for 40 hours. It was purified using HPLC (XBridge C18, 5μ, 100×30 mm, 50 mL/min, solvent: water with 0.1% formic acid-acetonitrile 90:10, 0-1 minute; 90:10→1:99, 1-7.5 minutes; 1:99, 7.5-10 minutes). 57.1 mg (27% of theor.) of product was isolated.

¹H-NMR (300 MHz, chloroform-d₁): δ=0.34-0.52 (m, 4H), 1.00-1.24 (m, 6H), 1.30 (m, 2H), 1.71 (mc, 1H), 1.95-2.39 (m, 12H), 2.43 (mc, 2H), 2.60 (t, 2H), 2.67 (t, 2H), 2.95-3.09 (m, 4H), 6.69-6.77 (m, 2H), 6.94 (ddd, 1H), 7.04 (ddd, 1H), 7.08-7.18 (m, 2H).

### Example 67

### 8-(3,5-Difluorophenyl)-9-[6-({4-[(4,4,4-trifluorobutyl)sulphonyl]butyl}amino)hexyl]-6,7-dihydro-5H-benzo[7]annulen-3-ol

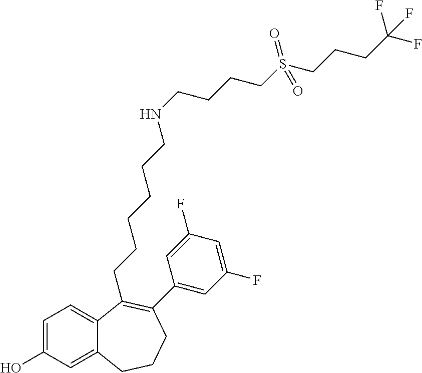 [SMILES: O=S(=O)(CCCCNCCCCCCC1=C(c2cc(F)cc(F)c2)CCCc2cc(O)ccc21)CCCC(F)(F)F]

130 mg (0.30 mmol) of 9-(6-bromohexyl)-8-(3,5-difluorophenyl)-6,7-dihydro-5H-benzo[7]annulen-3-ol was reacted with 103.4 mg (0.42 mmol) of 4-[(4,4,4-trifluorobutyl)sulphonyl]butan-1-amine according to general specification 11. It was purified using HPLC-Method 1. 48.4 mg (27% of theor.) of product was isolated.

¹H-NMR (400 MHz, chloroform-d₁): δ=1.00-1.23 (m, 6H), 1.37 (mc, 2H), 1.82 (mc, 2H), 1.92 (mc, 2H), 2.03-2.18 (m, 6H), 2.25-2.40 (m, 4H), 2.53-2.62 (m, 4H), 2.79 (t, 2H), 3.06 (q, 4H), 6.66-6.78 (m, 5H), 7.11 (d, 1H).

### Example 68

### 8-(3,5-Difluorophenyl)-9-[6-(methyl{5-[(3,3,3-trifluoropropyl)sulphonyl]pentyl}amino)hexyl]-6,7-dihydro-5H-benzo[7]annulen-3-ol

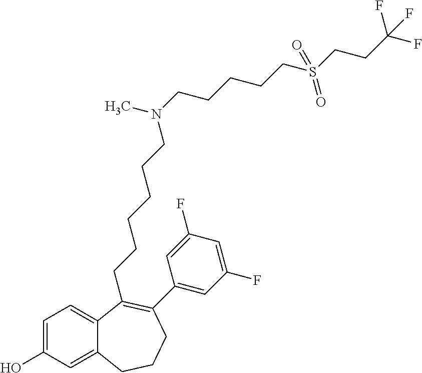 [SMILES: CN(CCCCCCC1=C(c2cc(F)cc(F)c2)CCCc2cc(O)ccc21)CCCCCS(=O)(=O)CCC(F)(F)F]

130 mg (0.30 mmol) of 9-(6-bromohexyl)-8-(3,5-difluorophenyl)-6,7-dihydro-5H-benzo[7]annulen-3-ol was reacted with 93.6 mg (0.36 mmol) of N-methyl-5-[(3,3,3-trifluoropropyl)sulphonyl]pentan-1-amine according to general specification 11. It was purified using HPLC-Method 1. 96.7 mg (53% of theor.) of product was isolated.

¹H-NMR (400 MHz, chloroform-d₁): δ=1.03-1.24 (m, 6H), 1.37 (mc, 2H), 1.47-1.56 (m, 2H), 1.65 (mc, 2H), 1.91 (mc, 2H), 2.02-2.15 (m, 4H), 2.36 (t, 2H), 2.42 (s, 3H), 2.46 (mc, 2H), 2.55-2.75 (m, 6H), 3.06 (mc, 2H), 3.20 (mc, 2H), 6.66-6.79 (m, 5H), 7.12 (d, 1H).

### Example 69

### 8-(3,5-Difluorophenyl)-9-[6-(methyl{4-[(3,3,4,4,4-pentafluorobutyl)sulphonyl]butyl}amino)hexyl]-6,7-dihydro-5H-benzo[7]annulen-3-ol

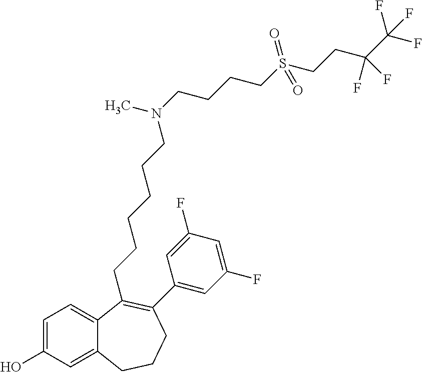 [SMILES: CN(CCCCCCC1=C(c2cc(F)cc(F)c2)CCCc2cc(O)ccc21)CCCCS(=O)(=O)CCC(F)(F)C(F)(F)F]

130 mg (0.30 mmol) of 9-(6-bromohexyl)-8-(3,5-difluorophenyl)-6,7-dihydro-5H-benzo[7]annulen-3-ol was reacted with 106.5 mg (0.36 mmol) of N-methyl-4-[(3,3,4,4,4-pentafluorobutyl)sulphonyl]butan-1-amine according to general specification 11. It was purified using HPLC-Method 1. 101.5 mg (52% of theor.) of product was isolated.

¹H-NMR (400 MHz, chloroform-d₁): δ=1.03-1.24 (m, 6H), 1.37 (mc, 2H), 1.79 (mc, 2H), 1.93 (mc, 2H), 2.03-2.15 (m, 4H), 2.36 (t, 2H), 2.43 (s, 3H), 2.47 (mc, 2H), 2.55-2.70 (m, 6H), 3.12 (mc, 2H), 3.23 (mc, 2H), 6.67-6.78 (m, 5H), 7.12 (d, 1H).

### Example 70

### 8-(3,5-Difluorophenyl)-9-[6-(methyl{3-[(5,5,5-trifluoropentyl)sulphonyl]propyl}amino)hexyl]-6,7-dihydro-5H-benzo[7]annulen-3-ol

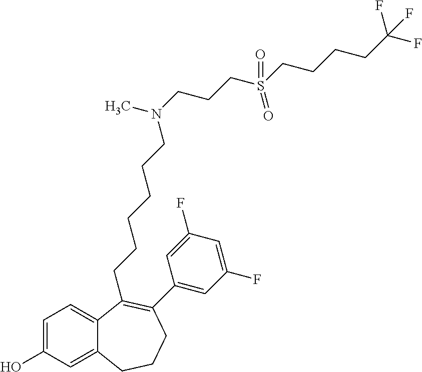 [SMILES: CN(CCCCCCC1=C(c2cc(F)cc(F)c2)CCCc2cc(O)ccc21)CCCS(=O)(=O)CCCCC(F)(F)F]

130 mg (0.30 mmol) of 9-(6-bromohexyl)-8-(3,5-difluorophenyl)-6,7-dihydro-5H-benzo[7]annulen-3-ol was reacted with 93.6 mg (0.36 mmol) of N-methyl-3-[(5,5,5-trifluoropentyl)sulphonyl]propan-1-amine according to general specification 11. It was purified using HPLC-Method 1. 80 mg (44% of theor.) of product was isolated.

¹H-NMR (500 MHz, chloroform-d₁): δ=1.04-1.16 (m, 4H), 1.21 (quin, 2H), 1.31 (mc, 2H), 1.75 (mc, 2H), 1.95 (mc, 2H), 2.05-2.22 (m, 8H), 2.32-2.40 (m, 7H), 2.61 (t, 2H), 2.70 (t, 2H), 3.02 (mc, 2H), 3.09 (t, 2H), 6.68-6.79 (m, 5H), 7.15 (d, 1H).

### Example 71

### 8-(3,4-Difluorophenyl)-9-[6-(methyl{5-[(3,3,3-trifluoropropyl)sulphonyl]pentyl}amino)hexyl]-6,7-dihydro-5H-benzo[7]annulen-3-ol

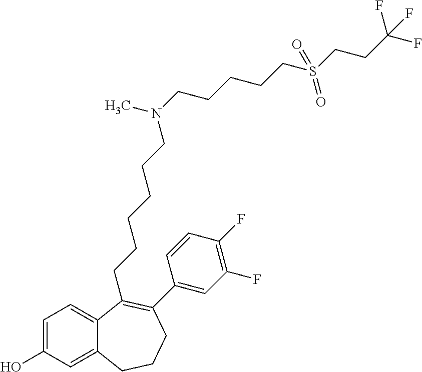 [SMILES: CN(CCCCCCC1=C(c2ccc(F)c(F)c2)CCCc2cc(O)ccc21)CCCCCS(=O)(=O)CCC(F)(F)F]

130 mg (0.30 mmol) of 9-(6-bromohexyl)-8-(3,4-difluorophenyl)-6,7-dihydro-5H-benzo[7]annulen-3-ol was reacted with 93.6 mg (0.36 mmol) of N-methyl-5-[(3,3,3-trifluoropropyl)sulphonyl]pentan-1-amine according to general specification 11. It was purified using HPLC-Method 1. 99.7 mg (54% of theor.) of product was isolated.

¹H-NMR (400 MHz, chloroform-d₁): δ=1.02-1.24 (m, 6H), 1.30-1.41 (m, 2H), 1.46-1.56 (m, 2H), 1.60-1.69 (m, 2H), 1.91 (mc, 2H), 2.02-2.15 (m, 4H), 2.35 (t, 2H), 2.38-2.48 (m, 5H), 2.54-2.64 (m, 4H), 2.64-2.75 (m, 2H), 3.06 (t, 2H), 3.20 (mc, 2H), 6.70-6.78 (m, 2H), 6.90-6.97 (m, 1H), 7.03 (mc, 1H), 7.09-7.18 (m, 2H).

### Example 72

### 8-(3,4-Difluorophenyl)-9-[6-(methyl{4-[(3,3,4,4,4-pentafluorobutyl)sulphonyl]butyl}amino)hexyl]-6,7-dihydro-5H-benzo[7]annulen-3-ol

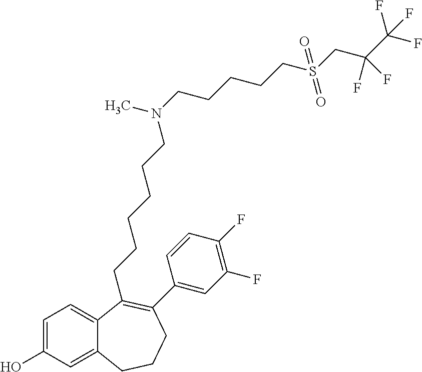 [SMILES: CN(CCCCCCC1=C(c2ccc(F)c(F)c2)CCCc2cc(O)ccc21)CCCCCS(=O)(=O)CC(F)(F)C(F)(F)F]

130 mg (0.30 mmol) of 9-(6-bromohexyl)-8-(3,4-difluorophenyl)-6,7-dihydro-5H-benzo[7]annulen-3-ol was reacted with 106.5 mg (0.36 mmol) of N-methyl-4-[(3,3,4,4,4-pentafluorobutyl)sulphonyl]butan-1-amine according to general specification 11. It was purified using HPLC-Method 1. 100.7 mg (52% of theor.) of product was isolated.

¹H-NMR (400 MHz, chloroform-d₁): δ=1.03-1.25 (m, 6H), 1.27-1.38 (m, 2H), 1.70-1.80 (m, 2H), 1.92 (mc, 2H), 2.03-2.15 (m, 4H), 2.30-2.44 (m, 7H), 2.51-2.71 (m, 6H), 3.12 (t, 2H), 3.20-3.27 (m, 2H), 6.71-6.78 (m, 2H), 6.91-6.97 (m, 1H), 7.04 (mc, 1H), 7.09-7.18 (m, 2H).

### Example 73

### 8-(3,4-Difluorophenyl)-9-[6-(methyl{3-[(5,5,5-trifluoropentyl)sulphonyl]propyl}amino)hexyl]-6,7-dihydro-5H-benzo[7]annulen-3-ol

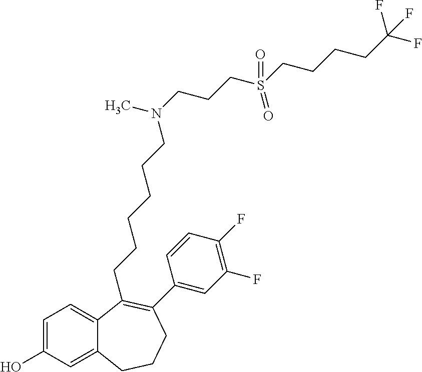 [SMILES: CN(CCCCCCC1=C(c2ccc(F)c(F)c2)CCCc2cc(O)ccc21)CCCS(=O)(=O)CCCCC(F)(F)F]

130 mg (0.30 mmol) of 9-(6-bromohexyl)-8-(3,4-difluorophenyl)-6,7-dihydro-5H-benzo[7]annulen-3-ol was reacted with 93.6 mg (0.36 mmol) of N-methyl-3-[(5,5,5-trifluoropentyl)sulphonyl]propan-1-amine according to general specification 11. It was purified using HPLC-Method 1. 79 mg (43% of theor.) of product was isolated.

¹H-NMR (300 MHz, chloroform-d₁): δ=1.00-1.25 (m, 6H), 1.28-1.40 (m, 2H), 1.68-1.80 (m, 2H), 1.94 (mc, 2H), 2.02-2.24 (m, 8H), 2.36 (t, 2H), 2.39-2.48 (m, 5H), 2.60 (mc, 2H), 2.80 (t, 2H), 3.03 (mc, 2H), 3.10 (t, 2H), 6.72-6.80 (m, 2H), 6.90-6.97 (m, 1H), 7.03 (ddd, 1H), 7.08-7.18 (m, 2H).

### Example 74

### 9-{6-[(2-Fluoroethyl){3-[(4,4,5,5,5-pentafluoropentyl)sulphonyl]propyl}amino]hexyl}-8-(4-fluorophenyl)-6,7-dihydro-5H-benzo[7]annulen-3-ol

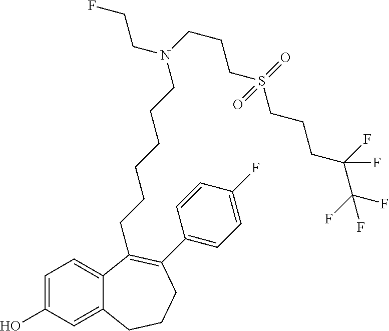 [SMILES: O=S(=O)(CCCN(CCF)CCCCCCC1=C(c2ccc(F)cc2)CCCc2cc(O)ccc21)CCCC(F)(F)C(F)(F)F]

100 mg (0.24 mmol) of 9-(6-bromohexyl)-8-(4-fluorophenyl)-6,7-dihydro-5H-benzo[7]annulen-3-ol was reacted with 94.7 mg (0.29 mmol) of N-(2-fluoroethyl)-3-[(4,4,5,5,5-pentafluoropentyl)sulphonyl]propan-1-amine according to general specification 11, but was stirred under reflux in 10 mL acetonitrile for 72 hours. It was purified using HPLC-Method 1. 16.1 mg (10% of theor.) of product was isolated.

¹H-NMR (300 MHz, chloroform-d₁): δ=1.02-1.34 (m, 8H), 1.93-2.47 (m, 14H), 2.57-2.79 (m, 5H), 2.84 (mc, 1H), 3.03-3.15 (m, 4H), 4.52 (mc, 2H), 6.72-6.79 (m, 2H), 7.04 (mc, 2H), 7.13-7.23 (m, 3H).

### Example 75

### 8-(4-Fluorophenyl)-9-[6-(methyl{4-[(3,3,4,4,4-pentafluorobutyl)sulphonyl]butyl}amino)hexyl]-6,7-dihydro-5H-benzo[7]annulen-3-ol

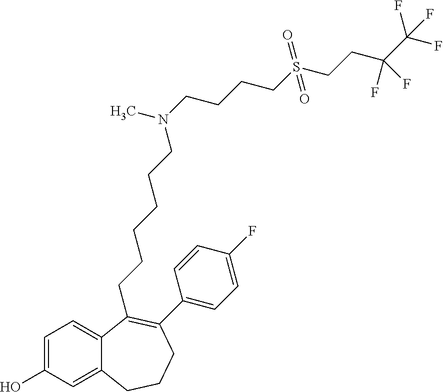 [SMILES: CN(CCCCCCC1=C(c2ccc(F)cc2)CCCc2cc(O)ccc21)CCCCS(=O)(=O)CCC(F)(F)C(F)(F)F]

130 mg (0.31 mmol) of 9-(6-bromohexyl)-8-(4-fluorophenyl)-6,7-dihydro-5H-benzo[7]annulen-3-ol was reacted with 111.1 mg (0.37 mmol) of N-methyl-4-[(3,3,4,4,4-pentafluorobutyl)sulphonyl]butan-1-amine according to general specification 11. It was purified using HPLC-Method 1. 106.2 mg (54% of theor.) of product was isolated.

¹H-NMR (300 MHz, chloroform-d₁): δ=1.01-1.25 (m, 6H), 1.27-1.39 (m, 2H), 1.76 (mc, 2H), 1.92 (mc, 2H), 2.03-2.17 (m, 4H), 2.30-2.45 (m, 7H), 2.53-2.73 (m, 6H), 3.11 (mc, 2H), 3.19-3.27 (m, 2H), 6.70-6.77 (m, 2H), 7.03 (tt, 2H), 7.11-7.22 (m, 3H).

### Example 76

### 8-(4-Fluorophenyl)-9-[6-(methyl{5-[(3,3,3-trifluoropropyl)sulphonyl]pentyl}amino)hexyl]-6,7-dihydro-5H-benzo[7]annulen-3-ol

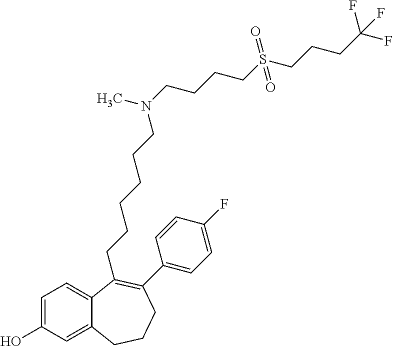 [SMILES: CN(CCCCCCC1=C(c2ccc(F)cc2)CCCc2cc(O)ccc21)CCCCS(=O)(=O)CCCC(F)(F)F]

130 mg (0.31 mmol) of 9-(6-bromohexyl)-8-(4-fluorophenyl)-6,7-dihydro-5H-benzo[7]annulen-3-ol was reacted with 97.7 mg (0.37 mmol) of N-methyl-5-[(3,3,3-trifluoropropyl)sulphonyl]pentan-1-amine according to general specification 11. It was purified using HPLC-Method 1. 92.6 mg (50% of theor.) of product was isolated.

¹H-NMR (300 MHz, chloroform-d₁): δ=1.00-1.25 (m, 6H), 1.28-1.41 (m, 2H), 1.52 (mc, 2H), 1.62-1.73 (m, 2H), 1.91 (mc, 2H), 2.03-2.16 (m, 4H), 2.35 (t, 2H), 2.40-2.51 (m, 5H), 2.55-2.78 (m, 6H), 3.07 (mc, 2H), 3.20 (mc, 2H), 6.71-6.78 (m, 2H), 7.03 (tt, 2H), 7.10-7.22 (m, 3H).

### Example 77

### 8-(4-Fluorophenyl)-9-[6-({4-[(4,4,4-trifluorobutyl)sulphonyl]butyl}amino)hexyl]-6,7-dihydro-5H-benzo[7]annulen-3-ol

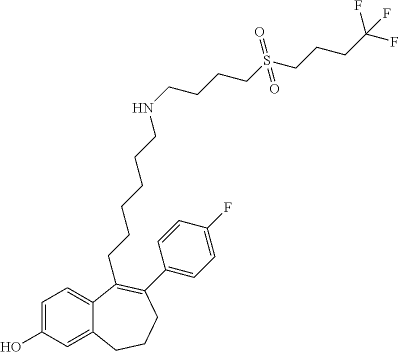 [SMILES: O=S(=O)(CCCCNCCCCCCC1=C(c2ccc(F)cc2)CCCc2cc(O)ccc21)CCCC(F)(F)F]

1500 mg (3.59 mmol) of 9-(6-bromohexyl)-8-(4-fluorophenyl)-6,7-dihydro-5H-benzo[7]annulen-3-ol was reacted at 80° C. with 1066.5 mg (4.31 mmol) of 4-[(4,4,4-trifluorobutyl)sulphonyl]butan-1-amine according to general specification 11. It was purified on Silica Gel 60 (solvent: dichloromethane, dichloromethane-methanol 95:5, 90:10 and 85:15). 1100 mg (52% of theor.) of product was isolated.

¹H-NMR (300 MHz, chloroform-d₁): δ=1.05-1.24 (m, 6H), 1.32 (mc, 2H), 1.63 (mc, 2H), 1.89 (mc, 2H), 2.02-2.20 (m, 6H), 2.24-2.41 (m, 4H), 2.48 (t, 2H), 2.61 (t, 4H), 2.97-3.08 (m, 4H), 6.67-6.73 (m, 2H), 7.03 (t, 2H), 7.12-7.23 (m, 3H).

### Example 78

### 8-(4-Fluorophenyl)-9-[6-(methyl{3-[(4,4,5,5,5-pentafluoropentyl)sulphanyl]propyl}amino)hexyl]-6,7-dihydro-5H-benzo[7]annulen-3-ol

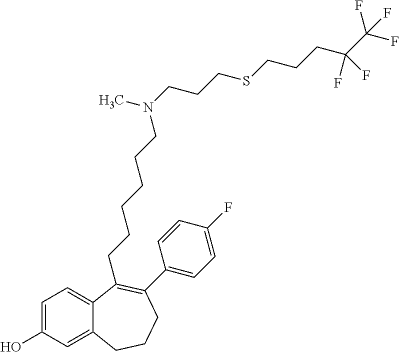 [SMILES: CN(CCCCCCC1=C(c2ccc(F)cc2)CCCc2cc(O)ccc21)CCCSCCCC(F)(F)C(F)(F)F]

130 mg (0.31 mmol) of 9-(6-bromohexyl)-8-(4-fluorophenyl)-6,7-dihydro-5H-benzo[7]annulen-3-ol was reacted with 99.2 mg (0.37 mmol) of N-methyl-3-[(4,4,5,5,5-pentafluoropentyl)sulphanyl]propan-1-amine according to general specification 11. It was purified using HPLC-Method 1. 76 mg (41% of theor.) of product was isolated.

¹H-NMR (300 MHz, chloroform-d₁): δ=0.99-1.26 (m, 6H), 1.37 (mc, 2H), 1.81-1.98 (m, 4H), 2.02-2.26 (m, 6H), 2.35 (t, 2H), 2.43-2.65 (m, 11H), 2.78 (mc, 2H), 6.72-6.80 (m, 2H), 7.03 (tt, 2H), 7.10-7.22 (m, 3H).

### Example 79

### 8-(4-Fluorophenyl)-9-[6-(methyl{3-[(5,5,6,6,6-pentafluorohexyl)sulphonyl]propyl}amino)hexyl]-6,7-dihydro-5H-benzo[7]annulen-3-ol

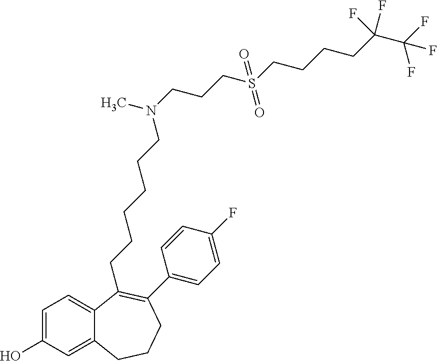 [SMILES: CN(CCCCCCC1=C(c2ccc(F)cc2)CCCc2cc(O)ccc21)CCCS(=O)(=O)CCCCC(F)(F)C(F)(F)F]

130 mg (0.31 mmol) of 9-(6-bromohexyl)-8-(4-fluorophenyl)-6,7-dihydro-5H-benzo[7]annulen-3-ol was reacted with 116.4 mg (0.37 mmol) of N-methyl-3-[(5,5,6,6,6-pentafluorohexyl)sulphonyl]propan-1-amine according to general specification 11. It was purified using HPLC-Method 1. 86 mg (43% of theor.) of product was isolated.

¹H-NMR (400 MHz, chloroform-d₁): δ=1.01-1.24 (m, 6H), 1.30 (mc, 2H), 1.74-1.84 (m, 2H), 1.96 (mc, 2H), 2.02-2.18 (m, 8H), 2.31-2.40 (m, 7H), 2.62 (mc, 2H), 2.70 (t, 2H), 3.04 (mc, 2H), 3.09 (t, 2H), 6.73-6.79 (m, 2H), 7.04 (tt, 2H), 7.14-7.22 (m, 3H).

### Example 80

### 8-(4-Fluorophenyl)-9-[6-(methyl{3-[(5,5,5-trifluoropentyl)sulphonyl]propyl}amino)hexyl]-6,7-dihydro-5H-benzo[7]annulen-3-ol

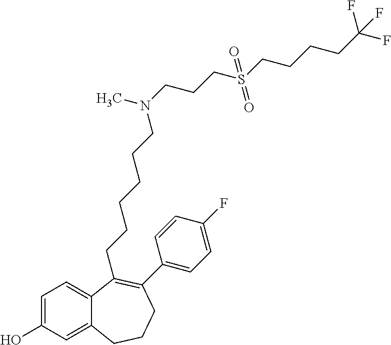 [SMILES: CN(CCCCCCC1=C(c2ccc(F)cc2)CCCc2cc(O)ccc21)CCCS(=O)(=O)CCCCC(F)(F)F]

130 mg (0.31 mmol) of 9-(6-bromohexyl)-8-(4-fluorophenyl)-6,7-dihydro-5H-benzo[7]annulen-3-ol was reacted with 97.7 mg (0.37 mmol) of N-methyl-3-[(5,5,5-trifluoropentyl)sulphonyl]propan-1-amine according to general specification 11. It was purified using HPLC-Method 1. 102 mg (55% of theor.) of product was isolated.

¹H-NMR (300 MHz, chloroform-d₁): δ=0.99-1.27 (m, 6H), 1.40 (mc, 2H), 1.68-1.81 (m, 2H), 1.88-2.01 (m, 2H), 2.03-2.30 (m, 8H), 2.36 (t, 2H), 2.47 (mc, 5H), 2.58-2.65 (m, 2H), 2.86 (t, 2H), 3.07 (mc, 2H), 3.17 (t, 2H), 6.76-6.83 (m, 2H), 7.04 (tt, 2H), 7.12-7.22 (m, 3H).

### Example 81

### Benzyl-N-{6-[8-(4-fluorophenyl)-3-hydroxy-6,7-dihydro-5H-benzo[7]annulen-9-yl]hexyl}-N-{4-[(4,4,4-trifluorobutyl)sulphonyl]butyl}glycinate

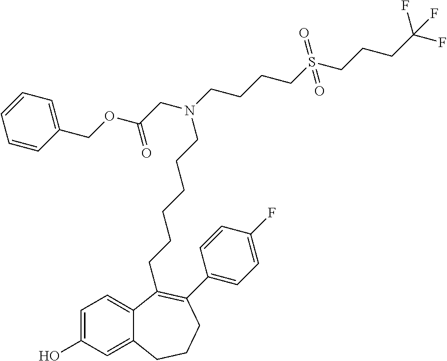 [SMILES: O=C(CN(CCCCCCC1=C(c2ccc(F)cc2)CCCc2cc(O)ccc21)CCCCS(=O)(=O)CCCC(F)(F)F)OCc1ccccc1]

96 mg (0.23 mmol) of 9-(6-bromohexyl)-8-(4-fluorophenyl)-6,7-dihydro-5H-benzo[7]annulen-3-ol was reacted for 30 hours at 80° C. with 100 mg (0.25 mmol) of benzyl-N-{4-[(4,4,4-trifluorobutyl)sulphonyl]butyl}glycinate according to general specification 11. It was purified using HPLC (XBridge C18, 5μ, 100×30 mm, 54 mL/min, solvent: water with 0.1% formic acid-acetonitrile 70:30→30:70, 0-12 minutes). The combined fractions were neutralized with ammonia and concentrated by evaporation. The residue was taken up in dichloromethane, washed with water twice, dried over magnesium sulphate and concentrated by evaporation. The product was dried at 50° C. in a drying cabinet. 15 mg (8% of theor.) of product was isolated.

¹H-NMR (300 MHz, chloroform-d₁): δ=1.00-1.31 (m, 8H), 1.53 (quin, 2H), 1.84 (mc, 2H), 2.02-2.19 (m, 6H), 2.22-2.43 (m, 6H), 2.51 (t, 2H), 2.57-2.65 (m, 2H), 2.97-3.06 (m, 4H), 3.27 (s, 2H), 5.11 (s, 2H), 6.69-6.77 (m, 2H), 7.03 (mc, 2H), 7.13-7.23 (m, 3H), 7.30-7.39 (m, 5H).

### Example 82

### Methyl-N-{6-[8-(4-fluorophenyl)-3-hydroxy-6,7-dihydro-5H-benzo[7]annulen-9-yl]hexyl}-N-{4-[(4,4,4-trifluorobutyl)sulphonyl]butyl}glycinate

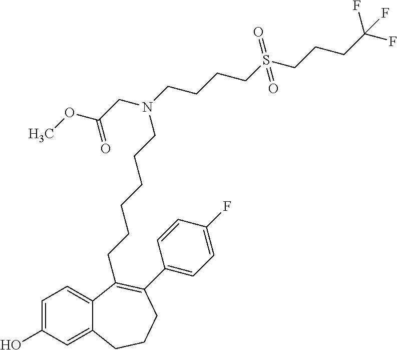 [SMILES: COC(=O)CN(CCCCCCC1=C(c2ccc(F)cc2)CCCc2cc(O)ccc21)CCCCS(=O)(=O)CCCC(F)(F)F]

130 mg (0.22 mmol) of 8-(4-fluorophenyl)-9-[6-({4-[(4,4,4-trifluorobutyl)sulphonyl]butyl}amino)hexyl]-6,7-dihydro-5H-benzo[7]annulen-3-ol was stirred at room temperature with 37.5 mg (0.25 mmol) of methyl bromoacetate and 92.3 mg (0.67 mmol) of potassium carbonate in 2.5 mL of DMF for 24 hours. It was evaporated to dryness, and after adding water it was extracted with ethyl acetate three times. The combined organic phases were washed three times with water, dried over magnesium sulphate and concentrated by evaporation. It was purified on Silica Gel 60 (solvent: dichloromethane, dichloromethane-methanol 95:5). 105 mg (64% of theor.) of product was isolated.

¹H-NMR (300 MHz, chloroform-d₁): δ=1.01-1.32 (m, 8H), 1.57 (mc, 2H), 1.87 (mc, 2H), 2.00-2.21 (m, 6H), 2.24-2.45 (m, 6H), 2.53 (mc, 2H), 2.62 (mc, 2H), 3.00-3.10 (m, 4H), 3.24 (s, 2H), 3.68 (s, 3H), 6.71-6.77 (m, 2H), 7.03 (tt, 2H), 7.14-7.22 (m, 3H).

### Example 83

### Methyl-N-{6-[8-(4-fluorophenyl)-3-hydroxy-6,7-dihydro-5H-benzo[7]annulen-9-yl]hexyl}-N-{4-[(4,4,4-trifluorobutyl)sulphonyl]butyl}-beta-alaninate

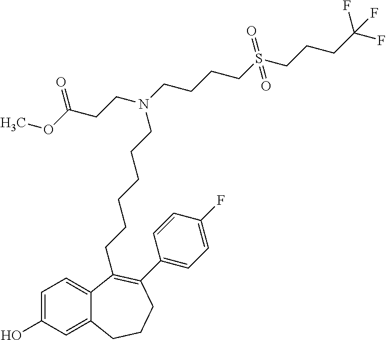 [SMILES: COC(=O)CCN(CCCCCCC1=C(c2ccc(F)cc2)CCCc2cc(O)ccc21)CCCCS(=O)(=O)CCCC(F)(F)F]

130 mg (0.22 mmol) of 8-(4-fluorophenyl)-9-[6-({4-[(4,4,4-trifluorobutyl)sulphonyl]butyl}amino)hexyl]-6,7-dihydro-5H-benzo[7]annulen-3-ol was stirred at room temperature with 40.9 mg (0.25 mmol) of 3-bromopropionic acid methyl ester and 92.3 mg (0.67 mmol) of potassium carbonate in 2.5 mL of DMF for 24 hours. It was evaporated to dryness, and after adding water it was extracted with ethyl acetate three times. The combined organic phases were washed three times with water, dried over magnesium sulphate and concentrated by evaporation. It was purified on Silica Gel 60 (solvent: dichloromethane, dichloromethane-methanol 98:2 and 95:5). 112 mg (75% of theor.) of product was isolated.

¹H-NMR (300 MHz, chloroform-d₁): δ=1.00-1.29 (m, 8H), 1.52 (quin, 2H), 1.75-1.88 (m, 2H), 2.03-2.43 (m, 16H), 2.62 (mc, 2H), 2.68 (t, 2H), 2.95-3.08 (m, 4H), 3.64 (s, 3H), 6.70-6.77 (m, 2H), 7.03 (tt, 2H), 7.14-7.23 (m, 3H).

### Example 84

### 4-Fluoro-8-(4-fluorophenyl)-9-{6-[{3-[(4,4,5,5,5-pentafluoropentyl)sulphonyl]propyl}(2,2,2-trifluoroethyl)amino]hexyl}-6,7-dihydro-5H-benzo[7]annulen-3-ol

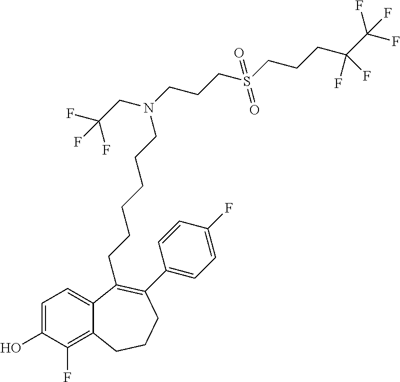 [SMILES: O=S(=O)(CCCN(CCCCCCC1=C(c2ccc(F)cc2)CCCc2c1ccc(O)c2F)CC(F)(F)F)CCCC(F)(F)C(F)(F)F]

100 mg (0.23 mmol) of 9-(6-bromohexyl)-4-fluoro-8-(4-fluorophenyl)-6,7-dihydro-5H-benzo[7]annulen-3-ol was reacted in 6.7 mL acetonitrile with 100.7 mg (0.28 mmol) of 3-[(4,4,5,5,5-pentafluoropentyl)sulphonyl]-N-(2,2,2-trifluoroethyl)propan-1-amine according to general specification 11, and was irradiated in a microwave with 250 W at 200° C. for 15 minutes. It was purified using HPLC (HPLC-Method 1 and XBridge C18, 5μ, 100×30 mm, 50 mL/min, solvent: water with 0.1% formic acid-acetonitrile 90:10, 0-1 minute; 90:10→0:100, 1-7.5 minutes; 0:100, 7.5-10 minutes). 14.4 mg (8% of theor.) of product was isolated.

¹H-NMR (300 MHz, chloroform-d₁): δ=0.99-1.36 (m, 8H), 1.81-1.99 (m, 2H), 2.02-2.39 (m, 10H), 2.46 (mc, 2H), 2.62-2.80 (m, 4H), 2.88-3.13 (m, 6H), 6.82-7.11 (m, 4H), 7.14-7.23 (m, 2H).

### Example 85

### 4-Fluoro-9-{6-[(2-fluoroethyl){3-[(4,4,5,5,5-pentafluoropentyl)sulphonyl]propyl}amino]hexyl}-8-(4-fluorophenyl)-6,7-dihydro-5H-benzo[7]annulen-3-ol

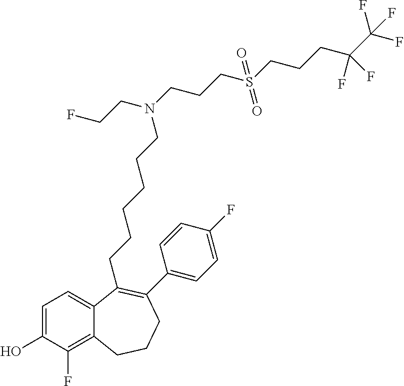 [SMILES: O=S(=O)(CCCN(CCF)CCCCCCC1=C(c2ccc(F)cc2)CCCc2c1ccc(O)c2F)CCCC(F)(F)C(F)(F)F]

100 mg (0.23 mmol) of 9-(6-bromohexyl)-4-fluoro-8-(4-fluorophenyl)-6,7-dihydro-5H-benzo[7]annulen-3-ol was stirred under reflux with 90.8 mg (0.28 mmol) of N-(2-fluoroethyl)-3-[(4,4,5,5,5-pentafluoropentyl)sulphonyl]propan-1-amine according to general specification 11 in 10 mL acetonitrile for 72 hours. It was purified using HPLC-Method 1. 12.8 mg (7% of theor.) of product was isolated.

¹H-NMR (300 MHz, chloroform-d₁): δ=1.02-1.25 (m, 6H), 1.34 (mc, 2H), 2.00-2.39 (m, 12H), 2.53 (mc, 2H), 2.67-2.88 (m, 5H), 2.94 (mc, 1H), 3.04-3.17 (m, 4H), 4.58 (dt, 2H), 6.90 (t, 1H), 6.98 (d, 1H), 7.05 (tt, 2H), 7.15-7.23 (m, 2H).

### Example 86

### 4-Fluoro-8-(4-fluorophenyl)-9-[6-(methyl{3-[(4,4,5,5,5-pentafluoropentyl)sulphanyl]propyl}amino)hexyl]-6,7-dihydro-5H-benzo[7]annulen-3-ol

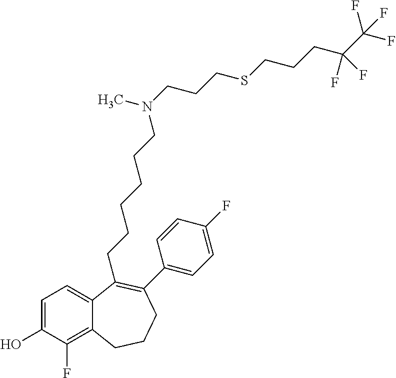 [SMILES: CN(CCCCCCC1=C(c2ccc(F)cc2)CCCc2c1ccc(O)c2F)CCCSCCCC(F)(F)C(F)(F)F]

130 mg (0.30 mmol) of 9-(6-bromohexyl)-4-fluoro-8-(4-fluorophenyl)-6,7-dihydro-5H-benzo[7]annulen-3-ol was reacted with 95.1 mg (0.36 mmol) of N-methyl-3-[(4,4,5,5,5-pentafluoropentyl)sulphanyl]propan-1-amine according to general specification 11. It was purified using HPLC-Method 1. 62 mg (34% of theor.) of product was isolated.

¹H-NMR (300 MHz, chloroform-d₁): δ=1.01-1.24 (m, 6H), 1.36-1.51 (m, 2H), 1.81-2.00 (m, 4H), 2.03-2.26 (m, 6H), 2.29-2.39 (m, 2H), 2.51 (s, 3H), 2.54-2.64 (m, 6H), 2.67-2.76 (m, 2H), 2.78-2.88 (m, 2H), 6.85-6.97 (m, 2H), 7.04 (tt, 2H), 7.15-7.22 (m, 2H).

### Example 87

### 4-Fluoro-8-(4-fluorophenyl)-9-[6-(methyl{3-[(5,5,5-trifluoropentyl)sulphonyl]propyl}amino)hexyl]-6,7-dihydro-5H-benzo[7]annulen-3-ol

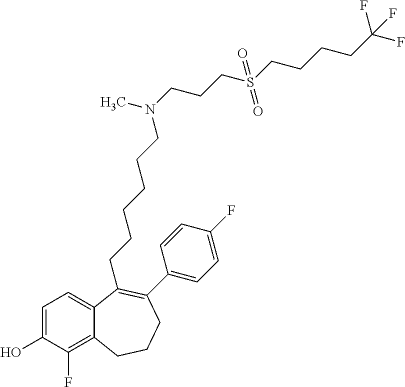 [SMILES: CN(CCCCCCC1=C(c2ccc(F)cc2)CCCc2c1ccc(O)c2F)CCCS(=O)(=O)CCCCC(F)(F)F]

130 mg (0.30 mmol) of 9-(6-bromohexyl)-4-fluoro-8-(4-fluorophenyl)-6,7-dihydro-5H-benzo[7]annulen-3-ol was reacted with 93.6 mg (0.36 mmol) of N-methyl-3-[(5,5,5-trifluoropentyl)sulphonyl]propan-1-amine according to general specification 11. It was purified using HPLC-Method 1. 67 mg (36% of theor.) of product was isolated.

¹H-NMR (500 MHz, chloroform-d₁): δ=1.04-1.14 (m, 4H), 1.15-1.22 (m, 2H), 1.31-1.39 (m, 2H), 1.75 (quin, 2H), 1.95 (mc, 2H), 2.05-2.20 (m, 8H), 2.32-2.37 (m, 5H), 2.40 (mc, 2H), 2.66-2.76 (m, 4H), 3.01 (mc, 2H), 3.08 (mc, 2H), 6.90 (t, 1H), 6.97 (d, 1H), 7.05 (mc, 2H), 7.16-7.21 (m, 2H).

### Example 88

### Methyl-4-({6-[8-(4-fluorophenyl)-3-hydroxy-6,7-dihydro-5H-benzo[7]annulen-9-yl]hexyl}{4-[(4,4,4-trifluorobutyl)sulphonyl]butyl}amino)butanoate

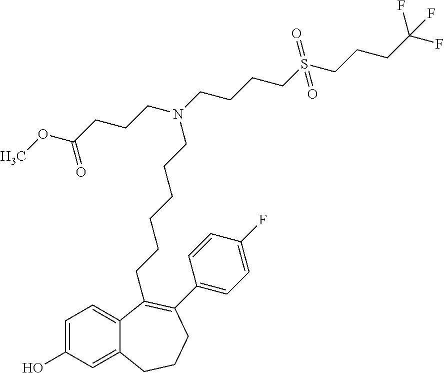 [SMILES: COC(=O)CCCN(CCCCCCC1=C(c2ccc(F)cc2)CCCc2cc(O)ccc21)CCCCS(=O)(=O)CCCC(F)(F)F]

130 mg (0.22 mmol) of 8-(4-fluorophenyl)-9-[6-({4-[(4,4,4-trifluorobutyl)sulphonyl]butyl}amino)hexyl]-6,7-dihydro-5H-benzo[7]annulen-3-ol was stirred at room temperature with 44.3 mg (0.24 mmol) of 4-bromobutyric acid methyl ester and 92.3 mg (0.67 mmol) of potassium carbonate in 2.5 mL of DMF for 72 hours. It was evaporated to dryness, and after adding water it was extracted three times with dichloromethane. The combined organic phases were washed three times with water, dried over magnesium sulphate and concentrated by evaporation. It was purified on Silica Gel 60 (solvent: dichloromethane, dichloromethane-methanol 98:2 and 95:5). 105 mg (69% of theor.) of product was isolated.

¹H-NMR (300 MHz, chloroform-d₁): δ=1.00-1.28 (m, 8H), 1.48-1.61 (m, 2H), 1.71 (mc, 2H), 1.84 (mc, 2H), 2.00-2.45 (m, 18H), 2.62 (mc, 2H), 2.95-3.09 (m, 4H), 3.66 (s, 3H), 6.71-6.78 (m, 2H), 7.03 (tt, 2H), 7.14-7.23 (m, 3H).

### Example 89

### N-{6-[8-(4-Fluorophenyl)-3-hydroxy-6,7-dihydro-5H-benzo[7]annulen-9-yl]hexyl}-N-{4-[(4,4,4-trifluorobutyl)sulphonyl]butyl}acetamide

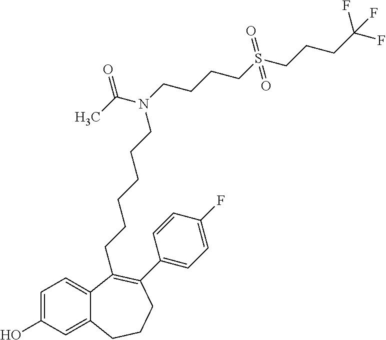 [SMILES: CC(=O)N(CCCCCCC1=C(c2ccc(F)cc2)CCCc2cc(O)ccc21)CCCCS(=O)(=O)CCCC(F)(F)F]

130 mg (0.22 mmol) of 8-(4-fluorophenyl)-9-[6-({4-[(4,4,4-trifluorobutyl)sulphonyl]butyl}amino)hexyl]-6,7-dihydro-5H-benzo[7]annulen-3-ol was stirred at room temperature with 46.4 mg (0.26 mmol) of acetic acid-4-nitrophenylester in 2.6 mL of DMF for 24 hours. It was evaporated to dryness, taken up in ethyl acetate, washed once with saturated sodium carbonate solution and three times with water, dried over magnesium sulphate and concentrated by evaporation. It was purified using HPLC-Method 1. 75.1 mg (54% of theor.) of product was isolated.

¹H-NMR (300 MHz, chloroform-d₁): δ=1.02-1.26 (m, 6H), 1.27-1.42 (m, 2H), 1.58-1.72 (m, 2H), 1.80 (mc, 2H), 1.97-2.21 (m, 9H), 2.24-2.41 (m, 4H), 2.56-2.66 (m, 2H), 2.97-3.31 (m, 8H), 6.70-6.79 (m, 2H), 7.04 (tt, 2H), 7.12-7.23 (m, 3H).

### Example 90

### ({6-[8-(4-Fluorophenyl)-3-hydroxy-6,7-dihydro-5H-benzo[7]annulen-9-yl]hexyl}{4-[(4,4,4-trifluorobutyl)sulphonyl]butyl}amino)acetonitrile

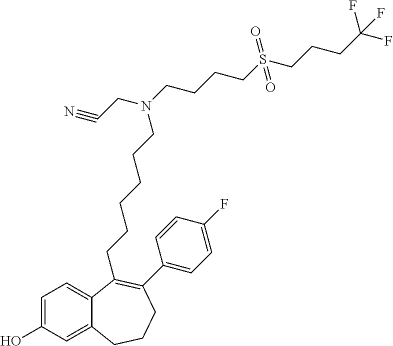 [SMILES: N#CCN(CCCCCCC1=C(c2ccc(F)cc2)CCCc2cc(O)ccc21)CCCCS(=O)(=O)CCCC(F)(F)F]

100 mg (0.17 mmol) of 8-(4-fluorophenyl)-9-[6-({4-[(4,4,4-trifluorobutyl)sulphonyl]butyl}amino)hexyl]-6,7-dihydro-5H-benzo[7]annulen-3-ol, 71 mg (0.51 mmol) of sodium carbonate and 22.6 mg (0.19 mmol) of bromoacetonitrile in 2 mL of DMF were stirred at room temperature for 5 hours. It was concentrated by evaporation, and after adding water it was shaken three times with dichloromethane. The combined organic phases were washed three times with water, dried over magnesium sulphate and concentrated by evaporation. It was purified using HPLC-Method 1. The product fraction was dissolved in dichloromethane, washed with 5% sodium hydrogen carbonate solution and water, dried over magnesium sulphate and concentrated by evaporation. It was purified on Silica Gel 60 (Biotage, Isolera; solvent: dichloromethane, gradient dichloromethane-methanol 100:0→80:20). 42.5 mg (40% of theor.) of product was obtained.

¹H-NMR (400 MHz, chloroform-d₁): δ=1.05-1.30 (m, 8H), 1.59 (mc, 2H), 1.87 (mc, 2H), 2.04-2.20 (m, 6H), 2.27-2.40 (m, 6H), 2.49 (t, 2H), 2.63 (t, 2H), 2.97-3.07 (m, 4H), 3.48 (s, 2H), 5.18 (s, 1H), 6.72 (d, 1H), 6.75 (dd, 1H), 7.04 (tt, 2H), 7.15-7.23 (m, 3H).

### Example 91

### N-{6-[8-(4-Fluorophenyl)-3-hydroxy-6,7-dihydro-5H-benzo[7]annulen-9-yl]hexyl}-N-{4-[(4,4,4-trifluorobutyl)sulphonyl]butyl}methanesulphonamide

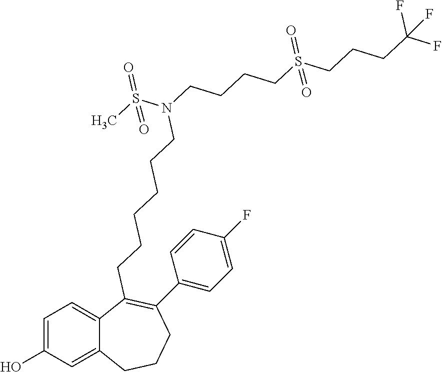 [SMILES: CS(=O)(=O)N(CCCCCCC1=C(c2ccc(F)cc2)CCCc2cc(O)ccc21)CCCCS(=O)(=O)CCCC(F)(F)F]

30.6 mg (0.27 mmol) of methanesulphonic acid chloride in 1 mL dichloromethane was added dropwise to 130 mg (0.22 mmol) of 6-(4-fluorophenyl)-5-{6-[4-(4,4,4-trifluorobutane-1-sulphonyl)-butylamino]-hexyl}-8,9-dihydro-7H-benzocyclohepten-2-ol and 27 mg (0.27 mmol) of triethylamine in 1.5 mL dichloromethane. It was stirred for 24 hours at room temperature. 27 mg (0.27 mmol) of triethylamine and 30 mg (0.26 mmol) of methanesulphonyl chloride were added and it was stirred at room temperature for 3 hours. It was diluted with dichloromethane, washed once with saturated sodium hydrogen carbonate solution and three times with water, dried over magnesium sulphate and concentrated by evaporation. It was purified on Silica Gel 60 (solvent: dichloromethane, dichloromethane-methanol 98:2). 120 mg (73% of theor.) of intermediate was isolated. 92.5 mg (0.13 mmol) of 8-(4-fluorophenyl)-9-{6-[(methylsulphonyl){4-[(4,4,4-trifluorobutyl)sulphonyl]butyl}amino]hexyl}-6,7-dihydro-5H-benzo[7]annulen-3-ylmethanesulphon at and 20 mg (0.50 mmol) of sodium hydroxide in 2.5 mL methanol were stirred at room temperature for 24 hours. 0.5 mL of 2M NaOH was added and it was stirred for 24 hours at room temperature and for 8 hours at 50° C. The volatile constituents were drawn off, the residue was taken up in water, neutralized with citric acid and shaken three times with dichloromethane. The combined organic phases were washed with water twice, dried over magnesium sulphate and concentrated by evaporation. It was purified using HPLC-Method 1. 42.8 mg (52% of theor.) of product was isolated.

¹H-NMR (400 MHz, chloroform-d₁): δ=1.07-1.24 (m, 6H), 1.39 (mc, 2H), 1.69 (mc, 2H), 1.88 (mc, 2H), 2.04-2.20 (m, 6H), 2.27-2.40 (m, 4H), 2.59-2.65 (m, 2H), 2.78 (s, 3H), 2.98-3.08 (m, 6H), 3.11 (t, 2H), 6.72 (d, 1H), 6.75 (dd, 1H), 7.04 (tt, 2H), 7.15-7.22 (m, 3H).

### Example 92

### 8-(4-Fluorophenyl)-9-{6-[(2-hydroxyethyl){4-[(4,4,4-trifluorobutyl)sulphonyl]butyl}amino]hexyl}-6,7-dihydro-5H-benzo[7]annulen-3-ol

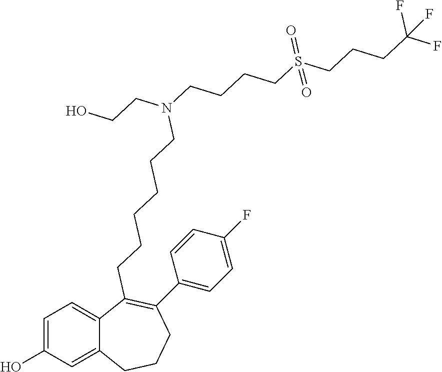 [SMILES: O=S(=O)(CCCCN(CCO)CCCCCCC1=C(c2ccc(F)cc2)CCCc2cc(O)ccc21)CCCC(F)(F)F]

130 mg (0.31 mmol) of 9-(6-bromohexyl)-8-(4-fluorophenyl)-6,7-dihydro-5H-benzo[7]annulen-3-ol was reacted at 80° C. with 108.9 mg (0.37 mmol) of 2-({4-[(4,4,4-trifluorobutyl)sulphonyl]butyl}amino)ethanol according to general specification 11. It was purified using HPLC-Method 1. 36 mg (18% of theor.) of product was isolated.

¹H-NMR (300 MHz, chloroform-d₁): δ=1.01-1.37 (m, 8H), 1.78 (mc, 2H), 1.91 (mc, 2H), 2.04-2.21 (m, 6H), 2.25-2.43 (m, 4H), 2.54 (mc, 2H), 2.59-2.65 (m, 2H), 2.67 (mc, 2H), 2.73 (mc, 2H), 2.81 (mc, 2H), 3.01-3.12 (m, 4H), 3.74 (mc, 2H), 6.73-6.80 (m, 2H), 7.04 (tt, 2H), 7.13-7.22 (m, 3H).

### Example 93

### 8-(4-Fluorophenyl)-9-[6-([(2S)-2-hydroxypropyl]{4-[(4,4,4-trifluorobutyl)sulphonyl]butyl}amino)hexyl]-6,7-dihydro-5H-benzo[7]annulen-3-ol

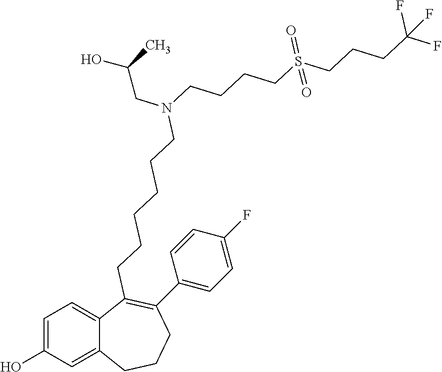 [SMILES: C[C@H](O)CN(CCCCCCC1=C(c2ccc(F)cc2)CCCc2cc(O)ccc21)CCCCS(=O)(=O)CCCC(F)(F)F]

130 mg (0.31 mmol) of 9-(6-bromohexyl)-8-(4-fluorophenyl)-6,7-dihydro-5H-benzo[7]annulen-3-ol was reacted at 80° C. with 114.1 mg (0.37 mmol) of (2S)-1-({4-[(4,4,4-trifluorobutyl)sulphonyl]butyl}amino)propan-2-ol according to general specification 11. It was purified using HPLC-Method 1. 31.6 mg (16% of theor.) of product was isolated.

¹H-NMR (300 MHz, chloroform-d₁): δ=1.00-1.40 (m, 11H), 1.65-1.96 (m, 4H), 2.02-2.21 (m, 6H), 2.25-2.42 (m, 4H), 2.45-2.79 (m, 8H), 2.98-3.11 (m, 4H), 3.95 (mc, 1H), 6.71-6.80 (m, 2H), 7.04 (tt, 2H), 7.11-7.23 (m, 3H).

### Example 94

### N-{6-[8-(4-Fluorophenyl)-3-hydroxy-6,7-dihydro-5H-benzo[7]annulen-9-yl]hexyl}-N-{4-[(4,4,4-trifluorobutyl)sulphonyl]butyl}glycine

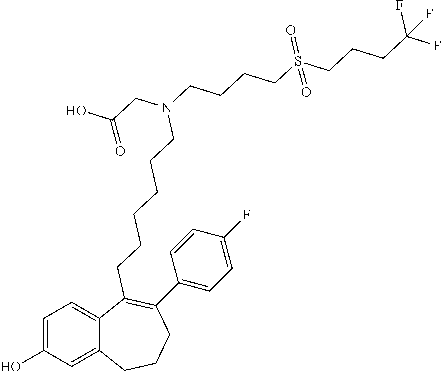 [SMILES: O=C(O)CN(CCCCCCC1=C(c2ccc(F)cc2)CCCc2cc(O)ccc21)CCCCS(=O)(=O)CCCC(F)(F)F]

100 mg (0.15 mmol) of methyl-N-{6-[8-(4-fluorophenyl)-3-hydroxy-6,7-dihydro-5H-benzo[7]annulen-9-yl]hexyl}-N-{4-[(4,4,4-trifluorobutyl)sulphonyl]butyl}glycinate was stirred overnight at room temperature with 40 mg (1.00 mmol) of sodium hydroxide in 4 mL methanol. It was adjusted to a pH of 5-6 with 10% aqueous citric acid solution, before being concentrated by evaporation. The residue was taken up in water and extracted with dichloromethane four times. The combined organic phases were dried over magnesium sulphate and concentrated by evaporation. It was purified using HPLC-Method 1. 50.7 mg (52% of theor.) of product was isolated.

¹H-NMR (300 MHz, chloroform-d₁): δ=0.96-1.34 (m, 8H), 1.77-2.00 (m, 4H), 2.02-2.19 (m, 6H), 2.26-2.43 (m, 4H), 2.59 (mc, 2H), 2.70 (mc, 2H), 2.88 (mc, 2H), 3.07-3.22 (m, 4H), 3.49 (s, 2H), 6.76 (d, 1H), 6.86 (dd, 1H), 7.03 (tt, 2H), 7.09-7.22 (m, 3H).

### Example 95

### N-{6-[8-(4-Fluorophenyl)-3-hydroxy-6,7-dihydro-5H-benzo[7]annulen-9-yl]hexyl}-N-{4-[(4,4,4-trifluorobutyl)sulphonyl]butyl}-beta-alanine

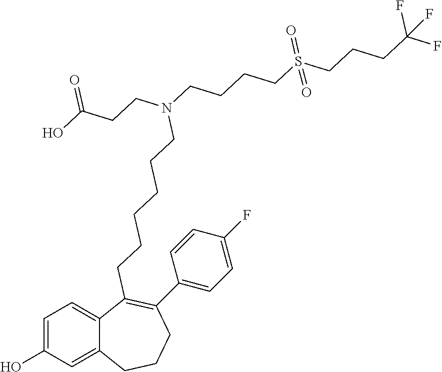 [SMILES: O=C(O)CCN(CCCCCCC1=C(c2ccc(F)cc2)CCCc2cc(O)ccc21)CCCCS(=O)(=O)CCCC(F)(F)F]

100 mg (0.15 mmol) of methyl-N-{6-[8-(4-fluorophenyl)-3-hydroxy-6,7-dihydro-5H-benzo[7]annulen-9-yl]hexyl}-N-{4-[(4,4,4-trifluorobutyl)sulphonyl]butyl}-beta-alaninate was stirred overnight at room temperature with 40 mg (1.00 mmol) of sodium hydroxide in 3 mL methanol. It was adjusted to a pH of 5-6 with 10% aqueous citric acid solution, before being concentrated by evaporation. The residue was taken up in water and extracted with dichloromethane four times. The combined organic phases were dried over magnesium sulphate and concentrated by evaporation. It was purified using HPLC-Method 1. 54 mg (55% of theor.) of product was isolated.

¹H-NMR (300 MHz, chloroform-d₁): δ=1.02-1.34 (m, 8H), 1.72-1.97 (m, 4H), 2.00-2.21 (m, 6H), 2.25-2.41 (m, 4H), 2.49 (mc, 2H), 2.54-2.66 (m, 4H), 2.78 (mc, 2H), 2.92 (mc, 2H), 3.03-3.15 (m, 4H), 6.77 (d, 1H), 6.82 (dd, 1H), 7.03 (mc, 2H), 7.11-7.23 (m, 3H).

### Example 96

### 4-({6-[8-(4-Fluorophenyl)-3-hydroxy-6,7-dihydro-5H-benzo[7]annulen-9-yl]hexyl}{4-[(4,4,4-trifluorobutyl)sulphonyl]butyl}amino)butanoic acid

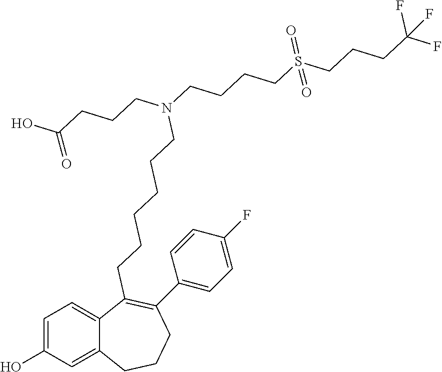 [SMILES: O=C(O)CCCN(CCCCCCC1=C(c2ccc(F)cc2)CCCc2cc(O)ccc21)CCCCS(=O)(=O)CCCC(F)(F)F]

98 mg (0.14 mmol) of methyl-4-({6-[8-(4-fluorophenyl)-3-hydroxy-6,7-dihydro-5H-benzo[7]annulen-9-yl]hexyl}{4-[(4,4,4-trifluorobutyl)sulphonyl]butyl}amino)butanoate was stirred overnight at room temperature with 40 mg (1.00 mmol) of sodium hydroxide in 4 mL methanol. It was adjusted to a pH of 5-6 with 10% aqueous citric acid solution, before being concentrated by evaporation. The residue was taken up in water and extracted with dichloromethane four times. The combined organic phases were dried over magnesium sulphate and concentrated by evaporation. It was purified using HPLC-Method 1. 49 mg (51% of theor.) of product was isolated.

¹H-NMR (300 MHz, chloroform-d₁): δ=1.02-1.39 (m, 8H), 1.72-1.96 (m, 6H), 2.01-2.21 (m, 6H), 2.25-2.43 (m, 4H), 2.46-2.66 (m, 6H), 2.69-2.85 (m, 4H), 3.02-3.15 (m, 4H), 6.75-6.83 (m, 2H), 7.03 (mc, 2H), 7.12 (d, 1H), 7.18 (mc, 2H).

### Example 97

### 8-(4-Fluorophenyl)-9-{6-[(2-hydroxyethyl){3-[(4,4,4-trifluorobutyl)sulphonyl]propyl}amino]hexyl}-6,7-dihydro-5H-benzo[7]annulen-3-ol

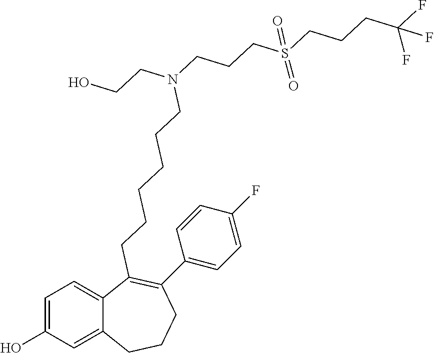 [SMILES: O=S(=O)(CCCN(CCO)CCCCCCC1=C(c2ccc(F)cc2)CCCc2cc(O)ccc21)CCCC(F)(F)F]

130 mg (0.31 mmol) of 9-(6-bromohexyl)-8-(4-fluorophenyl)-6,7-dihydro-5H-benzo[7]annulen-3-ol was reacted at 80° C. with 103.6 mg (0.37 mmol) of 2-({3-[(4,4,4-trifluorobutyl)sulphonyl]propyl}amino)ethanol according to general specification 11. It was purified using HPLC-Method 1. 46.3 mg (24% of theor.) of product was isolated.

¹H-NMR (300 MHz, chloroform-d₁): δ=0.99-1.36 (m, 8H), 2.03-2.22 (m, 8H), 2.25-2.42 (m, 4H), 2.50 (mc, 2H), 2.57-2.66 (m, 2H), 2.73-2.89 (m, 4H), 3.09 (mc, 4H), 3.71 (mc, 2H), 6.71-6.80 (m, 2H), 7.04 (tt, 2H), 7.13-7.23 (m, 3H).

### Example 98

### 8-(4-Fluorophenyl)-9-[6-([2R)-2-hydroxypropyl]{4-[(4,4,4-trifluorobutyl)sulphonyl]butyl}amino)hexyl]-6,7-dihydro-5H-benzo[7]annulen-3-ol

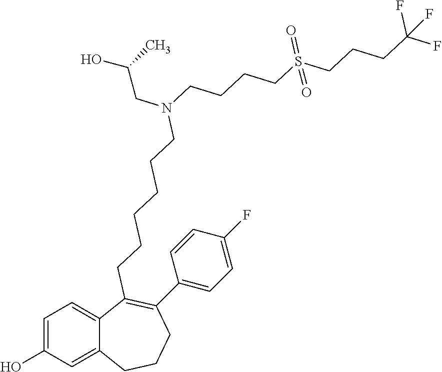 [SMILES: C[C@@H](O)CN(CCCCCCC1=C(c2ccc(F)cc2)CCCc2cc(O)ccc21)CCCCS(=O)(=O)CCCC(F)(F)F]

130 mg (0.31 mmol) of 9-(6-bromohexyl)-8-(4-fluorophenyl)-6,7-dihydro-5H-benzo[7]annulen-3-ol was reacted at 80° C. with 114.1 mg (0.37 mmol) of (2R)-1-({4-[(4,4,4-trifluorobutyl)sulphonyl]butyl}amino)propan-2-ol according to general specification 11. It was purified using HPLC-Method 1. 67.2 mg (34% of theor.) of product was isolated.

¹H-NMR (300 MHz, chloroform-d₁): δ=1.01-1.38 (m, 11H), 1.62-1.77 (m, 2H), 1.79-1.96 (m, 2H), 2.02-2.21 (m, 6H), 2.25-2.75 (m, 12H), 2.98-3.10 (m, 4H), 3.84-3.96 (m, 1H), 6.70-6.79 (m, 2H), 7.04 (tt, 2H), 7.12-7.23 (m, 3H).

### Example 99

### 2-Fluoro-8-(4-fluorophenyl)-9-[6-(methyl{3-[(4,4,5,5,5-pentafluoropentyl)sulphonyl]propyl}amino)hexyl]-6,7-dihydro-5H-benzo[7]annulen-3-ol

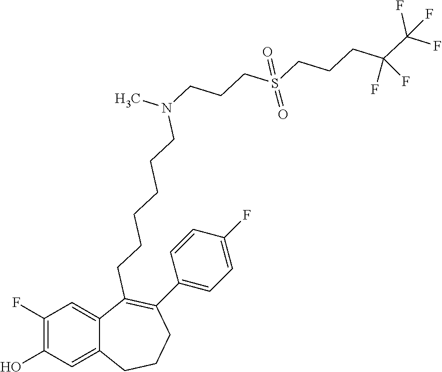 [SMILES: CN(CCCCCCC1=C(c2ccc(F)cc2)CCCc2cc(O)c(F)cc21)CCCS(=O)(=O)CCCC(F)(F)C(F)(F)F]

130 mg (0.30 mmol) of 9-(6-bromohexyl)-2-fluoro-8-(4-fluorophenyl)-6,7-dihydro-5H-benzo[7]annulen-3-ol was reacted with 124.3 mg (0.42 mmol) of N-methyl-3-[(4,4,5,5,5-pentafluoropentyl)sulphonyl]propan-1-amine according to general specification 11. It was purified using HPLC-Method 1. 52.0 mg (27% of theor.) of product was isolated.

¹H-NMR (300 MHz, chloroform-d₁): δ=1.03-1.25 (m, 6H), 1.33 (mc, 2H), 2.01-2.41 (m, 17H), 2.52-2.68 (m, 4H), 3.04-3.15 (m, 4H), 6.85 (d, 1H), 6.95-7.09 (m, 3H), 7.14-7.22 (m, 2H).

### Example 100

### 2-Fluoro-8-(4-fluorophenyl)-9-{6-[(2-hydroxyethyl){3-[(4,4,5,5,5-pentafluoropentyl)sulphonyl]propyl}amino]hexyl}-6,7-dihydro-5H-benzo[7]annulen-3-ol

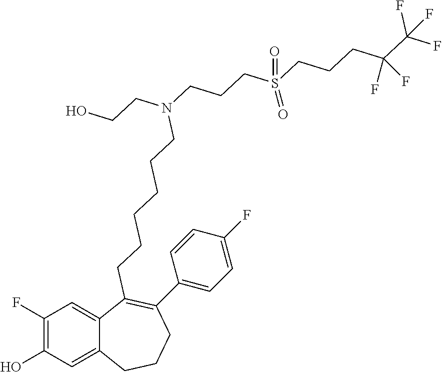 [SMILES: O=S(=O)(CCCN(CCO)CCCCCCC1=C(c2ccc(F)cc2)CCCc2cc(O)c(F)cc21)CCCC(F)(F)C(F)(F)F]

130 mg (0.30 mmol) of 9-(6-bromohexyl)-2-fluoro-8-(4-fluorophenyl)-6,7-dihydro-5H-benzo[7]annulen-3-ol was reacted with 136.8 mg (0.42 mmol) of 2-({3-[(4,4,5,5,5-pentafluoropentyl)sulphonyl]propyl}amino)ethanol according to general specification 11. It was purified using HPLC-Method 1. 25.0 mg (12% of theor.) of product was isolated.

¹H-NMR (300 MHz, chloroform-d₁): δ=1.02-1.25 (m, 6H), 1.32 (mc, 2H), 2.01-2.38 (m, 12H), 2.46-2.62 (m, 4H), 2.75 (t, 2H), 2.81 (t, 2H), 3.03-3.14 (m, 4H), 3.68 (t, 2H), 6.85 (d, 1H), 6.96-7.09 (m, 3H), 7.14-7.22 (m, 2H).

### Example 101

### 2-Fluoro-8-(4-fluorophenyl)-9-[6-(methyl{4-[(4,4,4-trifluorobutyl)sulphonyl]butyl}amino)hexyl]-6,7-dihydro-5H-benzo[7]annulen-3-ol

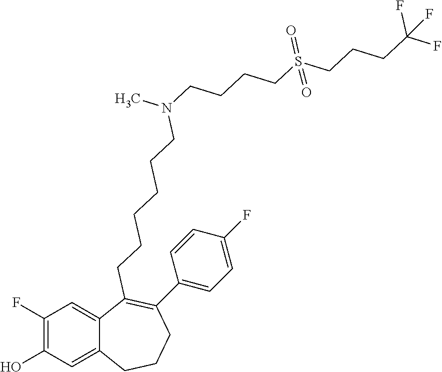 [SMILES: CN(CCCCCCC1=C(c2ccc(F)cc2)CCCc2cc(O)c(F)cc21)CCCCS(=O)(=O)CCCC(F)(F)F]

130 mg (0.30 mmol) of 9-(6-bromohexyl)-2-fluoro-8-(4-fluorophenyl)-6,7-dihydro-5H-benzo[7]annulen-3-ol was reacted with 109.2 mg (0.42 mmol) of N-methyl-4-[(4,4,4-trifluorobutyl)sulphonyl]butan-1-amine according to general specification 11. It was purified using HPLC-Method 1. 66.7 mg (36% of theor.) of product was isolated.

¹H-NMR (300 MHz, chloroform-d₁): δ=1.02-1.25 (m, 6H), 1.38 (mc, 2H), 1.69-1.82 (m, 2H), 1.89 (mc, 2H), 2.01-2.20 (m, 6H), 2.23-2.41 (m, 7H), 2.46 (mc, 2H), 2.55 (mc, 2H), 2.62 (mc, 2H), 3.00-3.10 (m, 4H), 6.83 (d, 1H), 6.93-7.08 (m, 3H), 7.13-7.21 (m, 2H).

### Example 102

### 2-Fluoro-8-(4-fluorophenyl)-9-[6-(methyl{3-[(5,5,5-trifluoropentyl)sulphonyl]propyl}amino)hexyl]-6,7-dihydro-5H-benzo[7]annulen-3-ol

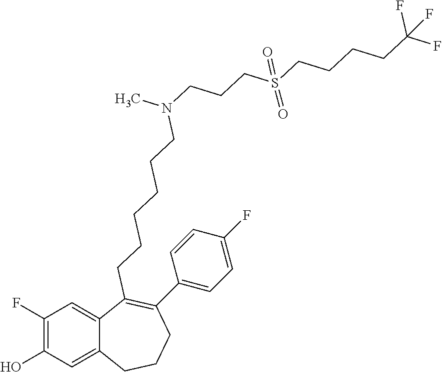 [SMILES: CN(CCCCCCC1=C(c2ccc(F)cc2)CCCc2cc(O)c(F)cc21)CCCS(=O)(=O)CCCCC(F)(F)F]

130 mg (0.30 mmol) of 9-(6-bromohexyl)-2-fluoro-8-(4-fluorophenyl)-6,7-dihydro-5H-benzo[7]annulen-3-ol was reacted with 109.2 mg (0.42 mmol) of N-methyl-3-[(5,5,5-trifluoropentyl)sulphonyl]propan-1-amine according to general specification 11. It was purified using HPLC-Method 1. 40.0 mg (22% of theor.) of product was isolated.

¹H-NMR (300 MHz, chloroform-d₁): δ=1.01-1.25 (m, 6H), 1.27-1.41 (m, 2H), 1.67-1.80 (m, 2H), 1.93 (mc, 2H), 2.01-2.23 (m, 8H), 2.31 (t, 2H), 2.36 (s, 3H), 2.41 (mc, 2H), 2.57 (mc, 2H), 2.70 (t, 2H), 3.01 (mc, 2H), 3.08 (mc, 2H), 6.84 (d, 1H), 6.95-7.08 (m, 3H), 7.14-7.21 (m, 2H).

### Example 103

### 2-Fluoro-8-(4-fluorophenyl)-9-{6-[(2-hydroxyethyl){3-[(4,4,4-trifluorobutyl)sulphonyl]propyl}amino]hexyl}-6,7-dihydro-5H-benzo[7]annulen-3-ol

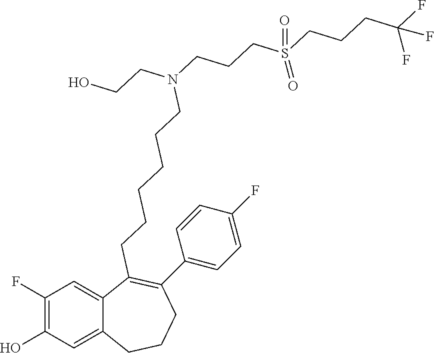 [SMILES: O=S(=O)(CCCN(CCO)CCCCCCC1=C(c2ccc(F)cc2)CCCc2cc(O)c(F)cc21)CCCC(F)(F)F]

130 mg (0.30 mmol) of 9-(6-bromohexyl)-2-fluoro-8-(4-fluorophenyl)-6,7-dihydro-5H-benzo[7]annulen-3-ol was reacted with 115.9 mg (0.42 mmol) of 2-({3-[(4,4,4-trifluorobutyl)sulphonyl]propyl}amino)ethanol according to general specification 11. It was purified using HPLC-Method 1. 23.0 mg (12% of theor.) of product was isolated.

¹H-NMR (300 MHz, chloroform-d₁): δ=1.01-1.25 (m, 6H), 1.26-1.39 (m, 2H), 2.01-2.21 (m, 8H), 2.25-2.42 (m, 4H), 2.51 (mc, 2H), 2.58 (mc, 2H), 2.74 (t, 2H), 2.80 (t, 2H), 3.08 (t, 4H), 3.67 (t, 2H), 6.85 (d, 1H), 6.96-7.09 (m, 3H), 7.14-7.22 (m, 2H).

### Example 104

### 2-Fluoro-8-(4-fluorophenyl)-9-{6-[(2-hydroxyethyl){4-[(4,4,4-trifluorobutyl)sulphonyl]butyl}amino]hexyl}-6,7-dihydro-5H-benzo[7]annulen-3-ol

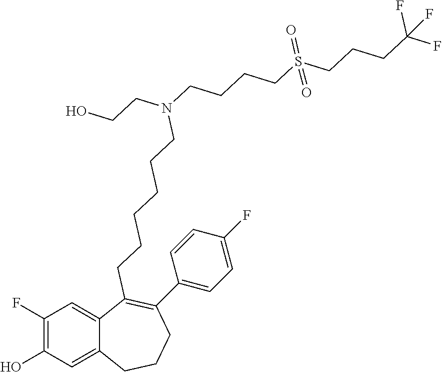 [SMILES: O=S(=O)(CCCCN(CCO)CCCCCCC1=C(c2ccc(F)cc2)CCCc2cc(O)c(F)cc21)CCCC(F)(F)F]

130 mg (0.30 mmol) of 9-(6-bromohexyl)-2-fluoro-8-(4-fluorophenyl)-6,7-dihydro-5H-benzo[7]annulen-3-ol was reacted with 121.8 mg (0.42 mmol) of 2-({4-[(4,4,4-trifluorobutyl)sulphonyl]butyl}amino)ethanol according to general specification 11. It was purified using HPLC-Method 1. 30.0 mg (16% of theor.) of product was isolated.

¹H-NMR (300 MHz, chloroform-d₁): δ=1.02-1.40 (m, 8H), 1.67-1.80 (m, 2H), 1.82-1.95 (m, 2H), 2.03-2.21 (m, 6H), 2.25-2.42 (m, 4H), 2.52 (mc, 2H), 2.58 (mc, 2H), 2.68 (t, 2H), 2.75 (t, 2H), 3.00-3.11 (m, 4H), 3.68 (t, 2H), 6.86 (d, 1H), 6.97-7.09 (m, 3H), 7.14-7.22 (m, 2H).

### Example 105

### 2-Fluoro-8-(4-fluorophenyl)-9-[6-(methyl{4-[(4,4,5,5,5-pentafluoropentyl)sulphonyl]butyl}amino)hexyl]-6,7-dihydro-5H-benzo[7]annulen-3-ol

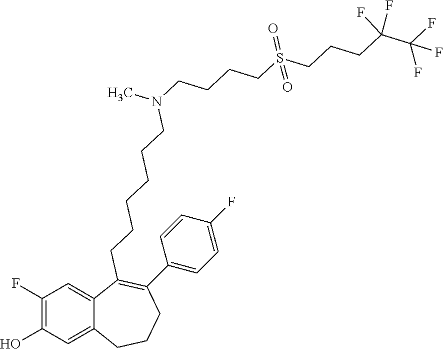 [SMILES: CN(CCCCCCC1=C(c2ccc(F)cc2)CCCc2cc(O)c(F)cc21)CCCCS(=O)(=O)CCCC(F)(F)C(F)(F)F]

130 mg (0.30 mmol) of 9-(6-bromohexyl)-2-fluoro-8-(4-fluorophenyl)-6,7-dihydro-5H-benzo[7]annulen-3-ol was reacted for 22 hours with 110.0 mg (0.42 mmol) of N-methyl-4-[(4,4,5,5,5-pentafluoropentyl)sulphonyl]butan-1-amine according to general specification 11. It was purified using HPLC-Method 1. 40.0 mg (20% of theor.) of product was isolated.

¹H-NMR (400 MHz, chloroform-d₁): δ=1.05-1.24 (m, 6H), 1.36 (mc, 2H), 1.67-1.77 (m, 2H), 1.89 (mc, 2H), 2.02-2.44 (m, 15H), 2.49-2.62 (m, 4H), 3.01-3.11 (m, 4H), 6.83 (d, 1H), 6.98 (d, 1H), 7.00-7.08 (m, 2H), 7.15-7.21 (m, 2H).

### Example 106

### 2-Fluoro-8-(4-fluorophenyl)-9-[6-(methyl{4-[(3,3,3-trifluoropropyl)sulphonyl]butyl}amino)hexyl]-6,7-dihydro-5H-benzo[7]annulen-3-ol

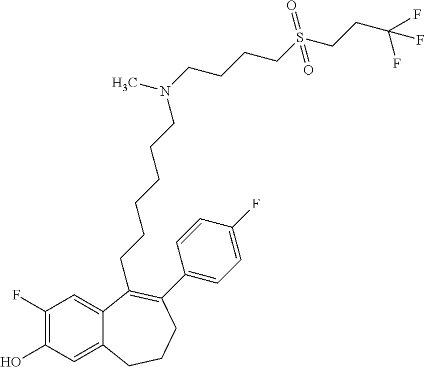 [SMILES: CN(CCCCCCC1=C(c2ccc(F)cc2)CCCc2cc(O)c(F)cc21)CCCCS(=O)(=O)CCC(F)(F)F]

130 mg (0.30 mmol) of 9-(6-bromohexyl)-2-fluoro-8-(4-fluorophenyl)-6,7-dihydro-5H-benzo[7]annulen-3-ol was reacted for 22 hours with 88.6 mg (0.36 mmol) of N-methyl-4-[(3,3,3-trifluoropropyl)sulphonyl]butan-1-amine according to general specification 11. It was purified using HPLC-Method 1. 46.0 mg (24% of theor.) of product was isolated.

¹H-NMR (400 MHz, chloroform-d₁): δ=1.04-1.23 (m, 6H), 1.37 (mc, 2H), 1.68-1.79 (m, 2H), 1.90 (mc, 2H), 2.01-2.13 (m, 4H), 2.30 (t, 2H), 2.34 (s, 3H), 2.41 (mc, 2H), 2.51-2.60 (m, 4H), 2.61-2.74 (m, 2H), 3.09 (mc, 2H), 3.19 (mc, 2H), 6.82 (d, 1H), 6.97 (d, 1H), 7.03 (tt, 2H), 7.14-7.20 (m, 2H).

### Example 107

### 2-Fluoro-8-(4-fluorophenyl)-9-[6-(methyl{5-[(3,3,3-trifluoropropyl)sulphonyl]pentyl}amino)hexyl]-6,7-dihydro-5H-benzo[7]annulen-3-ol

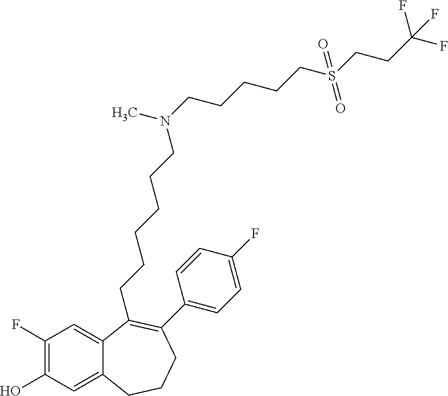 [SMILES: CN(CCCCCCC1=C(c2ccc(F)cc2)CCCc2cc(O)c(F)cc21)CCCCCS(=O)(=O)CCC(F)(F)F]

130 mg (0.30 mmol) of 9-(6-bromohexyl)-2-fluoro-8-(4-fluorophenyl)-6,7-dihydro-5H-benzo[7]annulen-3-ol was reacted with 93.6 mg (0.36 mmol) of N-methyl-5-[(3,3,3-trifluoropropyl)sulphonyl]pentan-1-amine according to general specification 11. It was purified using HPLC-Method 1. 34.5 mg (19% of theor.) of product was isolated.

¹H-NMR (400 MHz, chloroform-d₁): δ=1.04-1.24 (m, 6H), 1.31-1.42 (m, 2H), 1.45-1.55 (m, 2H), 1.62 (mc, 2H), 1.90 (mc, 2H), 2.03-2.15 (m, 4H), 2.30 (t, 2H), 2.35 (s, 3H), 2.40 (mc, 2H), 2.50-2.60 (m, 4H), 2.62-2.75 (m, 2H), 3.06 (mc, 2H), 3.16-3.23 (m, 2H), 6.84 (d, 1H), 6.96-7.08 (m, 3H), 7.15-7.21 (m, 2H).

### Example 108

### 8-(4-Fluorophenyl)-9-{6-[(2-hydroxyethyl)(3-{[3,4,4,4-tetrafluoro-3-(trifluoromethyl)butyl]sulphonyl}propyl)amino]hexyl}-6,7-dihydro-5H-benzo[7]annulen-3-ol

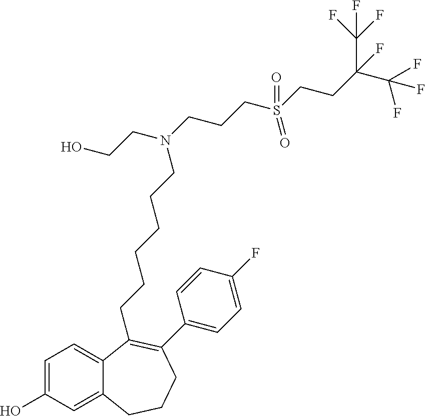 [SMILES: O=S(=O)(CCCN(CCO)CCCCCCC1=C(c2ccc(F)cc2)CCCc2cc(O)ccc21)CCC(F)(C(F)(F)F)C(F)(F)F]

130 mg (0.31 mmol) of 9-(6-bromohexyl)-8-(4-fluorophenyl)-6,7-dihydro-5H-benzo[7]annulen-3-ol was reacted with 135.8 mg (0.37 mmol) of 2-[(3-{[3,4,4,4-tetrafluoro-3-(trifluoromethyl)butyl]sulphonyl}propyl)amino]ethanol according to general specification 11. It was purified using HPLC-Method 1. 26.4 mg (12% of theor.) of product was isolated.

¹H-NMR (300 MHz, chloroform-d₁): δ=1.01-1.28 (m, 6H), 1.47 (mc, 2H), 2.01-2.19 (m, 4H), 2.33-2.49 (m, 4H), 2.58-2.79 (m, 6H), 3.06 (mc, 2H), 3.22 (mc, 2H), 3.29-3.40 (m, 4H), 3.94 (mc, 2H), 6.79-6.85 (m, 2H), 7.00-7.09 (m, 2H), 7.13-7.22 (m, 3H).

### Example 109

### 8-(4-Fluorophenyl)-9-{6-[(2-hydroxyethyl)(4-{[3,4,4,4-tetrafluoro-3-(trifluoromethyl)butyl]sulphonyl}butyl)amino]hexyl}-6,7-dihydro-5H-benzo[7]annulen-3-ol

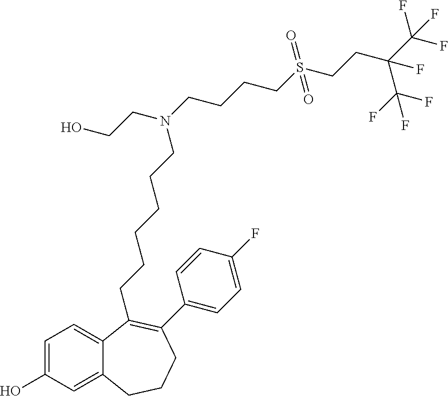 [SMILES: O=S(=O)(CCCCN(CCO)CCCCCCC1=C(c2ccc(F)cc2)CCCc2cc(O)ccc21)CCC(F)(C(F)(F)F)C(F)(F)F]

130 mg (0.31 mmol) of 9-(6-bromohexyl)-8-(4-fluorophenyl)-6,7-dihydro-5H-benzo[7]annulen-3-ol was reacted with 141.0 mg (0.37 mmol) of 2-[(4-{[3,4,4,4-tetrafluoro-3-(trifluoromethyl)butyl]sulphonyl}butyl)amino]ethanol according to general specification 11. It was purified using HPLC-Method 1. 47.1 mg (21% of theor.) of product was isolated.

¹H-NMR (300 MHz, chloroform-d₁): δ=1.03-1.28 (m, 6H), 1.49 (mc, 2H), 1.93-2.19 (m, 8H), 2.37 (mc, 2H), 2.57-2.75 (m, 4H), 2.82 (mc, 2H), 3.02-3.15 (m, 4H), 3.20-3.33 (m, 4H), 3.98 (mc, 2H), 6.79-6.86 (m, 2H), 7.04 (tt, 2H), 7.14-7.22 (m, 3H).

### Example 110

### 8-(4-Fluorophenyl)-9-{6-[methyl(3-{[3,4,4,4-tetrafluoro-3-(trifluoromethyl)butyl]sulphonyl}propyl)amino]hexyl}-6,7-dihydro-5H-benzo[7]annulen-3-ol

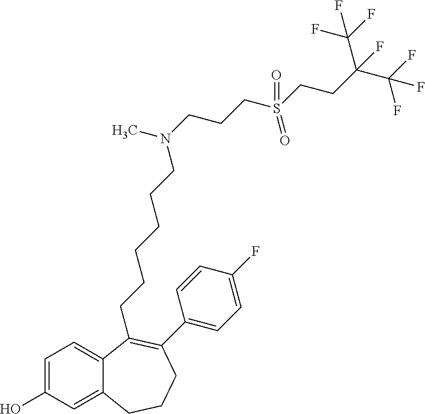 [SMILES: CN(CCCCCCC1=C(c2ccc(F)cc2)CCCc2cc(O)ccc21)CCCS(=O)(=O)CCC(F)(C(F)(F)F)C(F)(F)F]

130 mg (0.31 mmol) of 9-(6-bromohexyl)-8-(4-fluorophenyl)-6,7-dihydro-5H-benzo[7]annulen-3-ol was reacted with 124.6 mg (0.37 mmol) of N-methyl-3-{[3,4,4,4-tetrafluoro-3-(trifluoromethyl)butyl]sulphonyl}propan-1-amine according to general specification 11. It was purified using HPLC-Method 1. 46.1 mg (22% of theor.) of product was isolated.

¹H-NMR (300 MHz, chloroform-d₁): δ=1.02-1.34 (m, 8H), 1.98-2.16 (m, 6H), 2.21-2.30 (m, 5H), 2.35 (t, 2H), 2.55 (t, 2H), 2.58-2.74 (m, 4H), 3.14 (mc, 2H), 3.17-3.25 (m, 2H), 6.70-6.77 (m, 2H), 7.03 (tt, 2H), 7.12-7.22 (m, 3H).

### Example 111

### 8-(4-Fluorophenyl)-9-{6-[methyl(4-{[3,4,4,4-tetrafluoro-3-(trifluoromethyl)butyl]sulphonyl}butyl)amino]hexyl}-6,7-dihydro-5H-benzo[7]annulen-3-ol

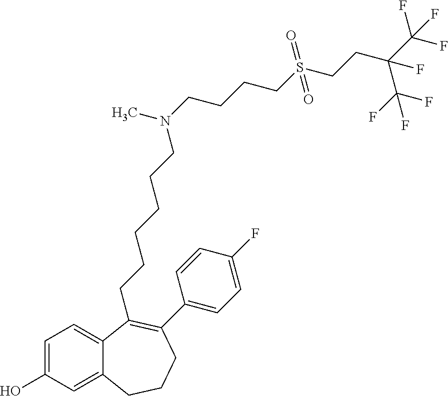 [SMILES: CN(CCCCCCC1=C(c2ccc(F)cc2)CCCc2cc(O)ccc21)CCCCS(=O)(=O)CCC(F)(C(F)(F)F)C(F)(F)F]

130 mg (0.31 mmol) of 9-(6-bromohexyl)-8-(4-fluorophenyl)-6,7-dihydro-5H-benzo[7]annulen-3-ol was reacted with 129.8 mg (0.37 mmol) of N-methyl-4-{[3,4,4,4-tetrafluoro-3-(trifluoromethyl)butyl]sulphonyl}butan-1-amine according to general specification 11. It was purified using HPLC-Method 1. 61.2 mg (28% of theor.) of product was isolated.

¹H-NMR (300 MHz, chloroform-d₁): δ=1.02-1.39 (m, 8H), 1.65 (mc, 2H), 1.82-1.95 (m, 2H), 2.00-2.16 (m, 4H), 2.17-2.29 (m, 5H), 2.31-2.45 (m, 4H), 2.56-2.75 (m, 4H), 3.09 (mc, 2H), 3.14-3.24 (m, 2H), 6.69-6.77 (m, 2H), 6.99-7.08 (m, 2H), 7.12-7.23 (m, 3H).

### Example 112

### 8-(4-Fluorophenyl)-9-[6-({3-[(5,5,5-trifluoropentyl)sulphonyl]propyl}amino)hexyl]-6,7-dihydro-5H-benzo[7]annulen-3-ol

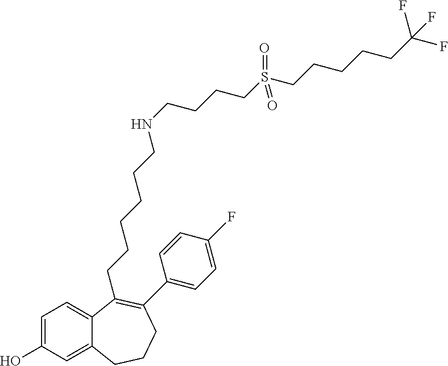 [SMILES: O=S(=O)(CCCCCC(F)(F)F)CCCCNCCCCCCC1=C(c2ccc(F)cc2)CCCc2cc(O)ccc21]

500 mg (1.20 mmol) of 9-(6-bromohexyl)-8-(4-fluorophenyl)-6,7-dihydro-5H-benzo[7]annulen-3-ol was reacted with 385.1 mg (1.56 mmol) of 3-[(5,5,5-trifluoropentyl)sulphonyl]propan-1-amine according to general specification 11. It was purified on Silica Gel 60 (solvent: dichloromethane, dichloromethane-methanol 95:5 and 90:10). 330 mg (47% of theor.) of product was isolated.

¹H-NMR (300 MHz, chloroform-d₁): δ=1.03-1.35 (m, 8H), 1.74 (mc, 2H), 1.88-2.21 (m, 10H), 2.35 (mc, 2H), 2.46 (t, 2H), 2.62 (mc, 2H), 2.73 (t, 2H), 3.01 (mc, 2H), 3.09 (mc, 2H), 6.70-6.77 (m, 2H), 7.00-7.07 (m, 2H), 7.14-7.22 (m, 3H).

### Example 113

### 8-(4-Fluorophenyl)-9-[6-({3-[(4,4,5,5,5-pentafluoropentyl)sulphonyl]propyl}amino)hexyl]-6,7-dihydro-5H-benzo[7]annulen-3-ol

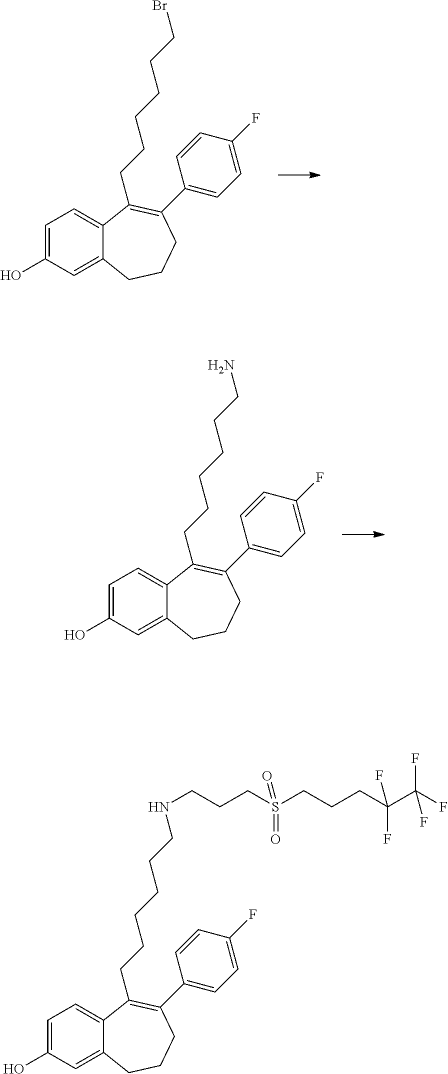 [SMILES: Oc1ccc2c(c1)CCCC(c1ccc(F)cc1)=C2CCCCCCBr ; NCCCCCCC1=C(c2ccc(F)cc2)CCCc2cc(O)ccc21 ; O=S(=O)(CCCNCCCCCCC1=C(c2ccc(F)cc2)CCCc2cc(O)ccc21)CCCC(F)(F)C(F)(F)F]

### Stage 1

2 g of 9-(6-bromohexyl)-8-(4-fluorophenyl)-6,7-dihydro-5H-benzo[7]annulen-3-ol was reacted in 30 mL methanol at 50° C. in an ammonia atmosphere (3 bar) within 5 hours within the scope of a pressure reaction. Saturated aqueous sodium hydrogen carbonate solution was added, it was extracted with ethyl acetate, the organic phases were washed with saturated sodium chloride solution, dried over sodium sulphate and concentrated by evaporation. After purification by silica gel column chromatography (dichloromethane/methanol), 474 mg of 9-(6-aminohexyl)-8-(4-fluorophenyl)-6,7-dihydro-5H-benzo[7]annulen-3-ol was obtained. MS (ESIpos) mass found: 353.00

### Stage 2

318 mg of 9-(6-aminohexyl)-8-(4-fluorophenyl)-6,7-dihydro-5H-benzo[7]annulen-3-ol, 272 mg (1.0 equiv.) of 3-chloropropyl-4,4,5,5,5-pentafluoropentylsulphone, 143 mg potassium iodide and 286 mg sodium carbonate were heated in 5 mL of DMF at 80° C. for 18 hours. Water and ethyl acetate were added to the reaction mixture, the phases were separated and the aqueous phase was re-extracted with ethyl acetate three times. The combined organic phases were washed with saturated sodium hydrogen carbonate solution and concentrated by evaporation. After purification by preparative HPLC (acetonitrile/water/formic acid), 180 mg of the title compound was obtained (can optionally be partially or completely in the form of formate salt). ¹H-NMR (300 MHz, DMSO-d₆, selected signals): δ 0.93-1.16 (m), 1.17-1.33 (m), 1.71-2.04 (8H), 2.64 (t, 2H), 3.08-3.25 (4H), 6.58-6.67 (2H), 7.05-7.26 (5H), 8.27 (s). MS (ESIneg) mass found: 619.26.

### Examples 114 to 123

Similarly to general specification 11, examples 114 to 123 were prepared starting from 9-(6-bromohexyl)-8-(4-fluorophenyl)-6,7-dihydro-5H-benzo[7]annulen-3-ol by reaction with amines:

| | | Name of | | |
|---|---|---|---|---|
| Ex. | Amine | example | Structure | Analytical data |
| 114 | N-methyl- 4- [(4,4,5,5,5- pentafluoropentyl) sulphonyl] butan-1-amine | 8-(4- fluorophenyl)-9- [6-(methyl{4- [(4,4,5,5,5- pentafluoropentyl) sulphonyl]butyl} amino)hexyl]- 6,7-dihydro-5H- benzo[7]annulen- 3-ol | 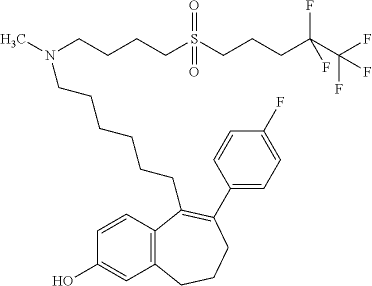 [SMILES: CN(CCCCCCC1=C(c2ccc(F)cc2)CCCc2cc(O)ccc21)CCCCS(=O)(=O)CCCC(F)(F)C(F)(F)F] | ¹H-NMR (600 MHz, chloroform-d₁) δ 1.06- 1.16 (4H), 1.16-1.23 (2H), 1.23-1.30 (2H), 1.5- 1.62 (2H), 1.81-1.88 (2H), 2.06-2.13 (4H), 2.13 (s, 3H), 2.15-2.33 (8H), 2.35 (t, 2H), 2.40 (t, 2H), 2.62 (t, 2H), 2.99- 3.06 (4H), 6.69-6.71 (1H), 6.71-6.75 (1H), 7.00-7.06 (2H), 7.15- 7.22 (3H). MS (ESIpos) mass found: 647.3 |
| 115 | N-methyl- 3- [(4,4,5,5,5- pentafluoropentyl) sulphonyl]propan- 1-amine | 8-(4- fluorophenyl)-9- [6-(methyl{3- [(4,4,5,5,5- pentafluoropentyl) sulphonyl]propyl} amino)hexyl]- 6,7-dihydro-5H- benzo[7]annulen- 3-ol | 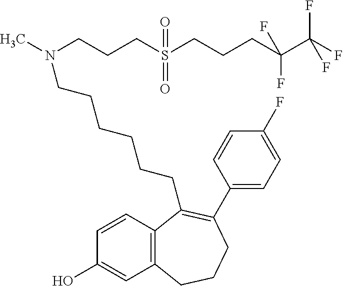 [SMILES: CN(CCCCCCC1=C(c2ccc(F)cc2)CCCc2cc(O)ccc21)CCCS(=O)(=O)CCCC(F)(F)C(F)(F)F] | ¹H-NMR (600 MHz, chloroform-d₁) δ 1.05- 1.15 (4H), 1.15-1.23 (2H), 1.23-1.30 (2H), 1.92-2.00 (2H), 2.05- 2.12 (4H), 2.14 (s, 3H), 2.14-2.32 (6H), 2.35 (t, 2H), 2.40 (t, 2H), 2.62 (t, 2H), 3.00-3.08 (4H), 6.69-6.71 (1H), 6.71- 6.75 (1H), 7.00-7.06 (2H), 7.15-7.22 (3H). MS (ESIpos) mass found: 633.3 |
| 116 | N-methyl- 3- [(3,3,4,4,4- pentafluorobutyl) sulphonyl]propan- 1-amine | 8-(4- fluorophenyl)-9- [6-(methyl{3- [(3,3,4,4,4- pentafluorobutyl) sulphonyl]propyl} amino)hexyl]- 6,7-dihydro-5H- benzo[7]annulen- 3-ol | 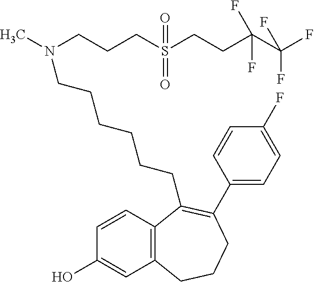 [SMILES: CN(CCCCCCC1=C(c2ccc(F)cc2)CCCc2cc(O)ccc21)CCCS(=O)(=O)CCC(F)(F)C(F)(F)F] | ¹H-NMR (300 MHz, DMSO-d₆, selected signals): δ 0.93-1.25 (8H), 1.76 (mc, 2H), 2.12 (t, 2H), 2.20-2.35 (4H), 3.17 (mc, 2H), 3.40 (mc, 2H), 6.58-6.65 (2H), 7.06-7.26 (5H), 8.12 (s, 1H). MS (ESIneg) mass found: 619.3 |
| 117 | N-methyl- 3-[(3,3,3,- trifluoropropyl) sulphonyl]propan- 1-amine | 8-(4- fluorophenyl)-9- [6-(methyl{3- [(4,4,4- trifluorobutyl) sulphonyl]propyl} amino)hexyl]- 6,7-dihydro-5H- benzo[7]annulen- 3-ol | 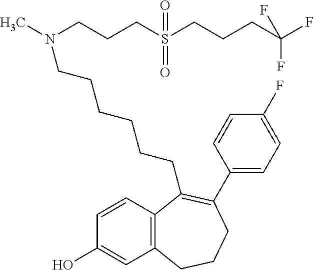 [SMILES: CN(CCCCCCC1=C(c2ccc(F)cc2)CCCc2cc(O)ccc21)CCCS(=O)(=O)CCCC(F)(F)F] | ¹H-NMR (300 MHz, DMSO-d₆, selected signals): δ 0.90-1.26), 1.71 (mc, 2H), 2.01 (s, 3H), 2.09 (t, 2H), 2.20- 2.23 (4H), 3.04 (mc, 2H), 3.15 (t, 2H), 6.58-6.66 (2H), 7.05-7.26 (5H), 9.28 (s, 1H). MS (ESIpos) mass found: 583.3. |
| 118 | N-methyl- 3- [(4,4,5,5,5- pentafluoropentyl) sulphinyl]propan- 1-amine | 8-(4- fluorophenyl)-9- [6-(methyl{3- [(4,4,5,5,5- pentafluoropentyl) sulphinyl]propyl} amino)hexyl]- 6,7-dihydro-5H- benzo[7]annulen- 3-ol | 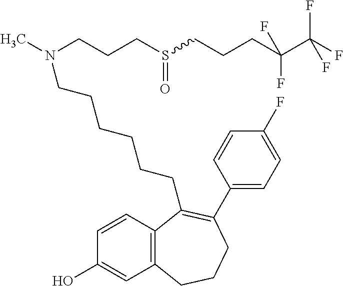 [SMILES: CN(CCCCCCC1=C(c2ccc(F)cc2)CCCc2cc(O)ccc21)CCCS(=O)CCCC(F)(F)C(F)(F)F] | ¹H-NMR (500 MHz, chloroform-d₁, selected signals): δ 1.04-1.14 (4H), 1.15-1.24 (4H), 1.89 (mc, 2H), 2.32-2.44 (4H), 2.58-2.66 (2H), 2.68-2.86 (4H), 6.71- 6.74 (1H), 6.75 (dd, 1H), 7.00-7.07 (2H), 7.15 (d, 1H), 7.17-7.22 (2H). MS (ESIpos) mass found: 617.3. |
| 119 | 2-methyl-1- ({3-[(3,3,3- trifluoropropyl) sulphonyl]propyl} amino)propan- 2-ol | 8-(4- fluorophenyl)-9- {6-[(2-hydroxy- 2- methylpropyl){3- [(3,3,3- trifluoropropyl) sulphonyl]propyl} amino]hexyl}- 6,7-dihydro-5H- benzo[7]annulen- 3-ol | 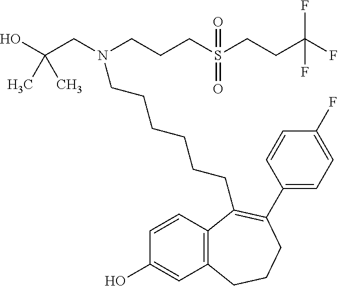 [SMILES: CC(C)(O)CN(CCCCCCC1=C(c2ccc(F)cc2)CCCc2cc(O)ccc21)CCCS(=O)(=O)CCC(F)(F)F] | ¹H-NMR (300 MHz, DMSO-d₆, selected signals): δ 0.98 (s, 6H), 1.64-1.78 (m, 2H), 2.16 (s, 2H), 2.20-2.35 (4H), 2.59-2.77 (m, 2H), 3.11- 3.20 (m, 2H), 3.8-4.1 (br s), 6.56-6.65 (2H), 7.05-7.26 (5H). MS (ESIpos) mass found: 627.3 |
| 120 | 2-methyl-1- ({3-[(3,3,3- trifluoropropyl) sulphinyl]propyl} amino)propan- 2-ol | 8-(4- fluorophenyl)-9- {6-[(2-hydroxy- 2- methylpropyl){3- [(3,3,3- trifluoropropyl) sulphinyl]propyl} amino)hexyl}- 6,7-dihydro-5H- benzo[7]annulen- 3-ol | 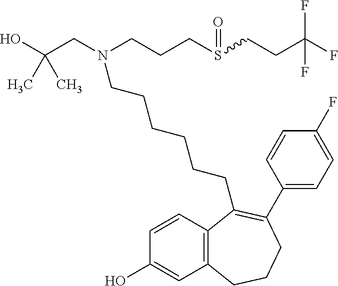 [SMILES: CC(C)(O)CN(CCCCCCC1=C(c2ccc(F)cc2)CCCc2cc(O)ccc21)CCCS(=O)CCC(F)(F)F] | ¹H-NMR (300 MHz, DMSO-d₆, selected signals): δ 0.98 (s), 1.65 (mc, 2H), 2.21-2.35 (4H), 2.55-2.85 (5H), 2.89-3.03 (1H), 3.91 (s), 6.58-6.67 (2H), 7.05- 7.26 (5H), 9.26 (s). MS (ESIpos) mass found: 611.3 |
| 121 | 3-[(4,4- difluoro- cyclohexyl) sulphonyl]-N- methylpropan- 1-amine | 9-{6-[{3-[(4,4- difluorocyclo- hexyl)sulphonyl] propyl}(methyl) amino]hexyl}-8- (4-fluorophenyl)- 6,7-dihydro-5H- benzo[7]annulen- 3-ol | 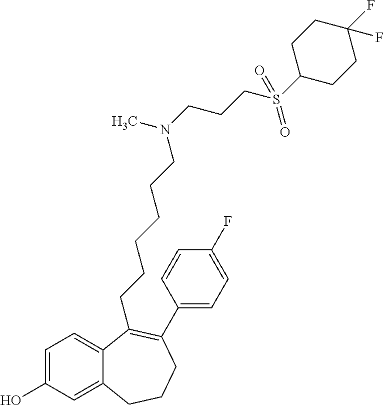 [SMILES: CN(CCCCCCC1=C(c2ccc(F)cc2)CCCc2cc(O)ccc21)CCCS(=O)(=O)C1CCC(F)(F)CC1] | ¹H-NMR (300 MHz, DMSO-d₆): δ 0.91-1.28 (m, 8H), 1.48-1.67 (m, 2H), 1.67-2.19 (17H, contains s at 2.04 ppm), 2.21-2.36 (m, 4H), 2.48- 2.56 (m), 2.98-3.09 (m, 1H), 3.19-3.32 (m, 1H), 6.58-6.66 (m, 2H), 7.06- 7.26 (m, 5H), 8.14 (s). MS (ESIpos) mass found: 591.30 |
| 122 | 4-[(4,4- difluoro- cyclohexyl) sulphonyl]-N- methylbutan- 1-amin | 9-{6-[{4-[(4,4- difluorocyclo- hexyl)sulphonyl] butyl}(methyl) amino]hexyl}-8- (4-fluorophenyl)- 6,7-dihydro-5H- benzo[7]annulen- 3-ol* | 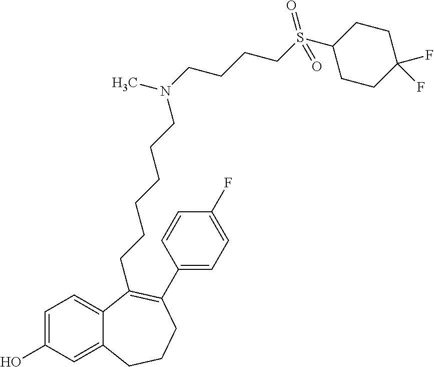 [SMILES: CN(CCCCCCC1=C(c2ccc(F)cc2)CCCc2cc(O)ccc21)CCCCS(=O)(=O)C1CCC(F)(F)CC1] | ¹H-NMR (500 MHz, chloroform-d₁): 1.05- 1.14 (m, 4H), 1.15-1.23 (m, 2H), 1.26-1.34 (m, 2H), 1.63-1.71 (m, 2H), 1.72-1.92 (m, 2H), 1.92- 2.02 (m, 2H), 2.05- 2.14 (m, 4H), 2.19-2.38 (m, 11H), 2.47 (t, 2H), 2.61 (t, 2H), 2.88-2.96 (m, 1H), 2.96-3.03 (m, 2H), 6.70-6.76 (m, 2H), 7.00-7.06 (m, 2H), 7.15 (d, 1H), 7.17-7.22 (m, 2H), 8.62 (s). MS (EIpos) mass found: 605 [M+]. |
| 123 | 3-{[(4,4- difluoro- cyclohexyl) methyl] sulphonyl}-N- methylpropan- 1-amine | 9-{6-[(3-{[(4,4- difluorocyclo- hexyl)methyl] sulphonyl} propyl)(methyl) amino]hexyl}-8- (4-fluorophenyl)- 6,7-dihydro-5H- benzo[7]annulen- 3-ol* | 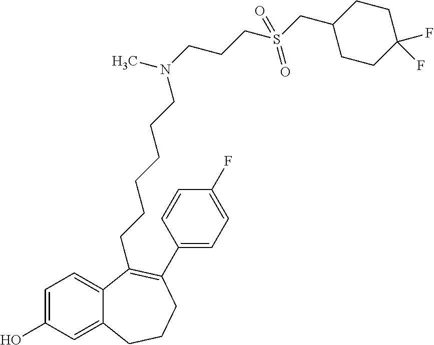 [SMILES: CN(CCCCCCC1=C(c2ccc(F)cc2)CCCc2cc(O)ccc21)CCCS(=O)(=O)CC1CCC(F)(F)CC1] | ¹H-NMR (400 MHz, DMSO-d₆, selected signals): 0.93-1.38 (m, 10H), 1.67-2.13 (m, contains s at 2.01 ppm), 2.22-2.32 (m, 4H), 2.96- 3.04 (m, 2H), 3.06 (d, 2H), 6.59-6.66 (m, 2H), 7.09 (d, 1H), 7.11-7.19 (m, 2H), 7.19-7.26 (m, 2H), 9.30 (s, 1H). MS (ESIneg) mass found: 605.32 |

*This example compound was purified by HPLC with addition of formic acid and subsequent freeze-drying. The example compound can be partially or completely in the form of formate salt.

### Examples 124 and 125

Similarly to general specification 11, examples 124 and 125 were prepared starting from 9-(6-bromohexyl)-8-(3-fluorophenyl)-6,7-dihydro-5H-benzo[7]annulen-3-ol by reaction with amines:

| | | Name of | | |
|---|---|---|---|---|
| Ex. | Amine | example | Structure | Analytical data |
| 124 | N-methyl- 4- [(4,4,5,5,5- pentafluoro- pentyl) sulphonyl] butan- 1-amine | 8-(3- fluorophenyl)-9- [6-(methyl{4- [(4,4,5,5,5- pentafluoropentyl) sulphonyl]butyl} amino)hexyl]-6,7- dihydro-5H- benzo[7]annulen- 3-ol | 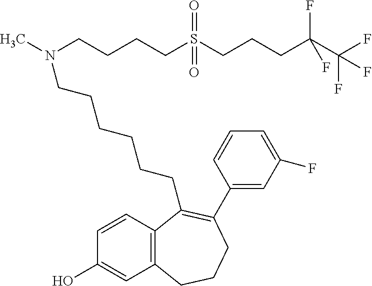 [SMILES: CN(CCCCCCC1=C(c2cccc(F)c2)CCCc2cc(O)ccc21)CCCCS(=O)(=O)CCCC(F)(F)C(F)(F)F] | ¹H-NMR (300 MHz, DMSO-d₆, selected signals): δ 0.9-1.25 (m, 8H), 1.36-1.49 (2H), 1.54-1.68 (2H), 1.82-2.11 (11H, contains singlet at δ = 1.99), 2.17 (t, 2H), 3.07 (mc, 2H), 3.16 (mc, 2H), 6.56-6.66 (2H), 6.96-7.13 (4H), 7.32- 7.44 (1H), 9.30 (s). MS (ESIpos) mass found: 647.3 |
| 125 | N-methyl- 3- [(4,4,5,5,5- pentafluoro- pentyl) sulphonyl] propan- 1-amine | 8-(3- fluorophenyl)-9- [6-(methyl{3- [(4,4,5,5,5- pentafluoropentyl) sulphonyl]propyl} amino)hexyl]-6,7- dihydro-5H- benzo[7]annulen- 3-ol | 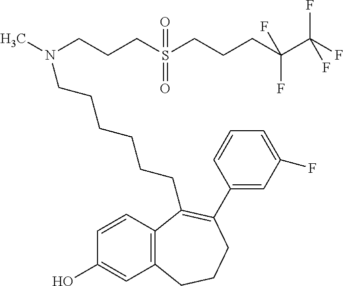 [SMILES: CN(CCCCCCC1=C(c2cccc(F)c2)CCCc2cc(O)ccc21)CCCS(=O)(=O)CCCC(F)(F)C(F)(F)F] | ¹H-NMR (300 MHz, DMSO-d₆, selected signals): δ 0.89-1.26 (m, 8H), 1.71 (quin. 2H), 2.00 (s, 3H), 2.09 (t, 2H), 3.08 (mc, 2H), 3.18 (t, 2H), 6.57- 6.67 (2H), 6.94-7.13 (4H), 7.32-7.42 (1H), 9.30 (s). MS (ESIpos) mass found: 633.3 |

### Example 126

Similarly to general specification 11, example 126 was prepared starting from 9-(6-bromohexyl)-8-(2-fluorophenyl)-6,7-dihydro-5H-benzo[7]annulen-3-ol by reaction with an amine:

| | | Name of | | |
|---|---|---|---|---|
| Ex. | Substrate | example | Structure | Analytical data |
| 126 | N-methyl-3- [(4,4,5,5,5- pentafluoropentyl) sulphonyl]propan- 1-amine | 8-(2- fluorophenyl)-9- [6-(methyl{3- [(4,4,5,5,5- pentafluoropentyl) sulphonyl]propyl} amino)hexyl]-6,7- dihydro-5H- benzo[7]annulen- 3-ol* | 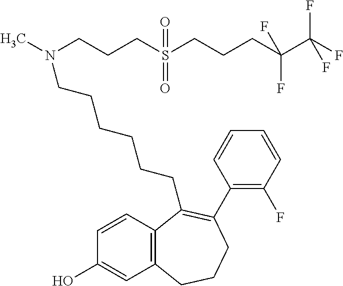 [SMILES: CN(CCCCCCC1=C(c2ccccc2F)CCCc2cc(O)ccc21)CCCS(=O)(=O)CCCC(F)(F)C(F)(F)F] | ¹H-NMR (300 MHz, DMSO-d₆, selected signals): δ 0.90-1.25 (8H), 1.67-1.80 (2H), 1.81-2.08 (9H, contains singlet at δ 2.06), 2.10- 2.25 (4H), 2.49- 2.59 (m, 2H), 3.05 (mc, 2H), 3.19 (t, 2H), 6.59-6.67 (2H), 7.07-7.13 (1H), 7.13-7.34 (4H), 8.14 (s). MS (ESIpos) mass found: 633.27 |

*This example compound was purified by HPLC with addition of formic acid and subsequent freeze-drying. The example compound can be partially or completely in the form of formate salt.

### Example 127

Similarly to general specification 11, example 127 was prepared starting from 9-(5-bromopentyl)-8-(4-fluorophenyl)-6,7-dihydro-5H-benzo[7]annulen-3-ol by reaction with an amine:

| Ex. | Substrate | Name of example | Structure | Analytical data |
|---|---|---|---|---|
| 127 | N-methyl- 3- [(4,4,5,5,5- pentafluoro- pentyl) sulphonyl] propan-1- amine | 8-(4-fluorophenyl)- 9-[5-(methyl{3- [(4,4,5,5,5- pentafluoropentyl) sulphonyl]propyl} amino)pentyl]-6,7- dihydro-5H- benzo[7]annulen-3- ol* | 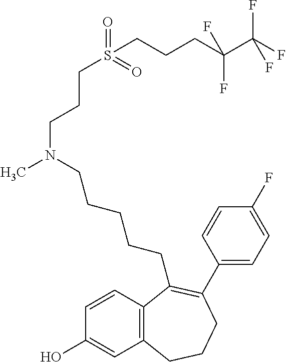 [SMILES: CN(CCCCCC1=C(c2ccc(F)cc2)CCCc2cc(O)ccc21)CCCS(=O)(=O)CCCC(F)(F)C(F)(F)F] | ¹H-NMR (300 MHz, DMSO-d₆, selected signals): δ 0.91-1.25 (6H), 1.71 (mc, 1H), 1.81-2.12 (11H, contains singlet), 2.97- 3.08 (m, 2H), 3.18 (t, 2H), 6.57-6.66 (m, 2H), 7.05-7.25 (m, 5H), 8.15 (s). MS (ESIpos) mass found: 619.26 |

*This example compound was purified by HPLC with addition of formic acid and subsequent freeze-drying. The example compound can be partially or completely in the form of formate salt.

### Example 128

### 8-(2,4-Difluorophenyl)-9-[6-(methyl{3-[(4,4,5,5,5-pentafluoropentyl)sulphonyl]propyl}amino)hexyl]-6,7-dihydro-5H-benzo[7]annulen-3-ol

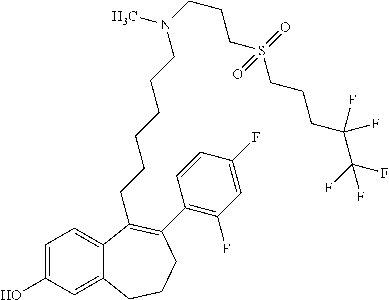 [SMILES: CN(CCCCCCC1=C(c2ccc(F)cc2F)CCCc2cc(O)ccc21)CCCS(=O)(=O)CCCC(F)(F)C(F)(F)F]

130 mg (0.30 mmol) of 9-(6-bromohexyl)-8-(2,4-difluorophenyl)-6,7-dihydro-5H-benzo[7]annulen-3-ol was reacted with 106.5 mg (0.36 mmol) of N-methyl-3-[(4,4,5,5,5-pentafluoropentyl)sulphonyl]propan-1-amine according to general specification 11. It was purified using HPLC-Method 1. 100.5 mg (52% of theor.) of product was isolated.

¹H-NMR (300 MHz, chloroform-d₁): δ=0.97-1.36 (m, 8H), 1.98-2.39 (m, 17H), 2.58-2.72 (m, 4H), 3.03-3.15 (m, 4H), 6.71-6.79 (m, 2H), 6.80-6.92 (m, 2H), 7.10-7.19 (m, 2H).

### Example 129

### 8-(2,4-Difluorophenyl)-9-[6-(methyl{3-[(5,5,5-trifluoropentyl)sulphonyl]propyl}amino)hexyl]-6,7-dihydro-5H-benzo[7]annulen-3-ol

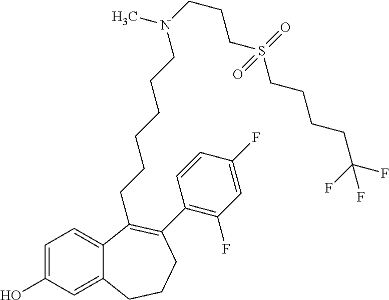 [SMILES: CN(CCCCCCC1=C(c2ccc(F)cc2F)CCCc2cc(O)ccc21)CCCS(=O)(=O)CCCCC(F)(F)F]

130 mg (0.30 mmol) of 9-(6-bromohexyl)-8-(2,4-difluorophenyl)-6,7-dihydro-5H-benzo[7]annulen-3-ol was reacted with 93.6 mg (0.36 mmol) of N-methyl-3-[(5,5,5-trifluoropentyl)sulphonyl]propan-1-amine according to general specification 11. It was purified using HPLC-Method 1. 100.4 mg (55% of theor.) of product was isolated.

¹H-NMR (300 MHz, chloroform-d₁): δ=0.97-1.37 (m, 8H), 1.68-1.80 (m, 2H), 1.88-2.21 (m, 10H), 2.28 (t, 2H), 2.32-2.41 (m, 5H), 2.64 (mc, 2H), 2.72 (t, 2H), 3.02 (mc, 2H), 3.08 (t, 2H), 6.72-6.79 (m, 2H), 6.80-6.92 (m, 2H), 7.10-7.19 (m, 2H).

### Example 130

### 8-(2,4-Difluorophenyl)-9-[6-(methyl{3-[(3,3,3-trifluoropropyl)sulphonyl]propyl}amino)hexyl]-6,7-dihydro-5H-benzo[7]annulen-3-ol

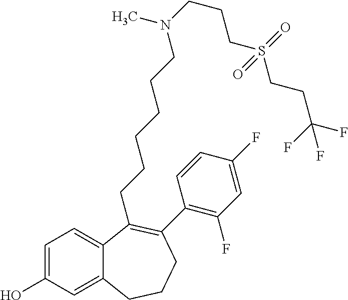 [SMILES: CN(CCCCCCC1=C(c2ccc(F)cc2F)CCCc2cc(O)ccc21)CCCS(=O)(=O)CCC(F)(F)F]

130 mg (0.30 mmol) of 9-(6-bromohexyl)-8-(2,4-difluorophenyl)-6,7-dihydro-5H-benzo[7]annulen-3-ol was reacted with 83.6 mg (0.36 mmol) of N-methyl-3-[(3,3,3-trifluoropropyl)sulphonyl]propan-1-amine according to general specification 11. It was purified using HPLC-Method 1. 79.7 mg (45% of theor.) of product was isolated.

¹H-NMR (300 MHz, chloroform-d₁): δ=0.98-1.37 (m, 8H), 1.98-2.19 (m, 6H), 2.28 (t, 2H), 2.32-2.41 (m, 5H), 2.58-2.77 (m, 6H), 3.16 (t, 2H), 3.22 (mc, 2H), 6.72-6.79 (m, 2H), 6.80-6.92 (m, 2H), 7.10-7.20 (m, 2H).

### Example 131

### 8-(2,4-Difluorophenyl)-9-[6-(methyl{4-[(3,3,3-trifluoropropyl)sulphonyl]butyl}amino)hexyl]-6,7-dihydro-5H-benzo[7]annulen-3-ol

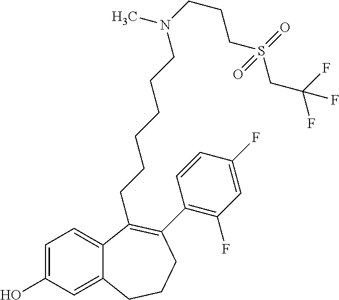 [SMILES: CN(CCCCCCC1=C(c2ccc(F)cc2F)CCCc2cc(O)ccc21)CCCS(=O)(=O)CC(F)(F)F]

130 mg (0.30 mmol) of 9-(6-bromohexyl)-8-(2,4-difluorophenyl)-6,7-dihydro-5H-benzo[7]annulen-3-ol was reacted with 88.6 mg (0.36 mmol) of N-methyl-4-[(3,3,3-trifluoropropyl)sulphonyl]butan-1-amine according to general specification 11. It was purified using HPLC-Method 1. 96.0 mg (53% of theor.) of product was isolated.

¹H-NMR (300 MHz, chloroform-d₁): δ=0.96-1.23 (m, 6H), 1.27-1.40 (m, 2H), 1.69-1.83 (m, 2H), 1.85-1.97 (m, 2H), 1.99-2.18 (m, 4H), 2.27 (t, 2H), 2.34-2.47 (m, 5H), 2.55-2.77 (m, 6H), 3.09 (mc, 2H), 3.20 (mc, 2H), 6.70-6.78 (m, 2H), 6.80-6.92 (m, 2H), 7.09-7.19 (m, 2H).

### Example 132

### 8-(2,4-Difluorophenyl)-9-[6-(methyl{4-[(4,4,4-trifluorobutyl)sulphonyl]butyl}amino)hexyl]-6,7-dihydro-5H-benzo[7]annulen-3-ol

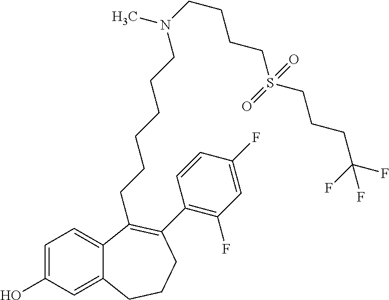 [SMILES: CN(CCCCCCC1=C(c2ccc(F)cc2F)CCCc2cc(O)ccc21)CCCCS(=O)(=O)CCCC(F)(F)F]

400 mg (0.92 mmol) of 9-(6-bromohexyl)-8-(2,4-difluorophenyl)-6,7-dihydro-5H-benzo[7]annulen-3-ol was reacted with 288.1 mg (1.10 mmol) of N-methyl-4-[(4,4,4-trifluorobutyl)sulphonyl]butan-1-amine according to general specification 11. It was purified using HPLC (XBridge C18, 5μ, 100×30 mm, 50 mL/min, solvent: water with 0.1% formic acid-acetonitrile 90:10, 0-1 minute; 90:10→20:80, 1-8.0 minutes; 0:100, 8.1-10 minutes). It was taken up in dichloromethane, washed once with saturated sodium hydrogen carbonate solution and twice with water, dried over magnesium sulphate and concentrated by evaporation. It was digested with diethyl ether and pentane. The residue was washed twice with ethyl acetate, twice with diethyl ether and once with pentane and concentrated by evaporation. The residue was dried overnight at 50° C. in a drying cabinet. 295.0 mg (52% of theor.) of product was isolated.

¹H-NMR (400 MHz, chloroform-d₁): δ=1.03-1.21 (m, 6H), 1.24-1.34 (m, 2H), 1.61 (quin, 2H), 1.85 (mc, 2H), 1.99-2.06 (m, 2H), 2.07-2.38 (m, 15H), 2.63 (mc, 2H), 2.98-3.06 (m, 4H), 6.68-6.73 (m, 2H), 6.81-6.90 (m, 2H), 7.12-7.19 (m, 2H).

### Example 133

### 8-(2,4-Difluorophenyl)-9-[6-(methyl{5-[(3,3,3-trifluoropropyl)sulphonyl]pentyl}amino)hexyl]-6,7-dihydro-5H-benzo[7]annulen-3-ol

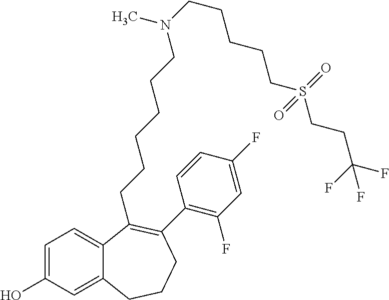 [SMILES: CN(CCCCCCC1=C(c2ccc(F)cc2F)CCCc2cc(O)ccc21)CCCCCS(=O)(=O)CCC(F)(F)F]

130 mg (0.30 mmol) of 9-(6-bromohexyl)-8-(2,4-difluorophenyl)-6,7-dihydro-5H-benzo[7]annulen-3-ol was reacted with 93.6 mg (0.36 mmol) of N-methyl-5-[(3,3,3-trifluoropropyl)sulphonyl]pentan-1-amine according to general specification 11. It was purified using HPLC-Method 1. 110.9 mg (60% of theor.) of product was isolated.

¹H-NMR (300 MHz, chloroform-d₁): δ=0.98-1.23 (m, 6H), 1.27-1.40 (m, 2H), 1.44-1.57 (m, 2H), 1.58-1.70 (m, 2H), 1.90 (mc, 2H), 1.98-2.18 (m, 4H), 2.27 (t, 2H), 2.35-2.47 (m, 5H), 2.53-2.77 (m, 6H), 3.06 (mc, 2H), 3.20 (mc, 2H), 6.71-6.78 (m, 2H), 6.80-6.91 (m, 2H), 7.10-7.19 (m, 2H).

### Example 134

### 8-(2,4-Difluorophenyl)-9-[6-(methyl{3-[(4,4,4-trifluorobutyl)sulphonyl]propyl}amino)hexyl]-6,7-dihydro-5H-benzo[7]annulen-3-ol

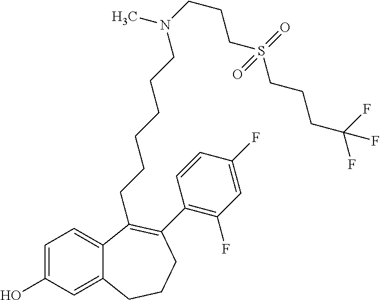 [SMILES: CN(CCCCCCC1=C(c2ccc(F)cc2F)CCCc2cc(O)ccc21)CCCS(=O)(=O)CCCC(F)(F)F]

130 mg (0.30 mmol) of 9-(6-bromohexyl)-8-(2,4-difluorophenyl)-6,7-dihydro-5H-benzo[7]annulen-3-ol was reacted with 88.6 mg (0.36 mmol) of N-methyl-3-[(4,4,4-trifluorobutyl)sulphonyl]propan-1-amine according to general specification 11. It was purified using HPLC-Method 1. 94.3 mg (52% of theor.) of product was isolated.

¹H-NMR (300 MHz, chloroform-d₁): δ=0.97-1.37 (m, 8H), 1.98-2.21 (m, 8H), 2.23-2.41 (m, 9H), 2.58-2.72 (m, 4H), 3.02-3.14 (m, 4H), 6.71-6.78 (m, 2H), 6.80-6.91 (m, 2H), 7.09-7.19 (m, 2H).

### Example 135

### 8-(2,4-Difluorophenyl)-9-[6-(methyl{3-[(6,6,6-trifluorohexyl)sulphonyl]propyl}amino)hexyl]-6,7-dihydro-5H-benzo[7]annulen-3-ol

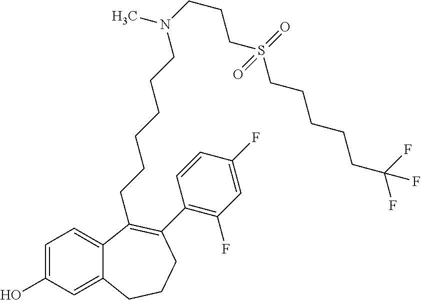 [SMILES: CN(CCCCCCC1=C(c2ccc(F)cc2F)CCCc2cc(O)ccc21)CCCS(=O)(=O)CCCCCC(F)(F)F]

130 mg (0.30 mmol) of 9-(6-bromohexyl)-8-(2,4-difluorophenyl)-6,7-dihydro-5H-benzo[7]annulen-3-ol was reacted with 98.7 mg (0.36 mmol) of N-methyl-3-[(6,6,6-trifluorohexyl)sulphonyl]propan-1-amine according to general specification 11. It was purified using HPLC-Method 1. 100.3 mg (53% of theor.) of product was isolated.

¹H-NMR (300 MHz, chloroform-d₁): δ=0.96-1.24 (m, 6H), 1.25-1.38 (m, 2H), 1.47-1.69 (m, 4H), 1.82-1.94 (m, 2H), 1.98-2.21 (m, 8H), 2.28 (t, 2H), 2.34-2.45 (m, 5H), 2.63 (mc, 2H), 2.77 (t, 2H), 3.01 (mc, 2H), 3.08 (t, 2H), 6.72-6.79 (m, 2H), 6.80-6.92 (m, 2H), 7.09-7.19 (m, 2H).

### Example 136

### 8-(2,4-Difluorophenyl)-9-{6-[(²H₃)methyl{3-[(5,5,5-trifluoropentyl)sulphonyl]propyl}amino]hexyl}-6,7-dihydro-5H-benzo[7]annulen-3-ol

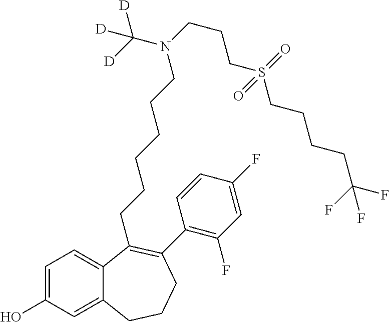 [SMILES: [2H]C([2H])([2H])N(CCCCCCC1=C(c2ccc(F)cc2F)CCCc2cc(O)ccc21)CCCS(=O)(=O)CCCCC(F)(F)F]

130 mg (0.30 mmol) of 9-(6-bromohexyl)-8-(2,4-difluorophenyl)-6,7-dihydro-5H-benzo[7]annulen-3-ol was reacted with 94.7 mg (0.36 mmol) of N-(²H₃)methyl-3-[(5,5,5-trifluoropentyl)sulphonyl]propan-1-amine according to general specification 11. It was purified using HPLC-Method 1. 88.6 mg (48% of theor.) of product was isolated.

¹H-NMR (300 MHz, chloroform-d₁): δ=0.98-1.36 (m, 8H), 1.67-1.81 (m, 2H), 1.87-2.37 (m, 14H), 2.58-2.70 (m, 4H), 2.96-3.11 (m, 4H), 6.71-6.79 (m, 2H), 6.80-6.92 (m, 2H), 7.10-7.20 (m, 2H).

### Example 137

### 8-(2,5-Difluorophenyl)-9-{6-[(²H₃)methyl{3-[(5,5,5-trifluoropentyl)sulphonyl]propyl}amino]hexyl}-6,7-dihydro-5H-benzo[7]annulen-3-ol

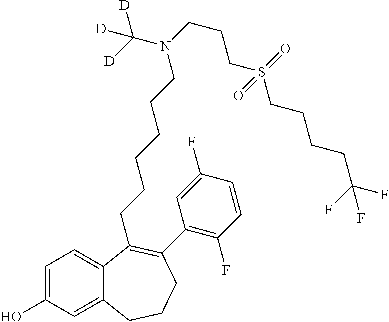 [SMILES: [2H]C([2H])([2H])N(CCCCCCC1=C(c2cc(F)ccc2F)CCCc2cc(O)ccc21)CCCS(=O)(=O)CCCCC(F)(F)F]

130 mg (0.30 mmol) of 9-(6-bromohexyl)-8-(2,5-difluorophenyl)-6,7-dihydro-5H-benzo[7]annulen-3-ol was reacted with 94.7 mg (0.36 mmol) of N-(²H₃)methyl-3-[(5,5,5-trifluoropentyl)sulphonyl]propan-1-amine according to general specification 11. It was purified using HPLC-Method 1. 102.9 mg (56% of theor.) of product was isolated.

¹H-NMR (300 MHz, chloroform-d₁): δ=0.98-1.37 (m, 8H), 1.67-1.80 (m, 2H), 1.87-2.24 (m, 10H), 2.25-2.40 (m, 4H), 2.57-2.73 (m, 4H), 2.96-3.12 (m, 4H), 6.71-6.79 (m, 2H), 6.85-6.97 (m, 2H), 7.04 (dt, 1H), 7.15 (d, 1H).

### Example 138

### 2-Fluoro-8-(4-fluorophenyl)-9-{6-[(2-hydroxyethyl)(4-{[3,4,4,4-tetrafluoro-3-(trifluoromethyl)butyl]sulphonyl}butyl)amino]hexyl}-6,7-dihydro-5H-benzo[7]annulen-3-ol

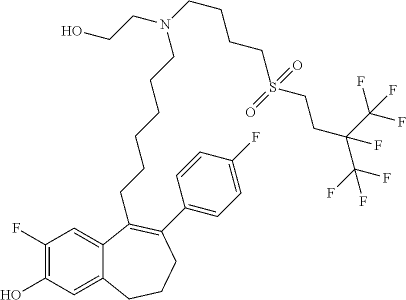

120 mg (0.28 mmol) of 9-(6-bromohexyl)-2-fluoro-8-(4-fluorophenyl)-6,7-dihydro-5H-benzo[7]annulen-3-ol was reacted with 124.8 mg (0.33 mmol) of 2-[(4-{[3,4,4,4-tetrafluoro-3-(trifluoromethyl)butyl]sulphonyl}butyl)amino]ethanol according to general specification 11. It was purified using HPLC-Method 1. 16.0 mg (8% of theor.) of product was isolated.

¹H-NMR (300 MHz, chloroform-d₁): δ=1.02-1.41 (m, 6H), 1.60-1.98 (m, 4H), 2.01-2.18 (m, 4H), 2.31 (t, 2H), 2.41-2.79 (m, 10H), 3.02-3.28 (m, 6H), 3.65 (mc, 2H), 6.78-6.93 (m, 1H), 6.96-7.10 (m, 3H), 7.13-7.22 (m, 2H).

### Example 139

### 8-(4-Fluorophenyl)-9-{6-[(²H₃)methyl{3-[(5,5,5-trifluoropentyl)sulphonyl]propyl}amino]hexyl}-6,7-dihydro-5H-benzo[7]annulen-3-ol

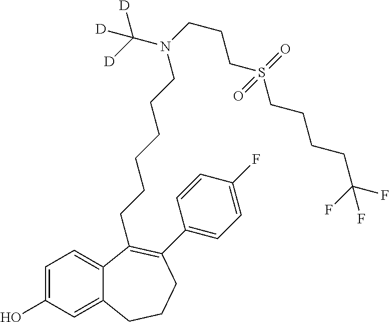 [SMILES: [2H]C([2H])([2H])N(CCCCCCC1=C(c2ccc(F)cc2)CCCc2cc(O)ccc21)CCCS(=O)(=O)CCCCC(F)(F)F]

130 mg (0.31 mmol) of 9-(6-bromohexyl)-8-(4-fluorophenyl)-6,7-dihydro-5H-benzo[7]annulen-3-ol was reacted with 98.8 mg (0.37 mmol) of N-(²H₃)methyl-3-[(5,5,5-trifluoropentyl)sulphonyl]propan-1-amine according to general specification 11. It was purified using HPLC-Method 1. 92 mg (49% of theor.) of product was isolated.

¹H-NMR (300 MHz, chloroform-d₁): δ=0.99-1.36 (m, 8H), 1.67-1.80 (m, 2H), 1.87-2.00 (m, 2H), 2.02-2.23 (m, 8H), 2.29-2.40 (m, 4H), 2.56-2.72 (m, 4H), 2.96-3.11 (m, 4H), 6.71-6.79 (m, 2H), 7.03 (tt, 2H), 7.12-7.23 (m, 3H).

### Example 140

### 2-Fluoro-8-(4-fluorophenyl)-9-[6-(methyl{3-[(6,6,6-trifluorohexyl)sulphonyl]propyl}amino)hexyl]-6,7-dihydro-5H-benzo[7]annulen-3-ol

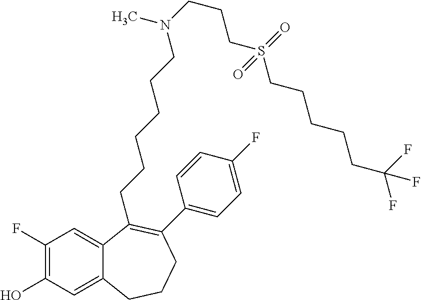 [SMILES: CN(CCCCCCC1=C(c2ccc(F)cc2)CCCc2cc(O)c(F)cc21)CCCS(=O)(=O)CCCCCC(F)(F)F]

120 mg (0.28 mmol) of 9-(6-bromohexyl)-2-fluoro-8-(4-fluorophenyl)-6,7-dihydro-5H-benzo[7]annulen-3-ol was reacted with 91.1 mg (0.33 mmol) of N-methyl-3-[(6,6,6-trifluorohexyl)sulphonyl]propan-1-amine according to general specification 11. It was purified using HPLC-Method 1. 42.5 mg (24% of theor.) of product was isolated.

¹H-NMR (300 MHz, chloroform-d₁): δ=1.00-1.25 (m, 6H), 1.27-1.40 (m, 2H), 1.47-1.68 (m, 4H), 1.88 (mc, 2H), 2.01-2.19 (m, 8H), 2.26-2.45 (m, 7H), 2.57 (mc, 2H), 2.71 (t, 2H), 3.00 (mc, 2H), 3.07 (mc, 2H), 6.85 (d, 1H), 6.95-7.09 (m, 3H), 7.13-7.21 (m, 2H).

### Example 141

### ({6-[8-(4-Fluorophenyl)-3-hydroxy-6,7-dihydro-5H-benzo[7]annulen-9-yl]hexyl}{3-[(5,5,5-trifluoropentyl)sulphonyl]propyl}amino)acetonitrile

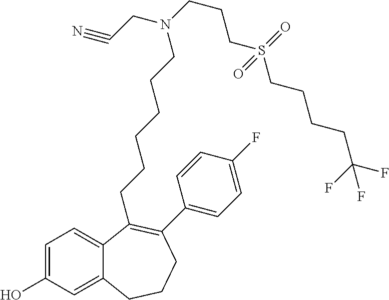 [SMILES: N#CCN(CCCCCCC1=C(c2ccc(F)cc2)CCCc2cc(O)ccc21)CCCS(=O)(=O)CCCCC(F)(F)F]

300 mg (0.51 mmol) of 8-(4-fluorophenyl)-9-[6-({3-[(5,5,5-trifluoropentyl)sulphonyl]propyl}amino)hexyl]-6,7-dihydro-5H-benzo[7]annulen-3-ol was stirred at room temperature with 67.8 mg (0.57 mmol) of bromoacetonitrile and 213.1 mg (1.54 mmol) of potassium carbonate for 5 hours in 10 mL of DMF. It was concentrated by evaporation, and after adding water it was extracted three times with dichloromethane. The combined organic phases were washed with water three times, dried over magnesium sulphate and concentrated by evaporation. It was purified using HPLC (XBridge C18, 5μ, 150×19 mm, 25 mL/min, solvent: water with 0.1% formic acid-acetonitrile 60:40, 0-1 minute; 60:40→0:100, 1-12 minutes; 0:100, 12-15 minutes). The residue was dissolved in dichloromethane, washed once with saturated sodium hydrogen carbonate solution and three times with water, dried over magnesium sulphate and concentrated by evaporation. Diethyl ether and pentane were added to the residue and it was concentrated by evaporation. 80 mg (25% of theor.) of product was isolated.

¹H-NMR (400 MHz, chloroform-d₁): δ=1.05-1.31 (m, 8H), 1.70-1.80 (m, 2H), 1.90-2.00 (m, 4H), 2.04-2.22 (m, 6H), 2.32-2.40 (m, 4H), 2.57-2.66 (m, 4H), 2.96-3.02 (m, 4H), 3.48 (s, 2H), 6.72 (d, 1H), 6.75 (dd, 1H), 7.04 (tt, 2H), 7.15-7.23 (m, 3H).

### Example 142

### 2-Fluoro-8-(4-fluorophenyl)-9-{6-[(2-hydroxyethyl)(3-{[3,4,4,4-tetrafluoro-3-(trifluoromethyl)butyl]sulphonyl}propyl)amino]hexyl}-6,7-dihydro-5H-benzo[7]annulen-3-ol

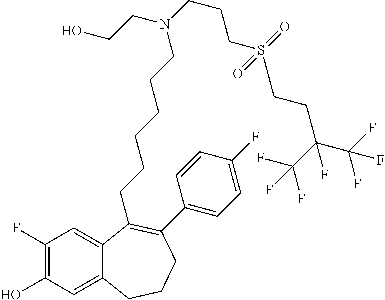 [SMILES: O=S(=O)(CCCN(CCO)CCCCCCC1=C(c2ccc(F)cc2)CCCc2cc(O)c(F)cc21)CCC(F)(C(F)(F)F)C(F)(F)F]

100 mg (0.23 mmol) of 9-(6-bromohexyl)-2-fluoro-8-(4-fluorophenyl)-6,7-dihydro-5H-benzo[7]annulen-3-ol was reacted with 100.1 mg (0.28 mmol) of 2-[(3-{[3,4,4,4-tetrafluoro-3-(trifluoromethyl)butyl]sulphonyl}propyl)amino]ethanol according to general specification 11. It was purified using HPLC-Method 1. 2.1 mg (1% of theor.) of product was isolated.

¹H-NMR (400 MHz, chloroform-d₁): δ=1.04-1.33 (m, 8H), 1.97-2.16 (m, 6H), 2.31 (t, 2H), 2.39 (m, 2H), 2.55-2.74 (m, 8H), 3.11 (t, 2H), 3.20 (m, 2H), 3.58 (t, 2H), 6.86 (d, 1H), 6.96-7.09 (m, 3H), 7.15-7.22 (m, 2H).

### Example 143

### 8-(2,5-Difluorophenyl)-9-[6-(methyl{4-[(4,4,4-trifluorobutyl)sulphonyl]butyl}amino)hexyl]-6,7-dihydro-5H-benzo[7]annulen-3-ol

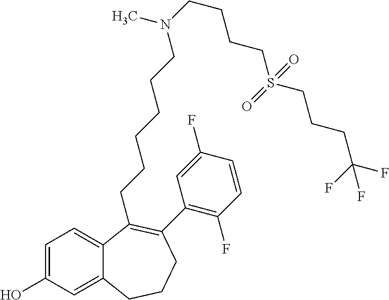 [SMILES: CN(CCCCCCC1=C(c2cc(F)ccc2F)CCCc2cc(O)ccc21)CCCCS(=O)(=O)CCCC(F)(F)F]

300 mg (0.69 mmol) of 9-(6-bromohexyl)-8-(2,5-difluorophenyl)-6,7-dihydro-5H-benzo[7]annulen-3-ol was reacted with 252.0 mg (0.96 mmol) of N-methyl-4-[(4,4,4-trifluorobutyl)sulphonyl]butan-1-amine according to general specification 11. It was purified using HPLC (XBridge C18, 5μ, 100×30 mm, 50 mL/min, solvent: water with 0.1% formic acid-acetonitrile 90:10, 0-1 minute; 90:10→20:80, 1-8 minutes; 20:80→0:100, 8-8.1 minutes; 0:100, 8.1-10 minutes). Then it was filtered on Silica Gel 60 (solvent: dichloromethane and dichloromethane-methanol 9:1). 21 mg (5% of theor.) of product was isolated.

¹H-NMR (400 MHz, chloroform-d₁): δ=1.00-1.38 (m, 8H), 1.77-1.97 (m, 4H), 2.03-2.21 (m, 6H), 2.27-2.40 (m, 4H), 2.44-2.52 (m, 5H), 2.64 (mc, 2H), 2.70 (mc, 2H), 3.03-3.12 (m, 4H), 6.74-6.80 (m, 2H), 6.86-6.97 (m, 2H), 7.04 (dt, 1H), 7.15 (d, 1H).

### Example 144

### 9-{6-[{4-[(4,4-Difluorocyclohexyl)sulphonyl]butyl}(methyl)amino]hexyl}-2-fluoro-8-(4-fluorophenyl)-6,7-dihydro-5H-benzo[7]annulen-3-ol

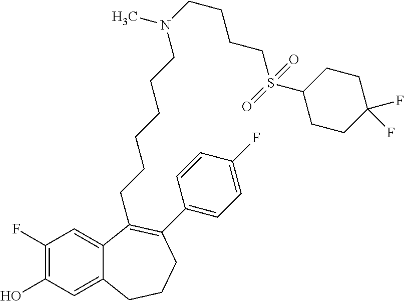 [SMILES: CN(CCCCCCC1=C(c2ccc(F)cc2)CCCc2cc(O)c(F)cc21)CCCCS(=O)(=O)C1CCC(F)(F)CC1]

536.4 mg (1.23 mmol) of 9-(6-bromohexyl)-2-fluoro-8-(4-fluorophenyl)-6,7-dihydro-5H-benzo[7]annulen-3-ol was reacted with 431.4 mg (1.60 mmol) of 4-[(4,4-difluorocyclohexyl)sulphonyl]-N-methylbutan-1-amine according to general specification 11. It was purified using HPLC (XBridge C18, 5μ, 50×30 mm, 54 mL/min, solvent: water with 0.1% formic acid-acetonitrile 60:40→30:70, 0-9 minutes). 321 mg (42% of theor.) of product was isolated.

¹H-NMR (400 MHz, chloroform-d₁): δ=1.03-1.23 (m, 6H), 1.24-1.38 (m, 2H), 1.56-1.67 (m, 2H), 1.69-2.13 (m, 10H), 2.15-2.40 (m, 13H), 2.53-2.62 (m, 2H), 2.85-3.03 (m, 3H), 6.80 (d, 1H), 6.96-7.09 (m, 3H), 7.15-7.22 (m, 2H).

### Example 145

### 8-(2,4-Difluorophenyl)-9-[6-(methyl{3-[(3,3,4,4,4-pentafluorobutyl)sulphonyl]propyl}amino)hexyl]-6,7-dihydro-5H-benzo[7]annulen-3-ol

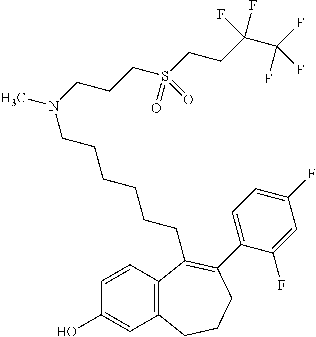 [SMILES: CN(CCCCCCC1=C(c2ccc(F)cc2F)CCCc2cc(O)ccc21)CCCS(=O)(=O)CCC(F)(F)C(F)(F)F]

Preparation was carried out similarly by reaction of 9-(6-bromohexyl)-8-(2,4-difluorophenyl)-6,7-dihydro-5H-benzo[7]annulen-3-ol with N-methyl-3-[(3,3,4,4,4-pentafluorobutyl)sulphonyl]propan-1-amine according to general specification 11.

¹H-NMR (300 MHz, DMSO-d₆): δ=0.97-1.15 (m, 6H), 1.17-1.25 (m, 2H), 1.76-1.84 (m, 2H), 1.93 (t, 1H), 2.01-2.07 (m, 2H), 2.07 (s, 3H), 2.13-2.17 (m, 2H), 2.20-2.23 (m, 2H), 2.32-2.36 (t, 2H), 2.53-2.71 (m, 4H), 3.19-3.23 (m, 2H), 3.42-3.46 (m, 2H), 6.66-6.68 (m, 2H), 7.07-7.12 (td, 1H), 7.14 (d, 1H), 7.23-7.26 (td, 1H), 7.28-7.33 (m, 1H).

### Example 146

### 8-(2,4-Difluorophenyl)-9-[6-(methyl{4-[(3,3,4,4,4-pentafluorobutyl)sulphonyl]-butyl}amino)hexyl]-6,7-dihydro-5H-benzo[7]annulen-3-ol

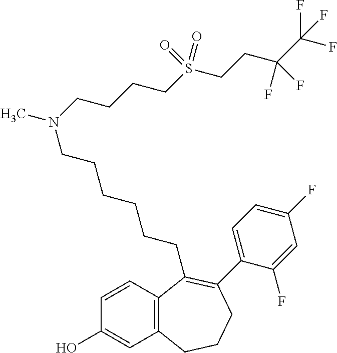 [SMILES: CN(CCCCCCC1=C(c2ccc(F)cc2F)CCCc2cc(O)ccc21)CCCCS(=O)(=O)CCC(F)(F)C(F)(F)F]

Preparation was carried out similarly by reaction of 9-(6-bromohexyl)-8-(2,4-difluorophenyl)-6,7-dihydro-5H-benzo[7]annulen-3-ol with N-methyl-4-[(3,3,4,4,4-pentafluorobutyl)sulphonyl]butan-1-amine according to general specification 11.

¹H-NMR (300 MHz, DMSO-d₆): δ=0.97-1.14 (m, 6H), 1.19-1.27 (m, 2H), 1.46-1.53 (m, 2H), 1.64-1.71 (m, 2H), 1.91-1.94 (m, 2H), 2.01-2.08 (m, 2H), 2.01 (s, 3H), 2.18-2.23 (m, 4H), 2.30-2.33 (m, 2H), 2.54-2.72 (m, 4H), 3.23-3.27 (m, 2H), 3.38-3.42 (m, 2H), 6.66-6.68 (m, 2H), 7.07-7.12 (td, 1H), 7.14 (d, 1H), 7.23-7.28 (td, 1H), 7.28-7.33 (m, 1H).

### Example 147

### 8-(2,4-Difluorophenyl)-9-[6-(methyl{4-[(4,4,5,5,5-pentafluoropentyl)sulphonyl]butyl}amino)hexyl]-6,7-dihydro-5H-benzo[7]annulen-3-ol

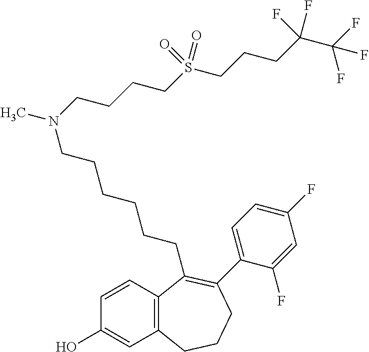 [SMILES: CN(CCCCCCC1=C(c2ccc(F)cc2F)CCCc2cc(O)ccc21)CCCCS(=O)(=O)CCCC(F)(F)C(F)(F)F]

Preparation was carried out similarly by reaction of 9-(6-bromohexyl)-8-(2,4-difluorophenyl)-6,7-dihydro-5H-benzo[7]annulen-3-ol with N-methyl-4-[(4,4,5,5,5-pentafluoropentyl)sulphonyl]butan-1-amine according to general specification 11.

¹H-NMR, 300 MHz, (DMSO-d₆): δ=0.96-1.15 (m, 6H), 1.19-1.26 (m, 2H), 1.46-1.53 (m, 2H), 1.61-1.69 (m, 2H), 1.89-1.96 (m, 4H), 2.01-2.07 (m, 2H), 2.09 (s, 3H), 2.16-2.23 (m, 4H), 2.27-2.46 (m, 4H), 2.55-2.58 (m, 2H), 3.10-3.14 (m, 2H), 3.18-3.22 (m, 2H), 6.66-6.68 (m, 2H), 7.07-7.12 (td, 1H), 7.14 (d, 1H), 7.23-7.28 (td, 1H), 7.27-7.33 (m, 1H).

### Example 148

### 8-(4-Fluorophenyl)-9-[6-(methyl{3-[(6,6,6-trifluorohexyl)sulphonyl]propyl}amino)hexyl]-6,7-dihydro-5H-benzo[7]annulen-3-ol

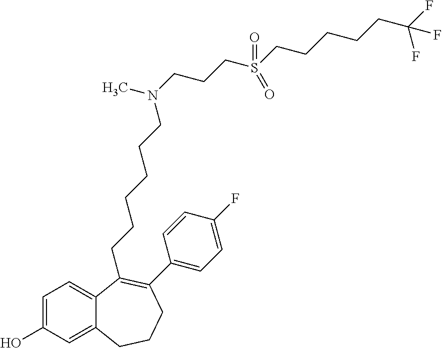 [SMILES: CN(CCCCCCC1=C(c2ccc(F)cc2)CCCc2cc(O)ccc21)CCCS(=O)(=O)CCCCCC(F)(F)F]

130 mg (0.31 mmol) of 9-(6-bromohexyl)-8-(4-fluorophenyl)-6,7-dihydro-5H-benzo[7]annulen-3-ol was reacted with 102.9 mg (0.37 mmol) of N-methyl-3-[(6,6,6-trifluorohexyl)sulphonyl]propan-1-amine according to general specification 11. It was purified using HPLC-Method 1. 75.3 mg (40% of theor.) of product was isolated.

¹H-NMR (400 MHz, chloroform-d₁): δ=1.00-1.24 (m, 6H), 1.32 (mc, 2H), 1.50-1.67 (m, 4H), 1.88 (mc, 2H), 2.04-2.20 (m, 8H), 2.33-2.44 (m, 7H), 2.61 (mc, 2H), 2.78 (t, 2H), 3.01 (mc, 2H), 3.09 (t, 2H), 6.74 (d, 1H), 6.77 (dd, 1H), 7.04 (tt, 2H), 7.14 (d, 1H), 7.16-7.22 (m, 2H).

### Example 149

### 4-Fluoro-8-(4-fluorophenyl)-9-[6-(methyl{3-[(4,4,4-trifluorobutyl)sulphonyl]propyl}amino)hexyl]-6,7-dihydro-5H-benzo[7]annulen-3-ol

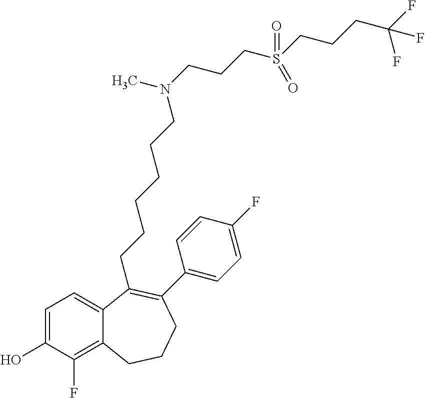 [SMILES: CN(CCCCCCC1=C(c2ccc(F)cc2)CCCc2c1ccc(O)c2F)CCCS(=O)(=O)CCCC(F)(F)F]

120 mg (0.28 mmol) of 9-(6-bromohexyl)-4-fluoro-8-(4-fluorophenyl)-6,7-dihydro-5H-benzo[7]annulen-3-ol was reacted with 98.3 mg (0.33 mmol) of N-methyl-3-[(4,4,4-trifluorobutyl)sulphonyl]propan-1-amine according to general specification 11. It was purified using HPLC-Method 1. 65.7 mg (40% of theor.) of product was isolated.

¹H-NMR (300 MHz, chloroform-d₁): δ=1.03-1.41 (m, 8H), 2.01-2.21 (m, 8H), 2.24-2.43 (m, 9H), 2.53-2.68 (m, 4H), 3.02-3.14 (m, 4H), 6.74 (d, 1H), 6.84 (d, 1H), 6.96-7.10 (m, 3H), 7.14-7.22 (m, 2H).

### Biological Examples

### Abbreviations and Acronyms

ER oestrogen receptor
E2 17β-estradiol
E1 estron
SERM selective oestrogen receptor modulator
d day
OVX ovariectomized animals
SHAM sham operation
n.d. not determined
Ex. example

### Example 150

### Effect on Stability of ERα Protein

As well as inhibition of the transcriptional activity of the ER, anti-oestrogens influence the expression level of the ER in the target tissues through stimulation of the proteolytic degradation of the ER. In comparison with an ER-E2 complex, the ER bound in a complex with the pure anti-oestrogen fulvestrant has a substantially shorter half-life. Conversely, the ER stability is enhanced by the SERM tamoxifen, so that overall there is ER stabilization. All things considered, it can be assumed that the capacity of pure anti-oestrogens and certain SERMs for inducing ER degradation contributes significantly to the overall action of the compounds. Compounds that have a destabilizing property, but at the same time display the desired tissue-specific agonistic qualities, e.g. bone protection, should on the whole present a superior pharmacological profile, as they have a lower potential for side-effects, such as stimulation of the endometrium.

The effect of the claimed pharmacological compounds on the stability of the ER was analysed in T47D breast cancer cells (see Table 1, column Standardized ER-destabilization [%]). These cells express the ER in functional form. The cells are incubated for 24 hours with the claimed compounds at a concentration of 1 μM. After lysis of the cells, the content of ER protein was determined by ELISA. Treatment with the complete destabilizer fulvestrant (0% ER), the stabilizer tamoxifen (100% ER) and the control medium (approx. 30% ER) was used as comparison. Compounds with ER content below 30% are classified as destabilizing.

As described, the claimed pharmacological substances were investigated for their action on the stability of the ERα protein (see Table 1). Over most of the claimed structural range, the pharmacological substances display a destabilizing action on the ERα content (remaining relative ERα content of less than or equal to 30%).

TABLE 1

| | Standardized ER- |
|---|---|
| | destabilization |
| Ex | [%] |
| 1 | 1 |
| 2 | n.d. |
| 3 | n.d. |
| 4 | 0 |
| 5 | 40 |
| 6 | n.d. |
| 7 | 12 |
| 8 | 5 |
| 9 | 0 |
| 10 | 18 |
| 11 | 17 |
| 12 | 0 |
| 13 | 4 |
| 14 | 43 |
| 15 | 2 |
| 16 | 4 |
| 17 | 2 |
| 18 | 12 |
| 19 | 17 |
| 20 | 0 |
| 21 | 54 |
| 22 | 51 |
| 23 | 12 |
| 24 | 2 |
| 25 | 0 |
| 26 | 23 |
| 27 | 16 |
| 28 | 2 |
| 29 | 5 |
| 30 | 55 |
| 31 | 0 |
| 32 | 5 |
| 33 | 0 |
| 34 | 11 |
| 35 | 5 |
| 36 | 42 |
| 37 | 56 |
| 38 | 17 |
| 39 | 3 |
| 40 | 3 |
| 41 | 3 |
| 42 | 10 |
| 43 | 4 |
| 44 | 5 |
| 45 | 4 |
| 46 | 7 |
| 47 | 5 |
| 48 | 3 |
| 49 | 8 |
| 50 | 11 |
| 51 | 11 |
| 52 | 30 |
| 53 | 45 |
| 54 | 0 |
| 55 | n.d. |
| 56 | 11 |
| 57 | 42 |
| 58 | 4 |
| 59 | 0 |
| 60 | 0 |
| 61 | 21 |
| 62 | 35 |
| 63 | 46 |
| 64 | 4 |
| 65 | 9 |
| 66 | n.d. |
| 67 | 8 |
| 68 | 32 |
| 69 | 0 |
| 70 | 8 |
| 71 | 24 |
| 72 | 2 |
| 73 | 4 |
| 74 | 1 |
| 75 | 0 |
| 76 | 20 |
| 77 | 10 |
| 78 | 2 |
| 79 | 0 |
| 80 | 6 |
| 81 | 2 |
| 82 | 1 |
| 83 | 6 |
| 84 | 15 |
| 85 | 2 |
| 86 | 1 |
| 87 | 5 |
| 88 | 1 |
| 89 | 19 |
| 90 | 16 |
| 91 | 23 |
| 92 | 0 |
| 93 | 0 |
| 94 | 4 |
| 95 | 0 |
| 96 | 3 |
| 97 | 7 |
| 98 | 4 |
| 99 | 0 |
| 100 | 0 |
| 101 | 3 |
| 102 | 4 |
| 103 | 2 |
| 104 | 2 |
| 105 | 0 |
| 106 | 8 |
| 107 | 21 |
| 108 | 4 |
| 109 | 3 |
| 110 | 6 |
| 111 | 2 |
| 112 | 4 |
| 113 | 0 |
| 114 | 0 |
| 115 | 2 |
| 116 | 19 |
| 117 | 32 |
| 118 | 6 |
| 119 | 11 |
| 120 | n.d. |
| 121 | 53 |
| 122 | 14 |
| 123 | 11 |
| 124 | 5 |
| 125 | 8 |
| 126 | 2 |
| 127 | 17 |
| 128 | 2 |
| 129 | 9 |
| 130 | 52 |
| 131 | 12 |
| 132 | 5 |
| 133 | 32 |
| 134 | 16 |
| 135 | 4 |
| 136 | 7 |
| 137 | 11 |
| 138 | 0 |
| 139 | 6 |
| 140 | n.d. |
| 141 | 7 |
| 142 | n.d. |
| 143 | n.d. |
| 144 | n.d. |
| 145 | n.d. |
| 146 | n.d. |
| 147 | n.d. |
| 148 | 0 |
| 149 | 13 |

### Example 151

### Anti-Oestrogenic Action in MVLN Cells

The anti-oestrogenic action of the claimed pharmacological compounds was investigated in so-called MVLN cells in vitro. MVLN cells are derivatives of the hormone-responsive MCF7 breast cancer cells known by a person skilled in the art. These MVLN cells express, along with the functional oestrogen receptor (ER), a reporter construct, which under ER-activation expresses luciferase. Determination of the activity of the induced luciferase permits a direct conclusion on the oestrogen properties of substances. To investigate the anti-oestrogenic properties of the pharmacological compounds, they were investigated in the presence of oestrogen for their potential for inhibiting the luciferase signal induced by estradiol.

The claimed pharmacological test substances were investigated in MVLN cells for their anti-oestrogenic potential, as described (see Table 2). Over the entire structural range, these compounds show high potency (IC50 values below 0.6 μM) and primarily even double-digit or single-digit nanomolar IC50 values for the inhibition of estradiol-induced luciferase activity.

TABLE 2

| | Antioestrogenic action in vitro: Ex. |
|---|---|
| | MVLN transactivation antagonism |
| | IC50 |
| Ex. | (nM) |
| 1 | 48 |
| 2 | 8 |
| 3 | 180 |
| 4 | 6 |
| 5 | 46 |
| 6 | 181 |
| 7 | 24 |
| 8 | 12 |
| 9 | 17 |
| 10 | 38 |
| 11 | 84 |
| 12 | 15 |
| 13 | 20 |
| 14 | 168 |
| 15 | 26 |
| 16 | 27 |
| 17 | 46 |
| 18 | 34 |
| 19 | 62 |
| 20 | 9 |
| 21 | 48 |
| 22 | 222 |
| 23 | 22 |
| 24 | 8 |
| 25 | 28 |
| 26 | 28 |
| 27 | 35 |
| 28 | 9 |
| 29 | 10 |
| 30 | 88 |
| 31 | 16 |
| 32 | 33 |
| 33 | 11 |
| 34 | 27 |
| 35 | 10 |
| 36 | 35 |
| 37 | 53 |
| 38 | 27 |
| 39 | 8 |
| 40 | 170 |
| 41 | 42 |
| 42 | 13 |
| 43 | 20 |
| 44 | 5.6* |
| 45 | 20 |
| 46 | 36 |
| 47 | 26 |
| 48 | 17 |
| 49 | 181 |
| 50 | 16 |
| 51 | 30 |
| 52 | 521 |
| 53 | 85 |
| 54 | 27 |
| 55 | 22 |
| 56 | 29 |
| 57 | 111 |
| 58 | 184 |
| 59 | 16 |
| 60 | 15 |
| 61 | 24 |
| 62 | 84 |
| 63 | 95 |
| 64 | 86 |
| 65 | 116 |
| 66 | 46 |
| 67 | 20 |
| 68 | 106 |
| 69 | 19 |
| 70 | 18 |
| 71 | 56 |
| 72 | 17 |
| 73 | 18 |
| 74 | 28 |
| 75 | 11 |
| 76 | 31 |
| 77 | 10 |
| 78 | 31 |
| 79 | 17 |
| 80 | 8 |
| 81 | 40 |
| 82 | 13 |
| 83 | 13 |
| 84 | 252 |
| 85 | 51 |
| 86 | 25 |
| 87 | 10 |
| 88 | 24 |
| 89 | 54 |
| 90 | 14 |
| 91 | 67 |
| 92 | 5 |
| 93 | 5 |
| 94 | 21 |
| 95 | 19 |
| 96 | 21 |
| 97 | 7 |
| 98 | 6 |
| 99 | 15 |
| 100 | 9 |
| 101 | 6 |
| 102 | 10 |
| 103 | 5 |
| 104 | 7 |
| 105 | 12 |
| 106 | 10 |
| 107 | 123 |
| 108 | 69 |
| 109 | 26 |
| 110 | 73 |
| 111 | 33 |
| 112 | 7 |
| 113 | 18 |
| 114 | 10 |
| 115 | 13 |
| 116 | 34 |
| 117 | 18 |
| 118 | 9 |
| 119 | 80 |
| 120 | 56 |
| 121 | 41 |
| 122 | 6 |
| 123 | 13 |
| 124 | 13 |
| 125 | 13 |
| 126 | 9 |
| 127 | 69 |
| 128 | 6 |
| 129 | 5 |
| 130 | 16 |
| 131 | 3 |
| 132 | 4 |
| 133 | 7 |
| 134 | 5 |
| 135 | 5 |
| 136 | 9 |
| 137 | 6 |
| 138 | 21 |
| 139 | 7 |
| 140 | 4 |
| 141 | 21 |
| 142 | |
| 143 | 9 |
| 144 | 10 |
| 145 | 22 |
| 146 | 15 |
| 147 | 32 |
| 148 | 17 |
| 149 | 14 |

*mean value

The suitability of the compounds according to the invention for the treatment of endometriosis can be demonstrated in the following animal models. The influence of the compounds according to the invention on the uterus was investigated in the uterus growth test (oestrogenic action) and in the anti-uterus growth test (anti-oestrogenic action), both conducted in the rat.

### Example 152

### Oestrogenic Action—Uterus Growth Test in the Infantile Rat

Both the uterus and the vagina show a weight increase that is dependent on oestrogenic efficacy, in infantile animals when they are treated with a substance with oestrogenic action. In the uterus, under oestrogenic action there is also proliferation and increase in height of the luminal epithelium.

Immature, intact rats (n=5-6 animals/group; body weight 40-50 g) receive the substance s.c. for 3 days (d1-d3). On day 4 (d4) the animals are killed with CO₂. The uteri are removed and weighed. For histological assessment, a piece of uterus, preferably a uterine horn, is fixed in formaldehyde and embedded in paraffin. The stimulation of the organ weights (relative to mg/100 g body weight) and the height of the epithelium are shown as percentage stimulation in comparison with the reference compound 17β-estradiol (E2). (Substitution dose of E2 0.3 μg/animal).

The compounds according to the invention have no or only slight stimulating action on the uterus.

The selected claimed substances were investigated in juvenile female rats as described for their oestrogenic, stimulating action on uterus weight. They show a slight to marginal oestrogenic action in vivo (Table 3).

TABLE 3

| | maximum uterotropic action (% estradiol action) in | |
|---|---|---|
| Example | the dose range from 0.03 to 3 mg/kg | |
| Raloxifene | 18% | [dose 0.03 mg/kg] |
| 44 | 6% | [dose 0.1 mg/kg] |
| 51 | 22% | [dose 0.1 mg/kg] |
| 115 | 3% | [dose 0.03 mg/kg] |
| 117 | 18% | [dose 0.3 mg/kg] |
| 118 | 8% | [dose 0.03 mg/kg] |
| 122 | 7% | [dose 0.3 mg/kg] |
| 123 | 8% | [dose 0.3 mg/kg] |

### Example 153

### Anti-Uterus Growth Test in the Adult Rat

The uterus of oestrogen-substituted rats can be used as a test model for detecting a direct action of substances with anti-oestrogenic properties. The parameter of oestrogenic action is estradiol-induced uterus growth in rats, which is inhibited by simultaneous administration of a substance with anti-oestrogenic action.

The experimental animals (n=5-6 animals/group) were ovariectomized before the start of the test, to rule out the influence of endogenous oestrogens. After a phase of 6 to 10 days, the test substances are administered s.c. on 3 consecutive days (d1-d3) in combination with a substitution dose of 1.5 μg/kg/day 17β-estradiol. 17β-estradiol alone serves as positive control, and the excipients serve as negative control. On day 4 (d4) the animals are killed, and the uteri and vaginae are removed and weighed. The organ weights are converted to mg/100 g body weight, then the mean value and the standard deviation are calculated for each dosage. The inhibition of uterus growth induced by 17β-estradiol is shown as percentage inhibition.

The compounds according to the invention largely show very pronounced inhibition of uterus growth induced by 17β-estradiol.

The compounds according to the invention are therefore superior in the sense of the present invention, with respect to their action on the uterus, to the compounds of the prior art, in that they have less or even no oestrogenic action on this organ.

The selected claimed substances were investigated in adult female rats as described for their anti-oestrogenic, inhibitory action on the weight of the uterus. At the dosage used, the substances show a definite anti-oestrogenic action in vivo (Table 4).

TABLE 4

| | anti-oestrogenic activity in vivo in % |
|---|---|
| Example | at a dose of 0.3 mg/kg (rat) |
| 7 | 23 |
| 23 | 33 |
| 27 | 10 |
| 44 | 37* |
| 45 | 73 |
| 46 | 30 |
| 48 | 79 |
| 51 | 59 |
| 80 | 59 |
| 114 | 49 |
| 115 | 50 |
| 116 | 15 |
| 117 | 65 |
| 118 | 78 |
| 122 | 52 |
| 123 | 56 |
| 124 | 67 |
| 125 | 75 |

*mean value; the value of 60% given in the priority application had to be corrected to 40% as a result of an error during evaluation. Another measurement gave a value of 34%. Accordingly, the mean of both measurements is 37%.

### Example 154

### Hepatic Oestrogenicity in the Ovariectomized Adult Rat

Substances with oestrogenic action influence the synthesis of various plasma proteins, of coagulation factors and fibrinolytic factors in the liver. This hepatic oestrogenicity is discussed as a causal factor with respect to the slightly increased thromboembolic risk that is observed in some forms of oestrogen therapy. In the present investigations, lowering of the peripheral cholesterol level is used as a surrogate parameter for analysis of the hepatic oestrogenicity of the claimed pharmacological compounds. Adult ovariectomized rats were, after a pause of 6-10 days, treated daily for 6 days with the substances by subcutaneous application. The plasma cholesterol levels were determined and compared before and after the respective treatment.

Compared with the SERM raloxifene, the selected claimed pharmacological compounds show a reduced lowering of the peripheral cholesterol level (i.e. lowering only occurring at higher dosages) and therefore also lower hepatic oestrogenicity.

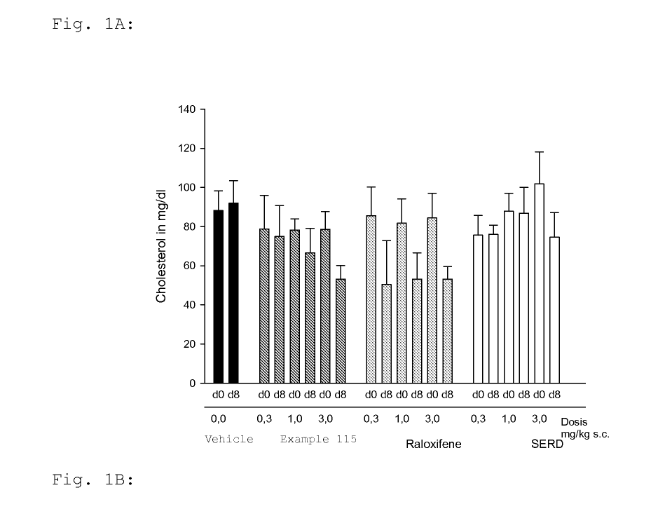

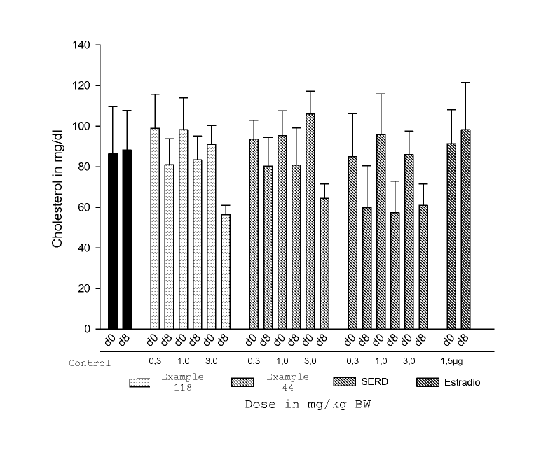

The selected claimed pharmacological substances were investigated as described in ovariectomized female rats for their oestrogenic action on the hepatic parameter cholesterol. As can be seen from FIG. 1A and FIG. 1B, the compounds show only a lowering of the peripheral cholesterol action at the higher dosages (which corresponds to slight hepatic action), in contrast to the control compound raloxifene, which demonstrates hepatic oestrogenicity at all dosages tested. As expected, the pure anti-oestrogen SERD shows no hepatic oestrogenicity.

FIG. 1A

### Example 155

### Stimulation of Ovarian Oestrogen Synthesis

The clinical use both of pure anti-oestrogens and of various SERMs for the treatment of premenopausal women is limited by their property of stimulating the ovaries through activation of the hypothalamic-pituitary-gonadal axis (HPG axis), which leads to the increase in peripheral estradiol levels (Palomba, S., Orio, F., Jr., Morelli, M., Russo, T., Pellicano, M., Zupi, E., Lombardi, G., Nappi, C., Panici, P. L., and Zullo, F. (2002). Raloxifene administration in premenopausal women with uterine leiomyomas: a pilot study. J Clin Endocrinol Metab 87, 3603-3608). This stimulation of the HPG axis is associated with penetration of the blood-brain barrier and penetration of the brain. To investigate the ovary-stimulating properties of the claimed pharmacological compounds, hormonally intact adult rats were treated with the substances daily for a period of 10 days. The study end point is the quotient of peripheral estradiol values after and before treatment.

In comparison with pure anti-oestrogens and the classical SERMs such as raloxifene or bazedoxifene, the selected claimed pharmacological compounds show markedly less stimulation of the HPG axis at equal dosage. They therefore display superior properties for clinical use in premenopausal women.

Selected claimed pharmacological substances were investigated as described for their stimulating action on the HPG axis or ovarian estradiol synthesis. The selected substances show markedly less stimulation of the ovaries than the control compound raloxifene at equal dosages (cf. Table 5).

TABLE 5

| | Stimulation of ovarian oestrogen synthesis: |
|---|---|
| Example | factor at a dose of 3 mg/kg rat |
| 80 | 2.3 |
| 44 | 1.2 |
| 114 | 1.4 |
| 115 | 2.7 |
| 117 | 2.1 |
| 118 | 2.3 |
| 122 | 2.4 |
| 123 | 2.5 |
| 124 | 2.0 |
| Raloxifene | 3.1 |
| hydrochloride | |

### Example 156

### Determination of Activity in the Endometriosis Model in the Rat

Using intact adult female rats, experimental endometriosis was induced in the autologous transplantation model based on Vernon M. W. and Wilson E. A., 1985 (Fertil Steril. 44(5):684-694). A uterine horn was removed from animals that were exclusively in oestrus, the myometrium was separated from the endometrium and biopsies with dimensions of 4×4 mm were obtained from the endometrial tissue. Two uterine fragments were transplanted onto the inside of the abdominal wall (peritoneum) and two uterine fragments were transplanted onto the mesenterium of the same animal (4 fragments per animal). After 21 days the animals with endometriosis were laparotomized, and the size of the transplants was determined. The animals were treated after laparotomy and then daily in the morning with the stated dosages of the selected claimed substances by subcutaneous administration. Finally, 28 days after the start of treatment (49 days after transplantation), all the animals were laparotomized again and the size of the lesion was measured and was correlated with the size before the start of treatment.

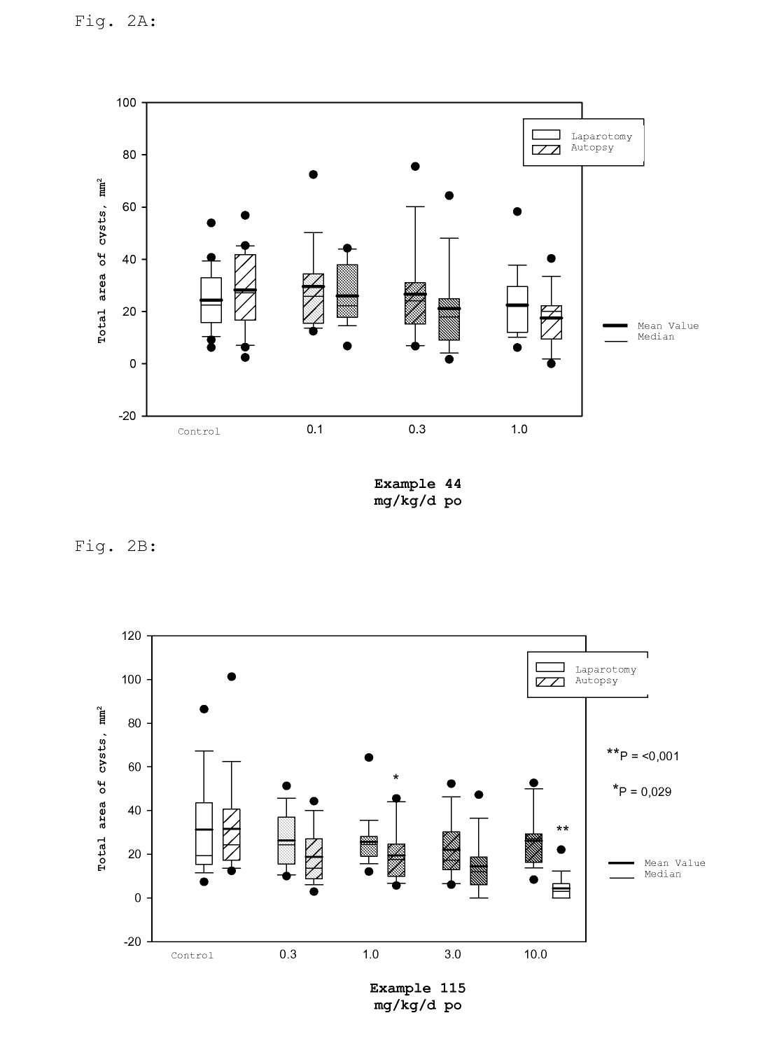

Treatment with the selected claimed pharmacological substances (FIG. 2A: compound according to example 115; FIG. 2B: compound according to example 44) shows a significant dose-dependent reduction of lesion size over the treatment period of 4 weeks. FIG. 2C: compound according to example 44 shows a significant dose-dependent reduction of lesion sizes in an independent experiment according to identical experimental design. FIG. 2D: in the experimental animals tested (from FIG. 2C), application of example compound 44 in the dose range used does not lead to an increase in peripheral estradiol level above the physiological range.

### Example 156

### Investigation of the Bone-Protective Properties

3-months-old female rats are ovariectomized and immediately after the operation they are treated with the test compound once daily for 56 days. Application is oral in peanut oil/ethanol. The animals are killed on the day after the last application and the tibia and the uteri are removed. The uteri are weighed, fixed and prepared for histological investigations. Bone density is determined ex vivo on prepared long bones by pQCT (peripheral quantitative computed tomography).

Ovariectomy leads to a decrease in density of the trabecular bone in the measured region. By treatment with a compound of general formula I according to the present invention (dosages of 1-10 mg/kg/day) the decline in bone density is prevented or inhibited. Bone density was measured on the proximal tibia.

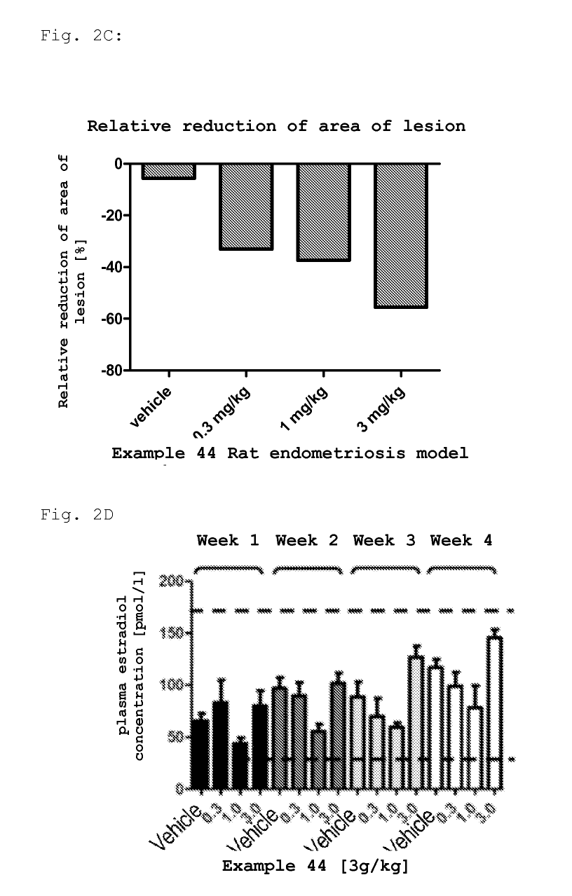

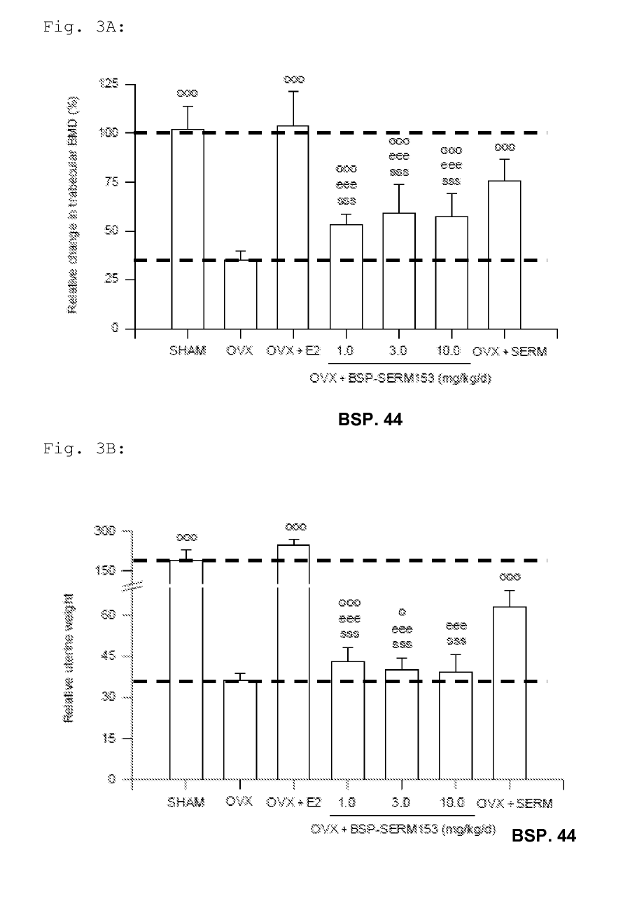

The bone-protective action in adult ovariectomized female animals (rat) was investigated as described. The control groups comprised a group of animals with sham operations (ovaries not removed), ovariectomized animals (which have a marked loss of uterus weight and bone density), animals treated with estradiol (no loss of bone mass, marked stimulation of uterus weight), and animals that were treated with the SERM raloxifene (marked bone protective action, marked stimulation of uterus weight). The selected claimed pharmacological example compound 44 was applied orally at dosages from 1-10 mg/kg. Clear protection of bone mass can be seen at all dosages (FIG. 3A). In contrast to estradiol or the SERM raloxifene, however, the selected compound 44 shows only a marginal and greatly reduced stimulation of uterus weight (FIG. 3B).

### Example 156a

### Investigation of Antagonistic Action on Bone Mass and Uterus Weight

To investigate the potential antagonistic action on bone mass in comparison with the effect on uterus weight, adult, hormonally intact Sprague-Dawley rats were treated with dosages between 1 and 10 mg/kg for a period of 2 months. Application was oral, in peanut oil/ethanol. The animals were killed on the day after the last application and the tibia and uteri were removed. The uteri were weighed, fixed and prepared for optional histological investigations. Bone density was determined on long bones by pQCT (quantitative computed tomography), once before the treatment, once on the day of autopsy. The variations for this parameter were shown correspondingly between these two reference points (values below 100 correspond to a decrease in bone mass density, values above 100 to an increase). The control group was a group of animals from which the ovaries had been removed (OVX) (due to ovariectomy, as expected there is a decrease in density of the trabecular bone in the measured range over the period of the experiment). All other animals underwent a SHAM-OP (a sham operation) (without the ovaries being removed) in addition to the application treatment. The changes in relative uterus weights served as the reference for the anti-oestrogenic action.

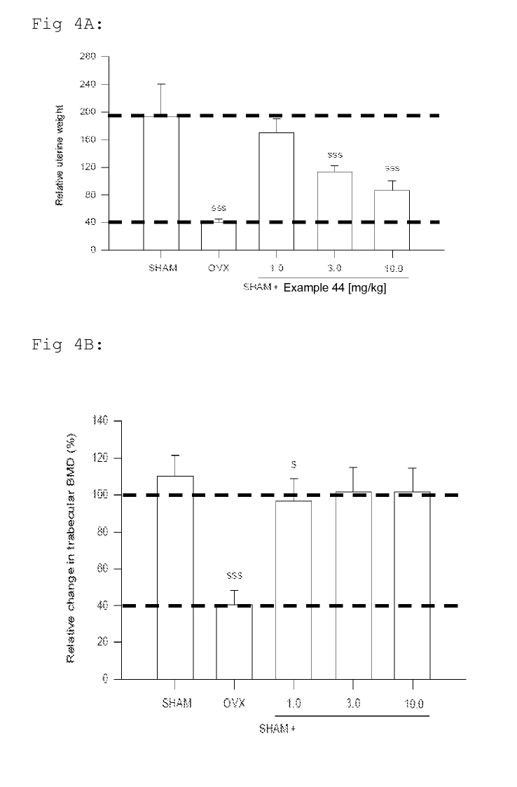

As can be seen from FIG. 4A, ovariectomy of the animals leads to a decrease in relative uterus weight. Example compound 44 shows a dose-dependent anti-oestrogenic action on the uterus.

FIG. 4B shows that, parallel to the decrease in uterus weight (FIG. 4A), the trabecular bone mass density of the tibia is, surprisingly, not reduced over the treatment period, as is the case for example with the ovariectomized animals (OVX). Accordingly, example compound 44 shows dissociation between antagonism on the uterus and antagonism on bone mass.

### Example 156b

### Investigation of the Action on the Mammary Gland in the Juvenile Rat

The formation of secretory units in the mamma is dependent in particular on gestagens and oestrogens. Juvenile female rats have been found to be especially sensitive in such experiments. To investigate the stimulating action of test compounds, animals are ovariectomized at the age of 21 days and after a treatment-free interval of 6 days treated either with a combination of the test compound and an oestrogen (for example 70 μg/kg E1) or a combination of the test compound and a gestagen (for example promegestone 0.3 mg/kg) in each case for a period of 6 days. To investigate the antagonistic potential of a test compound, the test compound is given for a period of 6 days together with an oestrogen (see above) and a gestagen (see above). Finally one of the abdominal-inguinal mammary glands is prepared and submitted to so-called whole-mount staining. The number of secretory units on an area of approx. 1.0 mm² serves as the end point (moreover, this can also vary according to requirements).

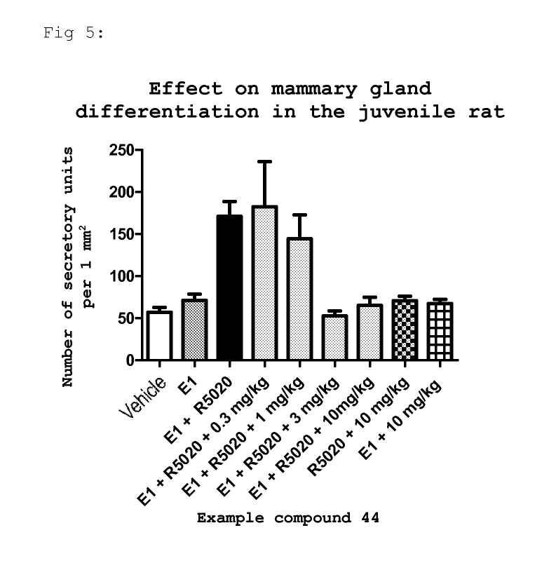

As is clear from FIG. 5, the combined administration of oestrogen E1 and gestagen promegestone (R5020) induces the formation of secretory units. Example compound 44 causes dose-dependent inhibition of said formation in the selected dose range. When example compound 44 is given either alone with gestagen R5020 or alone with oestrogen E1, there is no induction of mammary gland differentiation significantly different from the group given excipients. To summarize, these results demonstrate that example compound 44 has a dose-dependent antagonistic action on mammary gland differentiation and does not exert any agonistic, stimulating potential on this organ in the rat.

### Example 157

### Bioavailability in the Rat

Determination of bioavailability after intragastric application of test substances was carried out in conscious female rats with a body weight from min. 0.2 kg to max. 0.25 kg. For this, the test substances were applied in dissolved form both for intravenous, and for intragastric application, wherein compatible solubilizers such as PEG400 and/or ethanol were used in a compatible amount.

a) Intravenous Application:

The test substances were applied at a dose of 0.5-1 mg/kg as quick infusion taking 15 minutes. At the time points 2 min, 8 min, 15 min (infusion) and 5 min, 15 min, 30 min, 45 min, 1 h, 2 h, 4 h, 6 h, 8 h, 12 h, 16 h, 20 h, 24 h after the end of infusion, approx. 150 μL blood samples were taken via a catheter from the jugular vein. Lithium-heparin was added as anticoagulant to the blood samples and they were stored in a refrigerator until required for further processing. After centrifugation of the samples for 15 min at 3000 rpm, an aliquot of 100 μL was taken from the supernatant (plasma) and was precipitated by adding 400 μL of cold ACN or methanol (absolute). The precipitated samples were frozen-out overnight at −20° C., then centrifuged once again for 15 min at 3000 rpm, before taking 150 μL of the clear supernatant for determination of concentration. Analysis used an Agilent 1200 HPLC system coupled to LCMS/MS detection.

Calculation of the PK parameters (using PK calculation software, e.g. WinNonLin®): CLₚₗₐₛₘₐ total plasma clearance of the test substance (in L/kg/h); CL_{blood}: total blood clearance of the test substance (in L/kg/h), where (CL_{blood}=CLₚₗₐₛₘₐ*Cₚ/C_{b}); Vₛₛ: apparent steady-state distribution volume (in L/kg); t_{1/2}: half-life within a specified interval (here: terminal t_{1/2}, in h); AUCₙₒᵣₘ: area under the plasma concentration time profile from time point zero extrapolated to infinity divided by the dose normalized for body weight (in h*kg/L); AUC₍₀₋ₜₙ₎ₙₒᵣₘ: integrated area under the plasma concentration time profile from time point zero until the last time point at which a plasma concentration was measurable, divided by the dose normalized for body weight (in h*kg/L); Cₘₐₓ: maximum concentration of the test substance in the plasma (in μg/L); C_{max,norm}: maximum concentration of the test substance in the plasma divided by the dose normalized for body weight (in kg/L); C_{b}/Cₚ: ratio of blood to plasma concentration distribution.

b) Intragastric Application:

The test substances were applied to fasting female rats at a dose of 2-5 mg/kg intragastrically as a bolus using a feeding tube. At time points 8 min, 15 min, 30 min, 45 min, 1 h, 2 h, 4 h, 6 h, 8 h, 12 h, 16 h, 20 h, 24 h, approx. 150 μL blood samples were taken via a catheter from the jugular vein. Lithium-heparin was added as anticoagulant to the blood samples and they were stored in a refrigerator until required for further processing. After centrifuging the samples for 15 min at 3000 rpm, an aliquot of 100 μL was taken from the supernatant (plasma) and was precipitated by adding 400 μL of cold ACN or methanol (absolute). The precipitated samples were frozen-out overnight at −20° C., then centrifuged for 15 min at 3000 rpm before 150 μL of the clear supernatant was drawn off for determination of concentration. Analysis was carried out using an Agilent 1200 HPLC system coupled to LCMS/MS detection.

Calculation of the PK parameters (using PK calculation software, e.g. WinNonLin®):

AUCₙₒᵣₘ: area under the plasma concentration time profile from time point zero extrapolated to infinity divided by the dose normalized for body weight (in h*kg/L);

AUC₍₀₋ₜₙ₎ₙₒᵣₘ: integrated area under the plasma concentration time profile from time point zero until the last time point at which a plasma concentration was measurable, divided by the dose normalized for body weight (in h*kg/L); Cₘₐₓ: maximum concentration of the test substance in the plasma (in μg/L); C_{max,norm}: maximum concentration of the test substance in the plasma divided by the dose normalized for body weight (in kg/L); t_{1/2}: half-life within a specified interval (here: terminal t_{1/2}, in h); F_{obs%}: observed oral bioavailability, AUC₍₀₋ₜₙ₎ₙₒᵣₘ after i.g. administration divided by AUC₍₀₋ₜₙ₎ₙₒᵣₘ after i.v. administration. Tₘₐₓ: time point at which the maximum concentration of the test substance is measured in the plasma.

### Examples of Pharmaceutical Compositions

The compounds according to the invention can be transformed as follows into pharmaceutical preparations. The claimed compounds can be administered as a tablet. A possible composition for such a tablet can have the following appearance:

### Tablet:

### Composition:

100 mg of the compound of example 1, 50 mg lactose (monohydrate), 50 mg maize starch (native), 10 mg polyvinylpyrrolidone (PVP 25) (from BASF, Ludwigshafen, Germany) and 2 mg magnesium stearate.

Tablet weight 212 mg. Diameter 8 mm, radius of convexity 12 mm.

### Production:

The mixture of compound according to the invention, lactose and starch is granulated with a 5% solution (w/w) of the PVP in water. After drying, the granules are mixed with the magnesium stearate for 5 minutes. This mixture is compacted with a usual tablet press (see above for tablet format). A compressing force of 15 kN is used as a guide value for compaction.

Recipe, ingredients, amount of substance and manner of preparation can deviate from this.

The claimed compounds can also be administered as suspension for oral application. A possible composition for such a suspension can have the following appearance:

### Suspension for Oral Application

### Composition:

1000 mg of the compound of example 1, 1000 mg ethanol (96%), 400 mg Rhodigel® (xanthan gum from the company FMC, Pennsylvania, USA) and 99 g water.

An individual dose of 100 mg of the compound according to the invention is equivalent to 10 ml of oral suspension.

### Production:

The Rhodigel is suspended in ethanol, and the compound according to the invention is added to the suspension. The water is added with stirring. It is stirred for approx. 6 h until the Rhodigel ceases to swell.

Recipe, ingredients, amount of substance and manner of preparation can deviate from this.

The claimed compounds can also be administered as solution for oral application. A possible composition for such a solution can have the following appearance:

### Solution for Oral Application:

### Composition:

500 mg of the compound of example 1, 2.5 g polysorbate and 97 g polyethylene glycol 400. An individual dose of 100 mg of the compound according to the invention is equivalent to 20 g of oral solution.

### Production:

The compound according to the invention is suspended in the mixture of polyethylene glycol and polysorbate with stirring. Stirring is continued until the compound according to the invention has dissolved completely. Recipe, ingredients, amount of substance and manner of preparation can deviate from this.

### Illustrations

FIG. 1A: Determination of the hepatic oestrogenicity of example compound 115 in comparison with the SERM raloxifene and a pure anti-oestrogen (SERD). In each case, the cholesterol levels on day 0 (before the treatment) are shown in comparison with the cholesterol levels on day 8 (after ending the treatments). Compared with raloxifene, which induces a marked lowering of the cholesterol level at all dosages, in the case of example compound 115 this is only observed at high dosage.

FIG. 1B: Determination of the hepatic oestrogenicity of example compound 44 in comparison with the SERM raloxifene. In each case the cholesterol levels on day 0 (before the treatment) are shown in comparison with the cholesterol levels on day 8 (after ending the treatments). Compared with raloxifene, which induces a marked lowering of the cholesterol level at all dosages, in the case of example compounds 44 and 118 this is only observed at high dosage.

FIG. 2A: Testing of the compound from example 115 in the rat endometriosis model at dosages from 0.1 mg/kg to 1 mg/kg. It shows the average sizes of lesions per animal before the start of treatment (in each case boxplot on left) and the average sizes of lesions after 28 days of treatment (in each case boxplot on right). At a dosage of 1 mg/kg there is, in comparison with the excipients group, a significant reduction in sizes of lesions as a result of the treatment.

FIG. 2B: Testing of the compound from example 44 in the rat endometriosis model at dosages from 0.3 mg/kg to 10 mg/kg. It shows the average sizes of lesions per animal before the start of treatment (in each case boxplot on left) and the average sizes of lesions after 28 days of treatment (in each case boxplot on right). Starting from a dosage of 1 mg/kg, a significant reduction in size of lesion can be seen, on comparing the sizes of the lesions before and after the treatment.

FIG. 2C: Testing of the compound from example 44 in the rat endometriosis model at dosages from 0.3 mg/kg to 13 mg/kg in an independent experiment for example 2C. It shows the relative change in sizes of lesions per treatment group, comparison before and after treatment (all three dosages lead to a significant reduction of the lesion).

FIG. 2D: Estradiol level of the treated animals from the experiment shown in 2C. It shows the blood estradiol level of the respective dose groups sorted according to weeks. The dotted line represents the estradiol level described during oestrus of the rat. None of the groups treated with example compound 44 in the stated dosages shows levels above or below the naturally occurring estradiol levels (characterized by the dashed lines).

FIG. 3A: Bone-protective action (trabecular bone mass density in the distal tibia). Example compound 44 shows, in comparison with the ovariectomized animals, a marked conservation of bone mass, already beginning with the 1 mg/kg dosage. o=statistically significant difference from OVX control, e=statistically significant difference from OVX+E2, s=statistically significant difference from OVX+SERM (raloxifene)

FIG. 3B: Effect on uterus weight. In the tested dosages, example compound 44 shows, in comparison with estradiol and the control SERM, only a marginal uterotropic action. o=statistically significant difference from the OVX control, e=statistically significant difference from OVX+E2, s=statistically significant difference from OVX+SERM (raloxifene).

FIG. 4A: Effect on uterus weight in adult, hormonally intact female rats in long-term application. In the doses tested, example compound 44 showed a dose-dependent decrease after oral application. In comparison with the SHAM control (sham operation without removal of ovaries), this decrease is statistically significant (indicated with “sss”) at dosages of 3 mg/kg and 10 mg/kg. The ovariectomized animals (OVX) show a significant decrease in uterus weight, as was expected. The dashed lines show the relative uterus weight in the SHAM control group (top) and the relative uterus weight after ovariectomy (OVX) (bottom).

FIG. 4B: Effect on trabecular bone mass density in the distal tibia in the animals from FIG. 4A after treatment with example compound 44 for a period of 2 months. It shows the relative change in bone mass density during the period of the experiment. 100% corresponds to no increase or decrease in bone mass density, values below 100% correspond to a decrease, values above 100% correspond to an increase in this parameter. Ovariectomized animals show, after 2 months, a decrease in bone mass density, as was expected (significant in comparison with the SHAM control (indicated by “sss”). Surprisingly, example compound 44 does not show a significant decrease in bone mass density at any of the tested dosages (at 1 mg/kg there is a small significant difference from the SHAM control, due to the fact that in the SHAM group there is a slight increase in bone mass during the period of the experiment). The dashed lines clarify the decrease in bone mass density through ovariectomy (bottom) or the maintenance of bone mass at 100% (top).

FIG. 5: Effect on mammary gland differentiation in the juvenile rat. The number of mammary secretory units per square millimetre is shown as the end point. The treatment of juvenile, ovariectomized rats with the oestrogen E1 and the gestagen R5020 leads to induction of mammary gland differentiation (compare vehicle with E1+R5020). The combined administration of E1 and R5020 and increasing dosages of the example compound 44 can cause a dose-dependent reduction of this effect (compare E1+R5020 with the grey-shaded bars directly adjacent). The administration of example compound 44 either alone with the gestagen R5020 or alone with the oestrogen did not show any agonistic, inducing potential (compare vehicle with the two bars far right).

1. Compound of general formula (I), 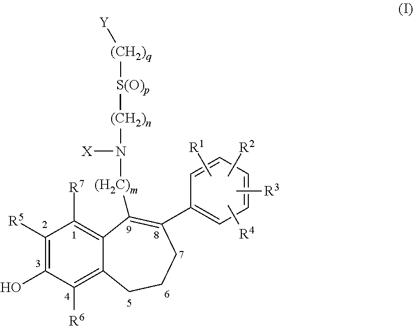
in which
R¹, R², R³ and R⁴ independently of one another stand for hydrogen or fluorine, wherein at least one substituent selected from R¹, R², R³ and R⁴ stands for fluorine,
R⁵, R⁶ and R⁷ independently of one another stand for hydrogen, fluorine, chlorine, bromine, methyl, ethyl, trifluoromethyl or nitrile is selected from the group comprising H, C₁-C₆-alkyl-, C₃-C₈-cycloalkyl-, C₂-C₆-alkenyl, C₂-C₆-alkynyl, C₁-C₆-alkyl-S(O)₂—, C₁-C₆-alkylcarbonyl-, phenyl-C₁-C₆-alkyl-, which optionally can be substituted once, twice or multiply with —OH, halogen, —CN, —NR⁸R⁹, —C(O)NR¹⁹R¹¹, —N(R¹⁹)C(O)NR¹⁹R¹¹, —C₁-C₆-haloalkoxy, —C₁-C₆-alkoxy, —C(O)OH, —C(O)OC₁-C₆-alkyl or —C(O)OBenzyl, and optionally hydrogen atoms can also be replaced with deuterium atoms,
R⁸ and R⁹ stand for C₁-C₆-alkyl, C₃-C₇-cycloalkyl, phenyl or benzyl, optionally substituted with halogen or deuterium,
R¹⁰ and R¹¹ stand for hydrogen or C₁-C₆-alkyl, C₃-C₇-cycloalkyl, phenyl or benzyl optionally substituted with halogen or deuterium,
Y stands for a per- or partially-fluorinated-C₁-C₄-alkyl or per- or partially-fluorinated C₃-C₈-cycloalkyl,
m stands for 4, 5, 6 or 7,
n stands for 2, 3, 4, 5 or 6,
P stands for 0, 1 or 2,
q stands for 0, 1, 2, 3, 4, 5 or 6
and their salts, solvates or solvates of the salts, including all crystal modifications.

2. Compound according to claim 1, in which
R¹, R², R³, R⁴, R⁵, R⁶ or R⁷ independently of one another stand for hydrogen or fluorine, wherein at least one substituent 1V, R², R³ and R⁴ stands for fluorine.
X is selected from the group comprising hydrogen, C₁-C₆-alkyl-, C₃-C₈-cycloalkyl-, C₁-C₆-alkyl-S(O)₂—, C₁-C₆-alkylcarbonyl-, phenyl-C₁-C₆-alkyl-, which optionally can be substituted once, twice or multiply with —OH, halogen, deuterium, —CN, —NR⁸R⁹, —C(O)NR¹⁰R¹¹, —N(R¹⁰)C(O)NR¹¹, alkoxy, —C(O)OH, —C(O)OC₁-C₆-alkyl or —C(O)OBenzyl,
R⁸ and R⁹ stand for C₁-C₆-alkyl or benzyl,
R¹⁰ and R¹¹ stand for hydrogen, C₁-C₆-alkyl or benzyl,
Y stands for —CF₃, —C₂F₅, —C₃F₇, —C₄F₉ or —C₃-C₇-cycloalkyl with 2-4 fluorine atoms,
m stands for 4, 5 or 6,
n stands for 2, 3, 4, 5 or 6,
P stands for 0, 1 or 2,
q stands for 0, 1, 2, 3, 4, 5 or 6
and their salts, solvates or solvates of the salts, including all crystal modifications.

3. Compound according to claim 2, characterized in that
R³, R², R³, R⁴ independently of one another stand for hydrogen or fluorine, wherein at least one and at most two fluorine atoms should be contained,
R⁵ and R⁶ independently of one another stand for hydrogen or fluorine,
R⁷ stands for hydrogen,
X is selected from the group comprising hydrogen, —C₁-C₄-alkyl, cyclopropyl-, which can optionally be substituted singly with —OH, —CN, methoxy, —C(O)OH, —C(O)OCH₃ or —C(O)OBenzyl or singly or multiply with —F or deuterium, or X is selected from methyl-S(O)₂— or methylcarbonyl-
Y stands for —CF₃, —C₂F₅, —CF₂CF₂CF₃, —CF(CF₃)₂ or 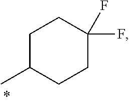
m stands for 5 or 6,
n stands for 3, 4 or 5,
P stands for 0, 1 or 2,
q stands for 0, 1, 2, 3, 4 or 5
and their salts, solvates or solvates of the salts, including all crystal modifications.

4. Compound according to claim 3, characterized in that
R¹, R², R³ and R⁴ independently of one another stand for hydrogen or fluorine, wherein at least one and at most two fluorine atoms should be contained,
R⁵ and R⁶ independently of one another stand for hydrogen or fluorine, with the restriction that R⁵ and R⁶ do not mean fluorine simultaneously,
X stands for C₁-C₄-alkyl-, optionally substituted with deuterium,
Y stands for —CF₃, —C₂F₅, 4,4-difluorocyclohexyl,
m stands for 5 or 6,
n stands for 3 or 4,
P stands for 1 or 2,
q stands for 2, 3, 4 or 5
or in the special case in which Y stands for 4,4-difluorocyclohexyl,
q stands for 0 or 1,
and their salts, solvates or solvates of the salts, including all crystal modifications.

5. Compounds according to claim 4 of formula (II) 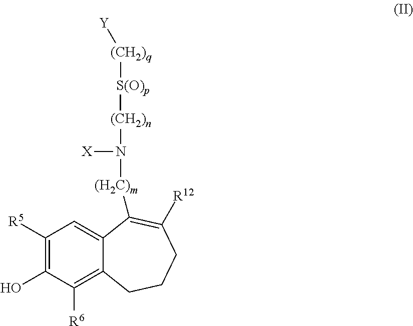
in which
R¹² stands for 3,5-difluorophenyl-, 3,4-difluorophenyl, 2,4-difluorophenyl-, 4-fluorophenyl
R⁵ and R⁶ independently of one another stand for hydrogen or fluorine, where R⁵ and R⁶ do not mean fluorine simultaneously,
X stands for C₁-C₄-alkyl-, optionally substituted with deuterium,
Y stands for —CF₃, —C₂F₅, 4,4-difluorocyclohexyl,
m stands for 6,
n stands for 3 or 4,
P stands for 1 or 2,
q stands for 2, 3, 4 or 5
or in the special case in which Y stands for 4,4-difluorocyclohexyl,
q stands for 0 or 1
and their salts, solvates or solvates of the salts, including all crystal modifications.

6. A Compound according to claim 1 that is
8-(3,5-Difluorophenyl)-9-[6-(methyl{3-[(4,4,5,5,5-pentafluoropentyl)sulphonyl]propyl}amino)hexyl]-6,7-dihydro-5H-benzo[7]annulen-3-ol;
8-(3,5-Difluorophenyl)-9-[6-(methyl{3-[(3,3,4,4,4-pentafluorobutyl)sulphinyl]propyl}amino)hexyl]-6,7-dihydro-5H-benzo[7]annulen-3-ol;
8-(3,5-Difluorophenyl)-9-[6-(methyl{3-[(3,3,4,4,4-pentafluorobutyl)sulphonyl]propyl}amino)hexyl]-6,7-dihydro-5H-benzo[7]annulen-3-ol;
8-(3,5-Difluorophenyl)-9-[6-(methyl{4-[(4,4,5,5,5-pentafluoropentyl)sulphinyl]butyl}amino)hexyl]-6,7-dihydro-5H-benzo[7]annulen-3-ol;
8-(3,5-Difluorophenyl)-9-[6-(methyl{3-[(3,3,3-trifluoropropyl)sulphinyl]propyl}amino)hexyl]-6,7-dihydro-5H-benzo[7]annulen-3-ol;
8-(3,5-Difluorophenyl)-9-[6-(methyl{3-[(3,3,3-trifluoropropyl)sulphonyl]propyl}amino)hexyl]-6,7-dihydro-5H-benzo[7]annulen-3-ol;
8-(3,5-Difluorophenyl)-9-[6-(methyl{3-[(4,4,4-trifluorobutyl)sulphonyl]propyl}amino)hexyl]-6,7-dihydro-5H-benzo[7]annulen-3-ol;
8-(3,5-Difluorophenyl)-9-[6-(methyl{3-[(4,4,5,5,5-pentafluoropentyl)sulphinyl]propyl}amino)hexyl]-6,7-dihydro-5H-benzo[7]annulen-3-ol;
8-(3,5-Difluorophenyl)-9-[6-(methyl{4-[(4,4,5,5,5-pentafluoropentyl)sulphonyl]butyl}amino)hexyl]-6,7-dihydro-5H-benzo[7]annulen-3-ol;
8-(3,5-Difluorophenyl)-9-{6-[(2-hydroxy-2-methylpropyl) {3-[(3,3,3-trifluoropropyl)sulphinyl]propyl}amino]hexyl}-6,7-dihydro-5H-benzo[7]annulen-3-ol;
8-(3,5-Difluorophenyl)-9-{6-[(2-hydroxy-2-methylpropyl) {3-[(3,3,3-trifluoropropyl)sulphonyl]propyl}amino]hexyl}-6,7-dihydro-5H-benzo[7]annulen-3-ol;
8-(3,5-Difluorophenyl)-9-{6-[(2-hydroxy-2-methylpropyl) {3-[(4,4,5,5,5-pentafluoropentyl)sulphinyl]propyl}amino]hexyl}-6,7-dihydro-5H-benzo[7]annulen-3-ol;
8-(3,5-Difluorophenyl)-9-{6-[(2-hydroxy-2-methylpropyl) {3-[(4,4,5,5,5-pentafluoropentyl)sulphonyl]propyl}amino]hexyl}-6,7-dihydro-5H-benzo[7]annulen-3-ol;
8-(3,5-Difluorophenyl)-9-[6-(ethyl {3-[(3,3,3-trifluoropropyl)sulphonyl]propyl}amino)hexyl]-6,7-dihydro-5H-benzo[7]annulen-3-ol;
8-(3,5-Difluorophenyl)-9-{6-[(2-methoxyethyl) {3-[(4,4,5,5,5-pentafluoropentyl)sulphonyl]propyl}amino]hexyl}-6,7-dihydro-5H-benzo[7]annulen-3-ol;
8-(3,5-Difluorophenyl)-9-{6-[(3-methoxypropyl) {3-[(4,4,5,5,5-pentafluoropentyl)sulphonyl]propyl}amino]hexyl}-6,7-dihydro-5H-benzo[7]annulen-3-ol;
8-(3,4-Difluorophenyl)-9-[6-(methyl{3-[(4,4,5,5,5-pentafluoropentyl)sulphonyl]propyl}amino)hexyl]-6,7-dihydro-5H-benzo[7]annulen-3-ol;
8-(3,4-Difluorophenyl)-9-[6-(methyl{3-[(3,3,4,4,4-pentafluorobutyl)sulphinyl]propyl}amino)hexyl]-6,7-dihydro-5H-benzo[7]annulen-3-ol;
8-(3,4-Difluorophenyl)-9-[6-(methyl{3-[(3,3,4,4,4-pentafluorobutyl)sulphonyl]propyl}amino)hexyl]-6,7-dihydro-5H-benzo[7]annulen-3-ol;
8-(3,4-Difluorophenyl)-9-[6-(methyl{4-[(4,4,5,5,5-pentafluoropentyl)sulphinyl]butyl}amino)hexyl]-6,7-dihydro-5H-benzo[7]annulen-3-ol;
8-(3,4-Difluorophenyl)-9-[6-(methyl{3-[(3,3,3-trifluoropropyl)sulphinyl]propyl}amino)hexyl]-6,7-dihydro-5H-benzo[7]annulen-3-ol;
8-(3,4-Difluorophenyl)-9-[6-(methyl{3-[(3,3,3-trifluoropropyl)sulphonyl]propyl}amino)hexyl]-6,7-dihydro-5H-benzo[7]annulen-3-ol;
8-(3,4-Difluorophenyl)-9-[6-(methyl{3-[(4,4,4-trifluorobutyl)sulphonyl]propyl}amino)hexyl]-6,7-dihydro-5H-benzo[7]annulen-3-ol;
8-(3,4-Difluorophenyl)-9-[6-(methyl{3-[(4,4,5,5,5-pentafluoropentyl)sulphinyl]propyl}amino)hexyl]-6,7-dihydro-5H-benzo[7]annulen-3-ol;
8-(3,4-Difluorophenyl)-9-[6-(methyl{4-[(4,4,5,5,5-pentafluoropentyl)sulphonyl]butyl}amino)hexyl]-6,7-dihydro-5H-benzo[7]annulen-3-ol;
8-(3,4-Difluorophenyl)-9-{6-[(2-hydroxy-2-methylpropyl) {3-[(3,3,3-trifluoropropyl)sulphinyl]propyl}amino]hexyl}-6,7-dihydro-5H-benzo[7]annulen-3-ol;
8-(3,4-Difluorophenyl)-9-{6-[(2-hydroxy-2-methylpropyl) {3-[(3,3,3-trifluoropropyl)sulphonyl]propyl}amino]hexyl}-6,7-dihydro-5H-benzo[7]annulen-3-ol;
8-(3,4-Difluorophenyl)-9-{6-[(2-hydroxy-2-methylpropyl) {3-[(4,4,5,5,5-pentafluoropentyl)sulphinyl]propyl}amino]hexyl}-6,7-dihydro-5H-benzo[7]annulen-3-ol;
8-(3,4-Difluorophenyl)-9-{6-[(2-hydroxy-2-methylpropyl) {3-[(4,4,5,5,5-pentafluoropentyl)sulphonyl]propyl}amino]hexyl}-6,7-dihydro-5H-benzo[7]annulen-3-ol;
8-(3,4-Difluorophenyl)-9-[6-(ethyl {3-[(3,3,3-trifluoropropyl)sulphonyl]propyl}amino)hexyl]-6,7-dihydro-5H-benzo[7]annulen-3-ol;
8-(3,4-Difluorophenyl)-9-{6-[(2-methoxyethyl) {3-[(4,4,5,5,5-pentafluoropentyl)sulphonyl]propyl}amino]hexyl}-6,7-dihydro-5H-benzo[7]annulen-3-ol;
8-(3,4-Difluorophenyl)-9-{6-[(3-methoxypropyl) {3-[(4,4,5,5,5-pentafluoropentyl)sulphonyl]propyl}amino]hexyl}-6,7-dihydro-5H-benzo[7]annulen-3-ol;
4-Fluoro-8-(4-fluorophenyl)-9-[6-(methyl{3-[(4,4,5,5,5-pentafluoropentyl)sulphonyl]propyl}amino)hexyl]-6,7-dihydro-5H-benzo[7]annulen-3-ol;
4-Fluoro-8-(4-fluorophenyl)-9-[6-(methyl{3-[(3,3,4,4,4-pentafluorobutyl)sulphonyl]propyl}amino)hexyl]-6,7-dihydro-5H-benzo[7]annulen-3-ol;
4-Fluoro-8-(4-fluorophenyl)-9-[6-(methyl{4-[(4,4,5,5,5-pentafluoropentyl)sulphinyl]butyl}amino)hexyl]-6,7-dihydro-5H-benzo[7]annulen-3-ol;
4-Fluoro-8-(4-fluorophenyl)-9-[6-(methyl{3-[(3,3,3-trifluoropropyl)sulphinyl]propyl}amino)hexyl]-6,7-dihydro-5H-benzo[7]annulen-3-ol;
4-Fluoro-8-(4-fluorophenyl)-9-[6-(methyl{3-[(3,3,3-trifluoropropyl)sulphonyl]propyl}amino)hexyl]-6,7-dihydro-5H-benzo[7]annulen-3-ol;
4-Fluoro-8-(4-fluorophenyl)-9-[6-(methyl{3-[(4,4,4-trifluorobutyl)sulphonyl]propyl}amino)hexyl]-6,7-dihydro-5H-benzo[7]annulen-3-ol;
4-Fluoro-8-(4-fluorophenyl)-9-[6-(methyl{4-[(4,4,4-trifluorobutyl)sulphonyl]butyl}amino)hexyl]-6,7-dihydro-5H-benzo[7]annulen-3-ol;
4-Fluoro-8-(4-fluorophenyl)-9-{6-[(2-methoxyethyl) {3-[(4,4,5,5,5-pentafluoropentyl)sulphonyl]propyl}amino]hexyl}-6,7-dihydro-5H-benzo[7]annulen-3-ol;
4-Fluoro-8-(4-fluorophenyl)-9-{6-[(3-methoxypropyl) {3-[(4,4,5,5,5-pentafluoropentyl)sulphonyl]propyl}amino]hexyl}-6,7-dihydro-5H-benzo[7]annulen-3-ol;
4-Fluoro-8-(4-fluorophenyl)-9-[6-(methyl{3-[(4,4,5,5,5-pentafluoropentyl)sulphinyl]propyl}amino)hexyl]-6,7-dihydro-5H-benzo[7]annulen-3-ol;
4-Fluoro-8-(4-fluorophenyl)-9-[6-(methyl{4-[(4,4,5,5,5-pentafluoropentyl)sulphonyl]butyl}amino)hexyl]-6,7-dihydro-5H-benzo[7]annulen-3-ol;
8-(3,5-Difluorophenyl)-9-[6-(methyl{4-[(4,4,4-trifluorobutyl)sulphonyl]butyl}amino)hexyl]-6,7-dihydro-5H-benzo[7]annulen-3-ol;
8-(3,4-Difluorophenyl)-9-[6-(methyl{4-[(4,4,4-trifluorobutyl)sulphonyl]butyl}amino)hexyl]-6,7-dihydro-5H-benzo[7]annulen-3-ol;
8-(3,5-Difluorophenyl)-9-[6-(methyl{4-[(3,3,3-trifluoropropyl)sulphonyl]butyl}amino)hexyl]-6,7-dihydro-5H-benzo[7]annulen-3-ol;
8-(3,4-Difluorophenyl)-9-[6-(methyl{4-[(3,3,3-trifluoropropyl)sulphonyl]butyl}amino)hexyl]-6,7-dihydro-5H-benzo[7]annulen-3-ol;
8-(4-Fluorophenyl)-9-[6-(methyl{4-[(4,4,4-trifluorobutyl)sulphonyl]butyl}amino)hexyl]-6,7-dihydro-5H-benzo[7]annulen-3-ol;
8-(4-Fluorophenyl)-9-[5-(methyl{4-[(4,4,4-trifluorobutyl)sulphonyl]butyl}amino)pentyl]-6,7-dihydro-5H-benzo[7]annulen-3-ol;
4-Fluoro-8-(4-Fluorophenyl)-9-[6-(methyl{4-[(3,3,3-trifluoropropyl)sulphonyl]butyl}amino)hexyl]-6,7-dihydro-5H-benzo[7]annulen-3-ol;
8-(4-Fluorophenyl)-9-[6-(methyl{4-[(3,3,3-trifluoropropyl)sulphonyl]butyl}amino)hexyl]-6,7-dihydro-5H-benzo[7]annulen-3-ol;
8-(4-Fluorophenyl)-9-[5-(methyl{4-[(3,3,3-trifluoropropyl)sulphonyl]butyl}amino)pentyl]-6,7-dihydro-5H-benzo[7]annulen-3-ol;
8-(4-Fluorophenyl)-9-[6-(methyl{3-[(3,3,3-trifluoropropyl)sulphonyl]propyl}amino)hexyl]-6,7-dihydro-5H-benzo[7]annulen-3-ol;
8-(4-Fluorophenyl)-9-{6-[(2-hydroxyethyl) {3-[(4,4,5,5,5-pentafluoropentyl)sulphonyl]propyl}amino]hexyl}-6,7-dihydro-5H-benzo[7]annulen-3-ol;
8-(4-Fluorophenyl)-9-{6-[(3-hydroxypropyl) {3-[(4,4,5,5,5-pentafluoropentyl)sulphonyl]propyl}amino]hexyl}-6,7-dihydro-5H-benzo[7]annulen-3-ol;
8-(4-Fluorophenyl)-9-{6-[(2-hydroxyethyl) {3-[(3,3,3-trifluoropropyl)sulphonyl]propyl}amino]hexyl}-6,7-dihydro-5H-benzo[7]annulen-3-ol;
8-(4-Fluorophenyl)-9-{6-[(3-hydroxypropyl) {3-[(3,3,3-trifluoropropyl)sulphonyl]propyl}amino]hexyl}-6,7-dihydro-5H-benzo[7]annulen-3-ol;
9-{6-[(4-Fluorobenzyl) {3-[(4,4,5,5,5-pentafluoropentyl)sulphonyl]propyl}amino]hexyl}-8-(4-fluorophenyl)-6,7-dihydro-5H-benzo[7]annulen-3-ol;
4-Fluoro-8-(4-fluorophenyl)-9-{6-[(2-hydroxyethyl) {3-[(4,4,5,5,5-pentafluoropentyl)sulphonyl]propyl}amino]hexyl}-6,7-dihydro-5H-benzo[7]annulen-3-ol;
4-Fluoro-8-(4-fluorophenyl)-9-{6-[(3-hydroxypropyl) {3-[(4,4,5,5,5-pentafluoropentyl)sulphonyl]propyl}amino]hexyl}-6,7-dihydro-5H-benzo[7]annulen-3-ol;
4-Fluoro-8-(4-fluorophenyl)-9-{6-[(2-hydroxyethyl) {3-[(3,3,3-trifluoropropyl)sulphonyl]propyl}amino]hexyl}-6,7-dihydro-5H-benzo[7]annulen-3-ol;
4-Fluoro-8-(4-fluorophenyl)-9-{6-[(3-hydroxypropyl) {3-[(3,3,3-trifluoropropyl)sulphonyl]propyl}amino]hexyl}-6,7-dihydro-5H-benzo[7]annulen-3-ol;
9-[6-(tert-Butyl {3-[(4,4,5,5,5-pentafluoropentyl)sulphonyl]propyl}amino)hexyl]-4-fluoro-8-(4-fluorophenyl)-6,7-dihydro-5H-benzo[7]annulen-3-ol;
9-{6-[(2,2-Difluoroethyl) {3-[(4,4,5,5,5-pentafluoropentyl)sulphonyl]propyl}amino]hexyl}-4-fluoro-8-(4-fluorophenyl)-6,7-dihydro-5H-benzo[7]annulen-3-ol;
4-Fluoro-9-{6-[(4-fluorobenzyl) {3-[(4,4,5,5,5-pentafluoropentyl)sulphonyl]propyl}amino]hexyl}-8-(4-fluorophenyl)-6,7-dihydro-5H-benzo[7]annulen-3-ol;
9-[6-(Cyclopropyl {3-[(4,4,5,5,5-pentafluoropentyl)sulphonyl]propyl}amino)hexyl]-8-(3,4-difluorophenyl)-6,7-dihydro-5H-benzo[7]annulen-3-ol;
8-(3,5-Difluorophenyl)-9-[6-({4-[(4,4,4-trifluorobutyl)sulphonyl]butyl}amino)hexyl]-6,7-dihydro-5H-benzo[7]annulen-3-ol;
8-(3,5-Difluorophenyl)-9-[6-(methyl{5-[(3,3,3-trifluoropropyl)sulphonyl]pentyl}amino)hexyl]-6,7-dihydro-5H-benzo[7]annulen-3-ol;
8-(3,5-Difluorophenyl)-9-[6-(methyl{4-[(3,3,4,4,4-pentafluorobutyl)sulphonyl]butyl}amino)hexyl]-6,7-dihydro-5H-benzo[7]annulen-3-ol;
8-(3,5-Difluorophenyl)-9-[6-(methyl{3-[(5,5,5-trifluoropentyl)sulphonyl]propyl}amino)hexyl]-6,7-dihydro-5H-benzo[7]annulen-3-ol;
8-(3,4-Difluorophenyl)-9-[6-(methyl{5-[(3,3,3-trifluoropropyl)sulphonyl]pentyl}amino)hexyl]-6,7-dihydro-5H-benzo[7]annulen-3-ol;
8-(3,4-Difluorophenyl)-9-[6-(methyl{4-[(3,3,4,4,4-pentafluorobutyl)sulphonyl]butyl}amino)hexyl]-6,7-dihydro-5H-benzo[7]annulen-3-ol;
8-(3,4-Difluorophenyl)-9-[6-(methyl{3-[(5,5,5-trifluoropentyl)sulphonyl]propyl}amino)hexyl]-6,7-dihydro-5H-benzo[7]annulen-3-ol;
9-{6-[(2-Fluoroethyl) {3-[(4,4,5,5,5-pentafluoropentyl)sulphonyl]propyl}amino]hexyl}-8-(4-fluorophenyl)-6,7-dihydro-5H-benzo[7]annulen-3-ol;
8-(4-Fluorophenyl)-9-[6-(methyl{4-[(3,3,4,4,4-pentafluorobutyl)sulphonyl]butyl}amino)hexyl]-6,7-dihydro-5H-benzo[7]annulen-3-ol;
8-(4-Fluorophenyl)-9-[6-(methyl{5-[(3,3,3-trifluoropropyl)sulphonyl]pentyl}amino)hexyl]-6,7-dihydro-5H-benzo[7]annulen-3-ol;
8-(4-Fluorophenyl)-9-[6-({4-[(4,4,4-trifluorobutyl)sulphonyl]butyl}amino)hexyl]-6,7-dihydro-5H-benzo[7]annulen-3-ol;
8-(4-Fluorophenyl)-9-[6-(methyl{3-[(4,4,5,5,5-pentafluoropentyl)sulphanyl]propyl}amino)hexyl]-6,7-dihydro-5H-benzo[7]annulen-3-ol;
8-(4-Fluorophenyl)-9-[6-(methyl{3-[(5,5,6,6,6-pentafluorohexyl)sulphonyl]propyl}amino)hexyl]-6,7-dihydro-5H-benzo[7]annulen-3-ol;
8-(4-Fluorophenyl)-9-[6-(methyl{3-[(5,5,5-trifluoropentyl)sulphonyl]propyl}amino)hexyl]-6,7-dihydro-5H-benzo[7]annulen-3-ol;
Benzyl-N-{6-[8-(4-fluorophenyl)-3-hydroxy-6,7-dihydro-5H-benzo[7]annulen-9-yl]hexyl}-N-{4-[(4,4,4-trifluorobutyl)sulphonyl]butyl}glycinate
Methyl-N-{6-[8-(4-fluorophenyl)-3-hydroxy-6,7-dihydro-5H-benzo[7]annulen-9-yl]hexyl}-N-{4[(4,4,4-trifluorobutyl)sulphonyl]butyl}glycinate
Methyl-N-{6-[8-(4-fluorophenyl)-3-hydroxy-6,7-dihydro-5H-benzo[7]annulen-9-yl]hexyl}-N-{4[(4,4,4-trifluorobutyl)sulphonyl]butyl}-beta-alaninate
4-Fluoro-8-(4-fluorophenyl)-9-{6-[{3-[(4,4,5,5,5-pentafluoropentyl)sulphonyl]propyl}(2,2,2-trifluoroethyl)amino]hexyl}-6,7-dihydro-5H-benzo[7]annulen-3-ol;
4-Fluoro-9-{6-[(2-fluoroethyl) {3-[(4,4,5,5,5-pentafluoropentyl)sulphonyl]propyl}amino]hexyl}-8-(4-fluorophenyl)-6,7-dihydro-5H-benzo[7]annulen-3-ol;
4-Fluoro-8-(4-fluorophenyl)-9-[6-(methyl{3-[(4,4,5,5,5-pentafluoropentyl)sulphanyl]propyl}amino)hexyl]-6,7-dihydro-5H-benzo[7]annulen-3-ol;
4-Fluoro-8-(4-fluorophenyl)-9-[6-(methyl{3-[(5,5,5-trifluoropentyl)sulphonyl]propyl}amino)hexyl]-6,7-dihydro-5H-benzo[7]annulen-3-ol;
Methyl-4-({6-[8-(4-fluorophenyl)-3-hydroxy-6,7-dihydro-5H-benzo[7]annulen-9-yl]hexyl}{4-[(4,4,4-trifluorobutyl)sulphonyl]butyl}amino)butanoate
N-{6-[8-(4-Fluorophenyl)-3-hydroxy-6,7-dihydro-5H-benzo[7]annulen-9-yl]hexyl}-N-{4-[(4,4,4-trifluorobutyl)sulphonyl]butyl}acetamide
({6-[8-(4-Fluorophenyl)-3-hydroxy-6,7-dihydro-5H-benzo[7]annulen-9-yl]hexyl}{4-[(4,4,4-trifluorobutyl)sulphonyl]butyl}amino)acetonitrile
N-{6-[8-(4-Fluorophenyl)-3-hydroxy-6,7-dihydro-5H-benzo[7]annulen-9-yl]hexyl}-N-{4[(4,4,4-trifluorobutyl)sulphonyl]butyl}methanesulphonamide
8-(4-Fluorophenyl)-9-{6-[(2-hydroxyethyl) {4-[(4,4,4-trifluorobutyl)sulphonyl]butyl}amino]hexyl}-6,7-dihydro-5H-benzo[7]annulen-3-ol;
8-(4-Fluorophenyl)-9-[6-([(2S)-2-hydroxypropyl]{4-[(4,4,4-trifluorobutyl)sulphonyl]butyl}amino)hexyl]-6,7-dihydro-5H-benzo[7]annulen-3-ol;
N-{6-[8-(4-Fluorophenyl)-3-hydroxy-6,7-dihydro-5H-benzo[7]annulen-9-yl]hexyl}-N-{4-[(4,4,4-trifluorobutyl)sulphonyl]butyl}glycine
N-{6-[8-(4-Fluorophenyl)-3-hydroxy-6,7-dihydro-5H-benzo[7]annulen-9-yl]hexyl}-N-{4[(4,4,4-trifluorobutyl)sulphonyl]butyl}-beta-alanine
4-({6-[8-(4-Fluorophenyl)-3-hydroxy-6,7-dihydro-5H-benzo[7]annulen-9-yl]hexyl}{4-[(4,4,4-trifluorobutyl)sulphonyl]butyl}amino)butanoic acid
8-(4-Fluorophenyl)-9-{6-[(2-hydroxyethyl) {3-[(4,4,4-trifluorobutyl)sulphonyl]propyl}amino]hexyl}-6,7-dihydro-5H-benzo[7]annulen-3-ol;
8-(4-Fluorophenyl)-9-[6-([(2R)-2-hydroxypropyl]{4-[(4,4,4-trifluorobutyl)sulphonyl]butyl}amino)hexyl]-6,7-dihydro-5H-benzo[7]annulen-3-ol;
2-Fluoro-8-(4-fluorophenyl)-9-[6-(methyl{3-[(4,4,5,5,5-pentafluoropentyl)sulphonyl]propyl}amino)hexyl]-6,7-dihydro-5H-benzo[7]annulen-3-ol;
2-Fluoro-8-(4-fluorophenyl)-9-{6-[(2-hydroxyethyl) {3-[(4,4,5,5,5-pentafluoropentyl)sulphonyl]propyl}amino]hexyl}-6,7-dihydro-5H-benzo[7]annulen-3-ol;
2-Fluoro-8-(4-fluorophenyl)-9-[6-(methyl{4-[(4,4,4-trifluorobutyl)sulphonyl]butyl}amino)hexyl]-6,7-dihydro-5H-benzo[7]annulen-3-ol;
2-Fluoro-8-(4-fluorophenyl)-9-[6-(methyl{3-[(5,5,5-trifluoropentyl)sulphonyl]propyl}amino)hexyl]-6,7-dihydro-5H-benzo[7]annulen-3-ol;
2-Fluoro-8-(4-fluorophenyl)-9-{6-[(2-hydroxyethyl) {3-[(4,4,4-trifluorobutyl)sulphonyl]propyl}amino]hexyl}-6,7-dihydro-5H-benzo[7]annulen-3-ol;
2-Fluoro-8-(4-fluorophenyl)-9-{6-[(2-hydroxyethyl) {4-[(4,4,4-trifluorobutyl)sulphonyl]butyl}amino]hexyl}-6,7-dihydro-5H-benzo[7]annulen-3-ol;
2-Fluoro-8-(4-fluorophenyl)-9-[6-(methyl{4-[(4,4,5,5,5-pentafluoropentyl)sulphonyl]butyl}amino)hexyl]-6,7-dihydro-5H-benzo[7]annulen-3-ol;
2-Fluoro-8-(4-fluorophenyl)-9-[6-(methyl{4-[(3,3,3-trifluoropropyl)sulphonyl]butyl}amino)hexyl]-6,7-dihydro-5H-benzo[7]annulen-3-ol;
2-Fluoro-8-(4-fluorophenyl)-9-[6-(methyl{5-[(3,3,3-trifluoropropyl)sulphonyl]pentyl}amino)hexyl]-6,7-dihydro-5H-benzo[7]annulen-3-ol;
8-(4-Fluorophenyl)-9-{6-[(2-hydroxyethyl)(3-{[3,4,4,4-tetrafluoro-3-(trifluoromethyl)butyl]sulphonyl}propyl)amino]hexyl}-6,7-dihydro-5H-benzo[7]annulen-3-ol;
8-(4-Fluorophenyl)-9-{6-[(2-hydroxyethyl)(4-{[3,4,4,4-tetrafluoro-3-(trifluoromethyl)butyl]sulphonyl}butyl)amino]hexyl}-6,7-dihydro-5H-benzo[7]annulen-3-ol;
8-(4-Fluorophenyl)-9-{6-[methyl(3-{[3,4,4,4-tetrafluoro-3-(trifluoromethyl)butyl]sulphonyl}propyl)amino]hexyl}-6,7-dihydro-5H-benzo[7]annulen-3-ol;
8-(4-Fluorophenyl)-9-{6-[methyl(4-{[3,4,4,4-tetrafluoro-3-(trifluoromethyl)butyl]sulphonyl}butyl)amino]hexyl}-6,7-dihydro-5H-benzo[7]annulen-3-ol;
8-(4-Fluorophenyl)-9-[6-({3-[(5,5,5-trifluoropentyl)sulphonyl]propyl}amino)hexyl]-6,7-dihydro-5H-benzo[7]annulen-3-ol;
8-(4-Fluorophenyl)-9-[6-({3-[(4,4,5,5,5-pentafluoropentyl)sulphonyl]propyl}amino)hexyl]-6,7-dihydro-5H-benzo[7]annulen-3-ol;
8-(4-Fluorophenyl)-9-[6-(methyl{4-[(4,4,5,5,5-pentafluoropentyl)sulphonyl]butyl}amino)hexyl]-6,7-dihydro-5H-benzo[7]annulen-3-ol;
8-(4-Fluorophenyl)-9-[6-(methyl{3-[(4,4,5,5,5-pentafluoropentyl)sulphonyl]propyl}amino)hexyl]-6,7-dihydro-5H-benzo[7]annulen-3-ol;
8-(4-Fluorophenyl)-9-[6-(methyl{3-[(3,3,4,4,4-pentafluorobutyl)sulphonyl]propyl}amino)hexyl]-6,7-dihydro-5H-benzo[7]annulen-3-ol;
8-(4-Fluorophenyl)-9-[6-(methyl{3-[(4,4,4-trifluorobutyl)sulphonyl]propyl}amino)hexyl]-6,7-dihydro-5H-benzo[7]annulen-3-ol;
8-(4-Fluorophenyl)-9-[6-(methyl{3-[(4,4,5,5,5-pentafluoropentyl)sulphinyl]propyl}amino)hexyl]-6,7-dihydro-5H-benzo[7]annulen-3-ol;
8-(4-Fluorophenyl)-9-{6-[(2-hydroxy-2-methylpropyl) {3-[(3,3,3-trifluoropropyl)sulphonyl]propyl}amino]hexyl}-6,7-dihydro-5H-benzo[7]annulen-3-ol;
8-(4-Fluorophenyl)-9-{6-[(2-hydroxy-2-methylpropyl) {3-[(3,3,3-trifluoropropyl)sulphinyl]propyl}amino]hexyl}-6,7-dihydro-5H-benzo[7]annulen-3-ol;
9-{6-[{3-[(4,4-Difluorocyclohexyl)sulphonyl]propyl}(methyl)amino]hexyl}-8-(4-fluorophenyl)-6,7-dihydro-5H-benzo[7]annulen-3-ol;
9-{6-[{4-[(4,4-Difluorocyclohexyl)sulphonyl]butyl}(methyl)amino]hexyl}-8-(4-fluorophenyl)-6,7-dihydro-5H-benzo[7]annulen-3-ol;
9-{6-[(3-{[(4,4-Difluorocyclohexyl)methyl]sulphonyl}propyl)(methyl)amino]hexyl}-8-(4-fluorophenyl)-6,7-dihydro-5H-benzo[7]annulen-3-ol;
8-(3-Fluorophenyl)-9-[6-(methyl{4-[(4,4,5,5,5-pentafluoropentyl)sulphonyl]butyl}amino)hexyl]-6,7-dihydro-5H-benzo[7]annulen-3-ol;
8-(3-Fluorophenyl)-9-[6-(methyl{3-[(4,4,5,5,5-pentafluoropentyl)sulphonyl]propyl}amino)hexyl]-6,7-dihydro-5H-benzo[7]annulen-3-ol;
8-(2-Fluorophenyl)-9-[6-(methyl{3-[(4,4,5,5,5-pentafluoropentyl)sulphonyl]propyl}amino)hexyl]-6,7-dihydro-5H-benzo[7]annulen-3-ol;
8-(4-Fluorophenyl)-9-[5-(methyl{3-[(4,4,5,5,5-pentafluoropentyl)sulphonyl]propyl}amino)pentyl]-6,7-dihydro-5H-benzo[7]annulen-3-ol;
8-(2,4-Difluorophenyl)-9-[6-(methyl{3-[(4,4,5,5,5-pentafluoropentyl)sulphonyl]propyl}amino)hexyl]-6,7-dihydro-5H-benzo[7]annulen-3-ol;
8-(2,4-Difluorophenyl)-9-[6-(methyl{3-[(5,5,5-trifluoropentyl)sulphonyl]propyl}amino)hexyl]-6,7-dihydro-5H-benzo[7]annulen-3-ol;
8-(2,4-Difluorophenyl)-9-[6-(methyl{3-[(3,3,3-trifluoropropyl)sulphonyl]propyl}amino)hexyl]-6,7-dihydro-5H-benzo[7]annulen-3-ol;
8-(2,4-Difluorophenyl)-9-[6-(methyl{4-[(3,3,3-trifluoropropyl)sulphonyl]butyl}amino)hexyl]-6,7-dihydro-5H-benzo[7]annulen-3-ol;
8-(2,4-Difluorophenyl)-9-[6-(methyl{4-[(4,4,4-trifluorobutyl)sulphonyl]butyl}amino)hexyl]-6,7-dihydro-5H-benzo[7]annulen-3-ol;
8-(2,4-Difluorophenyl)-9-[6-(methyl{5-[(3,3,3-trifluoropropyl)sulphonyl]pentyl}amino)hexyl]-6,7-dihydro-5H-benzo[7]annulen-3-ol;
8-(2,4-Difluorophenyl)-9-[6-(methyl{3-[(4,4,4-trifluorobutyl)sulphonyl]propyl}amino)hexyl]-6,7-dihydro-5H-benzo[7]annulen-3-ol;
8-(2,4-Difluorophenyl)-9-[6-(methyl{3-[(6,6,6-trifluorohexyl)sulphonyl]propyl}amino)hexyl]-6,7-dihydro-5H-benzo[7]annulen-3-ol;
8-(2,4-Difluorophenyl)-9-{6-[(²H₃)methyl{3-[(5,5,5-trifluoropentyl)sulphonyl]propyl}amino]hexyl}-6,7-dihydro-5H-benzo[7]annulen-3-ol;
8-(2,5-Difluorophenyl)-9-{6-[(²H₃)methyl{3-[(5,5,5-trifluoropentyl)sulphonyl]propyl}amino]hexyl}-6,7-dihydro-5H-benzo[7]annulen-3-ol;
2-Fluoro-8-(4-fluorophenyl)-9-{6-[(2-hydroxyethyl)(4-{[3,4,4,4-tetrafluoro-3-(trifluoromethyl)butyl]sulphonyl}butyl)amino]hexyl}-6,7-dihydro-5H-benzo[7]annulen-3-ol;
8-(4-Fluorophenyl)-9-{6-[(²H₃)methyl{3-[(5,5,5-trifluoropentyl)sulphonyl]propyl}amino]hexyl}-6,7-dihydro-5H-benzo[7]annulen-3-ol;
2-Fluoro-8-(4-fluorophenyl)-9-[6-(methyl{3-[(6,6,6-trifluorohexyl)sulphonyl]propyl}amino)hexyl]-6,7-dihydro-5H-benzo[7]annulen-3-ol;
({6-[8-(4-Fluorophenyl)-3-hydroxy-6,7-dihydro-5H-benzo[7]annulen-9-yl]hexyl}{3-[(5,5,5-trifluoropentyl)sulphonyl]propyl}amino)acetonitrile
2-Fluoro-8-(4-fluorophenyl)-9-{6-[(2-hydroxyethyl)(3-{[3,4,4,4-tetrafluoro-3-(trifluoromethyl)butyl]sulphonyl}propyl)amino]hexyl}-6,7-dihydro-5H-benzo[7]annulen-3-ol;
8-(2,5-Difluorophenyl)-9-[6-(methyl{4-[(4,4,4-trifluorobutyl)sulphonyl]butyl}amino)hexyl]-6,7-dihydro-5H-benzo[7]annulen-3-ol;
9-{6-[{4-[(4,4-Difluorocyclohexyl)sulphonyl]butyl}(methyl)amino]hexyl}-2-Fluoro-8-(4-fluorophenyl)-6,7-dihydro-5H-benzo[7]annulen-3-ol;
8-(2,4-Difluorophenyl)-9-[6-(methyl{3-[(3,3,4,4,4-pentafluorobutyl)sulphonyl]propyl}amino)hexyl]-6,7-dihydro-5H-benzo[7]annulen-3-ol;
8-(2,4-Difluorophenyl)-9-[6-(methyl{4-[(3,3,4,4,4-pentafluorobutyl)sulphonyl]butyl}amino)hexyl]-6,7-dihydro-5H-benzo[7]annulen-3-ol;
8-(2,4-Difluorophenyl)-9-[6-(methyl{4-[(4,4,5,5,5-pentafluoropentyl)sulphonyl]butyl}amino)hexyl]-6,7-dihydro-5H-benzo[7]annulen-3-ol;
8-(4-Fluorophenyl)-9-[6-(methyl{3-[(6,6,6-trifluorohexyl)sulphonyl]propyl}amino)hexyl]-6,7-dihydro-5H-benzo[7]annulen-3-ol; or
4-Fluoro-8-(4-fluorophenyl)-9-[6-(methyl{3-[(4,4,4-trifluorobutyl)sulphonyl]propyl}amino)hexyl]-6,7-dihydro-5H-benzo[7]annulen-3-ol

7. Compound as defined in claim 1, for the treatment and/or prophylaxis of diseases.

8. Use of a compound as defined in claim 1, for the production of a medicinal product for the treatment and/or prophylaxis of diseases.

9. Compound of formula (I) as defined in claim 1, for use in a method of induction of ovulation, for inhibition of sperm maturation, for alleviating the symptoms of the andropause and menopause, i.e. for male and female hormone replacement therapy, for the prevention or prophylaxis and for the treatment of disorders accompanying dysmenorrhoea, of dysfunctional uterine bleeding, acne, cardiovascular diseases, hypercholesterolaemia and hyperlipidaemia, atherosclerosis, proliferation of arterial smooth muscle cells, respiratory distress syndrome of the newborn, primary pulmonary hypertension, osteoporosis, bone loss in postmenopausal women, in hysterectomized women or in women who have been treated with LHRH agonists or antagonists, rheumatoid arthritis, Alzheimer's disease, endometriosis, myomata, hormone-dependent tumours (also in premenopausal women), for example breast or endometrial carcinoma, infertility, prostatic diseases, benign diseases of the breast, e.g. mastopathy, stroke, Alzheimer's and other diseases of the central nervous system that are associated with cellular death of neurons.

10. Use of a compound as defined in claim 1, for the production of a medicinal product for induction of ovulation, for inhibition of sperm maturation, for alleviating the symptoms of the andropause and menopause, i.e. for male and female hormone replacement therapy, for the prevention or prophylaxis and for the treatment of disorders accompanying dysmenorrhoea, of dysfunctional uterine bleeding, acne, cardiovascular diseases, hypercholesterolaemia and hyperlipidaemia, atherosclerosis, proliferation of arterial smooth muscle cells, respiratory distress syndrome of the newborn, primary pulmonary hypertension, osteoporosis, bone loss in postmenopausal women, in hysterectomized women or in women who have been treated with LHRH agonists or antagonists, rheumatoid arthritis, Alzheimer's disease, endometriosis, myomata, hormone-dependent tumours (also in premenopausal women), for example breast or endometrial carcinoma, infertility, prostatic diseases, benign diseases of the breast, e.g. mastopathy, stroke, Alzheimer's and other diseases of the central nervous system that are associated with cellular death of neurons.

11. Medicinal product containing a compound as defined in claim 1, in combination with another active substance, in particular with LHRH analogues for the treatment of endometriosis.

12. Medicinal product containing a compound as defined in claim 1, in combination with an inert, non-toxic, pharmaceutically suitable excipient.

13. Medicinal product according to claim 1 for induction of ovulation, for inhibition of sperm maturation, for alleviating the symptoms of the andropause and menopause, i.e. for male and female hormone replacement therapy, for the prevention or prophylaxis and for the treatment of disorders accompanying dysmenorrhoea, of dysfunctional uterine bleeding, acne, cardiovascular diseases, hypercholesterolaemia and hyperlipidaemia, atherosclerosis, proliferation of arterial smooth muscle cells, respiratory distress syndrome of the newborn, primary pulmonary hypertension, osteoporosis, bone loss in postmenopausal women, in hysterectomized women or in women who have been treated with LHRH agonists or antagonists, rheumatoid arthritis, Alzheimer's disease, endometriosis, myomata, hormone-dependent tumours (also in premenopausal women), for example breast or endometrial carcinoma, infertility, prostatic diseases, benign diseases of the breast, e.g. mastopathy, stroke, Alzheimer's and other diseases of the central nervous system that are associated with cellular death of neurons.
